SEQUENCE LISTING (105 sequences)

SEQ ID NO: 1 - DNA, 1037 bp, Arabidopsis thaliana
aatcatggttttaatagtacgttagaatgtatttctttttaaaatatcaatttggagtttgattttaatttcaagttctgcgctacttattgttaccccacagactacgaataaattatgtaaatcttaagaagaatatatggggaataacaaataaacactaatgaataaaatagatttggtaaaattccacaataatagtaaactaatatctttgaccctagatccgcgatttcgcacacacgatcatgtgcatagttgcgaagtaccttaaccagtgacgccgagtcaataacacgtattaacagataaaagaaccatacaattaaacctagtaaaaataaacatttgttaaaggattcaagattctcaatgcgataagacttttctatcactatattttcacaaagtcaacactcaacatgaaatgttaatgttagatcagagtatatattaaaaatagtaaaatgtttaatcttatatggatcttaactcttgaattttttttctgagaatcttgacaaagagaagtacaagcaagtagatcattcgaagacatctttattttttcttaaccctaccaaacccaaaactcagtttccgattttcatatgttttgactatcctaatccatgtggcgtggaatggacctaaaatcacaccaaactaattcgtatgtatgctatagaaaagaactttaccatattaagtatattaatattaattcaatccaactaggatacactttttaggtcaaaatccaactagtatattacatattgaaaacaacccccaaaaaaactcgatgtagtaatgccgccgtatattagaggattgactttacaataaacatattaacatttcgttttaaaaaaagtaacgtgtgtgtgtttgtgtcggtgtgtacataatacttacgaaggaattattaaagtgtgcctaattaggaaattccaacttggtaagcatgcatgctttctataaaaagagacaacatgtggacacaataattcatttcccaagagaagtaaaaaaca

SEQ ID NO: 2 - DNA, 1500 bp, Arabidopsis thaliana
gaaacaaaacaaagatatttatactatggatcatgttacaaaacacaattactcctgattcgttattaaattatcactaattaagatacaagttgtgaaaaatagtcttaactgttttgaaaacaacgttgtaaaaaatcttcatgaaccataaaactaaagaaactctacttttatgactaaactatcattaagaatattatggtagtcagccacttcaaattttctcattttctgacatgagctaatcctactcctttcctgccgaattaatcgtaagaaaattggcacaaattatatggtttgatcctagttggtgatactgatctcaacctctgtttttttgtttatttcacctctgtgttgataatattaaagattagttggaaattatataattagagagactctgttagttattgtgctcaactgagttttgggatgaggacaaaaacgagacatcacctgctcatgtgtacagcagagatggctgtgctgtacatacaattatttccactaattaataactttttcctctaattttatacatgttcttgataattatcttctagttttatagtactatttagttagctttatgcaaaattagtagtgtatatgttctttaatgtcttctacaatttatatagataaaatcctccatgttcccctaaaatctaaattgtccacgtaccatgaaaacaaggcctattagtttatttttggataataattagctaacctattataactttccatgttcataactcataagctataaaagaggcaatacatataaattaaataaaaaccatgtctctgtctctttcttcttttgctcattaatcatttatgtgaaaatctcatcgtaatctaaagtcaagaggtattagtctgtactctgtttaataaaggaatttcaaactggagtaaccatatcattactatatatgtaagggatttctgtttcatccataatcatatcgtagcatacaatttaaacttaagattataaaatatatagttttactaatgccaaggattatacaaatgttcaataactatatataattccttaatctctattagtaacttttgttttttaaaacataaaaagtcagaagatggactaactgtaactaaaggatatttaaaattttcataaacgcattggttttgtgcatcccaatttttgtaagtgatattttcaatatggaaactataagattcaacggttgtacttatgcatgcttagcttttaaaaaataacataacgtaaaaatgaacgataaattaaatacaatagtcaactggtcactgttacgtttttttcattttcgttaaagaaaacaaatacatgggaaaatttagaaatagtataaatggaggcttaagctttccataatcttcacaactaaatcagaagattactactaaacagagtcttttttgacttgccaaaaacacatctgtctctctctctctctttgcggatctgaa

SEQ ID NO: 3 - DNA, 1380 bp, Arabidopsis thaliana
ataaattcactatatcaaataaataattaggccaaaatataggaaattaaataagaccatttaaatagatctaagacgggagatattaggattgattatcttcaaattatgttttcaatttcattagttaggatttaattacatataggaatagaaaaattaccatcaaatggggaagatgagcaatgttcatataggattataaacaagtaaaaaaaaaaaaaaaatttatttttgggtaagaaatatatgagaattttgtgcgcgttccaaaaactcttgtaaattttggcaagcttatgaatattacataaagtttggtatataactggaaacatatttatattactaaaactcgtttaatattaattgctaaagaaagaaaacaggtaacaaaagcgttaaagttaactttgattctatttattgatagatcccatcattagattatgaaatatcgtatccgattagataaattcattaccaacctttttttctttccttttggtaaactctttcattttaaacatattgaatgttaagtgggattgctaacataaaatagtaaaagtttttttactataggacaacgatccaattccaagctaaagattgatatataactagaaatggtatttaagagcttatgactaacactagagaccccatctaagcccataaaatgaggttatctaagcaagttatgtctcacgtaatgatcattattgggctttatgggtaatctatcccgttactccaaatctagttcagctgccacgtcactctaactaactcgttggaggggaaagagtgatattgaagcaaacaagaacaacgacgattatgggttttggggttttatccatttctttgtctaattttatcagattttgtctagatttttggatccaaattattagcggaaatcgaaattgggttttacggttaaatctcgttcttgaaagcttcccttccatggggctttttatctggacttaaaagcttgtgctagtctcacttgatactaatgtaaaggaaggagctttggaatgcttaattggcaagtgactagtggcacaactgtcagtttatatattttgattaaacaaatcaaacgtgggtctgtggatgggcaataaacaacaacataaattgaagaatatcatcaccccaaatggtcattggatagattagagaaatagaaaaatcaaaagaagctttggcaaagagctaaagatcaaagcttttctttgaggtaattcgtttgactctttttttttttttttttttgcttgaatcgtgttgttctctttgttctgctagagatttgatctgtaaattatgtgatttattaagaaattgaaagctctgtgtttcaga

SEQ ID NO: 4 - DNA, 658 bp, Arabidopsis thaliana
taataaaacataatatatagcttttgtatatatattgtaaacgttttcacgatcacatatatttggttttataaattttaaagatagagttgaatttctcgttttgagggtttcgctttgatatattatttatcaaattttaaaaactctgacaataacaaaaaaagacaatgttgcatataattgttcattttgtaagattattctgcatgaaagtggaggataaaattataagaattagcaactttgaaaaaaaaatcgcacaaactatttatcaaaattatgtataactttatgtcaacaatccaaccctatactcaatgtttagggacataattacaactttcacccatttatatactactgtttaaaattaatatacttaaattttcgttgacatttgatttaggtgacagcacgatagtatttttcttttctttttttttttttttatcatatgacagcacgatagtatagtcgttaaatataacaaatatatacaaaacttccgttaattaagaaatatagtatatgaataaatttaaaaaattgaacgtgactgaatcaccacatgactggcctttattctttataatggttgtggcaaatcaatacatatcctcatcctcacccccaaagtttttcagatctacaatta

SEQ ID NO: 5 - DNA, 353 bp, Arabidopsis thaliana
tttgtttttgttttttcgcactcgattagtgggcctggacttcgaacaaaatattcgataaggtccatttattttcggatttggcccatattaatcaaataagttgtacggagtattttaatagctcaacaagttctgacaatccttatagaacatggccgatcggatctataggttgattgattgcttttactttgagattatttagagatttatataatgcgacttgcaatagtacttattgacttacattcaaaaaatttgactttctaagccaaagacattactatatgtaaaagcttcagatcgtgaatcttgattaaaattatctgagtagcaagaattcaaaagaa

SEQ ID NO: 6 - DNA, 1019 bp, Arabidopsis thaliana
gtaattagttagttaattacacgtgctttaaagcattttaaacgcacaaatttgaaaatgatggcaaaggccctgattgattttgaccgagacacaaacacgcaaacatagaaaatttaatcagggatttgtactcggttacatagctgtcattctgtgttacgtattaattacggtatttaactaatcacgcacgcagtagatatactataactttacttgattaacaaagctaatagcatccataaattggggtcaagatgtttgaactcataacgagttactttgacatttgtagattgtgtgtagttggtacgaggtctactagtaagtcataaccaaatataactaatcgtagtagactaattcaatttcttacataagtcccaaattttctatacctgttctgcactaatcactcctctcattcttatcaccattggtccattgccatctcttgaatccgtcaatcttttattaatttaaaaaggccacctgctctattgttgacaatagaaggtgaggaaaaacaaaaccaatcaagccaaaaaaagacaacctgtctacatcattaagcaccaaccttatacaaacgtataaatcgaatttttaactcgtgtatgattgactaggcatccttcattttccacatgggccatcctgttaaaagatgtttcattattgcatggattactataataatcacaagaatttaaaaggcacaatcaagtggtgcttttctttatagttttcattggagattgtgataaaataacagagagcaccagagggggagcttgcactcgtcattgtcccttactcttttctctctcaatgctgcagaggcactgacaaacacaccatctttgcttatctctttctctgttttcttcaacgaaatttacatatcatccgtttcttctactcacatatggatcctatcagtctaatagtctaaaagaaagaagaagaagcacttaacttaatagaaacaggatcaagattcataagatctaaag

SEQ ID NO: 7 - DNA, 1215 bp, Arabidopsis thaliana
tattttttccccaaagaatattcgaaattattacttaaaaacgacttcttgtaaccgtaagtcttaagaattcgaatagacagtaaaacaatttccaaaacagaaaaaaaattaacagcaaaacagaaccaatagtgtctcctaaaagaagtaaaaataaggtggaggagaagtagtcaaatagaagaaataaaagcgacgtcgttgtcagaagaatataccacaagaatatgtcaaaacaaaaacagaaatatgattacgtataagtagccgttcccgacgctgcctctttttcattttcacaacaccatttcttctttctttctctcttacagtttctgaagaaacaacaaacaaacaaactaatactcttctcttcttcttcttttttctttctttaagacaatcccatattagagattgtttgcgagaaagtaagcaaaaaaacaaaaaaaactttctgtaagttaatgtcttcttcataactatgtgctattgaatcttttattaacttttattatgacttgttgttgaatgagaaatgaggttatgcaagcgtctggttagtgttgtttataaattatatagcttttgtgtctgttgtgtagcatgtgtttgtgttatgtctattaaatagagtagtttcctaagaaacattttctatgatacctgatcaatgttttgatagacgacaatatgtatgtggaagctaaaactcaaatcgtcttttacaaaattgttcagtaaactatgattatacagcttttttgttgttgaagttctgagattgaaacttaattcgtttacaactttctttcttggatcagtttattttggcacattcttggaacaaaaacaagataagtggtctgaaatattttgtgatttaaaggaaagttttgactgtcattgaactgtacattatggaatatgatcaaatcttttgtcggtgtgcgtcaccatcttacttcgtgtcccgtttttcatttcccacgcgtcaagtcaacctttacaaatacttacgccaaaaagatataaaaattgatgaatttgacgagtttttaaacctttttgatgattatagattaaaattttactatagtttattttctgcatataaaacgtgtctgaaaccagcttatgtatatgaatttgatactgattacttaatatgtgttacttaatattaacgcagaggaaaagagaaagagagca

SEQ ID NO: 8 - DNA, 1500 bp, Arabidopsis thaliana
acagctatttttttggatattaaaagaataataacagcaaaacagaattatcgtgtcctaataatacctaaggtcctaaacgaagcaaaaaagttggtaaataaggaagagaaaacctacaagatattaaaacggtgtcgttgttcggaagaatataccgaagtagcaaaaggaatatctcattagagagtcccttataaatgaccgttttaatacacttcaactctgtccttgttcataggcagcttcaacgatcattccacttccttcttcctctctctcaacattttcccctgaaaataaggaaactaaagattcttcctctctctttctacactcttctgacaatactaaaacactttatcagatcagatcccacataaactttctgtaagtgtttcttttaaactatgtctgttgatgttaatgtttatggatgcataatgattttagttaggacgttatcgtttttgttttgttaggtggtcctctctccctgttcaatctctaattcgtcatgtcttattcgtttctttcattggaaaaaaaacactgaaaactagtttttgtttttccaactttcttggatcattaaataggattgctctctcttcaaacgtatttttgttggatctctcctcttcagttctagaagacttttttttttttccttttgttgagtgcatgatttcaaaaaatttcaattgaagaagtttgtgatattttgttgttttctttcttcaataagaggtgtgtccgcaattaagacaaaacaactcgctgtcatactcagttaaagtctttgttgctcgtgttcagattttcactagacgtcacaagtcaacttttgtgggtaactgaagacaaatagtaatcgttgaaaactacaataataaaataagtacttccagtgtctccgttcacctttaaaaagtcgagataaacccaaaaaaataggaataaacaatcttttgtcatatccgttaataagttttaacaaatctttacaaattaataagaagaacgtatataataaatatttcaaaattaacgagtgtggactaatattgagttgacttttattatcacttcatttcagttccttgaatatgcatatactgtatatggtttctcttgcaataagtaagcatcatacatatgtacaggaacatgagaagttgtcgttaacaactacttcatctaaaaaacatacacatgttatttttctaatattgcctgacttcaagttcggtgaacatactttattgggttgtaattgtgagatcaatgaaatgagaaaaattaaatagaacttttatgatattttaatgttaattgatactatataaagcagggtctaattctatttgcttttaaaactgcctaacttatttcaagttcggtataactaactactttattgtttgtaatgagttatttaagggtacttatatgtcgaacttttttttgtgtgatgaaaaattgcagaggagaagaaga

SEQ ID NO: 9 - DNA, 1500 bp, Arabidopsis thaliana
tagtatcctagctccaatggaccgattagagctcatgagctgttagctagcttttttaaggattcttcacctcctttatattaaccaaaaaaaacatgcaatgtcaattcctacttaaattacataataaactaaagctataattataatccaaaccgatatataatctcgtttaattacaaaatctaaaccctaattcttctttgtaaacccaaaccgccatttaaactcatctaatcgtaaaaaaactaaatcctaatcgtaatttctaaacctaaaccgacatttaacctcgtttaattgtaaaatctaaatctgtttataaacctaaaccaatatttaacccgtttaatcataaaactaaaccctaatcgtactttctaaacccaaaccttttgaaataaactcaaaccgacatttaacctcgtttaaccctaggggtgaacccaagtatttttcatcattaatcaacataactaaaagggccaagagataattagtattgtacttaatcttaatcatctaacatagatattccacattggttgtgtgaacaaaatctaacaacatatatacaccaacatacggcaaatggtttgtcccgtcgaggacatctgataagattggaacgatacaaagaagatctgcaaggatcacactcacaaatcaagaaatggtccaaattgttttgctcttttttgtttctagaaaaagaaagtgtgtcatttacaatcttgaaagattcaagcaaaatcaattaatacaaacgatggtttggagaaaacttaacaccataaatcactttgaattttgtatgtcttataagaaagatgtaagatttggcttgagaatatataaacaatataattagagatatcttgggaagcaaggcgtttatcttgcgaaccaaatatagggtactatgaaatgaagcccgtgaaacccgagagaagaaaatcttaatatatgacagcgttggtagcatggtctcgtgatttgttgtttgacatgaatatttggcttcagaagatttgacttttgggaacgaaaacagagtttcaaacgtgagaatggtgcttagattttttttaaaacattttttgggcaataagaaaaatatttatgcatctgctgatacaaacgattttgacacaatattcgatcgtgcgttttttatttctttattaacattattcaaagtttctttttagaatatttttctgatgattatgaatactacgtactattgaagatttggtcaaataattttattgttttctataactgtcaccaacggtagatggactgtcagaatctgattaggtctttttgggaccaaacaagattagtaccaaatcactgcacctacataataattgttatggatttgcttatatatatagtttcaagtcttttatctaactccattataacacacaaacacaagtcaactttgcattataactccatttgccctcctggttcatcttgttctctacc

SEQ ID NO: 10 - DNA, 1500 bp, Arabidopsis thaliana
tctttaacattaaaaaacaagggggcgataaaagcaaccaaagctgtgattcatggtcgacactaacacatggaataaaaaaaggttatctcttttttccaacatttctctggtgactaagttaattgaataaattgtatgcatatatttatatgttctatatatatactgtccaaaaaggaattgaggaatttttaacttttaaggaccatattggaaaatctgccacttctcatccactcataggttcgaaattggtttattataattttaacgcacaaattcccttgttagtgctaaagaaggatacctacaaaaattcatatcgaattctagacgaagagacctatattgtagagctgaaaactgatgttttacaaaaactgatattttttcgggaatgatttggtttaagcaagaaatatcttagtttgcattattcctaagctggtactagaaaacaactatcgataaaacgaggttgtcttccctttggccacctattgacataagaaaacaagacacctatcttttttctctagcaaagatactcctagagtcctagtcctacgacgaatggcgatgctttcttaaataaatataaaatattagttctctacttctcctaacgtactagaagtagaaattctgctgaaaaaagaaaaagaaaaagctaaattcccgacgccaccatttataatgcgttcattctgtttaaataatggctagagaagaacatggccgtaaatccgtaataacacttccaggttctttcattctaacataaatttatgtataaaataaagtttttaatctatccacctgactaattaaatttttattttcataaaggaaagagttttcctataaaacaactataaaaaaaataatagtatcagatttgaaatgacgacttcagtgacgataattttactgaattaccatcacgcccattattatgttttattttataaagcaatcttttattgtgtcctaacaaaaaagctaacttctaaattcttactagggttcaaagacatatttttttatcgaatcatgtcgtatgtatacaaaaataatttattttggccaatacaacagatagttgattagtgttcagaaatacccaactctccaaaaaaaactactctcatttttttgtgtaaggtatgatcctagattaaagaaacgtctttttttttattctctctgaagcatcttattgacaagaaaataaataaattccttggatctaaacgtcaaatcaaatgcattacagccatttcttctcttaaaccctataaatatacatgttcttctcactttatgcttcatcaaatcaacacaaacatatcccccaaagcagaactaaagcaagcccaagcttgtctctccccattccaacacacacattttcatatcaaactcaaaacactatttccttaatttctttcacttattcttcatttcttcgtctaaattcttaaccaagcaatcgag

SEQ ID NO: 11 - DNA, 585 bp, Arabidopsis thaliana
cattgatagtaaatcgattagtctcaaatgagagagatctcattgcaaacaatattcagtgtttttgcaaaagacgtttcagttgcattatttttataaacaaaatttgattatttggatttggatccatgatttatacacaaactaaattggaacaatgataacttttccaatcaagttgtgtgaaacctgctatgaaaaaaatataaaaaagaaacctgctatgagctattttctgtaacagtataatacatttatacttagtattataagcaacatgcttttgatgattaatccatagatcatcatttaaataaaaatcatttacttttctttgttgttgataaactaatttaaattccgttgtacaaagaaaatggccatacagccgccagggtgttacactcagcctaactatttataatctttcacgaattaatatatgatttaccacaaagaccaatcaatatattaatattagcacgtggatcggatggaaacctaacgaagacacattgatacatttgcaagacaacgtataattaagaattagaagtggtttaggagaggagagagaagagagag

SEQ ID NO: 12 - DNA, 1500 bp, Arabidopsis thaliana
agcaacttttctctctttttttcatgagatgatttttttttcatataaatacatttgaatatgtattccataacctaatgtatctttgcaaattcttctacaattaaaggacatgatgcaaattaaataatctgataacttactaattatattcaatttctctgtaaagtaatattctatcttgatgagaaaacttcaagattatccttatattgaatttgacgtaagatgatttataattatgaggaaaatttttaatactaattagatggatcattctggacctgcaaataggacacatgtatatagagttgaaacatattataaataaaaataaaaatagaaattagtggaattgttagtgggtagtagttggatttgggtaagattactttatgatgaatttcatattgaggcttgaagatcttcttctcatccattgacggattggatcctccaagaacagagagaccaattagcttactctattgtgccattctccaccgtatcttttgcttcaaccacttacaattttacaaaggaaaattaactacaagacaatttacctaaaggtggaatttaagaatattctgaaaatatcactttaactacgtgtttgatgtatgaatcatatacaaatgatatattataagaggctttaaaaaaggatcaagtctttgttatattccttcgtttagagtataagcggtcctaaatgcacttgtagcattcataacaattattgtagcgcggtatacaagtattacagttatttacatacaaataactgtaaacgctctatggagttgagaaacttgtatgtaataagaccatcatccctacagttgggtttatacattttgtaaatgaaaattggatacatcttaggtccatgtagataaacctgcacttttcaatagagtgtgtgactaggtctgggagatagagtttccttcacttcttatgatgattataaaagtataggagggggaccatatttaaaatgtaaagattcggaggataataccattagaaagttggacatcattagtaaatagtgagtgttacccaaaacaaggcactatctatgcattgtttctgcttttatattacagtttttctttatatattttggacgattatggtttgtattcacataaatttcaattataattttgctatatgattcatcttcctctatcattcctcttagtcaaatagagttggctaactaaaccttttattttaatcaaattactccgttttaacttgttttggtgccaatcaaaaccaaattttatacactaattatttttactcgtttttttttacaaaaacattaagaatgatacaacataaaaaaatgttgtctccctttatccgcatttgttcctcaaagatgtagcactataaatagtctcatcaccctaagaccaaaaccccaacactcaaactcaactgctctcccataaccgaaaccgagagaaaatatagaaaaag

SEQ ID NO: 13 - DNA, 1500 bp, Arabidopsis thaliana
ttgtcatgttcttatttaactactatatacttatattgatcatgctcaatgggagtttgtgtatataaaaataaatttagaatttttctttgtttttttttttttgggcaaaagaatttttctttgttaattcacacatttccatcaccaaacattcgaagtatttgtctctatctcgacaacatttatatatatttttttcttttttttgaacacgactcgacaacatataatacactcctttgtactgtatactacaaaataaactactaaacaatattgactctataatatataatttatatatacatacaatacaagtctaattcaatcacttttggttcaaattggaaaaacattgaattcttctattattcttgatgaccaagtctatatcacatgtaataaaagtacacataaccggtctttctttcttattcgtaagctgccatgcaagtggggcttcaagctttcgtaaaatctctcatgtattccacattattaatcgaatttaaagtattttgcatgttatttagattagaaatgtcgttttcttaatcacactcctaaactgcggatcagatataatcaccaagattaaataatataaaaatataatcactacttgccaagacatgaatcatgaggaaagttctatagcgtgtactaacatacaataaatcatatttatgcgtatacatttactagaaccataggatcgatgaatcagatgcaactcatgcagtcatattacaatatatatttagatatgttcatcttgatctttcaacttaaaactcgttcatattcatcatttaaaaaaatatatatatgaatgtaaaaaaatggtaatttaaaattagatgagaatggggaacaaattaatcaccaaacaaaaaaaacggaatagagtaaggaagagagaaagagtcaatgagtgggggcggctaagaaaacattataagtttataacttatgagttttatgattaggaaaattataataaaacttgttgtcgaatagtaaaattataagaaaactaagatattatttctcagtcaaattataaatgatattgaatatctagattctgatgtaggagcaaagacaaaggagaaaaacattagtctccccccttctctgtcatttcttaactctactaatattttatcacataatttatcatccaattttccaattccataaagtcactttttgattcaattttattttaattccctatactttattgacatcttccatcacatgaaggactatctcctccagctattatatactctctctctctctcctttattttttctcaattcacatataattttcacctttacacacatttccatgtaagtatatataaacctcacacacgcattatcatacaccatcctcctcattctcttcatcatcaacataagagagagagaagaaaaaaagaattacaattaataagaacaagatcaagaatcaagaatcaagaaa

SEQ ID NO: 14 - DNA, 1500 bp, Arabidopsis thaliana
tcaacttaaaaattcatgagctaaacagagttgctctgtttcggaaaatcattctaattacgtagaataaaactcacttatataaatctactaatcaaagagttgtccagtttactgattaagtagccaaatcatgaattaatagtgatcgtcgtcgagcaaattaaaatccaaatctataacaattattgacgccacttctagttactcctcaataaaggaagttgctaatacttaattaaaaaatagtgtgactatcctcaatatactcggagcgtgacgtaaacgcgccgccttatagcttccgtcgtcaactctgtgaggtataaaagacgtaaggaaaagcagagaaactggaggaaaaaaaagaacagagagaaagagagaagagtgaattgtgagacagatcgaaacgtgtcaatctgtgtgtcattcgatagtaactcggattcggtctcagaaagaaaacaaacgtgcacacacagagcagagagacagagtcgccattaaaatatgtccaacttaaaaatgtgagaaagagagagaagagaagagaagagaagagatgaatataatatacattgacctctctgtctcttgagcaagcacttgtcctctctctctctctctttctcttctccgtaaaaaaaattaccaatctttcacaattcattcacttgctcctctctctttcttcattggctattctcttcccattgctcatttgaagtttatttactctactcccgacgaccacgactcttcttcagatctactcactccttctttctctccttcttcttcttcatttttccggtgaccggagtcggagaaggtaaagcttctgatcccttcccctttcctccggcatactcgttcctctgctttgtctttcgtttttttcatttctttctgtttcagccttttgcatctcgagacttcatgattacaatttctttatgtttctcaatgcaagattttcgtttataatatatgatcactgatgtcaagaataaatcaccaaagatttttttctttccatatattttttccgatcatcgctcaaaaaaaagtttcatcgtttaaatttattttcagattttatcgtttattggtgaattttatgatcctagttgataattcaatctgaaaaaagaaactgatacagttttctttttgaagctctgcaaatttctgatttttaagctcgaatcatataatttgaagtttcccgctaatgttcaatcagaattggtccacacacttagttgttgtggaccctttgcaaaattctattttagttcattatttttcacattttacagatcaagattctctgatggagatggtttgtcctgactctaacctctgtgtttgaaaatatctatacaggttctttattcagatcaaggttctggcttaaagaaaaaagttgtttgaattttgagatttgtctggtccattgtgttgctgttgttgtttgaagagaaaccttgatca

SEQ ID NO: 15 - DNA, 1017 bp, Arabidopsis thaliana
tttcgtacttaaatactatcatcaattcatcatggatttcagagcggatactccaatcttctttgcattttatcttttttttaccgtttaaggttaacataaaaaaaaatatattctaaattttttttttactgaaaaaagattctattagaaatctaagtgcaaaaaaaaatgttgattgatggaggttaagatcaaaatatgaattttgtcttcttgcatattaaaaatctaacctatattttacatgaacgtaaatagatgattttatgagtggcaaaaacgaactcaccataaaaccattagtaacacatgtagtacaaacccaatattgaataggaaccgtactacttttctaattatttaaaattgcaggagtggtatgtcttggagaatcgaggagacacgcatcgagtttaaatgtgtggtcggagagaataaaagtgtgtgcagatttgcttaaagagtgaagggaaggtgctataaaaaaggatcttgttggccacgagcttcttccttttcgtcttctccgccgcaccctttgggtcaacagttaaaccctctccattacgcgtgtgaatatacacatataatattcacattatataagattatgtttcataacgtagatcctgcctgttctatatacaacaggttttggtatgagacgttagatactttaattcatctggtcaaatgcctagctagaataaatatctttaatcacgttcaaaattgattttatacgaatacacttataattaatttaattatgatatatggtacttgctttaccggccttcttgtaaccttccttcaaactctagctgctcatcaattgaattaacaaacaaaaacaaatccaagtcaagtgatcttaaatccagctcattcttattcacgtgccatgcaaaatgatgcatacagtatatataagggacacacacaaagacatgtaaatattataccatccaccgattaagctacatatcctctcagttaattctctaattcgcc

SEQ ID NO: 16 - DNA, 1500 bp, Arabidopsis thaliana
aagtagcccccataattcatcagtccattcaactcatttatttcatcaacttattaaaataatataaaatgatagtcaattcattggttgagttaagtcaatgatctactaattttgatcgactttgatactcctacatttacatcaatgtttctttttctttctgaaatattctttatttaccttccctttgcatatatgtatgttctcaatttcaattcatattcactcctcctatttttttggcaccatacatcatgcacatctggatcgatacgtaacatgaaagtctttaagatttgtcttgtcaccattaaccagcatacgtgtttacatgtggaattaacattgctaatttactgataattcaaattgtacattataaagaaacttcattttttctctggtggcccgtggccacaaccctacattctaacatccattaaagagggttcgaacttaacctaatctagagatgacattttgagtattgtcgcaaataaaacattctgatcataattaacatttagttatatgttagtatggattcaaacctgaggttgatatgatcggatccaaacagtacactacattaagtttacggatcgcaatcaagattgttacgttggcatcaaggtatatataaaatatcttgaaattttttagtaatttttataatgttgtgtacatatgtacacatgcacaactataagctaaataaagtatgtccgtattacccgcttggtagttgaagacttaaagaggccatcatcaattccatctagtgacatttttcaatttctcataaatgatatagtttattctatagtttgcatgacataattttgttttgtttatatgatacgaacaaatttgggtgtaacaacatatgaccggtgactttatatccgaaattcagaattcgatatgagatgttctaaatgaatacttatgtgaaatacaaacaacgcataaaatgacgtatatatacttatgtgattataaatgaatatttggaaagtagaataaaaatttaaaaacaaaaagtagattgcaagtatgcaacatttgaatcctaggcgcggatggttatgcatgtacgacatgcacgaatgatataggctctctgcacaacacacaagatcaaaacaaacaaattctgcatgtagcattctaaaattttcgataaattttatatttgttgatctaagaaaaaattgtaatccctgaaaattgaatttgagacctggtatagaatctccacaaacataggaatttattattattaaaaaagctttattaacaatatatcatcttcttctttttttcaattaataaactggtaatattaaaatcgtcaaagaagtctctctctcccccatgtttgccaataaataggagataatcgctaccaaacttctcattctcacaagtcataaacagtcgtttatagttcaatccttttcctcttctttgtttctaaaaagagaaaagtcactt

SEQ ID NO: 17 - DNA, 1500 bp, Oryza sativa
aacaccacccttgtttgagtagtacctatgcaatcctcacccaaaataaccatataaaaatcttatatgcagctaccaaattatccttctaaagtgaaagaaggaagagcataaaactcttatatacagctaccaaattatccttcatcgctgttatgttgatgtatgagtggaattggtatatataattctagtttgagtttaccttgtctatggataaactgattgatatacatggactcagcgcttacctaattatagaacctattggtaattaaacaggaccatatgttttacaattccaacctatgttgggagtctcaaatgaagagtatataattgatctagactttatcctcttacatgatattgcacaaaacttacttaaatatttatactattttaaaataagatagtgatggtggtagtaaatctgtcatcccacccactttattttaaattacaattacccattaaacttctttgtactaaaactcaaatcactttagatagccatgtataaggtcaaattaagttgaataccggtaaacattggtgatacattccataatcaaaacacgagcaaactatatttttttacacaagagcaagagctctgccttttattcaaggagagcaaactataattcctccagaaataccttttttttattgtaatttttggtacctcaacacgtgagaaaccaagagatactaattaaagtttgcactacaatggtggtaactctgagtacgactgtacgagtacctcctactaatgattattaagcatatgaaattatcccggttgtttgttctttgatatgccttccacttccacgtcagctttcccacactggtctaagttcttggcacaatattctcttctggtttccatgttttttccctaatgattagcctctaaaagacctatatatttagctgttgatagaaaatgttggactaaagaatttatagaagttggcatgcacgagccaaccgtcctacacctcgagcaagtttcctataattgaggcagattaagaacaatagcacttgtacacgctgaatctgaaattctgaatgcattttctatttcttctcaccctcttgcattgacccttcgcgcattctttaatcccaaataacccaaaactagccatcaatcatacatagaaaaatgcatcacaagaaccaaagtaaacaccggccagaatgatgccattcccttgtaaaccaattcaaagtttcaaaccccaaaagccattccctgaaattggagcaagctttcacacagggaatttgatcaattaaaaaaaccatttgagcacctaacttgtgcaactataaaaggggtaggcctcacagccccctatgcaacaacaacttcaagccatctcctcttcattctctcctagctttctcccaacttccagccattgctctcatagctgatccaccaaggagctagaagtaattccagggttcttaattgcggca

SEQ ID NO: 18 - DNA, 1500 bp, Oryza sativa
tgtgttaagtttaatcgtgttttattgagttggagggatggagtacttgttaaaatactggtaagtcaatgctgtttttagctacatccaccagcctcgttgaatgttgatccaaacagttgttctgttccattttatatattactctctccgtccctcagtagatcggatcagagttgagatttcacaaaagaataccttacgattggggaaagtaggaagcggtttgggcatgcgctgcacatgtgagcagttgtcagttgagcaccatgtttatgatgataagctcggacgtactttcttagtcccaaaataaaaatcaataaagatgtaacatatttcagtactgagaatcttcacgtggagatgttaagcaccataaatctagacagatttgtgatagttgtacgagataatctcttatatgccactgaaaattggtctgattccttatatgccactgaaaattggctcttcccttatataccattggtctaaatttgtgcaccatctcatgccactaccatcagttgaccatgtgttgaccgttaactctcaagtaaaaaagacatatttgccctctctgagttgttaggcatgccttataattagacgggtaaatatgtcttttttccttgagaattaacggtcaacacatggtcaactgacgggagtggcatgagagggtgtgcaaatttggatcaacggcatataagggaagagccaattttctatggcatataagggatcaaactaattttcagtggcatataagagattctgcctagttgtatatagcagtgcggtagtattatctgttcagtaggcccgaactacaaaaaccacattgaatctagtcccatgaaagttttcatatttttttcaaaacttctcaagttgttgaatttttttattttcctgaaattttcaatacaacttttttctaaaaaagataatttctactacctccatattttaatgtatgacgccgttgactttttatctaatgtttgactattcgtcttatttaaaaaatttatgtaattattatttattttattatgatttgattcgtcatcaaatgttctttaagcatgacataaatattcatatttacacaaaatttttaaataaaacgaatggtcaaacattgatcgaaaagttaacggcttgatacattaaaaaatattgagggagtactatatctaaacggcgacgtggcagcgggtcgcgtgagcgtgaccaaagtgttttgttttaaaaataatttaatttttggaatgtttttcttaagaaaagttatcgatcgaagagttcgattttaaatcttaatggcataaaatttccaagatgttatcaccaatcatcccatggcataacggatctgaattaaggaatctactactaaaaaaaaaacttggccattagttaaccagtcgggtataaataggagcaagaaagcaaagagaagaggaaacagagcagagaagcagagcaacc

SEQ ID NO: 19 - DNA, 1500 bp, Oryza sativa
ctggcaaggagggagaaggaggccggcatccggccggagcccgaggtcgacctcttcatgaaggtaattaattaattaactggagtgattattttttttgttgataatgagtgattaattactactatttcgattagcagcttaattaaagccgcacatggtacaagtacactcactttagtttgcagggtctaaatcagatggtccttgtattattatatacagtggtcactggaaatttccacaggaatctaactatatattttccgtgcaatttgtaccttcgtaaaggacatgaaattatgaaaatgagattcgagcagggttaatgacaaacacgatctttgcgaaaacgacatctcacaatcggttagacgtacgtgtaccacatggggtgttaatagtggtgatagtagttgctgattatgctttgtttcaatcgaaaaaaaaagaggaaaaactatgctttgtttcgaagataattaattttgatttgtttacgtgacgaatgattaattcctccttgcaggctacttcgatggaaggagtcgagagcagccttcagacagattacaccctcagagtaatgagcttttcacgactttgttgaatttgctattattatttttcgcttccataatactccaaattaagctgatagtaacatcattgacataatatgatttaacaacaaacaatttagcacaagatttcctcatcattatgttctccaataaaaaaaaattggttcagccttgatagagaaagcacaagggagaaacactacaaaattaagctgatagcaacatcattcacacatggattaacaagcgattagcgcaagatttcctcaccattatgtcatcaaaccggcaaaaaaaaaaagaaagaaagattcagtcttgacattggaaaacacaagggacaaaccatcccaattccaatcaatcatgggcaaatcatataggacttggttgcacatttgtccacaagcaaacaactagtactaaccttgacccaggttggtgaacactgcacaccaattgggaaaaggggaatttttttttcttttttttttcaaggatccaatcaaccaccaagtccactactttgattcttgggcacatacttctcctactgattagtagcagcagcagcagcaggcaggagggagtgggtttgtttgctgactctcttgtgtcttgaccacaaaggttggttctttcttccctcttcttcttcctccctcctcctcacctactgctaattgggagttggagctcgaattgagttcttggtggggcatccttgtagctttgggaggggggattggggggttggtgcacccacttggcctggtggaattggcgcgcacccgtggtgttgttgcccaccacttgtttgttgatttgcccagccaaagcttctgcttcttatcagctttactgcgagtttccgagcactggccacgtagtaacataattacattgggcaggttgg

SEQ ID NO: 20 - DNA, 1500 bp, Oryza sativa
caatatcttgatttgctattggctagaaaatagtggggatggtgcatgcattgagtttgttgctagagtaaatatagtatgagagagttattagcttttcttggtcttggtatacctataaaatatgtagatcaatttgaaatggagggagtagcatgtttaaatggatgataagtagaattatttatccttggtctgtataccaagatgaaatatgactatcaaaaatagctgaagaaagtatattacagtacactttgtactatttcacgcatacaatatatgtgtgtattatttcgtgaacgtatataaatactatctatcccggccacaaattataatattattaaaatgaaattggactactagacattttggtaggaaaaaataaaattgatgtgaatattacacatgtggctataatttatatatgccaaaggtggaatgaattttcacagatgaccctatatagttagggacacctacaaagattaattttcgcatgcatacgtcttaacctaaccatctacgaaaataggcctatgcaggcggctgactacccgcccataaaaatttctatgatctttattagtgacaggagaagcgtctgaccacatgtgaaaatagctttcatagtagtgattggtgttggttctaaaatttaaacattgtgactttccttatcagtgccggtatagcagtgccggttggcgatagcccttcccaaatggtactaatgaggtttgttgtgctatgctactgcgttcatattaaaattttgatcccaaattatactaattattttatttattctacatctgactttaggatctcgtttataccccgggatactataaagcttttcttgctactaagatcctacatacggtggtactatacgtatacacctatacttggtggcaggtggaacagttccaattaaagtgccaatgattggagcagccctgtataaactatcaactcatatattatttatatctaactttctccaataacctttctgataaatgagaggactagctagtaatgtcactgtgtgacattgtgagtaagaactaatagcactaaaataatactagctaggaaaatgaacctcacaaaggaagaaaaataataatgggtcatgaaggaatagtagctatagcagtgagtgacagctgaacgccaagatcatgggatatagtatataggtggccaactcgttcctctgttaataaccaattaattcgtgatcagctcatcattatagccaacctacactgtcactgtgtggatctttgtcctaactaccccttgcacagtgctcccccctgctgccactctcagtgaccacgtgcgctcgtgccaatgcactactattatattaatccatctcactaatcaattcatttgcaccataatattattgatttttcattgcagcttatatatatcagtcatatatatcgttgccattgtaacaagatcgatcgatcgatc

SEQ ID NO: 21 - DNA, 1500 bp, Oryza sativa
ttggctcgttgttcctaaaaacggattttgcgcatggacgagggtcttttttaaaaaaacaaaatacaaacttttactaaataatacggcatgtatcatgtttcccaaggagaaaaaactacgaaaactgaagaaatcggaattgcaggagtatagataggaaggctagctctatcatgtcaagaaggttgaaaagtctcctgacggaggcttaccgagtgctaaccaatgctccctcttgtgcgtgttgtttaacatgtttacacaatcttcaatacaaatattatatcatcattacaaagaaacctatttttcgcaatgaaaactttattgcaataagttgaattttttttctaatatatatttatgaaaattttcaaacatgcatggaagcatttttcatgataaatactaatatcttttgttcttgtcaaaacttaatgattttttatatattaataagtctatggtgatttcgttgtgaatctcaagtctcaagacatgtcggttcatacgtttttcagaggtacttatagaggcaggatgtacgtatgtgtttataatggtaagtgtccgcgtgtatatacgagcgtttgtgtatcaactgtgttccaaaaaaaataatttaaggttttaaggtttggccgtatatacttccatctcaaaacgatacatatttatgaatggagtaaaatagttacaagtttttcggtgttctacagccttttcaaagtgtttctatatcaagcaaaagaaaaaaaggaacacggcattttgataagcagatactccatatagtaccgtacacatcgttatcacaaaaggaaataaaccaatttttgcagaagacactcaagcaagtttcaatggcactctcaaagagagtgagatttaaactgtccttgctatacggactaactgtttcaggccttttatttagtgcggtatgtgtgaatctacctccggttgggacattgtaggcgcagatatagatggcaagacacttttcttcttttgagaaatgtaggttctatgccttgtttatcagcaagctcgtaaatactataactcgaacaaatgctctcatatggcacattttagaatcaggttattttcttgagattcaaacattgaaaaagagggtgaaaaaggaataaggttttaaaccttttggagttgtgtgtcagtttaataattaattcagctggtacatttaggcgaaattgaagttgggacaatttacatattttctaatactttacattgtttatacattttccttatcagttcgatccatcggctccacgcagctataaatagagccccggccggccctcccattgtcatcagcaagaccagcaaccactcgagactgtttatctcgtgtttacctacacaccacgagctactatacacacaccctctcgatctcatcaccggtattcaccttctctcccttcagtacgttctcaagagattagcctcttgcagctagccacc

SEQ ID NO: 22 - DNA, 1500 bp, Oryza sativa
taaccttaataagcgattgaatgctctatgtgatggaaatgtgcatgacgtctatagtcctttctaagactctaagagtgagttacaaaccgcctttgctacctgtacgttgattgagttttagaaaaatcgtagttgattgatcaatcatcaatctgttgttgatctaagggctatagctctatcttgtacctcgagccaagagggagacatgataaggagagggatatgtcagagaggactcgcgacaacgtcctggtggagatgggcgggcggtgggtgggggtgagatcatgtggttaggttgcaagggtgatagatctagcagcgtgagccaccccgagatgaaggagaaaacacatatggatttgttgatgaagagggtgatggcagtgatgtggagggaaagaggacagggataggaaaggcttgtccagcctcgccggtgtcagcgctggcagtgcaaagaggaatgccatcacacgcgagtcaaagttgggacttagaacgttggtggcacaagagacgggatcaggagagggggaggctatgttaaaattagattgttacacaaataacaatgtaatctgagctatttaataaactatatataaaacatcaggcaactaatgaatggtcaagccgttatcaattattctggatggtaactccctgccttcgatgcaggacggagtgtagcgggggcccctgtccgaccatcccttgcgataagagtttttttctttctcagtaacttatatttcctgtatttgtgttttttgtttcttttttttccttgacccctagcaacggccctctgaccgtttgcgttgtgtaaccaaactctgttgtcttcttctaatatattgacgtgcaatcatttagcgcgttcgcgagaaaaaaatggtttggatggttaaatcttactagaaaccagaacaagataggctacgcaattagcaatggatggttggcattaggtcatctaaggtcaaaccgacggagatacatttgctgcataaatagcaactagcgtaatatgtgatgtgatgtacttcctacgtcttataataagtttattttttagctacttatatttgtctagaaataagttaatttttagaataattatttgtatcggagtttatgaaagtaaaaagtaattgtatttggagtacataaagtgaggaagtattgagatttgataaagtagaggtattttagtttttttttattggtacgtatgagattagtgaaaaataaatttatttgggaagggatgtagtacctagctactcttgtaccagctagattaagtataaaccaaaaacaacgggagtagtagtagtaagaatgtaaaacggttccaacataacgcaacaacggaacggttggatattcggttgggaggagaagggagattccgtttgagtaggttataaatagcaaggaatacatacgtacgttccaaatcatcacaaagcaagcaattaagctggc

SEQ ID NO: 23 - DNA, 1500 bp, Oryza sativa
tggggaattgggcacgtaaaagtatgttcacaaggcgcaatataactgtgcaaacctggcaaaaatgcagggtcacttttagtgacgagatcaagtgggtgatcaccctaagcaagagcgactgatcgatgcttcggtttttctgcttctcttacttggaagcgttgcattggcgatatgtatatttttcttgtccttgcgtgccgtgcatgcgagtataaaacgtagacccggagttttggcatgcaaattaaccgtgtttctcttaagctgtcgatttggtagagacgccaggatgaaggacttggctgtgctggggcacttgttcgtggcggcgttcatgttccacttcgcctcctacatggtcatcccggcgatcaccgacgtgacgatggacgccgtctgccccggccgcgacgagtgctccgtcgccatctacctcagcggcttccagagcgccgtgagtatcgcgcgtttcggattatttaagtcacattttgcgggtgggatttgtgaaaatggaacggcttttggctaatttgcgcagatcacagggatgggggctcttgtggtgactcccatcgtggggaatttgtcggacaaatacggcaggaaggcgctgatgacactgccggtgaccgtggccattctgccgttgtgtatgctctttgcagcatgacttgggtcaccctagcttgatgaaactgcataatgcatctgtgcacattttcatatgttttccacttttttttctggcagttatactggcctgcaaccgctccaaggtgtacttctacgtgtactacgtcgttaaggtcctcgccggcatcttctgcgagggcagcatgcattgcctcttgctcgcctacgtggtaatgaaaacctcactatgatagatcgatcatccaaatacatatttcaaagttattgagatacgcatcctgaataacgaatacgtatatttaacgagaacatgtgacctgtacaattacgacaacagaaagaatgtatactaccacacaaaaaggagtaaaggacaaactaccaatacagtacaactgaacaaacggaaaatacggcgattaggcccactcgtcagcgtaacagaagtgggcggcccaccgcccaatcagggagtccttgaccaggtgaagtttgtggacgcaggccgaccaagtcggcgccaggcggcgagcggcggcgttcgggctcctctccggcgtgtcggcagcggggttcgtctccggaacactcaccgcgcggttcctccagacatcaaccaccttccaggtggcggcggccgtcgccgcggcgacggccatctatctcagggccgttgtcccagattccggcggagccaactccttcgttgacgaagcttgtgatcccttcctacaaggctcatcctgctccgccgcgacttcgtcttcgtcctcctccgttgaggagatctcaccgagacttccgccgcacaagggcggggtgccgtcgttgtcagac

SEQ ID NO: 24 - DNA, 1500 bp, Oryza sativa
ttcaaattgcaatgtgttaattgggaatatatatatatatatatatatatatatatatatatatatatatatatatatatatatatatatatatatagtaaatttgtttggtgctccatggtgcccgggcaccattgttgaaattgagtatatatccaattcaaatgagcatgaaactttttcaattatttaggtattaatattaactactttactaactagttgaaagcaagtttgaactgaatttgaacttgtttttgttagattttgattctaggtatatagcatccatacatataattagttaggtatgtaatcatggattgtgaaataaagttaaatttgagttgttttgttgataggtataggtatgaatcgaattcttataactaggtatatactcacaatttgaaaattttgaaaaatttgagtgattttgtgcatttgaaaccatggtgcccgggcaccatggtgccccatatgagtgtcctatatatatatatatatatatatatatatatatatatatatatatatatatatatatatatatatatatatgcagacaagcttcccatgcacactccgtacataccaattacaaatgtcacaaattctagaaaaaattagacacatactcctaatataatattacacatatctgcaaaatctcatattcaaattcattatattttagccataaaaaaagtggaaaatctgacaatttttagggtaaaaatctgttagaattttgttacggctaaaatataatgaatttgagataagatttcacagtgggtacaatactattagaaatatgtgtctaattttttctagaattttctatgatatttgctaattggtgtgtacgaagtgtgtacggaaagtttatgtgtatatgatatgttccttgttaattagtatctctcgtgtgtgcaagcgcatcaatggtgatgacatgtgtgtgatgaccgatggaaaggttccaaattaaagagaggaattttttttaaaaaaaaacgaggagatgatgaggaagaaccgaggaagaagatgacttagatttgtactacttttgtctctactggaaagctagtgttaagcaaaggaaagatgaatactacagatgattaaaactaaggtatcaccaattaattaattgggtcgatcgatctgatgctcacagctcactcacacactgactgacacgcacaaaatcagctaaaaattaacgaaaataaactgtaccccctcttcttccaattaattaaaagcttaaatcagcttaattatttcctaataacaacatgattatattatgtaaagctcatggctttccagtgcaactgtactcactcactcactcactcactatttatacccagctcagctcatcttcttctccgacgagttgcgacgacttctcttcgatctcgcgccaactcatcaccgccatagcttagcttagcttaaccataattaagctagcttagcttagcc

SEQ ID NO: 25 - DNA, 1359 bp, Oryza sativa
gttagttttcgttttctgatttgcaaaccagtgtagcatatggtatattattggccatgggcatgcactcgttcctttccacagtatgctgtctccaaaccaacaatcgcacctgctaaccagtacaatttgcatttatacataggctccacccccaacttccaactttccatcacatcacatcacatccaaaactttcctacacacataaactcctaacttttttttttccaaactaccaactttccccaaactttcaacttttttcagtaactaaacacagtcatagctgcgagtctaatgtgagatccaggaagtcaaaaccgtggagccattgctcctaatacactcatacatcttgctgtgccttgtgaacttaccatagccaaccaaatgatctgtaatttttccactattgcctggattgttttcgttgtcagtagagtaattactaattagctggtacactcaatccagtacggtattgttgaagtacattgatccagatatccggaatagcctattagatttcttggagcaacatcctgaagcaagatagcaacttgagatctagtgtgttttcatggacaataacacgtgcgtcccaggtcatgctttacgcctcacgatgaagattacaccgtactcgtgtcttcatcagttattccactcaaaaccgcattcctgaagattcgttgaacctttgtggccatgctcacttgggttaggacatattctcggcgtttaagtctagtgtggaaagattgttcaggtacccccccccccccaacccaacccccccccaaaaaaaaaaatggatgtgagtattggaagaatgggagtattggatttacgtttttgcgttagaatcaagatggacaagcagataaacactttatttttcgttagggaggaaaggaaaatataaataactatggaacaggctgataagggtgagttagggagaaaaggaaaatataaataactttggaacaggctgataagggtggatcctgcggttgccataacgtagagaagattgtactcgaaaccaagatcaagaacaagcaaccgcattccgttggccgggatgtcaagcgtggaatcgtttcatgtggagctcgccgcgcgagtgagagagaagttgacttccccagacttgattggcgaaaaagccatgcttaattccaacgacacacgcttggtcagagctcaatatgccaccaatagaccccgcctataaaaaagcgcgcgagcctgttccatcgtatcacaaccactaacgtgcgttcacccccctaacaactatctcacccccctaacaactatctcaccggctcgatcaatctctcaccggag

SEQ ID NO: 26 - DNA, 1500 bp, Oryza sativa
agaacacacccttagcttgttttcgcctagctaattaactagcacaactcgcgtaagagaaacatgcttgtaaatagttttactcttactctatataatatgtcggtattatctggtgctggttcgttaaaaaaaataattggccagtttaacgaagtgagcatagctcatttagttaggttcctttggtagaatcaatctacccgatttcaaaatcctagacttgacacggatgctcgcatttatggttatttattttttcagtgataggcgacgtacccgtcgttagcaagacattcatgataacttcatcaatctaaatatgtgtcggtccagtttttcggatatgcacgtagaaatatggtatacgtgtgtttataagggtgagtaaacgtgtgtttgtaaaaaaaagcataataaacatgcaccatagatcacacgagcgaagcttatcttgaaactgcaggctccagtttggccatcccaggctcccagcacacccgctcaaaaagacagaagtttagagagagagagagaatatgcggtaattttttttagtggagttggttacatacaaatttcttataaaaatgttgattgtgccacgttatgaacgaatcttgattttttttttataactcgcgactaattaaataaaatgttgaaatcattataaggaagaagtttatatataagactattttttctattgcaatgctctttatcataaagacgaacgacctgtcaatggtcatattactatataatacgctgaaacaattggtactagggattagggaaaggaaaaaaagtttgaattagacctccaaactagtgagtgagtaagaattaccacctagactttagattggttttgctcttatgtgatagactagtcaatattttttaaaaaaaaaattggtaggacccgtccatctcttcttacaggtgggttccactgatttattatgaaaattattgattgagctgtcataatataggaccaaaactattttggattgtgttaaggggattttaattcaataaaaaaattcaatattaatattaatatggcgttcatgaaaatatttatagttgagagtgaaaaatgtatgattttcgtgtctagaggttaatttgcactcatccaataatttagagggtgtaatagttgagagtgaaaaatgtatgatttttgtgtctaggaggttaatttgcactcgtctaataatttagcgggtgtaatttggacttttttccatacagaaatgggcactgagtcaaacaacaccacatcattaattactgcagtagactgtgagcctgtgaagatggtataaatgtatgctgagcgcctagaggcacgccgaagccagacaggcagacactaaccgtggagtagtctgatcgatctatagaaatactaagtagtagaacacagtactgtagccaaaaactaggccaagagagagattgggcgttctgagcgtagccaaagac

SEQ ID NO: 27 - DNA, 1500 bp, Oryza sativa
cgatgggtttgcttgggccaacggtggccgtaggatttttgtgatgacgatgtcttgtaatgctgctgctgttgatagttgtaatgcttgaatgatgttcaaatttttgttaatgtgttgatattgtgatgcttgaatgctatgttaatgtccattttcaattttttttcaactttctggatgagatggtagaaagggatttatttttttatggaaaattatttttactggcggttagcataacacaaccgccagcaaaaactattttcactggctgccttgataaggcaaccattagcgaagataactttcactggcggctgtctccagcgaaaatttgtttttgctggcggctgacttaagatgccagtgaaaatatttctttgctgaccgtcgataaccgctagcgaagatcttgatcttcactgccctttgctttgtggcggctctaaattccgctagtgaagacccaaaatggccaccgcgaaagatggttttcatagtagtgagaaggccatcggccctcgccacaccgggggggaggggggggagaccaccaccggtcaagaaccaccttcgcccgccgccgtcccgaccagcgtctacccactctatatggcgccctctgccgcgttcgctcgctgataaagagaagagaagaaaagagagctgaagcaggaagaagaacaatgtccaggggcatttcggtactttcaacgctctttctctctctatcttcatcgaaaataataaaataatgcattgggtgtccagctgctaattaccacatttttatagtgtccacgagaagatatatattttttgagtgtctcacaacaaagactaaaactttgagtgtccttaacagattttgccaaaacaaaatgaccctacaccatatagtaacttagggtgcgtttgttcctgggggattgggtaagaaagcctatcgtattccgcgcgcacgcttcccaaactactaaacggtgtattttttacaaaaattttctataggaaagttgctttaaaaaatcatattaatccatttttgaaatttaaaatagttaatactcaattaatcatacgctaatggttcacctcattttgcgtatcttcccaatccccttctccactcttatagtactccgttaaattcatgggtgcaactgagtatgtaagtcatcgctacgagcctgagcctacgatcacatgggcggtcaaattaaatgcacatgttctccgttgcagacagtaaaatgtcaactcccccgtatataggaataattatttttagttacttaggcgacttgcaagcaaattaaattgctgaagtcaagtcaattgcttgctcatctcaccctcactctagatcttcaggttataaaatgccaaatgctccaatcgatcgatcagagctccaaatcagtaagagacagctaggtaggtaccagcaaaattaccactaatataactacgagaattagcaaaatttgattagca

SEQ ID NO: 28 - DNA, 1500 bp, Oryza sativa
atcgcgcggccgcggatggagggggagctcgtccttcctcctccagttgcgcattctttttttcttttttttcttctccctcgcacatggcccgtgggcgcgattccttgcacccgtttttgccccccccccccccccgggcgcttggggggcattttatactatttccttcacatggccgcgcgccccccctccgctcgctgccattagatcctcgccgcaagctcgctctctctcgcctcctccgcgttgcgttcgcattgaccgtccctgctgttcttgcaacctgcgcttgtgggagtggagagtcgtcgagcgggaggaggaggaggagtggtggtactctgttcagttatttcttccaggtgggtttcctccttgattcttgcgctccctcgatcggaatcgccggtcaggtgtttcgtcgaacgggtggtgggcgtcgagctggtgggggttttgcgttgttcccggcgtgtttcttgtttgatcgggggtggttcgtgattcttgaagcggaagggttgatcttggcttactccggggttacttgttctatctgtcatttcttcaaatgggttgcgagcatgtgatgtgacaaacatagcagtgcaagatcgaacagcttgctgattttgcattctgttgctttcttgcttatttagttgtgacccatttgacttttgccaggtatatacttgatttgattcttagtgagtcaagcaagtcggatacttgggagttaattccagatatttgtgttcatgtagtcgcgattcatttttaccacccacattttctgtctgattttgtgatgtacccacattttctgtctgattttgtgatgtcaggatcatgagtgaatattagattcgttttttttaaaacttttcttcactgtgtcttcccttgatccaaagatgctccagctgcgtattaggaaggaaaggtgccatctttatggctttggaagaccgaatttcgtctaaaggagatcttcatgtctgcttttgatgccacagtgatgagtgttttttcttggttacatcaattcaccaaagctgaacgacattccagcaaagctgacttccttttggcattttagccacagaagagctcgttcggaaatcagaagtttccacgatccaaatgaaactttagacacatattttctgcacgcacacactgccttcgtctaatcatatttttgctctcatcatcctttcaatttcctgatcccatttttaatctgagaattgtccaactgatttgatacatttaatttattcaattggaatcgttttcttagtttggcactgagttaacctgatgtttatgcagacaagaagaagttgctgattcttgaggagttatactataaacctatgaccttatatcactcttactgtactttgaagtgaaaatttgtgctgcaatgattgcaacgttgagtgacatttcaatgtgacttggtaactgaagcgcttaatattgagaaag

SEQ ID NO: 29 - DNA, 1500 bp, Oryza sativa
gagagttttggaaatgaaacccatcccttttatctctgaaacttcccaacattatagtatagtgttgaacaatttacaggctcctctatattggaatactttagatgaaaagacaagaacaagggacgtgaactagatgacgcagctacttgaaattagttttgttgttgtgcgttgcaaacaaaaaaaagaaggcatactagtacttgttttcttgtgttcaaactgcagagacaacgtattgcgtactcctgaatcaccattatggcattatccctgaaactctgaaaggaaccaaaaatcctaatgagctaagcagaaacggtttttcttctgtgagatttaacacaaatgacttagcattatgaaataaacatgtagaatgtcaaacaactttttagctgaattacttctatataaataacaaattcttttggaactgattgctatgaaagagactagctcccattaaaaatttctgaaagtgactagtgcgaacgttgcagcctaaactatatcatactatattactgacattccgtgcactccaaactcttctttattagtgattgtttcaatgaatacaatcagcaaaattacaacctttttttctcatgaaaagatagaaacttgattgagagtactcttttcttagattgatgcctatattagaaattagccaagaacttcaattccaacaccacaccaaaatactacagtaaaattgatgctgtttttttgtatattgatgcctatattagatagttgccaagaactccgatttttaataccacagtaaaattgacagattacgaaggggaaaaaaagcagtgaacgttttcttttcatgaagctaagagcaaagcttgtaccgtgactgtactggtgaggcatacacggaactagtctactttatgggcaatctgccaatgcattggtcagaaaatctttttgtcaaaacgggtcagcctgacagcgagacgcgcctgctgattaacgtgacacaaataatggtttgaagtaaactttattttattgtttatcttcatatggagatctttcttgcttgctatgcatactaaccaagtgttcagacttcagatactgatgtgtattaactattaaggcaaatcttgactggtaactgacatttgggcaccaccaaccattttatttttctgggagtcccacttgtcagtgacacatatccgcggtttgatgctgaatttctgaggaacctggaggacagattgcaaatttgcaagtagagtagtctttgaacaccttatatccgtgcacaaaacggtggtcaatccaggaaaccgagcaaagagttggacacttgcttgttcacaccgagctggaggccataaatagtagagaccaagctgcatcttcttgttatcttcagttattcagaagttcagagcagtagaggaagaacaggataagatcatagctagagatttcagagagagaatctagagagagagagaagag

SEQ ID NO: 30 - DNA, 1500 bp, Oryza sativa
ccaccgccgccgcgcgtttgccgtcgccaccaccgccgcgggctgccgtcgcctctccgttgcaagctcggggctagatctggccgccacgtcgatgggggaggccggagccgtcgccgccatgacggaggtggctggatccgctgccgcgacgacgggggaggccggatccgccgcctcgacgaccggggaggccggatctgccgccgacgccgacgaggcctcggcagcagcgcggggaagggggcggcggcggcggcagcagtggctctcagtgtcaacggcgcgggggaggaggcggtggggagcggatctggcgcctcctccctccccaccggcggcggtgaggtggcgacgacgagagatcagcggcggcacaaatctgacgagctcggctaggatttttttttgcatgtttccagatgatccagatgatgcagatgttttgtgcatgttgtgatgatctgtgtacatgatgtatgatctgtgctaatgatgattttgtattttgtgtttgaattcggaaatgagcagccatatgcatatgaattggatttgaatctagatctctagatgtggatttggattttaaaaatgaatcaatgggcttttctccaaatcttcgttggcggctgggtagctgcatgcgtttttttttttttgcccaaaatagatttttgctggcggcctatttaacacagccgccagcgaaaatgaatttttgttggcggctagtgtaagacgaccgccaacaaagatccatttttgctggcagctggtttaagacgaccgctagtgaaaatgaattatcgccggcggtcgataaccgccaacgaaaatcatggtcttcattgacctttgctttatggcagctccaaatttcgccagaaaaacctaaaatggccgtcaaaaaaatagttttcgtagtagtgaataatgaatggaagataacagaagtactggttaaacgttcgatagtgttttgaatataattccctccatacaacagtatacgatacagtaaaatggacttttaaagttctaattaattaagtatgtatttgtaaggcacagtatcagtttgcttaattactaatccccaatacgaaaagtatagttgacttgcttttgttgtgcccaattccgatcactattttggcaacactgacttgctcaggtcatcttttttcgacataaacatgcaactatttctttatttcctggataaattttgggattcttgattacaaacgtgcatggataattaggtgatagtgacaagtcatgagatgagagttgagttaacgaaacaaaaagtcatatgattggttcatacgccagcagcctagctagccaccggcctaagcaacaaaaaataatattattttatgttaatcatcatgttactcatccctatataacgctccaagacggccaattagttagctccacgtcaaagttgtcaaaatccggcgcggagcaagcaagcaaactccactc

SEQ ID NO: 31 - DNA, 1500 bp, Oryza sativa
tatggaaatatagaaagtttttttaatggctcaattaaggagaacatatgatctttaaacattgaaatgacatctgagcactagacagcacccctacacattgaagtggtggcagcactgcgttcccttcctttattctcttccatggtgttttgccatagtcctccaaagaagagaataaacagataataatggtaatccaagtagtatcacagtttggggaaagaatctaacagcaattaaactcatcaaattctcttttccttatcagaaaaagaggacgctgaatggggcttacctaaataaacaaggtgtgcaaagatgaacatttgagggcgcagacacttccacgcaggtggtgaagcatttttttctgctagataacaaaaagaataattagaaaataagaagataagaatatcaagcaaggcaagctttaatgtttaacaagtaatcaccaaatcgaacacgcaaaaaatgaattgatagttttggcaaataagaatcacttgaaagtgctaagattccttttttttttcttttgccctataagggcatggttcccaatttttcacaaaaaaactccatcaagatgttaacatttcatattaaatttttttaattgccctctccaaaaactagatgttactgtaatgccattataaatgttacagagtaatttctgtttttcatttagaaaaccataggatgatacataaaaataatcataacatgctaaagagtatattgtcatgcagactctgcagatcggcagacaggaggacgaacactgtttattgaccgataagattgctcagcaacgtaatcttcatagatccacttgcctcaaggtttcttatgtttcagaggaatagcataaatctagtgatctataatattttttgagtaaactactgtggacaaactatcaaactatcagcttgaaagaaaaaatcatatcatgaaaaaaaataatggtcttctactgacaaaatgtttgtattaataaccaaaaagaaaggacctgaaaagaggtgtattaacacctccagagagttcaatagcactggtgacttgcatctttcccacgcaaacagttcttttaagggggaaaaaaacaaatgcagagggattatccttagaacttgatggaagatacatgaagtacagcgtcagcgtgtatagattctattgcaaactgttatagaagaaaataagacccttggttggttatgctgcaaaagttgttagctactttctgtagccttttcagttattttccacggcatacctgactaaaaaacacacatactactcatgtctcacattgtccattgaggttgcaagacacttgaccagcaggctgctggccactataaatagaggctcgtactaccattgccaagcaatcagcccacagctcaccgaagtgcattagctcaccacgactagctagagcactcattggtgtgcaaaatattactgtctgctcctctaaac

SEQ ID NO: 32 - DNA, 1500 bp, Arabidopsis thaliana
acgtgcctatgatcttatctaactatcattttctcttccttactcaacatcctaaaagaactattcaaatttattagaattccattttcttgagacatgaatactatcaataataattaaactatagttaaacagaaaggcgtaataacataaccaaagccaatgggtttttgcttgacttcccggaaatttaaaagattatgtggttcgataatttacccccaaattattgatgagcaagtagtagtctttagaaaatggagatggacagacctttttcccatctttaaacttgtaattaataaagactttttagactattgcaatactataatgaatcgtgtagtgttgtgttgtgttgatctctgtgttggaacccaaatacaaaaaaatattgtgctaaaaagtgctgcattgatttacaacaatcaaatactcaaccatatttcaagtgggttgaattttaataaggatgaatgattcaagcatcaatctataaacaccaacaacgaaaagcttctattcacaatcgaatgttgtaatcacatacatatatataaactgaaaacaatttttagttaaatatttttagttattaaaagttggcaacttctacttaatgttatatatgttgcacctactattagacaatatctagttaaaacatagcaaaagaaagcttgtggccggtgagaatacaatgaagaatgtaaccccaaaatcatcccaatttccactattttgcctcttaataatagtaaacaattaataagggaacgcatgataaaagttcgaagattatgtacatatgaatatttggttaaacaagacatatgatgttgttacaaacgaggattacataatgtaatagtaatctaaagtcttagggactaagagttcctcattttaatttttaatttgaataaaaattaaaatgatcaatataagagtttttaagatgcaagcaacacaaaaaaacaaaaaataagaaatgaaaaaggaatatttttcattagttagtgtacattttaatagtaataccgtgtatgaccacgacatcatcaaataataataatatgatatcgtatgtaatttttatatagtagtgtgttatttaaaagtttccaaagtgcgtgtaacatatgggttcctagtaccttccgggaatggttagcgtcgtcaagaaactactctgtaaaaagttagaattaggttttccaaactaagaaacatgcttgtaaccaaaatttaaacaatggacaaaatacgaatggaattatcttgcccatgtgatgtgacgtgttttttattttcaatttgtttttattccagaaaataaaataatgacacgttggagccaatacgaagaacacatctatccatcccaatcgctctccacttttctgctcttcgacgacaaaagtttcaaactctataaatacttgaacgcaacagttacaaaaatgtcaaatcgaaaacaaacacaaaaagcaaacaaat

SEQ ID NO: 33 - DNA, 1202 bp, Arabidopsis thaliana
ttgcagatgctagaaaacgacgtcgctttgtgtgtgtgagtgtgtgtgttttgctatttgcatgtttatgctttcatttcatcacatcatatgttttgagtgttttgccttgtactctgttgtgcttctgtttgttttgcgttgtcaagtctccaataaagttggagtgtttccgttgacaaacgtttttgttagttcttgttgtatttagctaaccaatgtttttagatttttttttctctttttctttgtgttaaacactaatgtacatgtttttgttttcataaagtttgatgcatattagttaaaaaggggttacttgtttgaacatacaacaaaagttaaaaaggttactagtaagctaactaaagtcttacaaatgtccactctagaaacaaaacccacaaattcacaaaagacggttagaagcccaactagtagagaccaaaaaggtagtaagccccacaggagtaataagccctgtcaagatgaagggagtgacgaggaaacagatagctaagggtcacttgttcatcgaagattttaatcgtggttgaatcaggttgacccaatgatctggtgatttagaagatagtccaacttaacgctcgaatcaggtttaaaaatattggtttatgtagtattttcttgaatcccatatagtatttttacttttggtaaataaatgtattattgaaatatttaacatgagggtctcgttacatatattatataataaaaatattttattgatgctatcgacatgtgatggtgagtatattacattattttagatcaaatgcaggtctcgttacatatattatataataaaaatattttattgatgctatcgacatacagttagtgagtatatttcaatactatagaccaaataataggtaggtgagtggatgcctggattagtggaaatattttaacaattattattctcttacgcgtggaaagttttaaattactaaaaaaggtaaacttggataatcattttattgtaaagatattgacaatttgacataaagtattttgatctatgtacccattccaatatataaaattgcatagtggttttttcttacaaaaaaaaaagagtaacatctagatgccctttcccttggagtgctataaattcagactccttccctaaataaatcacaaagcgagaaagaaagacgaactgatcagca

SEQ ID NO: 34 - DNA, 992 bp, Arabidopsis thaliana
tcaagttggctgatgaaaagatgtttccaagtctcttttgattatatgagtctctctcgcaactttgcttttccttttccctttttgtgttgtccaacaaataaattaaaatcttggaccgatatcgatagcgatatgaacaagcaatcgagcatgtcttttcttcgatttactggttgatattaatcaaaaataaagttttttacataggaaatgttttactagtagcccatgagcataataaaagcagagagaacagagaaatatgaggagactttcaaataattacaacaaagttggatgattatcctattttatatgattttaccaatcataaagctctacaaattaacaattaatctaggtgactaaaacaaaatgaatgctatcattctgcatggttatttcacttagccgttttttttttcatttttggtgagaacagagaaatatctcattttgttgcttccgaaactatcataaagtatacactgcaaggtataaagaaccggtccacaaatacagtgaaattgaaggtgtgctaaacattagataactggttatgcacccttacgctaacactttttcttgctaagatacaacatcaagagtcaagacatttccttgagatatataggccgactttgctagccgttaaaatgttaactttacgttcttttcaaatctgcagattttataataatacagttcttctagacggatcggtctctcccagataaaatccacgtgattgtgaatgagaaaaattggtaggtaaggtaataaaaaattgatgatgatcatactaatattttaagattctcgcttattatgtgggagattaaaatatatggatataatttttgcacttacgatcccatctacttattatgtagattataaaatagcaggcttgcataaaagcataacttgttgtaccaaacttgttagtcaaacaaaaaaaaaaaatatccaagaagctttgaagaacc

SEQ ID NO: 35 - DNA, 1211 bp, Arabidopsis thaliana
tgaaaacaactattggtttgaaaaaaaaaaaaaaaaaagaatcttagtatacaaaacaaagagtaaaattgtctattttgcaaattgtaaatagtagtgttactagttacataattatgtttccaaaagtaactatatttccaacaaacccttttttaatgtatttttttgatagatatattagtagattttttttaacaatggaaaacgaacaaggttataagggcagatgagcagagtacaactgttcttaaagtctcatggcaaatcttggcacacttgtgttatctgaaatagccagtctttagattttcttgtactttttattttgttgattggagtttgaagagtgttgagagaggagcgacgaggacaataacaaatttctcagtgattaaaggtagcagagaaccctatacaattatgggttccgcattatcctttcgtaacctagattttttttttatatatagttaaactcctagcctaaaactaaaacacagttgatgactaattggtggagaaccattaaaaaaaaaaaaaaaagacagagtagactaacgaaaccacacctttacatttttctatttatctcaccttcctaaataaaaaatatttattttaaacaaaaacacattttctgtacagacgtgggtttcgtaatgtccataagtccatccatagtctaccatataatttaacatctacttctctccagtggacgcaaaagcttcgaattgaatgtgggcgttgcttctctcgtgtctaatataaaatttggataatcatgaattcttcgatgatagtgatataccgttgacactgagtagttcattgcaataagtaagatcttgtttttgctaaaaagattcaatcatattttgaacacttgccacccaccacttccaaaacttccacaaattcatagatatagtaatacacaggttacttgacaaaaggaataaaagtacaatatgatgctttacacattctcagtcaaacaaaaacgataaaatgaatgtgtaagataatattgtttaaaaaagaaaaaaagaacaaaaaaaagataatattgttttcccgacgactaatccactttcagtattttataaaataactgaactcaagttgctcgatcaaacaagtacccttaaatgtctctctgtctatataaatgtacactcttcccaaacaaaaccatatctcaaaat

SEQ ID NO: 36 - DNA, 1500 bp, Arabidopsis thaliana
tttgtttttcaaaatctcattttcataccaaattgttaataatactcaagaacaccttataagctagatagattacaatctaaagttgcatgataaaagtggagaagaatcggctgataaaagtggagaagaaacggccttagactgaaactgaaactatagttcaaagtttttctataattaattaggaaacaaattttatagtaatcttccagtcatgatgttagatttttatttatcctattatttaggaaatctagttctacttcatctacgttattttttctcaaaaagcaaaactttttttttaaaaacaaaaatttaataacaaaaactaaaactaaaatctaaaatcgaaaaactaaaaaccaaattcagaaactaaattaaaaaaacagcatccatttcatggccaaaacattagatattaatgcttctaactgtctatgaagagtgagatataccatgaagaggttcaaagttaatagaggatgggtgaagtctctggtttggatctcgtctttagatggccttattatgactttaagtcatctttgtacagtttttagtatcaaatgatcttaaaatctatttcttagctttactttcaccaatctaatgtgatattccccatctagtccctcgaaatagtttttcatgccgaatttttcatatatagtatatcacattatccaaagaaaaactttcgaaccaaccctaaaagcatctataccaaaacagattccccatgagccatgagaaactatgttgaccaaatctatgggttcttaatattaatttaagattctgattttccctaatttgtgaaatcaacgtcttctgcccataaatcgaacctcgaaatcgacaatactattatatataattaaatcgttatggttccactttacggtgaagttaggcgaaaaaagaagtagttaagtgtcataaactcaagtatgaacagaaggggagtacatattcaggggagtaattaactaattaagtgttataaactcatcatgaggatttgtgaaagtgttttccagaacatgcatgtgtgcatatagaaaatctaaaaaacatataagtccacgtacgatattaataagtttaatttaaatgttacacaattaatcaaaacatattcatttgttttcaagctcaaaaacgttttggttaaatgtaagcctgtatataaaaaaaaaacacaacttgtaaattaatttgatatccaagcattataaatccatgttttttaagaaatagtttctttctatatcgcttgaatcgacgttattttaaaattaatgcatgcgtgtaagtgtagctaaatacttttaaaaggcgaaaataagaactgataaacatttttctataatgcctcataggccactagttataaactagtaatttccatatgtgaaagacccagaactgtgtgtataaataagaatcgtcagccatggcttcttcaccaatcaccacagcacagcgatc

SEQ ID NO: 37 - DNA, 1051 bp, Arabidopsis thaliana
tcatcacatatatatatatatatatatgtctcttctataccaccaagtttgtcacgtgtgatatcttcattaagttttaagttttctaatttttcttgttctgtttatctatgttttaagtgtctttaagtatggtgttttgtgaagttgttatgcctgtaatattatatgtgattggctttaataaaagtgggtgattattatacatatcagttgcacattaggagaaaagcttatattcatactcagtatctcggcatctagtgtatgatggaaacaattatctttaagactttagcattatttaaagtgagaaacaagaaaacttcgccataattggagaattggtcattttgatcaaaatccaacaccagactacgcgcagtccgtttcgaaggaggtacactccacttagtatctagatttaatttattatacagtatgtttaattttttttttttttcaaacacatatttttcttcttaagaatatatatggaaatataatatgttaattatgtaatgatcaaataatattaaaacacacatatatatatatatataaaccatatgagcaattggataacgggtaaaatctaaccaggtttcaaaaattgtaactattgttgatttttttttttttttgatcaacaactattgttgagaaaaacatgtattatgtgagcaaaggaaaaaaagggaaaaaaaactacagatcagtaatatcaactactccaattcaacttatttgcttcatattcacaacattctaatattttaatgactcgtggatcacgagattatattaacatttctatagattaattagtggcctacattcaatgatatcaataattacacattttatccagaaataaaaataatcaataaatacgcttgaaggtcattgtattgcatatcaataactcgttccctctctcctcaggtaaattctcagtctctcgtctgataatagttttgtagagtttggttaataaaactaatcgtgtacgctactaatacgtacaggtaactaattaatattcaaaaag

SEQ ID NO: 38 - DNA, 1500 bp, Arabidopsis thaliana
aaataaatttcttaaagtgtgtgttttaatctaaaacatcatataatttgaaatagaggaaatatcatctaataaagtaatggtatatttgtatagttaatgatttgtctttttattcgcgcaaaatgtgtcaattataaaatataaagaggatataatttagtttagagttttagacacgaggactatatattggaaaacaaaaaagtaatgtaaaccatatagatcatggaatgagtcatcctattaaacagttgtattatatatttatattttagtcactaacacattaataacttaacgtccataacaaaataagatccaaaactcgatctagatctatacgaggcactaaatgatccattgacttagggccggccgattggttcgaggactcctcatgctgtaaactttttttttggacatacatgatatatttttaagtcacgtttttatattatatgttccacgcccaatataatatgttccaaactaggaaaaataagtaagaattagtcaatgatcgagataatgcaatgaatcatcctatttattaaatagatttactaaactatatataatacaatgatcgagatcgtgccatgaagcatcctatatactataaaaatagtcttactaaatacatactcatatagtttagtcattcattagtccaaacattaaatgagagatcctttacttgctacctgaattttttcagaataaggtataactttttttcgaattagaaactgatttatgaaagattaagagtaatgttcgttaaacaagttaaaaaatatgtttttacaattaagttttgaaaaataataaagtctccaattatttgagtatcaaaaataggcttgttattatttagggttttcgttggtttaaatgcaacggggtgtggttgtcattgtggaagttaatggaagtaattggttgaggttttaaacgttatcggacattttaaatgactggtttacagttaaaaatatgtgtatttacggcaattttatgattggcttagcagtagatgcgacagtggtttaaaccaaaaattaccaaataaataatatacaattattaaattatataaaacaccaatattatatatttatatatatatgaacatagttaattatcgaaaccatagacaaagtacataagagttattccgaaaaaggtttattatgaaacacaaataatcatattgggagattatgatatccaaaatggactaatcaaataattaaatccaaaatggatgaagaacttatattagttccacgcacaatataatatgttccaaactaagtaagaacacaacggtcgaggtcatgcaatgaatcatcctatatataaaatagttttactaaacaattatattttagtcactcgttaacaaacaatcaaaatcgctatataaagaactccgattggatgtaaacaaatcatcataaacttgttctcttccagaagaaactaaaaacaaaa

SEQ ID NO: 39 - DNA, 1500 bp, Arabidopsis thaliana
aaaggccatgaagtgtagcattttgagaaatggtcattccaaatctaaatggactttctcattgagttgtttgaaagaaaaataatcaattacaccaccaattgatcgaccaagaaaaatgtttggtgaataatagttaccccaatttgtgtttttttgggcaaaaaagttgccccacatttatattttaattttgatgcctaaatttcgttgatcttaattaggctttaccgcactgtcctgacagcagtcagcatgcgactagaatgtgaccaattgtttgtccgaaaccgaagattactcattccaaactcccagtctaattggaggttgtgtcttgatatctaataacttgtttgataaccattataaggtcttttgcttaggttttacggtccataagttaccaatgtatcctatgatcaggacgactactacaggttcaagattttgaagatataggtcatacactagattttcttatttataattgtatagtttagcttagtcacccgggatctctcatcgaatgaatcatcctatttaaataatagtcaagcggaaaccaataatgaaagattatattaagttatgaaaacgtcaataataatcatgattaagataagatcgaaaacaccatttggatgattagatctatctatatagtaaaataaaataaacattatcaacgaggcctggttgtggcatattaaaaacaagacattaatgagagatcagtatcgctataaaaaacgacactaacatataaatgaaccatcctaaaattgttttcttaatcaagacaaaagaaaaaaaagaaaactaaaaaccaaaatgtcattgcaaaagtttctcatggggacacgttgctcctaaccaccatcgtatacaatagttaaataattgtatactatcttagtgggataagtaaatgaaagtttattcaataaatattaatatcttaaacgtcaataatacctaaagtaagatccaaaaactcgatctggatcattactatagaaaattaaagaaaatgattaacgaggccttgttggcatatctttttcttttcttgtaaaacagatctggttgcatatttgaaagtagacattaaatgagagatccgttgattatggaagattaaatgttaatattgttgctcgttaaacacgagaaatattattaagacgaaggtgcaacaatagatgaagacttcagttataggacatacacgatttttttttttttttgataggatacactatttatttaaaggcacgtttttattatatgttccacgcgtaatataatatgttccaaactttgaaaaataagtaagaacacacctatatataaaataaatttattaaacaaatatagtttagtgacttatttgtcattaccaaacaatcaaaatcactatataattaagaactctgtttagatgtaaacaaatcatcacaaacttgttctcttccaaaagaccaaaaattagaaacc

SEQ ID NO: 40 - DNA, 1500 bp, Arabidopsis thaliana
tctaaagcatctagaaatggaatccaaataatccagggaacggaaataaaggatatttcaatggataatgtctatatacacatttatctacaaaatttggtgaaatagggtttttaaaaataaaacccccaatgttacctctttttgtcgaaaatttaattaacaggattttgaatttatttaacagaaattttaacaaaaacaatatttcctaatttatttacaataattcagataaatttatggttttcaaattctctagttgaaatggagtcttctcctacacccttaaggccttaattaaaacctgtttttgcttttttcaatctcattatctgaaacttgaacaataaaacactcatctctttccgccggtgaagtgtttcgtggctaacaatataccaattcaacgtaagcttaacaaacaatatactatttaaacgtaatatttacaatattactttttcaaaaatacactttttgtatctaggaattctaaactttttagaatctaaaatctaaattcttttagaaaatgaattctaaaagtagaaaacattttttagtgtttttagaacatatagataaccgtaaattacattttcacacaaaaatgagaaaattaaagaaagtgtagaatacatggaaatttgttatccagttttctaaatttgttagaaagaaaaactagtctaaatatcgtggaacatgcacaaaagtttagaataaaaactctaatattagtagaacactaaaaaacattttctaacaaatttagacaaaaatttctctataaaagaatcatcttttgtcaactaaatcaccaattgctttttttacaaatcaattatattcttgtacatctacatgttttttttttctttttttaaaaaaaatcgtacatatatatagtattctgcaattacgcaaccttttagtataaaaataaacttttaaattaaatttcaaatattttaatgatttatatatacaaacaaaattgcatttatgtcattcataaatttagacataagtttacggttttactgtaaaatgtctataatatataaacgaatttatggatgttgaaatgcatatgttatatgtttgtatatattgtttaattttcaattggaaacacattggatttaataaataaaaaacattatatagaagttttcttatttaaaacttcgagatatttaagtttggtaagtttacttaataagaataaatagtatatgaattaaaagtgcttggcaagttgaatagtagaattaattaaatgaaaaataaaaaatgatgataataattgtgtgtttgacacataagtattagagtgtggggttccaacctaaaatcaattggcaataggtggagagactcatatcttatatataccacttaagatcctactcaacttccgatgtgggacattgtccctaatacgccccctcgagttgttggctcttctagccattgatctcgatttgtttgggc

SEQ ID NO: 41 - DNA, 1082 bp, Arabidopsis thaliana
tattaagtttggaattgaggtctgtcttttctcttaaacctttcaaatatcaaatctacttttatatatcttgtaagtgaaggaaattatattttagcaaacatatctaagatattcacatcacttatagcactgtctctttgctgctatatcatagcttagttctttctcttatagcactgtctctagctgctagactcattggctttagtttttgctgaaataaactcgtatttttagaataaatgatttgtcgagaccacaacatcacgaccaagcaatgtgacaatagggtcttgtggtgcatttaggttcagggcatatgttcttatccccagctgctatatacattctgaaatacattgatatcaacacaattggccactaacaatagggtcttcgcagataaacaagaaacgcaaaagagtagagatagtaagaacaagaccttagaactagagaatagtgagcctaaggacatgtcaattagtcttcacttacatggcagatgatgacttgagcaggctgattcgaggttcttctctttttttattgcaaactctaaattgtagaaattaaaaacacaaaacggggccagagtctttgttaatggagtgtttttagtgaatgtttagttcggtttagctctaaaccaagttattacaactctcataatgaaattagggacggatacaatgatcatccagaaaaaggaaaattgcaaaaacaggaaagtaaatcaaaaccaggctggagattctcttttctttaatccttctcagacacagcttagaaacagaacataattaaccgtttctctcgttaaaacctctaatttaataataaaatctttaattaatataatgacaacgcaccaaccaactagtgattagagtttgagacagagaggatttgtgtattatggccccacattctctgtgccatgttttactatttaaccaactctttagcttcctctgctcttccatcaatcggtcttttttccttgatttttctggtgagattctacaaaaccaaaagattccttcgccggaataatcaccggagataaagataaagga

SEQ ID NO: 42 - DNA, 513 bp, Arabidopsis thaliana
acatacgtaaagaaagcctctaagtaaatccacacgacaatacatggaatggcttgaagaaaccaacatacagatttataccctcgttaagttttcttagactttgtgttttgagtaggtcgtagagattcttcacagaaactttcatgcttttattagtttacatcatatattcatattctcgtttctatcttttagtattaccacaatgaaagtttgaacttcttttagtcaacgagacccaatgattaagacattaagtgattagaaaatgagagagaccacccaaccatcgtctaacgaatctctcgggacattgaccaaagacatcaaccacagtttgaccacgacacttctgacgtgtaacaccatgtattaagccatttcaccaaccatatcacaagggtttcaatatcatcaacgcaagtaatataacttaactatctctcctctcttttcttaagataacttctctctctctattcgttggtataaaaaaagaagactaggaaa

SEQ ID NO: 43 - DNA, 1374 bp, Arabidopsis thaliana
aggtaaaatcgagatcatcagtctcgggttagaatctgtgtgtttgccgcagaagaaagcgttgcgatttgctttatagagtagagttagattgtaatgcagcatgtggaatgttgctattcatatggatggattggattctctgtagtttttgtataaacatcctctcaagtatttgttaattatattagatcatcatttctcttaacatcatttctcaaaacgtagtaaataggagatttgccaagtgaaaaatatatataatgagacagttattatgaacacaatttacagttattatgaacacaattttggtgatgtttctactgtttatgaaatttgttttttttttacctgccagtcttatctatttaccaccaacatacattagaatctaatgtaattacatcatcaatcatagacctaatgaatatttaataccacacagtttaaacattgctattaaagtttgaaaagattatactttagtaaaataatgttttcaaacttgtattcaattatactacgtaagcagtgatccaattattacgaacataatacttttccaaatcaaaattttgtttttgttcaccttaccatcagtttcataatagaaatattatatatatacaacacaaacaaatgaatgagaaatgtttttctattattatgacttttatttaagattcaaaagaacgaagaagagttatatatatgcttgtttgtttcaggacaaatacaataaataagtggataagaaaaccacaaaccttgaactagcctgaaatgtgtgaaccaagctatacaaactgtacgtgattctgcatcgtggaagtcacaaacagacttctgacttctgctaaagataaaactcactgtttgcaagcaatttagtataaatatgaagcattgacctaccaagatcgacacaatcacacagtttcttcttaattaaacctagctatttttgtctttagcagaagtcagaaagctagttttgtatttttcttatttcttaaatatatggcggccaatgttatgagacggtttatgatttaaagatacacaatggacccacgacgttgccataatacaattattaggctgttaattaacgtgatcgttacgtttgctacaaactagctagttgtgtcttctttattttagtagataagtataagagtctaaaaatattacaacccaatattggttacattcttggtcgacttagaagttgaaagttgatttcttaaaaaaaaaacacgcacaataaataaaccaccaattcacaaatacaagaaatttaataacctcgagcagttaccctatttaaatcccctctagccttgttttccttctcacacctttgaagca

SEQ ID NO: 44 - DNA, 781 bp, Arabidopsis thaliana
tttgtgtggtttctttcttctcaatacatgttttgatcagtttaagaccccacgctcgccggaaaaaacaggtgagatctttcttgtaatcatttattgaaaaatgttaagagttaatatcatacttgtaatcgaaattagattcagttttattactctgttttctttttcttggtttgtgctttttcttttggtgccttagttcattattcatttggaagagttcaagatgaggaaaaagaaaagaatatgttgaattgtactctttatagatagtcttggaatgaaaagtttaatttaaataatcaagtagttgtcaaataatcattattattattattgtattttatcatgttggtggtttactttaatgggaacagatgagcatttcaaaatagatatttaaaaaatatcacactaatttatacattgaaaaatgacatttaaggatatttcccatttttcatacgtagtcaatcatataaaatttgagattttaatacgttgacagagtcatatgatcctggatcgtgtcaaaatgtctcttgtgtaagtaagaagttttccaaaagaaaaaacagttatgagctgtcttgatgattatatcactgcctagttgttaattaaggaagagctcatctgtttggttgcgtgcacgaagaagagagtatggtacaactactttcacacaaatctataaatacacagacacactcataagttcaaaaaacacaagataaatatattttctattcaattttgtttaaagaataagaaaaag

SEQ ID NO: 45 - DNA, 1500 bp, Arabidopsis thaliana
tcaaaataagtacatactaataactttagacttgaatatccaaattttttacttaaatacccgcaatataagtacatacacaaatttttatccaaaacctagatatttacctaaattgttttgggtatttgcataatattttgagtatttgtgtacccaaaaagaatcgggtatttaatagttctaaaaatttatacatgattttatccaaactcgaaaaaaccaaaaccgaaatcgaaccatttttatatttaccctattagattttattttatttttcctatttcgaacacggatggttcctaccagattctgccttaagtttcttaatgttcagacctattgataagtatgagttttatcaatgagcatcaagtaaaagtgagagtcttgattctgctttaaaacatgaaagtttttattgtataatatggatatcgaagaggctaattcagatatataaaattacataatacctgatttattagagtccatataatttacataagactttccacgactaatttatatagttaaaatactatatttgattatatttttagtaatcgtgaagactcgttcatatattgatatacagtataacctctataaattaatactctataaattaataacctctataaattaataaattattccggtcccgagttaggaccagtgtaaaaaatgacataaatcgataaattaataagataatattttttttgaaaattctatgtaaatctatggtcctatcaatatcataaattaataattgtataaatgtatcaactatatatatatatatattatgtaaaaaaattctttataatatatttattatacttttttgcttaaattcatatttgttctttgtttgattatagttttcatattttactgtatcaaaacttttagtgttgttttctaaacattattattatgttttacttgtaactggttctagaaatatatattctataatgaaggaaatcttatagaaatttttaaaatgttaccaaattaggaaaatctctctataaattgataaatattaatttatcgataaattaatacctctctaaattaataaaatttcgcagtcccaacattactaatttatagaggttttactgtaattcgtaaattttgagagtagtttttaaccaacttacaccaccaatttattagagtacatattttgattttcgatatgcaatcttgttaccaactaagttgttaattaggaccatatactcttagaaattgccagctaatttgaacaatgaccatttaagtgttttcaaataatcaaatcatatccatataaaaataattagtcaactaatcccctattaagaaaaatcaacaattaaaacattctcatttcctcctaatgaataccctataaatacccactaaaaacttacatttctcccaccaatctcaaagcaaattaaatacactactacttcttgagcttttaaactacacaaacaa

SEQ ID NO: 46 - DNA, 724 bp, Arabidopsis thaliana
tgagaaaactgtacggccggccaaggtcacgccactctctcgaccaatggttttataatcacgaggctgactcagctaatctcccgcaaatcacgagatttttcaatgttactcaattacaaaactattaacaaactcaatactacaaaatcatgtaatgtgattatttagttagtcggatcactaattaatgttttttcatgcattatactatggtcaacgacgttggagaagtaatcttgaatacttgacaacaaccaaaatattgattctttttttcctctcataaaaaaataatgtttaacaattgtaaatgatctaatatattatctccattcttgttagattaatttaatttatctctgttcatattttcacaatctaaatttcttttaaaaataatataaatgaaaacaaattagttagttaccaaagaaacagagacaaaacagatatttaaatatacatcccgccataaatatgatataagctatagttggttttcagctgcaacgtctcagtcacttctaatataataaaactcaaaatctcaaattgattaaacaaactggcttacgcagaagagtgacgactacgaaaatgtatcatgcctttaaaaaccttctctactcctttaatcggttccttcttctttcccttttctcctttcgcttcacgacgacgaaatttttgatttcagtcaccgttattgttgttgcggtgg

SEQ ID NO: 47 - DNA, 1500 bp, Arabidopsis thaliana
tgcaataaataaagactaaagagccttgaattgatgaagaaagattatgagtgtgtcatgagtgtttgtttactatcgtgaaagttgttagttttgaaaagtgacgaaataactcaaaatttatgctaagatttatggacgttcaatccaaaatcatcaattgacaatgaatgaatcaatattccaaaatcaaagagaacttacatatgtattagaaaacatgtgtttttactttttaacaagacaatatatataggccttgtctttcagccgcaaatatttaagaacccaatttttatctcattgtatagagattaattttattttacatctttaaattgacaagatctaattcataactaattcttactatatggaataacgggagttagtggagtcgaactccagtaaaaaagacagagtcaaatggactataccgtatttcatgtatgttagttattaataacactaaaaagcaggtgagatatgatttgataaataagcatcgtcttcaatttctgccttcatatatttaagaaccatttcgtatcatattgtattgagagattcaatgaagcatttataccaagataagaaaaagatatgtaacttgtacgatttagctcgcttggcctccaaaaaggaagaaaagtcagcacaaattatggctcaaataaagattttgtcaactaaataatgcgaataaaagattctctcaagattattaatcatcattgtattaaccaaagggccaaggccatgtgattattatactgtaataatcaaagtagaagactataggcataatgatgaaaatcaaagtgttaagtgataaatcaggttttgagttgtttttgacgacaggtttagttgttttaactattcatttttatttctaatatttttagatactattgcatgttttaatattttcatatcaatatatatatatatatatatatatatatagatatatcatatttgatgaaaaaagaaaaaaaaagatatatcatatttagcgtgaaacactatatattctttactgtcattttatctttttcttatttcaatcgttttgtaacgttgcatttaaggttgaaatggtaacattaggtgataaaaaagaggaaatggtaacaataattatatgtttaagtacaatagaaatgtatgtcagcaacaacctactaccacttaacaaaatcatatatacaagcagagacttagaaacagggaaaacgaaagttacaacgtacgtcgtgtatcataattcaaataattaaaagattagataattttatgaacaattcaaataattctacggctgagtcataatgatgggatcggaatcacatgaaacacaactaatcataaaattagtttaatgaataaaattcttaatgtcttcaacagttgctcaaccattccactacaaatagaggctaaagaactaagtagagtattcactcctctctcaaaaccctaactgtttccaagata

SEQ ID NO: 48 - DNA, 1500 bp, Arabidopsis thaliana
ttgaaaccttacaaaataaatattttaatattccatattgcccccttacttttggttacataaattaaaaagataataacgtgtatcaataatcacgcttgcatattaaattaacatatccaagttaatttaatttaagttgtttggcatcgtgtgccaactttttataataattggacgcagaaaattaattaaaagaaataaaccctactgcccctttctacaccttcctctttcgccctaagtttttttttggtagatacgagaagctctataatggtattgatacaacatgcaagtagaaagcaatcaactatatgtgaataatcagtttttagtttagtcttttttcatctaaactccttcaatttcgcaatgactattagtatgtacttttagtttcaatatcagaagttttgttatcggtcaaaaacaaaaatctaaccttttggtcggcaaaccaaaatctttattaaagctggggagcaaagaaaagaagttggtcaaacctaatgtagcaagggaatcagccaaataattggcttctctcgataaaccaaaaataaattatactatgaagagaagagaagacgacgtaaattttgttatattggatctagaacgaatcatcataaaagattagctagtgactattgactccgagtccgattctactccttatgcacttatgattgatgaagcgtggagcttctagtatcgtcatgccttctcccaccaatgcttgtacaagagaatgaaacaacaatttttaggcctcattcacggacttgtggcacccggccgcatacacgtgtggcggaggcatatcttggtaacacaggttggccaaatggtaaataccaagtctgatgacaatgtacattatgccagagatatgtctccttgtggcggaggcataaattattccagtaactttctggaaataaaagtgatatttaaaatctttgaaacttagattaatctccaaaatctataatgttatctagaatatcatggttgaaaaggaaataattattactaataaaaagattaaccatttttattattgtcgatcaattagttagtatatgatatatatgctgtttagggaaactcaagttttgagttagttgtgtgactctgcatgttgtcctgtccaaagattaatatgctagcttctatagaattttaactgattgtaaaatattccaagatccatctagttgccgaaaaatgtgacaagttcatgtagaggtcaaacacatcttcaattgacgtacgatcttacgcagaccgcatataacacatccttcggtggcttatatataactatatgtaatttaaatgtaacgtagtggacgtaccccactagcatataatattgtcaccaccaccgatcgtatcatatatacactttatattctctattgcacatatcctagactcttaaccaacaaaataaaagaaagaagcaaaaagaaaaagctcgaagataggaaa

SEQ ID NO: 49 - DNA, 1500 bp, Arabidopsis thaliana
ctaattcatatttctatatctaaaaaatattacattttatttttcttaaccaaaagaaaaattaacaaaaaaaaatcttattatataaagtatggttttcaaagttactaactcataagattatgccacgtgtcaagtctatcgatcagatgttgccatgtgtaagaaaataattaactttttttttcttaaagaaatagaaaactaaaaaaagaaatcattattaacatatactaattcatatttctatatctaaaaaatattacattttatttttcttaaccaaaaggaaaattaacaaaaaaaatatatatatatgttattggtcaaatttttaatatatatggtattaacaaatgttttattattatgaattatgttttgttaattgttaataatttttaatattaactaagatatagatttgatcttatatatatatattattttgacatgaaatcattatcaagtaagatgaagtgtgtaaaattaaaatcgagttttttatgaaatagatagttaaattgtattacaaaattgaatgtataagataaaattttattttttcttctaaatactaaattacaataaagaaaaagtgtgtaggttactatttagacttcaaatatcatcaataagattgaatgggtataacaaaataaaatgcataagactactattttactattcaaaattatatatcaagaacaatgtatcgattattattttactattcaaaatttaaatattatcgataaaaattacatttacatcttatacccgcttatttaggcgggctttatctagtaagaaataaaatatagataaccaagatatattgtaacaacgttacatgtcacgcatccatacagacagagacactatacgttacgaatttcaaatgtaacaacgagtatttcatttttcttctatattcgatctcaattattagattcttttttgtagtaaatataaaaacaatgatttcgattgagacaaaactacaagtgaggctgatgtgaacggggaaaaatagtgacggcttatatctgaattacgttgtgaacacgtttgtttatttcatggattgctaaactttgaggatatagcaaagaagctttaagtaaagcaaacgaatcattaatattttgggaggatgtatatatatatttgaagttgttgttgttgttgtttaaaactgttttttttttcttgtaattaaggttgaaacagattttgaaaggacgttagcacgaaactcgtaaataacgcatgtttattttccaaataaatttataataaaactattatacatcaaaacccattaattcatcatagtggcaaccaagtgtatatagcaatataaatcaattttttaaaaacaacttctgtaaacaaaaggaaagtgaaggcactatgtgtccccacacggttccattgagacaaaggcatatgtatgtgtgtgagtataaatataagttggttcttgaacatcattgc

SEQ ID NO: 50 - DNA, 1018 bp, Arabidopsis thaliana
aaattaaataaacataagttatgcctttgtggctacaaatcataaggtatttctattcatcgagtcgggccctaaaactttagaaaaacaatttttataaacgccaaaccgatcggttcaaacgcaaaccgatcggtttcatactttggtaccaaccggtacaaatatctaacaggtgggtattgatcttcaatcatttattatcttgatgcattcttctccacttctactcttaaattccttttttactccaacaggtccaaaaatctgtaaagaatcaaaccatcgtaaaaagtaattagttctcgacaaaaaaaactcaagacacggtactaaaaatgactcttaaaatctgttaatactaaggcgtataatgtttgtttttcttccgcatgaggaggagaagacctgagaaaaaagagtgaagattgtaccgatgtcgtgagagatgccagcgaagaggatgaaaaatatcactttttctccattgtaaaagaatgagaaaaagttatttgaaaaaacgtgagaagtttttcttttaagatttgtaacaatcttaacattgaaagttatcaatcaataaccatgcaaaatattaggtagcattaatgataatgagatactccctccgtctaagatgaattttttagaaatttcacattttttaagaaatattgtataaagacaattttaacctcttaatatactttaaaaataataataaatatttatctaattatttagtaaaggtaaaacgggaaaacaattttaaaatttgcttggaaatataaaacttctatctttgtaggacaaatatttttcctagaaaatttatctttatgggacagagggagtatttatctttattaatgatacaagattaccaacgtacgtcttgatgaacaaagtcttattatataaaaatattacatatcgtataaaatacccaggaccgatatatgtgttataaatttgacggtgaatatggattcagtatcattttgtagtggaccaaaaa

SEQ ID NO: 51 - DNA, 1500 bp, Arabidopsis thaliana
aaactgagtttgaatatctctctgaatcgctttagattgagatatttaataataataataataatagtaaaattcaagtctgagatgtaccatctattagacatgaaaaaatggatttgttggttacatactttacaattgtagaaacgaaatatatgatgtttttgatacaattaatcggcgatatagcgaaaagaatgtggtaatagcgataatgtagacacaaaattgttgcaaaaaatgaaaatgtcaatgaaacaatatttacgcattgttaaaacttcaatcaaacaaacaaaatatcaaacaagattttctttgtttcaccaaaatattaattttaacaaattgcatgtacgcttaaaactttaatatacttattaaatcttacttttatggtccttcatatgtaacataataacagtaaaagattacagaaacgtttaaacctaacaattatgaaagtttagaaagtgttataaatcttaaatgaggaaatggagcagctcaatgcactcaaacaagtgcccttgattagttgatttcaatcacccaagtagataagcacgtaaacataaatctggttattgccaatatgaaaaagaatgaattttcggatctgactcagatacgttttgcaagaataaagttaggcatttttgaaaaaacaagaaagccgtttaaatagttcttatgtaatactctacccaaagaaaagaaaaagtaactatggaacaaaattcccatatagaccaaaataaaatatatcataccataaatgattgaacagatcttattttcttgaaagtataaatatttttaatgcttggtgtgaaccgcaaatttttgtccccaacatttttagtggataaatatttctatgttgcaatttattatcattttctatataaataatatttccagcttgtatatgctatattctcgttactaaaaatatatgtatatatgtatgtacatatatgtatatttaaaaagttattattaaaaatctactactttctcaactatactctctttacaagaaaatctgattttaaagaaatttcacagttttctagatagcaaccaactttcaccaatcatacttaatttgaggaattacttacaaataaaatctttaaatcataaactagaagccacagtatctgttaaaataataataattgaatttaggatctgccattagtcgacgaattttcttgaatattcgtttctatgttaaaatggaagtcgtgttaagaatacaaataaataagtatttgttttgaaaaagctgaaaacatttttgaaaaagtaaaaatgactaaataaaataacaagaataagaattatcgttttaactccactgcttcctctgcagcactataaatatatgctttggtactctgcaaggttgttccgtatctctcaccaaaccatctctttctctctttctcaaaatcttgtttatttttttaataaaacaaagaaattaaaaag

SEQ ID NO: 52 - DNA, 1048 bp, Arabidopsis thaliana
aaactaaaaagccattacaaatgctttatctttatgtgctattgaactagaatcatatatccatcttttgacaatccaacaaatatgttacctttattaatttggtcttgatttgaataatggagctataattaattcatgtagttgaaaagttcaaaatacgaaaagaaaacaatatgactagctaaatttatttattagaagttgtagcagaatataaaattgaccacattagtacaactgtgcacgttctatgctactagctagcatagtcaattacaagcaagtaaacatatttttaatgaccttttcacaaattttcgaaaccaaaaccagacaacacttgataatattatttgttgtaaagctacaaaacgtgagaatcccgatagctatctttaatacattgacttctaaccggttaaaaaagaaaagacatttggtatgtaccaccaacgagtcaccctaactaaaactataattgggaactccgatcaatttttcaaatccctctagtcatcttaaaatataaatttatttcactctataattgagcataatgcaagttcgttctgatccaaaaaaaaaaaaaagttccggtcgttttataaggcaagtcaactttgatagaaactaatttagcattcgcataactgtacattttctttaaaatacacattaaaaaggtttaaacgttcaagtctttattttttatgatgaggatatagacggtctttgatttcaaatattcgaaagtcatttttttttaatatcacaagttgtagtattttaaacaatatgttatttcttgacatagagattatttagtcttttatttatatgtgtttactattcttcttccttttatataatcatataacctatagtttgtatatttcaattattccatcatttaccatttagtcgtctccttggtgatccaaatacttcatcaccttccttcatagcaaattctcttcgtctatctcatacatttaaacaaacatagacatacactaatcaatcttccttaatttaaaaaaaaaac

SEQ ID NO: 53 - DNA, 1500 bp, Arabidopsis thaliana
aaataaaaatattgtcatgcggtataccgcgaattaaatcctaatagaatactataatcaatgactaattaacacatcaaatgaatttgcttcatttacatatctttaataaacgttactataatattttatattaaaatttatatattaaatatattgtgtctaatattaaatatgattgaattttatgactacattttttaatgatatattaacgtatttgcaacttacagtatgtgtaccccgaataatagcatacgatattttccattgtagattacgaaattctcatatttaaactcttctcttgtgggtagggttgtcaaaatgggtcaaaattcatcggtcaactcaactcaattcatgaaccctaatgagttgaaaatattgactcaaatgagttgatgggtcaaatgagttcttgaatcaattagtttgatgaataaaatgagttgagttgtaatggttaatagtttcaatggtttatccaattaacccatcaaattttgtaaaattgaattaaaccaactaaaatctctaaaccaataccaatttaagtttaaccaacacatctaaactaatttaataaaatcaatattttccaaatttcttaaatatacaagctatgaaattgaaaagaagtaaactcgtaattttccaccaaaaaacataaacccgtaattttcccgccaaaaaagtaaacccgtgattttcccgccaaaaacgaaaaactcgtgattttcccgccaaaaatgtaaacccgtaattttcccgccaaaaaacgtaaacccctaattttcccgcaaaaaaagtaaacccatgattttcccgccgaaaacgtaaacccctaattttcccgctaaaaacataaacccgtgattttctcgccaaaaacgtaaatccgtaattttcccgccaaaaatgttaacccgtaatttaccggccaaaaaacttaaacccatgattttcctgctaaaaacgtaaacccgtaattttcccgccaaaaaagtaaacccgtgatttttccgacaaaaacgtaaatctgtaatttttctgccaaaaacgtaaatccatgattttcccgccaaaaacgtaaaccccgtaaaaaatggaatccgaaaatatcttaagtttgatgataataaattaataattattaataatgataattatttattattgttttataataataattaattaaattattacgtgggataatctatttaacaactcaactcatcaaatgaaatgagttatgggttgggtcaacccatttaatcatttaactctaaactgatttgataatgagttgagttgagttgggttacccatttcgacactcctacttgtgggcttagtgaaatttcgtagatgtatatgtaagcccgttaaacattgtatacgatttaataaaacagaacgacttgtttcgtgcactcctcccactatcttaaatctaaaaaaaaaaactttggatctcatctctcgttgacttcg

SEQ ID NO: 54 - DNA, 1500 bp, Arabidopsis thaliana
gtttttaaaattgttgttagtgtttatattttgtgattgaacgaaattatattttggtttaggttgataaaataatggttagggtttagattttgtgattaaaacgaaattatattttggtttgggttgataaaatagtggttagggtttagattttgtgattaaaacgaaattatatttcggtttgggttgataaaatagcggttagagtttagattttgtgaacaaaattatattttggtttgggttgataaaatagtggttaagatttagattttgtgatttaatttatttattctttaaatagatgatatgtcaaattctaattggtaaattaaaaaggatgaatgaatggattcatccctaaggggtgaacccaagaattgtttttttcttcgtatgtgtgaaaacactaaaaaataaattatttggaaacagtaggagtaagaaacaaattagagaaaatgacccctatcatctagtagattggagaaactataccaatcttcaggaagctatatcattggaattagctctgtacaatctatttcaaacatatgacaatctattaaaaaatattcttcatgacctatattacactttcgatctccgaatcaaaaagaagttgaactcctccgttgagacattgcccctaatagcattatttatgcgtttacttgcaatcaaatccaacctaaaccactaatgaaatcagaaaacttccagaagccatcaatttgacatgtagttacttcgaaaataccattctaaaacataaatagtaagctggatcaacttgacccacatgtatctatgagaaaatatctcatcaacgactaaaggtaagggttatagtattatatatgctgtagttgggttgagaatcagtagaaactgatacggtcgcatatgatgattgttagaaatttttgatttgttaaatttaatggatgtgaactttaaaacactctagaaaagataaataagctttttttttaatggagttttcgatctcaatcctttcaattaatatgtgtagtagaatatcatgtaaataataaaaatacaaaagaccgtttagataatatacataaagacttttgaagcagagtaaattaaaatatatcaaccaaccttagctgtgtattaatttttaatttctcaaccgtacatagctgtatctgtttttgtcaaccagctaagcttagctgtatataaaatgtcaaaacaatgattttccttacgctaccgtaataatagcatttagctcataatttaaatatttgccactgaaactaaatgactcaataaacaattacgactaggtcatatcattatgaaatatatcgtaaagaaattgcaaatgggagttggaaagcttgaaaattaaaacggggacagcacatttcagtggtttgtttttatatatagacatatgtaaaggcaaaaggataccaatatcccaaaaccctctcataaacaatctccggttgagcagcc

SEQ ID NO: 55 - DNA, 1500 bp, Arabidopsis thaliana
gtattcctagagaaatctatacgtacaaacataatgggccgggtatgctaggaattcaaaaataccacaatcaaaaatacatttcaacgtacgttatgatagtcagccaaaatctcaagtcactgaaaattggctggccggttttgttgcaacaactacctaaccaacgatgaatattcactggaaaatcaaccgtaaaacactagtacgaagaaggctagtgcaatttggcattatattagaaacacaagacctcgtgtggactggtctcatttggtgtggataaaacagatagttcggattcttaaacacaaactggttctgcattaacaagaggccggccaaatgtcaaagatagattattaggatgagagacatcctttggatcccacttgcttcctctcgtttatttttaaattttacaattgagccctacaccagttccaacctatgaataatactgacgttgatcatacgaagagatcggcggaacacatgaaattattcaaccactttgttcgcactatgttgaatttttttgtttacttcattaaaatatacacatgtgtatgttatactaatacaatgtacctataaaataaaagaggatgaacgtgagccgtttactctaaagtctatgtgctatataagaatacaaacaaagccattgacttcgatatgtcaactttatgctaagtcaaccaaatgatgagttggacaaacctatcaagtgccaattcttcttcttaaataatcaagttcccttcaaataatcgaccattttaattagaagatattaattggccaataatatttagaaatataacatcaaacaacaaagctgacataatgtgtaattgtgtatataaggtatttttcacttattttatcaatgaatttttataatgatattcttatgcatgaatatattttaacaaggtatattttttttagtaatttttaacacaataattattgaatcacggtaaacaccactaaaaaggaaagataggtagatgtgatttagactcatatagtaggatctatatccatgagtgccgtatattttcatatatttatatttaatgaattcacaattgtctaaactgattattaaattactacataaactaggctaacatggcatacgtacgtactcacaaaatattttagattcacgtagctagcattaggtcgggaacaatttgttatatataaccaattaaatgtgacggattagttggttaatctgttgaaattaacaatgatctaaaaccaaaatcgagatatatgcacgctgatgcatataatatgagctgtggaatctcaataagtcaagtcaactaaatattatatgagctgtgcaatctcaataagtcaagtcaacaatatgaacatatactttaattatatcatctatctattgcgtattttgtttttcttttttgcttctatatattacgtatgtgtgcatgtaagtaagttaaatttagctcgttg

SEQ ID NO: 56 - DNA, 1500 bp, Arabidopsis thaliana
cgtaaatttcgaaacttcaaagttcgtttgggttcccgcttgtattttttactgtttgtgttaggttataagttgattttcttgttatttttagtctttttctcctatttatatctctacgtaaattttgtcgaaattttgaaaaatgataaagatgataaagatatgtaacataaaaaaaaaagtgttaagaaatttggaatctaagtgtagataaatttcaagtttatatttggaaaaataactgtacttgtaaatatgaggattgcaaaatctgtctactgttcatgcatgcttacttttttaattaactgaaaattatcgctacacatatcttgttcaaatgttttcgctttacttttacgatgaacaacactaaattttattaactgatcttacaaaacttcgataataaaaatgtagttacggcgactagtcatcacacaataattttagcactactttcaattcatccaaaataaaaatcgtaactaaattgtgtcttaaaatttacgattaagaaatcaggaccctagctagagtcaagactatagtatatatgtaccaacttcttttattttttgtcgatcatcattaggcatgacggattaacaaattaaatcagtagttttaagtcttcactattcatggatagataatttgacgactaaagtaatcagttatcgaacctctggagtctggatagatgactacataaggagtaatggagcaattgttgcaatgtccacatattgaggtatacttaattggttatttgtcttaatgaaaaacaaattcagaataatagtttatactagtaaaattaaaatattgcctcaactaataagttgttatgtgcaattatatgtggttgttgtacatgtgaccttaactaatattaagtgaaacatatacatttaaagataacgcctaaaatttaaatccttttttttttcttaagtccttcaaaacattctcaatcatatgaaagtcttgtcaatcaataaaagtccaaaatatatattttagacccaagtggttttagagaaaccccgttttaaatcattactatttcattaaatttacttttacatttctttcagaatcgaagttgccaagaatatgaaatcgaaatagattacgatgaacattaattacaattctactattatcgacttagctggatcaaattttcagcatatcatatacattgtaatataaattattaaatttacaacatggcaaggaatgttacaaagtcgctcgcacgcctttgcaaacgtttctaagcttaattatctaaacaaataatataacctttgactttcgcatgtattcggcatgtatatgaggtgtattaatcttttaggctctaaattatatttcgaaggttggctttatatatatatatataccttttggttgatcttaattgactcacaaccctttccacaagaagttataaaccaatacatagaagaaaacttccaaccccaaaaa

SEQ ID NO: 57 - DNA, 980 bp, Arabidopsis thaliana
taaaagtatagcattagatgttgaaaaaaaaagatttaactgtagtaataagcaagaagacagtgagccgcccttcaagcacggcgatgcatgtggcagattgattcttaggaattgactcaactttttcggtcttcgttgactatatattcaaaaattgtaaaatttaactcaaacaaaaatgtacctcttgttacatgtattttgttcctctaaacagtatgaaaaaaaaactaagttttctttttaatacgaatgattgcacctaaaattgatagacgactaagtctatactgttacattagatggaaacatggtttgaattatcgctgctaggtgtccaactcgaactttgtacagttgtactaggattctaatgatttgctatgcatataaaatacattgtggcgcaaaaattacgctagtcgcatatttcttttttggtttgtttgtttgattgcataattatgatttgatgtcaaaagttgtataatcagaatttgagaacaataatatgacaaagaaaaacattcttttttctcagttagatgtagatacaagctttgtccaaaaaaaaaaaaagatatagataataaaaataatatcaaaggttgccttctggctacgaagactatgaatttagacttacaattccttggctaaaaactgttttaaatctaaattaataccatatgtcaataaaatatatcacgacttcaattactaagttagaacttagaagtggtgatcgagttccagcaagctccagcacgaattgaagaatacaatttgtgcatatatatattttacattatggttaaatattataatgtaacaaaaatcaactcttcgaactcgagtcaacgtaaattttggctataaattagtcaacattccatgtcttgcattcgaacatcttagccttaatcattttcatcttactctttaaagcgtaacgattaattaacgggaagatttat

SEQ ID NO: 58 - DNA, 758 bp, Arabidopsis thaliana
caacttttatttagctaaaaagtttggtctagaggattttcattgtatccatgaatgcttgtgaatttgttaagcaatgatttgtacttctcttaatcattccacattatatcactgatataccacaaattcgtggtcttacattttgaaaatgttaatacattacttttgacaattcctttaacattcacgaaggattcatgttttagcacaaaaacttgaaaactaaatgatataaagtagcttaatgatttaaccattcaacaacaccccaaagttctttttaaaggaaacaacgacttcgctaaactaccaggttaaggaaatgtatatagctaagaactcaagccaaatgcgtaaattcttcatcttacgcctcatggtttagcttctataataaacaaacattttgagtcatcctcaggcaattagcttgcaaagaaaatgatataatacgtctaattgctcccataattttacaatttagttgaccaacgtagcttgatttaactaaaacaactgtctgtcaccaaacaaaccaagacggacgagtagatttgtatgtaatctctatacgaatccgtttgtgaacttcatttgcgtcaccgccttgtcacattcgtgtggacgcctcacgacgtgtttgcataatgttattgaaacagaaatattgcactcttcctccattttttgattacttccatttcctcaaaagtcaaagctctctttcttctctctcgagatttgctaacttgtca

SEQ ID NO: 59 - DNA, 645 bp, Arabidopsis thaliana
tatacatgttttggactaaaaaagctatctatatatatatagcctatataggtgaagtttgtatagtaatggcgagaacatggttcagtattagccattattagtaaactgacactaataatgttaaaatatcgtcctatactcgtgtctaactgcatgtctatcatgtacattaagatcaagactaatataaaactcacaaatcaatatactacttaagaaaaagaaaaaaatctggttcttttttattcatgcacacacatagtataagttaaaaaatgaccatattaatttgtaaactgaccaatcgtgtatataaaaggacaccttctctacctacttatatattatacatcatttctctacattgttcaccagctctctccatctctctactccaagcataagaggtaatctctcaatagtttgaaacaaccttttgtaaaacgtattgtaacttacttaaaattgtagaacgtgagaaatatcttaaatgtttaaagtcttcctttttcacccaagaactgaaaatgattttgcatatatattttctcaagtgggtataatggatataaagaaattatacaatgactaaggaacaaaataaaatctcttttattgaataatgatttgaatcagttctcg

SEQ ID NO: 60 - DNA, 1500 bp, Arabidopsis thaliana
ccctagctacgtgtttttgtggtgcagctgattagtgcatatagctaaataaataactaaacaatgagatgaccgatgaagtgttcaacgtcggtcggtgcaagtgaacgcgcctcccgaaatgccacgtgtgtgttgttttgttgatcaaagaacatgggcgtgtgtgtgatctctttgaccagtcaaatcactcatactttggctctttcctcatacacacaaaatgtatacgtatattaattaaaagaattactgtgtgtagattttttcttatacgtcgtcatgtatttttttttaaatttggtcatgtatgtttttttatattctgcattaaaaataccaaaacaattcttatattagagtagagggagtaaaaaaacaaaaaaaaaacaaaacaaattgaattacattgttaattaaatttctttttttttttttatataactaattggctgttcggtcatttttgataaaaacacactaaaaatgcttcagaaataatctgattttgtattgtataaaatgccggtttaaataacaatgccataaattataagttggtagtattaaaaaaaatataaggtacatgtgtggacgtggtagagcgaaatcccaaacatcgataatgtgaatttgtatcttaatagaagttgtgtttcgaggaatggataggcttattttacttataaagtatatataaaaaaaaaagtataatactttcttccttgataaacgcacaaaattcagttcaatatcttctttagtttaacattactcaccacagaagtaacaatactctaccatctttataacctttcaatatatacaaatgtttatttctgttacatgacgtccatgtctgttcgtataaaataaaaaatattaaaatgtttcttttcacttcctatttttttcgtaataaaaaaactaagttgaagatatataaattattaaggtaatgaagaatgttaaaaatagaccttagaagttagaactctaatatatgaactgcaaggtccgagtgtcaaattcaaagctcgaaacatattaataaattttacggtatagggtaatttttcatatatgttttggctagattcaacgatatatattattatacctagcaaatttagatctatgtacatttaaaatacgagattctctgaaatcgttctattataacaaaagttgtcaggagagggggcatataattcaatatttgaaaaataagattcgcaatatatttttttctaactaaaataagaaagcaattttatatatttttcataaacaaataataaaaagacctacataaatacaaccgtcacttcacttgtttccttcatactatcaacttttctctatctatctctctctcttctttttccggcataacttctgtgttaccctaaactccataacctgtttcaccgataaagtgcctttgcttctatctctgtcactcttactacttgttgaacaatattctacaaaaaa

SEQ ID NO: 61 - DNA, 1295 bp, Arabidopsis thaliana
ccaccttaaatcggcggttgaaatcaaaatccaaaacatatataattatgaagaaaaaaaaaataagatatgtaattattccgctgataagggcgagcgtttgtatatcttaatactctctctttggccaagagactttgtgtgtgatacttaagtagacggaactaagtcaatactatctgttttaagacaaaaggttgatgaactttgtaccttattcgtgtgagaattgcatcgagatcttgagtgtatgtgttcttcacttctgtcaaaaacttgtgtttgcttcacagtgaagaagcctacggcttattttgcaacagggacgtggctctctctctctctctgcgcgttttttctctcgtcgtaattaatttgtttttatcctaaacgcgtatggttggcatgggttttttgggcctatgtcggtcacattcggcccaataagcttagttcttaaagtcattgctgttaacgtgaccgacctcttccatattttctttggactaaattcacttccgtagttccgtcatccatgaaggatcccttaaactaatccacatttatataatgataacacttctttttttcccactaaaatgataatacttttagacattatatgtataaatataattcacatatagaatagaaaaagttacgctcgtatttatattaatttagatcaacagttccagacgccattgtcattaatcaccttttaacatttctgatttaaattaaatcatagtctatatactctgattcagcctacaagatgataaatagagaatcgtcagtgttaaaatgcactcttacgtaacgaggaaacataagcacgttctggtcggaccgaagctaaccagcaatttcatcactctatttcacattctagttaatccacaaaattttgatatacaaaattataatattattcattgtataatttaaaagtgaaaggtttgttaaatatagtagtagaattacaatttaattaccaacataaagtcaaatgccgaataaagcattcgaaataaaattgtaaagtccgatggagacgtagactagaaataacaagtaccatacgagttgacttgtgtagaaggcaacacaaactttctcgccgagtacggcggaggagcagccgcaagtggatccagccgacactaaggaagcttttaatttgcacaagatgcgatacacttacacctgacccttcaatttcctacttatataatacatagtttcagttttcacataatacacaaaaccctttcactaataacaatctccaa

SEQ ID NO: 62 - DNA, 855 bp, Arabidopsis thaliana
tttttctttttgtttttgaaattttatttctcgttgcaaaaccaaacgacgccgtataaatataaaagaacaaattgacttggagcgacgttgcttcgttagaacgttaggtcgcacgtgtaattgacttctctaagaatcaaattcattgggtttcagaaaaaaaaagttataaactttccatagagaataatacaaggtcgaaaaatgacaaattcttaattctttacatgaaatttaatatttctgaaattataatacttggttcacgtataaaatcatccatagataagtaataacaccgtccatgcatgaaccaatattataagattctaacaggtaacagattacatataacacatgctcaacaaaatggcatagtcatatttatgaggaccgagaaatgtggatcactctgctttctttgtaacaatttagaacttcgttcatacatgatacatatcgaaatctacaatgtctataattttttttttcttgacatatatatgtatttaccgttgtataaaacattaaatatctggatttatgatacagtttgttgtcattacaaaattaatcttaaaacgataccttaaactggagaaatagtcatactttaattttactttgaaacgaagtcaacgaaggttaagtaaaattgttaatggtgtcaccgcgaagttcgtccaaaggcacacagtctccaacatgtatctcaataattaattaacataatagcccctttattaagcaaggatttgacttttcaccactataaattcacaatcaacaattctcattatcatcaaaacaaaccaactttccttctctaatcaaaacaagaaagacacaaag

SEQ ID NO: 63 - DNA, 1500 bp, Arabidopsis thaliana
gatggattaaagcaaagctgttcttatttggaaaataaaatgatcggattaggagaaataaacaccactctatcaataaactaaaatctgaacagtaaaaaagttcacatggggtggttcagttaaaacccgaacagtaacgtggaaactgaggctctttaccggcacggtcctactcctccttgttcgaattggaacttctgagatgcgtcggtaaaagtaaattactaataatactattaaatactcttttttttgtcgttaacactaaaattaggaaaaaagcaaatctaaattctattatatactagtactatatagttttggaaaaggaaaagagtgtgggagatccaactgcttagattcatgtgaactgaaactgtgtgaagccgtttgctttggaaactgaagccttccccatgtttcgtgtttcttttcttttctctgaccacaacaacactaccacttcactttctttcttacattcgaagttatttcttttagcttctagtatttattcagtacgtatgacagtaagactattttaaaattctctttctattatactataagtaaacaattatcgtttcaagattcaatgtatattttaggtagaccatatgtaatttatctctatttaaaactaaatattgtaaagaaagaacgtaagtctaacagggttgtttgtttcaaagatatatgtataaatataaactttttatttatttattcatgtggatcaaaacgatgaaacacagatgtaaaatatgaaaaatgttaggtacgcaccaacattaattaatatgtaacagaaatagactaatcactcagtaccaaattcttagttaaaagctcataattcatttcatccgatgatattatagtccaataagtttaaacaaatgtaactgattcaaaagaaagtaggaatttgttcgaaatgaatgaacagaaagagtcatcatctcgaatgaaaatcttattaggattctacataaaaataatattttgcattcaatattttattttacaatttttacgacgataaaatggctgaaagagtaaactaaatataaatctcatcttttcttttctttttttttgttatcaaagaatataaatctgaatttattgtggtgttgtcaataacaaaagtgttaagtacttgcgaatttgaaacgcaattccaaaagcataaacaaacatggccttaataacgacttaagttaagagaaggtcaaacaagatgtaataccattaactagaggtttccaacttggttaataagaaaatcaaaagcatatggttagtcgaatattttagacatttaatttcaaaactaatctataaattaattaaaacaagattcccaaatgtcatcagcacctctatatatctccctaatctccaaaaacccaccatgtctccattacacaacacacagagatagaaacaccaagaacagagccagtttcttcttcttcttctttagtttccgcc

SEQ ID NO: 64 - DNA, 1500 bp, Arabidopsis thaliana
tatgtgttaaaatgtcttgttaatatttttatttgttaaatacaaccttaatcatgcttggaatcagtggtcgagctcgaataatatgttatggaggtcatatattatataactgaataatagtattaaattatttatttttaagtaagcttagaaaataaattttgtaaaggttatggggtcgttgaccccctcctcattagctagctacgccactgcttggaagctggaatggccactaaggtaacgatatatcatcattatgcatactgatctggtaggagttatttggcagtattttggctcttgggttactctaaatttttattttttttaaatatcatcgataatttttgactgtatatttacagtcttagaaagttgttggaactttccgacgaatttccgactaaaattttcaagtttcatcaaaaataaacagaaaattctgatgcatgcctatccgacaaacctatctcgatacagataccacgtttagttggaaactcatcggaattttcgaacaacgttttgacaaaatctctcgatggaaaaatgtggttgaaactttctccaatattattttaacaatatttttttgacatctgcatcgacaaatttctggaataatcctagtttccgaccatcgtttgccaatgcacaccatgtatggagagaatataatgattgaagacactagaaggatggccatcctccatatcccagcccatctacaagattgacaaaaatgtaattgtagtcttatcagctcccttcaataccacgaatcaaacaaaactcgagggactccttacgggtttgttcagtacaaggggattagctaaaacttcatctccttgatgctcactttttttcttattttcgatcattcaattctattttctttcgttagattgtaaataaacctttttgatgagcaatagatttacatttagaaaagaaagtatattcattttggtaagaaaaaaaaaatatataggtcaaatggtaaattgaagcaacttgcccctcctaaattcatgatcaaatcccacgttagctattatatgaaatgtggataaatattattattttttcctatttagaatttaaataataataacataattgtttgtatctttaaaattagtatagtattttatcccctgcatgtaaattttatttctaaatccgcaactgcatgtacgcctttaggttaacttactttgatatccaaacattagcatccatattgttctttcattaagattacgtttacgagtctattggttcagtgaactttctttaagaaatagtattagtatatataacaaactacttaaaatgatgaaacttgagaactgagaaacattaatctattgccttagaggccactagttaaatatactagtaatatgcatttgtgaaagagctaaaacttggtgtataaataagcatttcgatttcagcttcttcatcaatcaccatagcacatcgatc

SEQ ID NO: 65 - DNA, 1455 bp, Arabidopsis thaliana
cgataaaaactatttgtgatacatccactttaagccactatgatttatttctactacaattgcaaattagtcactgagtcataggaattgtacattgtttaaagagttttgttttgtagaaccttaatgtacataaaatcaacatttgagagattaaggaataaaggcatgaaacttcttaaactccaaattaaaaggatgaatccattaatggctctgaaatcgatttgctagtttcaagacaagcttcgtaagatgaggacggaggaagcatacatacacatgcatgttagtacacattttacgtataaatacattttatgtcatataaatacattaggatatacagattcacatgatacaagcgagcttaattagtgataaagaattcatgtgtgattaatttgagtttctttctgttcccaatatgtgatctcttctagggatgcatcaccttgtttctcatggtttggagtaaatatcatttggtttgacttcacttacttttaactttagtttacaaaaactagttaatgctaattggaatcttgtgaggtataacttagaagcacactccttatgttttagttagtttaagaaatcaattatgtgctcgtgattgttggttttcttaattatagattctattgccatacaccaaattttaaagtcaacctatatatttatactattttacgtatgagggaacaaagaatttaatcatagatcaagatctacttttctatactgatttattttattttaatgtattttggaacttccatagtcaaatctgaaaaaattgtgtgcactactaccaagtcatttgctaccttactaatactaagtcaccctatcaacctagttctgatttttaaaaaatctaaacttgaattgcatatataagctattagtgtatagatcaactactggatcaataacacttctacaacacacattattgtatttaagaatataaattttgtattgcatgggtttatttttctcttcttttgactaattaaacgtttggttataatagtgaccgacactacttagtagggccaaatataactaatactcttgccgacaaagaaaaacttggtaatttgagaaattatagtagtacacacacatatataattacaaatctcatcatgtctactaaatcgcgtaaaatttaaattaaaaagttgtcaaggcaagcattttgcatgaagtacgccgcaaaagagcaattgaggcttttttgtgatgcatggggattcacgtcgtcctcgctgacaaaacttaataatatactaaaacacttacttagtcccatctctagcttcgtacatgtatataagatcgtcctgaggtggaacccataacacatacaaacaagtctaattcttgatcttgaatcttgtattaagagagatcaagattatatattctattcgtcttacaaaa

SEQ ID NO: 66 - DNA, 1249 bp, Arabidopsis thaliana
tgagaagaaggagaaagatggaaacttctttgatttttaaataaattcatggaaatttcgtgtttgtaactgttttgtttctggaaatattttatattcataaaaagtgatgtaataaaagccaaaaagataacatttggcctgtttgtgatgtttttgagggtttaagaaatgtaaaacatttcttgtgcaagaagcaaccccttgatttcttatcgttcctttgctgtattttgtgtacctatccgtttatcgaacaataaaaaaaaaaaaaactatatttgtgtgtatattcatgtatttgaatctgaaattaagtttcttggttccagtattttgtatattcttcactttgaaagcaagtgtttttttgggggttctcacgaacaactggagatgcttcgagagcactcttttcactaaaacttataaaccagtaggaaaaatcagatttaaataatcaaataagttggtgcataaagaaaaggtttccaattttctaggcatagttcatgtgatccaaggaaattaaactatgttactcatcacatgagtacatggcttcaataccatctaatcatatttctattgagtatatggaattaatatataaatgtcaactccacaatgatgattagttagtgcaaccactcgtagttttgaattgttttctaatcatgaatacaatgcatagatccgttatcgatggttgtcattagcttttcatcctttaattgtgtgaaaccattatttacaacaaggagagataaatgtggaaacaaattcgtgaaaaagatgaactgcacatttgtatatatttaattcaatgatgtccttagattaacaagcaggaaatgttaacatatatagttaatttttttttgtaaaattgttacagccgacctttggttaatgatttatattacaatttactctattttctatcggctgacttttccctgttagaaatgcagattttcaatttttgttaattattcatgtcaagaacttaatatttgaagctagtggtggttctcccaaagaatttaataaacaaagtcgaagctgtttctaacacggtcttcgattcttcatgtgattttgatcaactctggaccgtcaattaagccggtctctgctgcttacttccagcctctctggtctataagaacactccctccacttctatcaaatcaaaatccacagaaatatacaacaaaacactgtttctttcaaaaccacaaacaaa

SEQ ID NO: 67 - DNA, 1038 bp, Oryza sativa
ggcggcgcacaccggcgagaagattgtgttgttagagatagattatacggttgcattgatcattttggttgatttgtaaacagagggattaatttgtgcattcgtgaggtctatacaaattttgtcctgttttcccatctgcttttctcaagaaaaatggaaggataagtgagtttttttttctttttgggttgaagccgatgaaatcgaactgcctggttggtggaaatggttgaccaagtgaatcagtgatcaaaaaattctaaaggagttcgtaaatgttttgactttcccacatctctcatttcaaatggattataaaactcaacatatatgggacgggattttcatccctccaaaagatgttgtctcgttacatacatgttatttaaatagttataaaatttttttaaaaaaattgccatcacagattaatatgaaatgtatcactccataaacatttgtggagtgatatattacatattaatatatcttataatttttttaaaaaaaattataattatttagttgatatgcacgaaaggaggggatatgcatgtgtatgcctttgtaagtttatcgtgtgccatgcaaatacgccattcgcgtcgattggtactcattggtactatttacgagacctatttagttgatatgcaagaaaggaggggatatgcacgtgtatgcctttgttagtttatcgtgtgccatgaaagtacgccatttgcgtcgattgttactcgttggtactatttacgagatctgaacaagcaactgaattgcatgcgcgatgcgtgcgaaatgaaggacgaaacggaaaaaggcaagttttccgcctagtctccctctcgtcctctcgttgccgtccccgtcgccgtgcatcagatcatttcatcaccggcaagtgcagtcgcccctttaaatctcagctccacttgtctcgttgccgacgcgtgtgtttgctgagacgccattgtcacatcggttgctgcttccttctcggaagatagcaccaccagaggtcgtcgttgggttgta

SEQ ID NO: 68 - DNA, 1500 bp, Oryza sativa
gtccgcgacggctcgtcttcctcttcgctcgcgcgctgcgtgccttcgtagacgcctgcgaaaagtttcgaccgagcagccggtctttccgccacctcggtacgtgttcaatatgttgcgcagtttgatctgttcatgttatactgtgtagtttacatgtgtagatctaatgatgcgttcatgctatttttcatctgtaatgtcatgatctatttatggaatagattaaatcattatatgcttataatctcaacaacagatgcttaaatagaagaggctgtatctccatttgtattgtaatattttacaacactatgattctaccaaattgaaacgataccgatcccatctaatatttaaattagtttacatagtattttcatacggttctatggaatcttagatattacattgatttctatgatctttataatcctacaaaatattattaaaaataatatgaataaatatgcttaagtttacataaaactgattaatttttttaaaatcaatactatatactataatttatacacaacacacacacacacacacatatatatatatatatatatatatatatatatatatatatatatatatatatatatataatataaaaataagtactttctaaaatttgttagtattccaatcctacttagcatctcgatacggattacctttggatggagtggagccaattgtccctgtgtcacgaaaattgaattgacgaaaaataccctccgtgtgagtgtgacgcaccttacatcccagcacagagggaggtggagagcacgaatctcagcgcatggcctgcaccaaacatgatactactcggtgcagcagttagtgggcccgcattgccattggctgcagggaccagcggagtgggtagcaacgacggacggcgcaacgtgtgggcacgcgacaccgcacagcaaaaaaaaagggtaaaatgtcgggtgcaggcacaaccccaagtaaccaccactgaccaaagggccccacgtaccatcctaccccacctgtcagcgtgcgtaccgggtacagaacccggtgcaaaaaagaacaaaccagggccgtttcacgctttaccagcctggcgcgtcgcgtccgtccacatgcccccgcactacccctcccctccacatggcgcgccacacccacgagaccaccacgtgcttgaccacatgggcccacctgtcattcactccaagatctccggcccgggtcacgcgcgcgcggccgcattgattaataagggggaggggaaaaaaatctcccgtgggccgctgcaggtgggctccacgttagctccgggcccacgtgtcatcggctatgtaataataatataatataataataataattgcagtatataggggggatatagtcgcggtcgcggggagccctaggcgagcgagggagaaagagagaggcgttggtcggtggccgcccgtcgcttctcctcttccgacgccggcgcggccgagagaa

SEQ ID NO: 69 - DNA, 1500 bp, Oryza sativa
atcggcaccgttttaattaggtgcctatttgcttgtctaggttcaccgtccaccgtcaaaccgtcatctctcacggccatccctctaaacccacccgatgatctgcctcctcctcccacccccaaccaatcctaacttgggaccctaaaccattactccactgccattcgtcgaggttatcggagatgtaggagaagggttgccagatctagtgaaggagttgtagtcattggagcacaaatcgtgtaggaaatataatcctgtggtaaaaaaaattgaaagattttgattgaagcaattccaattaaaattacaacatcaggcaagcaatataagaattttttttacaaatatacttattttttcaataatttacatttatatatatgcatacaaattttttgtaaaaatatacccatcccgcagaacgaaaatgcgggatcgcgacggtcccgcagcttcaaaacacgggaccgtcggtcccgcggacggccgcgcggcactgcaacggtcccgcagctccataacacgggactgtcgcggtcccgcagctccaatacgcggcaccgtgttgccagcgatttttatactgtgcgcactgttgaaacactgtttgggtactgttttgaaccgtgcgtggagcgcttccgcacattcaaactgcgggatcgcccatgaatattgccaaacagtacgaaaacagtactaaacagtgccgcgcagtataaattttctggtatatagcaggcgcgtggtagaggatgggaacgcgcggtcccgctcaacgactacgcgcgaccggtgcggttccgtgcatcggcgacgcgccgcgcaacggcgatgcgggaccgtctcgggaccacgcgttcccgcgctgcttctctccgagccacgagagcgagccggtgccgcgcttccgcgccgcgggagggagacggtgccgcacggccgtactgcgcaaccaccggtcccatggaatggcaaggcggcactgtctcggtcccgcatccttgtcatgcggtggaggtatatttatgcaaatattttttatgtagatatataaacgtaaaatatttttaaaagaggtatattttcagttttgtttcagcaatataatgtatcactttagagactcagaagaaatttttatcggcagatcaaacatttccacatatttcaacaaatgcaactggtcaactcgcttcaattccccgtgatgagtcggcgatcgatttccacgaatatctgttctctcctggcaaaatcttgtcctatctctcgcgaacacctggcccatgcacctacaacaaccaagcaatcattcatcatgcatgcaactagcaaccgcaaatacatccattctagccataatccggctataaataccagacgcccaaatcaaacgatgcaaagcaacacaagccgaccgaagcaaacatagctatagctagcacacaccactagctaattaagctcgctcgatcacttcatcagttagca

SEQ ID NO: 70 - DNA, 1500 bp, Oryza sativa
ccaaccaaataatcagctcatgaaccacaaaaaaaaaacctatatcacatttgcaacctaccataattcagaagagacaatgctctgaacctctttgccatgaagaataccatacgtttcgcctctctttaaccaccacgttgaatgaaaatccatgtgttaattaggcgacaagacgaacggtatcaccgatcggtgagatggaaaggacgatgccgtagatccccaaatctgaaagaaccctgccaaaaactaatcaagtcaatgctgctgctgctcctgccattgccaaccgccagaagaaaaatgattagagcagcaatcacacaggctagctggttaagcagagctgaactgatgcagaaaatggagtagtttataatgcattagccggtacagcattattgttaagtgcgtgatcacatgttagaaggctcaacagccatgttctgatgtgaaacctatcctctgctccatcagccattgacatgctggattcctggccgtccaaaactgcaaaggatctgaaaatgtgaactgctgtagatctgtttcagaagcagagaaaattgtcaaatgccaactagccattgatttgaagcaataatgattggcaatggagtttaacagatttgtttgactgaatattttcaggtgaaggcagtatatgttttagatccaatttcttaggatctgaaaatctccaacccatgcttaacagactttttcagttatccgaaacagaaaaggcgaacagaatcgcctcattgactgaatcacgtaacactatctactttccataactcctaaaaatcatggtctacttaagctgatgaagataaagaaaaaaggttcactgcttaaccatttaaagccgaatgttgaccgtctcaattagcaaagatatttgttttctgccactagcttgttcaatcatctcatatttgcaaaattaattagtcatagtgctctaataaacatattgttttttttgccacttaacttgttcaatcatctcatatattccaaattaattagtcatacagctctaataaacataactgtagccctatgattatatataaaagaaaaatgaatttgcagggctattgctctgaagatctgcattaagccagactctggccagcattttccccaagatggcattagcaatcagaaatctattcagattccccgaactggcaaccccttttctttactgacctacctgcagaaacctctaatcaattaattattcagttaacaccattagttactcctagatcagaagaagggaaaaaacacatagaaaaataattaattagactattgtaaggtgggccccacacaagctccctccctataaattacccctccatgaacgcctccaaagcttcatcactgcacaaacgcaacaaacgaagcccaaaacgccaaagagagaagaaaaaaaacacgaaaaatttccccaaaaaagttagcgaccaagaaaccgcagaa

SEQ ID NO: 71 - DNA, 747 bp, Arabidopsis thaliana
taataaactacttaagaaaaatgggaaaataataattggaaccgactttaatttcacaatgactcttttgcccttccatcgttgacgacattgtttctccttctctcttccacccatttaaaaacgcaaacttggaaaatggagaactgtttaactacgccaacgggctgttttaatgacaaatgttagactgggctcagtaacggatgttgtttcttgtcactaaagaatacgattcatagatttaaaagtaatccatttactttgcttttactttttcatgtgaagagaaacaaaaaaaagttttgttggtgatcaagtttgtagtgtactagtaaaatagtaccatagtatgtgtgtgtgtgatgtggaaacaaaagaatatatagaaaattttatgttttgggtttcatgtattttcttttaacataaaaacaagaacagataagcataatgacgtaagcagtggcctaagcatctcttaaaactccatttaaaaaaacatatcgaattgaataacattgagttgacttgtttaacattcacaccttgcttgctacggtatcttagcttccaagattcaattaatttttcattttttatgttcacagtcaatagtaataagacatcagcatcgcatacaaatttcttatataactcgactagtttactctataaagaatcagcgattggagtatagaaaaggtttaagcaaaatccatcaaagaagagttccttttcacaata

SEQ ID NO: 72 - DNA, 1250 bp, Arabidopsis thaliana
acccgaaaatattcggtgctccatttgtttctgattttagttgtggatttcgaattgaaaattttattttattttattatacaacatttgccattttatttccacagaaaaatctgaatactgagagtgagaggtacatggtcatgaactcatgatcacatggttttggtagcacatggaactgcatttcagttctacatgtagttgtaaacatcagaataactacatgtatggctatatgcatgtaaaaccgcagttgtccgtattcacaaacgtaccaatttttaaccgctaaaatagtcgttcaaaatgctttaaaaccgcaatcgcaggcatccataaattctcgcagtcgtgatcgcacgtcagctccgtttaaacatgtcaagccttgagtcgatcattacaataatatctcgtgtaccacgttatgcttaaaaaaattaatcatatcttaaacacttgccttgccacccactacttctagatctcccacaaatccacagaaataatatatatggtacttagaaaaatcataaaaatatcaaataccaaattgaagtctctggcatgagtcggtcactacaataataactaaactgaaatacattttatggctaattgatgcagaacctgaaattaaaaaaaattaaagatacaacaaaccatacctttttgtctttttttttttttaattattttaaagataactcatttattttgaatgtatgtacgcagttttcggttccgtattgtgtattcatagtctaccataaaatttaacatctacctttcccaatggacccaaacgcttcgaattgaacatgggcgttgctttctcttgtgagttgtatctaatataaaatttggataattatgatttcgtcggtgattttgatatactatatctctcgtgtacaacgttttgcttaaaaatttgttcatatcttgaacacttgtgacttgccacccactacttcgagatctcctacaaatccgtagaaatatcgtttcacaatagacttgcatgttacttggaaaaaggattaaaatattaataaccaaatcgaaatctgatactttacacattcgaagtcaaacaaaataataaaaatgaatgtgtaacaaatatatttttttccccacgaagaatccacttttagtattaactaaactcgatcagtaagtaatccgtctatataaatgtggaggcttcccaaaacaaaacaaaacaaaaaagatctcaaaat

SEQ ID NO: 73 - DNA, 1100 bp, Arabidopsis thaliana
cttaattataaatttttttttgttagatttataaattaaatttccccaaactaaaaggttagaccaaatataatttcttgcagattttttctttgttttgagcattaatgtattatttttagtgtatcaaggcaattagatggtgatataagtctaagaatttgaagtaaggtaaatggttaaacgtccaactacgaaattcgtgtaattaattataatataccttaactgcctaccaaatactaaattatagtgcaactttactaaagtttacataggttcgagaagtcattagcatttgacatattaggatttcattttacacaagctttcgagaagataaacatgtttgacttgtatacggctgttcaacgactcttgacgaaaactcaatttttggtatttcaaattgcttgaaaatctgatgtaaaagggcaacaactacacgatatgattcaaactactatcaattttctttataaaaaaaaaaaaaaaaaaaaaaaaaaaaaactagactaatttttatttgtgtactgtatacattattttctaaaacaaatgtctagacattatagtattttataaacacttacacaacaccaaatgtttgtcttaaccccgactaaaaaggacatatgtgttgttaatgctggatcaccggtggttttgttagtgtctacccttatttgtatggaaaaacactgccatagcccataaggtaacttataaggacatcaaagcttttggtgtttgactgcttaaatgttcagtctccagattccagcacatgcatttctcgaatacttgactaatggtgaagctagcattttttaataactttgccttaccaatcatggtggttggcggaattaatcatggtggtaagcgagagactttattttatccaaaaaaaaaatgagagacgtaaaattagaactatttttagttataatagttatattttattattttgtctcaattattctccccgcccctctttgtagacgtgtgaatattagagagattgataacgaactcctcacaacatcatatctcaagttcaccacccaaaagaaaaaacacacacacatcttagagag

SEQ ID NO: 74 - DNA, 965 bp, Arabidopsis thaliana
ctatccagcttcatatttgcactttataatcttctcgtggatcttgtttagattatcttcctttgtctttcaagatgctttgtcttgttaccttgcaatttttgttctttccacacgagctacaaacttatcatttggtagtccaaaccccttaacattgtgttttctgatggttacaaatattatgacaccttgtatttgttgatcaatgatcattatagtactaattgcttctatcattgaaatgcaaatcttaaggtcttgtcttcttgatacattcggtcgctgcaaaagccttggtaaaaagtagtttcaacacacacacattgtcactttatgacatcatttttatgtaaaaatgagatttttcacttggcttctacggaccgacccaacgatatagattttaatttttttttgcgtttaggttttgcgagatttacagttgaattagcgttatggattttcctcttgttttccattgaccacgtccccaatgtaaaccacgtgaattcgtgtacttatgttcatttctcacgtttggtataacaaacataaagttaaataaaattgttgtagattgtagtttaatcaagattaaaagaaaacatatttattagctaacttttatagtatgtattaaccaaccacctgttcgaaatgtgttttcattagtacaatctgaaattaagcaaatgttttcattatcctcaaaaattcatcttaaataaattcttaaaatagaataaaaaataaagatacgtcctccccaacccctttctcattatagagaaaactttaaaaatagactagagacgtgcttaattattcagctactaaagtaagggattttgttagaaaattgagctattgacaaaaacaagagaatctttttggggagataatataagaagagcaagtgactctcactctctctctctcagatccctccatcattgttcacc

SEQ ID NO: 75 - DNA, 691 bp, Arabidopsis thaliana
tgagaatatttatattttgtatctctgaaaatcaagtttaaaggggaaatgattatgggttgaaattttgcaatcaaaagccctaatttgcaaaaactacataagttttttgtttgggctggcgctatcggatccttttaggcttacatttaacatctggtccacttagaaagagtcacgtagtatatggtaattgtcaacttgatttttcaagttaaaagaaatatgtatcaaaatgactaaaaagtagtgaaatattatgtatctaatttgtttatttaccaaattaatgctataaaaatgttcaactgtacaattggcatggaataatatgaacataaatcatacattattaagcacttttgcctacgaagggataccaacttcattagtttacattttcttttgtgttcaattgttagctcaaacccaattaagtggggaaagtaagaagcaacaactcctcttcccggacccctaacaaatcaactaaactcaatatcaaaccattttaaaagagctcatcattaactagctactaattattcttaatcaatcactgcttaatacaaagcactatatatacacttgtatcttccattagtttcccaccacaactacaaaacattccaatacacaacacacaaagcacacactttttctttcttttaaaccccaattgcaagagaaa

SEQ ID NO: 76 - DNA, 1406 bp, Arabidopsis thaliana
cgaagatttgattgcctagctagaaagtattcagaaaaaagtgtcgaatgctctggaatatgttgtaatgggcatagataaaattttggatcttcaaaatgaaatagtttctataactttttatagtttaatggtttgtgatcaacagttgtgttacacatctaaactcgtttattttcagcaaaaaaaatctaaactcgtttattacatgttgtaacccaaaaaaataaaaaaatgaaaaaccgtcgcaagtgatatcgacctttcgcctactttcgcactcatgttggtgtcttgacaaaacccactgtggacgcatataaccaccggaggagtacaggccaaaatttctattattaatatgagcttagttagataatccaagtctagtaaaagatttgaataaaagcaagttgtgtccacaaaactaaaataaaccaaatccaaagtcagtcacatttggaattatgttgtaatgacagtcactgctttgtcatcatctcaccttttaaggcattgaccttttgtttttgttcgacaaaaaccattttcgcattcatcactttcgaataaaattatataacagtctaagtaacaaactcatattaggataccaatttgaatttgacttttattggatatcgtagctaacgaaaaaaattcttaatcattagacgattaagattatcgataactcaaatgtttttttttctaagttatggtcgaaatattggtatacatcattcaattttaaagcataactagctagtgataatgactactacatacgccataagatgaaccagaatcctatgataatgatatatccaaaaaaaaactgaattcaaattgttgaattatctgagtttaactttttatcacacacaaacaacaactgaaacatatggaacaagaaaaagaacactttattttcttgaaaactctgtaaacttaagaattagtttatttaaaaattaaaccaaagaagcgttttaaatgaactaaaatccacatttatacagctcaatctgtacttctttttttctaaatttagcaacagaaaaataccaaattgaacaccttcacatataaaataaaagataaacatgaattgttttgtaagaaatttgataaccccacatgcatttggtcattatttaacaacaaaaagtagactcaattttaatagtatatctttagatcgaatcagcataatgacgtatctttctgacgtatagcttttgtcttggttgttctttacacatctataataaccaaacctgtgtgaacaagacctaaaacagagagaaattcacaacaaaacaggtacacttttgttgacttttatatatctctagtttgcttcatctttgttgacttttcttgctcttctccatatggcaggt

SEQ ID NO: 77 - DNA, 1720 bp, Arabidopsis thaliana
cgataagccttgcaccttcatttattcctttcagttcaatatagtagttgggctattataatcgcaacacctggcccataggccatatgtcttcttcttttttttcttctaggccatatgtctacgattgaccaaaagtaaaataaagtaaatgttgtttacggattagattggtgtgaaggagctgtgagcctatggcatatgtaccacgttaagatttaagaaaatctggaataaaaaatgcaactagatattatgttatacaaatctaggccagagtcggttgttttcggaccgttctgccgtgaaactgtgttccttaacattttgatttaacgtttccatatttctgacattccaataaattattgataactagtaattaattaaaacggtttactctgaactctgaagtgtgtgaggtcatcaaatgtcaacggctacaatttcattagttacgtgcgtgactgcgtgttgacatattaagcagacaaaattataattacgttagatcattagcaattagatacagtcacgaatgcattgaatgtaagttgattataaaactttaaagcggactaattgtatttaaaaagagagctatagttagattggaacttggaaccatcgatatgattttatccgttaaataatttgagaaaatgtttaatgctgacaaataattgaacaatttgtcaacatattgcaagtttgcaacaacgaaaaacgataaattaacacctaataagtcagttgtacaaatatacctacaccaaacatgcatttacatatttgttcaaatatttttctttgaaaggagtgttgcaccccccataaaataaaacgtcaatattctttcctgttttgtttctttcggtaattagagaaactgattgtgaattctttaccaacaaacaaacaaatttacctagcaaagttttaatacttcaacaagcttacatgatattaaatcatttgtctcgaaattccgtgttatgatgcactaatgtcaatcaaactattaataaaaacaaatatcataacaggtaacggctactattaataactcaattatcatcgagatacgcatatagatagacacataatcaaatactaatattttctctataaacgtatgaatataatgttctgcaataacagataggcatctttgaataagcagacagagtttccgatgattagtaaattactttctgcaaacaaacctacgcgtttttatgactcccaaacgagttgactcaggattatgatcgcattacaatatcatagttatcatacacatattaaaaaattaaaatggacctacctcatcctcattaatcatcaccacacttatataaaccacttaacctaaagtttgtattcacataaaactttccagtaccaggtacgtcacacaatcacatgttattaataataatgtctcgagatctcaaagttatttttcaataatatttaaaatttctcgagtacgtaaatagttgcctttaattattgcatttaaaactttaggtcaaaaaagttagaattgaaagtggtgaacaggggacaagatgcattaataatactgagtgtacataaccattttgttgatttttttagtacgacatagtttgacaatgcaggcattaaataaacgttatttttgggttcaagtgattctagtaactaatcgtgcacatgaatcttttattcaggt

SEQ ID NO: 78 - DNA, 2659 bp, Arabidopsis thaliana
ttaatataagattaattaaccattacaatatgatataaagtaatgaataaaaatggagattatatagactctccagcttttaataataaaccagctagtttaacattttcttttaatgtgactaagaggtttaattacgacgatgtgagtgtgagggaatacgtgtttaaatcacacataagaaaaaagaaatcctatgtaattgtaatctcttaagaaaatttaaaataagctatatatgtgtaatactgaaattctcctactttagaatattttatggctgttaattatgaaaaaaaaaattggactataaacagaaacaattaattaatggatatatcaaaaaaaaaaaacgcaaacgtaaaatagaataacatttaggtcaataaaattaaccaattgagttaataatatatataacgtctaaatatgtacattagtttccaactcgaggcaatgaaagtttttttttttttaattcaataaaggatgtaatgaaaataaggatgattggttttcaagatgaagttaaatttcataaaatagtatatttggtatcacgagattttcgccaatttccggcagaaaactgggtgatgaatattagtctcatctcatattgtataatgtacatgtacgtgctcggtgatgggtgaaatttgtcctctccgtctctggtttttataactagaccgatattaattaaaattatgtgtcctgtataaagttatcgttcttaattatacttgaaatgtgaatattggttcctttctttatcttaatctataactcttaactgtaaagtgaaatttgaataagacttttacaaaaaacataaaatacaattagtgaagtcaatcaaagttggaacgattcagtttgaattcactgttcgaaatgaatctaatcattggtggaaaatgattaaagggataatagtaaggataattatatatcagtggttgactttgacaataaagaaaaagacaagtcaatgatattatgccttcgaagttgtctcttggtggtttcattaaacaagacatcctcattcctcagtcaaaattatatgctctttttttttttacccaccgactatctagtaaacattaaacaagacatcaagcttatctctttacagctttgataaattcgtaacagttttgacaaaaaaaaataaaaaatactagtttcatacggttttcattaattcgtaacagttttgtttttagttaatatttttcaaaatggtaatttctctgcagcacacttttaaagtcgaattcatttacaagttgtgatttagcggtcacacacgtttttccgtcatcagaaaattacacatacacatacatatgcatgccagctgcaagtatatcatataatgttatactgtattactttgtaactcgttaattactttaattaagggaactgaataactaactacagccatcactatacatattctctttgttttgtactttccccatgcagaaacctacttagtctattattgttcaatggttcaatttttttattttatttgtatggttcaaattttccatggcggtgaatactttaatgttttttatccttcaaagaataaatagtagtattatatattggctaatccaaaagagtagagaaacaaatataccattataatagacgatttgatcattttaaaaagtcaactatgcccgttacattttcatcatccctaacgttctgaccaaaactttgtaatctatattatcgccacgtaactcattgatctaatctatgaagcaatatttgaccctaataaaacatcactattgtggtaccaactggtatcggagattttactttttaagccaactgattaatattttgtcacgaataatttgcgtttatatttttcaaaatcacattttagtttttacaaaaaaaaaatcacattttagtgtttcttgaatagatttgatcacttttcttaatgtatcgttcatttttttccaattaatcaactgcaattttggccgatttagctacatgtcgaaaaacactaatcgatttttcttccagagttttaagatgctttacgtttataccattcaccttatattagttttcattttgttttcgaccaaaactattgatccaaattattatagctaggtacgatttgaacaaactttgattctcttaccaatcatctacaacttaaaaccaagatagcttcgctcaaaaagaaaaaaataatgacaacatgtataatacatatatgttcaatggaccgactcatctacactgttattcaaactaattattttataaatgattactaaatcagcttattaaattcccataatttctgcgtcgtgtgccgaagttgctcgttacaattgttattcccacaactttttttgcctatatatacaaaccctttaacatcaaactcaaaacacacaacaaacacaacttctacaagactcaaatagtttctatttaattactaaaaagaaaacaggtaatatactgaaattattacatgcattacatatttcattcgatttcttactttcttatttatgtgtgtatttcacatgattaaaatatataacatatatatatggtcgtgtttagcggtaatttaatattggtttatgcaggt

SEQ ID NO: 79 - DNA, 3177 bp, Arabidopsis thaliana
tatacccgaagatttgatgagtaaacacatgcataaaatattttttagctatatttggtatctttacgatgtttatatttttttctttttgataaagtgtttttgaaagatatgcctccaatttgatataattaataatataagatgagatgtagttttgagttttattcccccgaatggaatctctcttcctaacaacgtgtaaagtttgtaaactacgtgtattcacgtgttctttcacaatgcatgtttattatctttcttactatttttgtttctttctgacatttacgttttgttctgtcatatactcatataggctttaagttgggttaagatgttcaaataaatatacatgtaagatgaaactaaaacaaattacattaatatcagatttttattgttaaaggtttgaagatattttttcctgaaaactaaaaagtatgttcttaacaaaaaaaaaaaaaaaaaaaaaaaaaactaacaagtatgttgtttactgaaaagattagtgactgattttgtgagccttgacgataaaaatgtagaaaagtaaacacttcagcaagtggataatttataggtacctcatattctcgtcttttgatcaaacgtttcttttcctataaaaattaattacagataaaaagcgtcatatattaattaaccaatataatagcatatttattttggtgtattactttttctatcaattaaatatataaagggaaactatctcaaatctaaaagggaatccgatttaaagaagcttaacttcttctgttctcttttcataaaaaataaatataattacagatataaagtttaaacaatacaatagtatatttattttggcacattacttttttccattaaagaaattaaaggaagctatttcaaaactaaaaggaatccgatttaaagaagcttaacttcttctgttctctcgtttgcactttggctctacgtgatcaagaaaggtcggcaacttatactcagtctctggcaacctataagttgaatgacctgagatccggcgcagaacgttgaagaatctcactgtttgtaggcaaaatgtacctccttgtggatttgaggattgtttggtagacaactaattgagaatcaaaattggcaacaaaaaaaaaaatatggttaatccaaaatagtaattacgttatggaaatttggtaactgtcagagaaaacagactgtacctacattttggatcgtctttctccggaaacttaaacctttgtcgttgactctatccctcgttaacctggatccttgtggacagatcacgctaaatattcaccaaataacgatcaagtagtactcgtaaaaacaaggtgtgaatacatgagtgactttcatgtggaccgtatatgtaacctcaccataaataaaaaaaataaaaaaaaaaaaataaaatgaaaaagggtttaacgtccattcaccatatcagctatttttttgtttgtctgaaaatgtatgaccatatcattatgacattatctgctaattaaactgccttaataaattaacttcgtatatattttgctaatgtatctctaataattattcgttattatcaacttctttaaattttattatgaagatacgatatcaactctagttaaaattttcataacacagaagcgactctgatgtgatgggagtgaaaactaatacagctccacattctcacatgatgcataaacaatgctaaaacctcagataccataacttttcatggaattatgtttctcgattacaactatgtacccttccagatttcagaccaaattttaccatattttttctaataataaaagaaaactttatcttacatatatgcatttagccttctgtatacgcatttgcatgcatgcattaatgagtttcttctgatatacatgtgtgaggatttgtatatttaattcaaaaatatttgcataatggtcttactttttttttgttttttttctttttgacggcacttactttttatatattaaaagagttttttaacatgtgggttatgacaattttctcagtaactttaatttattttaatcacaaaaatatactatatagtggaatataatgttgtttatgctttattgtatgtgagagtctgggtctagtttatttattttaataaatgtgttacatctttctctttgcaatattcttgcttattgtttatggcgtcgatgttgtttcatggaccaactgaaatatataaaaggaataagcatctaatcgactaatcactttaaaatactccatatagtactaatcaaatacgtaccccataaaatagtccaatgattcaccacgaacttaaaaaaactgtataaatgggattcccacttttcctaaagccttgtgaccggctgtaccaatttaaaacgattttatgctataattcatttattgaatgttagggtaagatttgattaaaattaccctaaaatcatggtaaattttgatttaatttataacaataacgacacaaaattagagacaatgaaataaattttgatttttagtatgtggtggagatgtcaaacagaatctccattcaatttttttaaaaaaatacttatatacatataaattagggcatataattatgtataaatacatacaaatatgccttatatttatcttatattctagatatggggttttattccaaaaaattatattgtttgtatcattcactaatcataagtcttaaaatatattgcaggcagaaccgtctatcttagtttcaagtgttacggattgtttatcgctgggctttcgcggaggacgacaaaataacatatattaaaaatatatactcttaataatttttaaaatagggctgcaaatcgtttgagacaactctgaacacggtgcatgtatgatctcttctatataaagagatcttaatttgcacgtatatcatcatagaaaataaaacaattactacattttacaaaacaacaggtattataaacgttcatgcatactaactagacttctcattttattttgtttttgaaaattaaacgaacttctaatttgcacattaagtgtttaatccgtatctatttctgtatacattaacatgtgatgataatgatgatgagttgatgatgatatatgatgatccatgaaggt

SEQ ID NO: 80 - DNA, 3036 bp, Arabidopsis thaliana
gaaaaaatggtccacacacatgtaaaatatcgtggacttacaaatgagactgagagcgtacgtacgtatagatgcatcagaattcatgtacaatatcaagagggcctctttcgatgacttcttatttgtctctgtttacaggaggacccctctactgctttttaccagtcaagactcttaatctcttagatcatagaatctatgaaaaaactgttgtcatgaaaacattttttttttcatttttgagttgaaatgtaaatgatgttttcattatggaaaatgaaaagctatttaaaaaatttaatgaataatatataagtttttttaggattataaaatactttatttttggttttggataatggttttctgttttataacaacaacttacatggatatagagtttttttcagatcaaaactagattagcccttttcagaccaagaagcaaaattggcaaaggaggcataatagttatgtgatattgactctcacgtcacggccgttgttgttcgctgtgaaaccaagtttgaagccagtggaaacctatagttcgtaaattgtgtgagtgatggaattggatgttctattccttatggtgaagtctatatttttgaaaacttgatctgaggctcttcgtttgcgcctgcactaagtctatatgtgtagaatgtagggtagataaaaaaatctaaattgaatgtgatgttcctttttctaaattaataatgtatacaatatctcatatatatatgaagagttttatgatgatgcgaaagcgagctagccgaaaaatagaactaacgtttcgtttatagtctgtagttagttaagtttgacgtaaacattgatcttgaaggcatggttcagaccactttttaaaatacctttaaagttactagtatagcggaaaaaaaaaatttgattaacatctggttcaaattataaaattaaacattttttatacagaaaaattggaaatagttttagataaaaaacaataccaattagcacatgaaaaatagcggctactgaataaaataagtaaaaacataagctcactcaagatcaaatccgatttgagttaaagtattgcgatagtcaagattagatttgatctgtgtatcgtggaatcatcctaaatgttataatttaagattacgaatcaaacaaacacatcttgaaaacaaccacatggctgttaaaaacaattagtattactattaactaacttcttacatcttgaaaaaacaaagagttccttcacatgaggccatgagcatgtgacatgcttgagttgactttgaaatttgaatggatagggccacgtcaacgcattcaaggatccttaattagcgcttaagtaaaacctctttatttgtcgtgactcgcgaaccccttttttattaacgttttagtcaacacaacatttcattaatgataattctactactattagtttgcaatgttaactaaactctttttacgtgagaaaacttaagattatcatttccagaccaccgcaagttccttgaaaagattgttatatatataacagctgcatatcttaatacggatttatgggctttaatttgaaatcaattgtatcaaataggtttgaaaaaaaaaatcgtatcacatacctttattttttgagtgtagtataagcaagcaatattgatgaatgcgtgagtctgcaaaatttaaccccaaaaaaaaagtaagcaacaatatatattcagcaatcatgttagaaagtattttaatcatgttgaactgaacgatctccgcgctaattagtattcctaagagacaccaatcagaaactattggatagttcgacggtttagaatttgtccagttgagaatggttttcaaactattttataaaatttttttagcgaatttctaaagttaagttgaccagcacatcttgtggttaaatgtttcactcgtcgttgaaaaagtcttttcaacaaaatcttactttctggatataattaatatcatatgtacaaaaattgattaatgggtcttaaactatttcatgtatttactatttagatagagacgtttaaaaaaaaactattttcgtgtctttactatttagatagagattacacgacatggaaataatagtacatggtcaagtttatatacggacgactctcatgaaatcctacaacaagaaaacaaagcaacatatagtataatgtgaaatatacactgttaagcaacatattacgtattatagttatttttatgttaatgacgtacaatgtacaaattctagtattcttcacctgaattatttgatgctaaactacgtacgtcgtggttattttcattgttctttaattagccatctcgaaatataattatttcaatgttacaagattttagtcgctctaataggatgtttatgaatttaaaccgacccaatccgacttgttttttcttctaaaaaatattatcttgaaaatgattttattaaattcgttttcgtcttagtctaattcagctataaagtataaacgttatgaccaagtccataatcaaatcatcatagtatttctccttaatcacaactacaagaaaaggaaatgggtcatgactttcttataaaacattaactaagatttgaccaaacataattttgtattatcaatattacaccataaatacggccacatatcctcctagtttcttcacacaactctcccctcaaaacattccatcaaaggaaaaaacaggtaagttcaccgtttactacattctattattctttttatgtatcagaaaatgtctaaaaaggaattattaaagtattatcatgtttaaaaatttatataaatatctgatttatcagtttgcattaaaaactttattttgataattgaagaaaaatatagagacaggaatattttcctcataagaaataaggatttgctagctatatatattaccaaaatgtgttccaagaatgtgtctaaaacaagtttaaaataaagtttatctgtttggttatgtaaaggt

SEQ ID NO: 81 - DNA, 1835 bp, Arabidopsis thaliana
agctaagctcatgacaaccacacacaaccatatgatttgagtaaccaactaggtgttgttatccgattgaaccgtgttttgcaaatttaattacatgtcaaaaaaatatgaatccgtatgaaatcttttaacgtaattgcatttttttgtcaacttaatggacggtaatccacaaaacaagctaaaacaaagtcaacattttcaagacatgacaaaaattgcctaactgtcattatccgccgattcgtttgactacgcaacatgccaacatgtaaattaatgtaggcatagtcgtcgaatcgtcattaagcaacgacggtcatattatggagaaacaaaataatcgtaattagtactacctctgtttcatattagttgtatttaaggtttttacacacaaattaagaaaacataaaaatctcatttaataaaaacatcgtttattataatcaattcaactaataaaaaaatagataattaaatataattggttaaaaaatattaaatacatttattatttgcatagaaatttaaaaacaacatttaaattgaaaaaaaataaaaacccttaaactacatttatatagaaacagagggagtattattaacgcaaccaaatttctaattagacttgactaatttaaggagtctcctttgatcacaatattttatcttcatataaagacatttcttcattgaaccaaaacaactattcaattccatcatgtgaaaaaataattgtaattagttgtaaactcaaccaccttatcttatcttataaaattcatactagtgtctaagcacgttacttccatattatattgtcatcgactaatcaaatgaatcttgttttgacaatcctttataagtatataaacacattttcttcattgacgctaaaaagaaaaaaaacacaaaactcatccatttggcttctctcttgcatattcgctgcaaggcaggtaacattggttaatatatccgttctttatatttgtatgaaaaatttatataaacaatcatatttgtgatatatttaatactttctaggccatcatatatatataaaattatttatttctagatattaacaaaaatatcatattaatatacatactgtatatacttaataactaactaaaactatttatcaaacataaatttctagttatctatatataggtacttaattactaatttatttatatcaaataagaaatgagttaagccgaaacttatacacgtccatgagtcctcaatggtttagacttaagagtctagcatgcaagaccggcccatgatttaagcagggtaaatcggtaaccaacgcttagttaggacccaaaatttgtagaccgtcatagattcaaaattatttttaatattttattaatatgagttttttcttttttcttctcctatagttttgcattatatagtttaggattttttttctgatatgtcttttaaggattgtaaattttttaattcatttacaaattgcttggacgttggacattatcacaagactaattaaattgacagcctaccaaaaaaaaaagactaattaaattgacatgcttatagttgcgggttattttttaaaaacatacatatctcctatataaacatacgcaaatataatctatatatgtaacaagagcgacaacatgacaatactaaattgacaggccaacttgcgggtcatttttttgacaaaagaaatggtgaaggaagtatcccactgttaaatcttttggcttttttttttttttttgggtaacaccgcatcttcttgataattctgttaacgtttttaccttttaactgcaggt

SEQ ID NO: 82 - DNA, 700 bp, Arabidopsis thaliana
atcctttaaattttatcggtcgaaagacaagtttctctagttagcgcttagcagcttccaagttccaacgattcgaaagcaactgatgctttgaccgatttgaaaatgcaaaagattggtctctgtgcatatactatcctttaaatttaaccaaattctttttgtgtaattcagaattttctttttacagttgtagctattcatgaattacaaatattttcaatcatttggttacgtgatggactaatatataaaccaatcaaccatgtgcttgtcgtcatttcttttgtttattaattgtgtttataaacacatatataataagtatgtgtttattctattgttaacacaacgtttatcatgcaatgcaagtcttagtctatgtttagtttattaacggaagataaaatatattattactaatagaaaagaggatacgaccgacgaagtttgaattttccaaaatggcaacctaacttaaataatgcaaaatcattaaacctacaattcttatattatcaataaaaccatataaagactctctatagaaaaaaactatataaagacaatcaaagactctctcgttacaataaccacaaacaacaatacacttctcttgacgcctatctctttctcaccaccaccattaccttcgtcacttctctcttccaagcaatttaaaccttcaactaatccagaa

SEQ ID NO: 83 - DNA, 2101 bp, Oryza sativa
aaacttccgctgtgtaatagtcttacttatgtttaattaggtgaatacctactattgtaaaatcccttagtattgtaaactttaacatatgcaatgaagaaccagctatattgtatgtataaatatttactgatccagggattgatacattaattccagtcggatccaagtcattagttttgggacccgacaggacctcgcctccaccagatccggcggcgtggaggcagctgcggcccagtgggggtgggaggggcagcgggccggacctcgcctccgccagatcaggcggcatggaggcggctgcggtctggcgggggcgtcgtctctttctctctctctctgtgtgcggcggatggcatggtggcagtggaggttcatgtgttcttggttgaaagttttgttatgtgtgttcttgatggaatgtttcatgtgtgttcttaatgaattccgactgggatttttctttggggggggacaaaaaaatccccgcacagatttgttttttcaatttttttccatcgctattgctaaccaaatcggattgcgtatggacgaaaacacggtgatgattgtggtagtgatttggttgtgccgattttgggaattgtttccctcgtatccgattctgagaggaattgggattcgggggctgcgggcgaaggcgcaccgcgagggacaaaggcgcccttcgtccgacctgtgacgataggaaaaatccgaatatttatattagttatagatatactttgtatacaactattcgtatcaatctataccattgtaattggtcccaccacccggcggtttagattttattttttttattaatgtggtcttacttttggatatttatagattatattttactatactcttcttggtatatgtaggatttacttgtttaattattttttataactcttcaaacatactaggtgttggatacttagatggttctatcattgtgttagaaaacagcaaatcttgatttcttatcattttcatgattcttttgaagactactagtatcattggtaaaaaaaacttttaaatcacccccttacaccttttgaattagtgatatgtgatcatgattttcataaagacggatgaaaagatatatgagttccaccaaggcttccatctagtttatttgagttatttttgaggagatttctatcaaatctcgtctttgtttagtatctttaacaaccatttgtcaaacaagcatattttcttcggcgtcaaggtcttaaatacctaagccactctgacataggaatctctctctcccatttatccatgaaactcttttaatcactcgagtgtggacacccacttatctatagcatcaactaaatgattataaaaaaatcacaaaaaattgataagatatattagtaactaatatatcactccataaggatggaaattcaaatttgatttctacaaattataacaaaaacaacaagtttgactgtgaatatatgtgtactaattataatttaatttgttatttttttacaactcatagaaatcgatttgaacttgcatgcttgtaaattgatatatcacatattaatctatcttgatattttttaaggattttttaaaataattatttagttgacatactcacatacaatagataagtcagatgattagaatcattctcccgtttatttatcattggtggcactttgctagacaaatatagataagaaaacactataccagatcagatcacctctgtcgaactcaaaccctcagctgtgacgagtccgaatcacctgtgacgggtgaaggccggtccgatgcaagagtcacctctaacggccttacgtgctaatgagaatgagacaccgtgcgtgacaaatgatgccaacatgtatgaagctctgatgacttaaaagaatacagattgccaacatgtatacccgtcggattagttgaccgtgaaataattctccgtcaagcaaggttgctcattggggactcaccaatcatcacttcaccttagctacaagctacagcgacaaatcacttatatatatggagatcaaatgagctaagtaagagtgattagaagcaactcaag

SEQ ID NO: 84 - DNA, 2565 bp, Oryza sativa
tgatcatgtggcatttcaggcatatgtaaaagtaataatttctttcggatacctactggtagttcttttctagggtgatccatttgcagtgaaaatgtatgcagatcacataccaatcttttttctcaagaaaaatactgatttttcagggataaaaatatatttatgtatgtagccgaatacctatttgaaatttttagataatggatttcaatgcagcctctgtaaccctcaaagatttgaggcgtactaagaattagccaaaagcccacgctcgttatgtcttacatatgaagtacatggtgaaaattttcctaccttaattagtgggcctccaatcctaggccatgggccatgccccgggtgacccaatgcctaagtctatctaaatagtgataatgtacttacttgtcatggatcagatacctttattttaagccatatgagttaaactaaaaggatatctagtaaaaaaagaatatcatttcgatcattagctcattaataagacattaaaacctgacaagaaattaaaaccgtggtttccatatcttcttaaaagcaccccctctcatctacacaatatacatgaacctgatgaagctcttaagtcttaacaatacattgcgctctagagagaaggatagatcaataccaataagctggaaattatcttatgtggttaggaaaacacttttgttctgaacatacaaaaagaaaaatgtgtattcgtaagattcataacaacataactcatgagcttgcgaatgtggctaggcaatcccagcacaattatcattgagctatgagtgtaatgttgtattacaactatgatatgatatattatgatagttcacgtactaactaataaatcttatttctccactaaaatccgaaaccacctcatagccggtcattttagaatgagaagagaatgagtatgtacaactcaattacatggaaattgattgtggttgttgtaaatatttgacgtctaagataagatttggtcaaacatgtgaaattttggcctttagttttatcttagtataattttacaagatctagtaagtttataatatttgatactagacataatatattttaaaagtaagcatttgagatattattaatggtcatagacttaatctatggtaaacgctaaatatttatgaagaggggagaaagaagggagatcgaaattggtaggatattgttgcatattattcgatgattttattgcaatcatttcttctttatttgtcttactcgttgcattcaaatgttgcatttgttccaatgttgcgccaattctttttagaaatattgcatagaattttgaatgttgcattactttttccaaaagttgtacttgtttccaagtgatttaaacttaatatttaacattttatttgcggaatgttgcaacaaagatgtagtgaaacatacatgtaatgtttcatctatttacttaatgtttcatcaagtcccatatactcccttcgtttcgaaatgtttgatgccgttgacttttagcacatgtttaaccgttcgtcttattcaaaaaatttaagtaattattaattcttttcttatcatttgattcattgctaaatatatttttatgtaggtatataattttacatatttcacaaaagtttttgaataagacgaacggtcaaacatgtgctaaaaagtcaacaatgtcaaacatttcgaaacggagggagtataaaacatttgactggttgttgaaacacttgaccgactgaagttttcacttttttggggtgttccacaaatgttttcgctcacttgttcatctttgatcatgctaaatatcttaagtgtacacaatttttttaatcaaagtgaccaaccttaagttttatatcgaaaacaacgtaggtccgctcattagagcagtgcttcttgtggaaaatatatttgttgtattctgtctctacgactttatttttctttagaattctatctctaccattatattttagctctataatttatatttgacttttatcgctaaccgatatatgtatgcttctaacacttcttacattccaagcttcagatccgtatattacatcagcgtgatgcacctcgtttacttccaagtttccaagccatcgttccatcggtccctcgtttacttgaagttccaagccaaggtagattattggtagaactacgacgtggctccgcgatgttcacacgcttgattttcccataacaactgacgcacccccgattgggccaagccaggcagcttccattcacgccgccgaacccgcaaatagactaattacaactatttctgccaccggctggtgctagcctttgaccgcgacttttccaccaaaccaccacggcagcatgttactactaagcaccaatacctctcctacgcgcgtttctcattttctgcaacacttgcaaccttcagtcgtacacgttaacgtcgcgcgtatataaacagccgatccggtagatgcaattcgtacgtgttcgtgtcatcactgaacgttagagtggacacaacatctcaca

SEQ ID NO: 85 - DNA, 2968 bp, Oryza sativa
gccaactctggtttggagggagttatagagaactaatatagatggagggaggaggagattgtccacgagtatttgctttgcccattcgctgtgggattggcggagccccgcaaggaggatagtccgagcatggcactaaggttaaggagctcacagtcgcctatgttgcctctcgagctcgggctgcggatctagaacgaggccaaggcaaaggtggaggtgggtggagctcaggctgcggatcatcaagttacacgcatcgtgggctcccttagtgcccagactaccttgagctccattgcataaagtcaccacaccaccgcttggaattcccgattggggcctagatgagctccggagagaggcgtggggcagcagcggttccactcagagctatgggcagcggtgctcgaggagcaccacgtcgaagacaaagcttaaagaagtggtgcactcagggatgcagagcacaagctcgattggaaggttgcagcggtgcttggggacattgaatttggcacaggcgagactcggggatgaggaacttgagcttgacgtcgaaaagagatgagatgagagaaagaggaggtaggcccatagttgtatataatactatatttgatgactagattgccatgtcagttgccacgttggtgaaaaccaactaatttttgacttaagggggtgttgtttgaacggtttcaaaagatgaggggtgtttagtaggcaatcaagtttaagagtgttttgtaggcaatcaaagaggtaggggaggtaatatggacttatcccaaataaaacaaagaagatgagaaattcagtcggtagctagcatccaacctgcgataagcgaagcgtagacgtttctgattcactatgaaatttaatgtcgtcggtcaaaccaattaatttcgatctcgcacttctcttcaatttgaatgagtaaaagtaaacttaaattaaatataaactagcatggtggcccgcacagattgcgcggctagcatcattatattttctcttatataatagcatatagtatgttttcccattatattattcaaatatattaaaatgacaacataattttaaattttgtagtaactttacaaaactactaatgtgtaatattcatattgtattttatatacgtgttagttattaattatttttaatatcaaattttagttatttgtaaattatacatattgactcttttttaatatttttttaattccaaattttctgtaaattgtatttctatatagactctatgctcttcttccaatattatttattttatttttgaatttttattatttctaattgtgtttctatgtggactctaaactaatttttaattttgaatttcagttacttctaaattgtattcctatattgactttaaactcttcttcccacgtttttcttaatttcgaattttagttatttgtaaattgtatttttatatggactctaaaatctacttttaattttattatgtttattccaaattttagttagttttaaattcctatatggactctatactctacttctaatattccttatttttaattccgaatttcagttatttcctaattgtacttctatatggactctatactatacttctaatattccttattttaatttcgaatttcagttatttcctaattgtattcctatatggactctatactatacttcttatattccttatttttaattccgaatttcagttatttcctaattgtatttctacatggactctatactctacttctaatattccttattttttaattctgaatttcagttatttactaattgtatttctatatgaactttagttccctcttctaatatttcttattttttaattccgaatttcagttatttcctaattgtatttctatatggactctgttttttctttttctccgattaatgtgggaatttctaggccataaaagggaacgtggaggctcatttttctattcctttaataatataatagattgtaggccccattatcttaagaaaatagagatgaattgcttgtacgaataagatgagaccaattgaaatgggatatatcaattcgcggtcgtcgtcgtctacagaatctgagatatatcgacatctatatatatatatatatatatatatatcctccccaactaaccttgctgcgtgtacaatagtacgaacttgcatatatatgattatgcagttataacatatatgcagcacaaggagctaaaaactaaaccaagaaatcaacttggattatcactagcttaaattgttgcttcatagttaacttatcgatcaataatctgtggtcacctatctatatataattaaagaatatttccctagcatcgtacaattggacattaattgacatggtctctgcaactaaaatttgtaatggactaagaatacatgctctttttcgtttgaaatgtagcatcaattactagaattaattagcttagtacgtaaatgcaaaaatgtcaaacaacgatagaaacgtactcatttaaaatttgttcggaccttttcttaagggatttgttataaaagaacttgagagtcaaattgaattattgcgcgcgagtcaactgtatttggaaaatggtaagtgggtcgagtaccttacaagttaacagaactcctaatctaaaatttatagaaagacaagtgatgtatatttaaaagaacatgcaatcaatattctcggtcgtgatggaggaggaggaggaacaattccaacacattttatatcataggagtatcaccttaatcaagctcaacttgtaggtaattaatggcgatcctgttagctagcgatcgagctataaaaatggcagcctccgatgcgtgccccatcattgaaagaaacaacaatcactggtgcgaaatagctaataattaagagacgatacaggcgctaacacac

SEQ ID NO: 86 - DNA, 3000 bp, Oryza sativa
tgtttgggattatgcatgagttttacaaaggttcctttgctccggagagcgttctcgctttccttctcctagagctcaggttttctgagagtcgtccccttgtctcggtgggcaaggtcctccttttatagctcaaggggataccacatgcaccgcctctacctacccttccatgggagggggaccccactccattttgaccgggcctcgatccgtgccttcccctggaagctaagcagcaaccggtccttgagtgggtcccagcgccgtcccccgcctgacacgggggacagccctatcattctctcataaatgagtgaagacttgcggcatccctccttcgagtgacgctccgatgtgacagatcacacttacccccactccgctggagacagaggcgacgtgggggcatggccgcccgttcaatcggacgtgaccgacaacaggccggtcacagactggtcacatccgattgacaggggtcagttaggcgcaccgcacgtctgcccttaccgcattaaatgcagtgagggacagttggggcactgcatatttatggctgtccagcctgggtccccctctcacactctccccctgccacgtggcagccgggcgtgggcctcggggggaagcagcggaagagcccaagggggtgacgtcgcgccccgaggcctcccccccccccccccccgccgccttcgcgactcgtgaggtgcgtgagtagggccagcttgcagagccacgtggctggacggcaaggtaactctcctttacttcacatacccccgggcccatatctccgacaagtacaaagggagtatgtatcactactagtcatccatggatgcacccattaatttaatctctctacaagctacacacacatctcccgcccacttgcatgcatatatacatgcacgaatgatttaaccttaataattacttccacacgttttctcgcggaaatgcttgttatcgatcatctagcggagaatagaaaatcggacgccgacgcttgtagaaataattttctctctcttcattctccttcccacgcatctactacatacatgctttatgctatagactttatgcatgcatgggactttatttataagctttaaatatctttttcttcaaaactgtttatccgattgtatcattgtatttgttgtaattaaatctttacaacaaggtctcagttgattgtgttctgacgaaatatatatatatatatgttttagtctgttaaaatttaatgtttcacgtgtgagaaataaatgtttcagttggaatttgactatgttgcacattttgtgttctatgttgcaattgatttttttaagtgtcgcagatggtgctatttgggtgatttaataaatttttttttatgtttcactagtttatttgcaaatgttgcatgtggtgtcatttggatgtttcaatatattatgatgtttcactaattcatttgtaattgttgcacgcggtgttgtttgaatgtttcattaattattttctaatgtttcattaattacacttcgttgtttcatgtataatatagaaatgtttcaataaagaatataattatatttatatttttttattataaacataatcaagtgagatcttgttgcaaagatttaattgtaacgaatatgatggtgaaatcaaattgtcgataagataagtaatttagaaataatttaaagttttacttaaaaatattgctaagagagatagaggcttgtactgtggtataggagctcataggcattcatcagttgtcagagaatagctcagaccatttcttttgcaacgtgtagcatgcttgccatgcagcctaggagacgttagacaagcatccaatatttcgtaaaatatggtacgtccgatccgtagctctttccgttttctttccgttttcttgagacataaaaaagctctcgaccttccgttctccgatctgcttatcctttattgttgaaatttccatgaagtccctcgcacgaatccgcagccaataaaacggccacttcactccctgtatatatatattttttttaaaaaaaaaaacagggtacattcacattcaccctgcaaataaaatatacacaagtatgttgtagttatacttttataaatatgatatcgatagattgactcggtatatatttaaattgacgaagatattatagatgtcttgttgtcgatgaccataccatttgatgctagcacatacttaaggaggacactgtcgaagatattgccacattaatggcctatttgtttttaaggagtggaatgatgaattaatttgccactctattctcaatgaatgatgagttgttagttcagtaatatggaatgggtttgtctccatatcacgcctcatttctcaagagtacatgctaagttcttggctgaagaatggacttatttgtcaaatttttaagatgaacccatgacagatcaaccattctagatgcatgttttgaaaagatcagcacaaaaatgacgagatagctagataagcagaatttaaataaggtcaacgaccctagcacttttactttgaccaaaagcacaaaagaacaacacaaacaccccacattttaatttggatccgaccaaaccaacccaaaacctcatcaattccccatcatgcattaagtcggtctctctgtcgaagccagctcgagtgcatgcatatgcttgacctacacgcctacacccacatttcccgtccacttgcatgcatgccatgaaatgatttatccttatttatttccacgcgttttttcgagacgtacgcaaaaagcgctagctcttgttgcccccagctttaatttgtactcgaaattaccatgaagcccctcccgttctgctgctctgatctcgcgcctataaaatggccacttcacttctcctctcgatcactcacccacatcacacgctgcgacgacagcattgcgagctatagctaagctagtagctgca

SEQ ID NO: 87 - DNA, 3000 bp, Oryza sativa
taaatgctgctacatctctcatacaaaacccacatataatagataaaacagtatatatcagtattcagcaacccacatataaacattcaacattcagccacatataaacattcagcattcagcattcaacatgaagcaacaatatatacttcatgtatacattcagcattcagtattcaaccacatgcccacatataaacattcagcattcaacatgaagcaatcatatatacttcatgtaaacattcaacactcaacattcagcgtaatgctcagactagagaacaatgcatctggaggtaaaggtttccatatgtcattagggctggacaaaaagctcttgactcgttagctcgactcggctcgttatgtttttgaaacgagccgagctgttgttttagctcgttacacgtaacgagccagctcgagctgattcgcaagccgctcatgagcttaaacgagctggaaggatataggcagacaagttagcaggccaaggcccatatgaaggaaacacggtaggccccaagaaaacagtcaaaaaggacagtgtagctctcatcttcctctttcccctcatcccaaattcccaatccagatcgatttccttctgccgcgcgccgccccggcttgttgccgcgcctcgcgtccttgcgtcacgctcctaccatccggcgcacctcctcaaggtccggcggtgacatctgttgcgcctcgaccacctgcacgtctctcccgttcttggcttggtcgagtggtgtcatggtcggggcaggaggagttgatagagatggagcgctgcaacgagccgccaatcccagccactgctgctgcctgctaggaggttgctggcacggggcgatggatcctgcaggtggttgagcacaaccacctgtgtgctcgatcttgtatattgcccaccagcaaacgagattatgatttgttaaaggtagagctaccatgaataatgatgcatttgttgaacacttgagctagcacatggtttcttggtttgatacaaagataccattgcattcttggacattttgtttgtataatttgtatttgcatcgcaagtttttttttccggtgtattggctatttggctcgagccgagaagcgagccgaggcagcatttataacgatccgagccagctcgttatcctaatgagctaagctagagcgagccgagccgagccagctcgatatccaccccctatatgtcatacacgtgatgttctctacgtgtggaaagagaagttaaacttggatcctaagtttgaattatatggagatgtgctcactggtttggacgtttggtagatttcatatgtgattaggagtcctaattctaatcgcctccgtgactagagacagtcatgttgtgggtataaatagaatagggattgaggctggaaaagattgatttttggagagaaagagttagagtttgctttaagatttcgattctagtttgtgagttgagagatgggtgctttagatggactttataaacatatatggatgcaataaaattaatctgctcttaaaagttttgtcgatacgcgtttttctatatcccgacagtttaaccggagttcatcatacggtctaaaaaggaacaagacgatagtcagaccaccggcgagcagacggttagaccggcgagagtgctgcggtcagactagagttaatctagaggtcagactgagctacggtgcgacttcatcgaatttgaggaaaacttttaattcctctgaatgtttttttttttgtttttggtgttcgtaccatatatttaccccctctctcgtagacttggttcttatattcaatcatctaattttgtttccgaagcagtaaaattcatatgaaaattttatctgcataattagagaaattttggtcagagtcagcaccacctcggtacaattaaaagattaattaattcgatggtttgcttatcctaatctccgcgtgcatcgggttcgatcacatctcaaatctgtgctgcgtggtcggattgcccacttgcttggctcattacgaacaagtcaaaagtcagttgcgaaaacttcgaggtcgccctctgcaatgtcaactgccataggtagcagaaatcttctgcccatcagaggatgtgcttgtttcgatctgtgaactcatgccactatgagttagtttaacccggttcttctactactactaatctacctcaattcctttctgacctatactcgaaaaaaacaacctacctaagtgtattggttgttttgatgagatggaagatgaaatggaggtaaattaattaggtttgatctctttaaaacaattcctccttccatcgatcctatgatctaattagcgatagagatatacacattcgtctagaatttagtaacttttcatccatccaaaatatataagcaacttttgattatctatttgaacatatacttgtccagatgtatagaaaaacttagccaagaagaaaaacatcttgtgggaaaaaaaaagggatatggaatggagtactcccatgtcccaaaaagaacccaatcctagatttgaatcaggataaatattatgtctagatttaaatctaggcttggtttttttttttttacgaaggttgtataggactagctaagtagctatgattagaagagcatctatatatacttaataaaaggaaaaccgagtattggaaacacaacaagtggtacgctatacgcagtaattgatatatataatcttcatggtcatctcgatcacttgtttgaattggtcttggtcaataagcactccattaattggtctaggttttctatcaccaccacatgtacatgatcccctaaagaagcttattagctgaaggttacttcactgggcagcactgtactctacttcgctagccttacgctctctataaatacgtactaaatccatgggttagcactcatctcaggttaacaaacctagctagccagtaagtccaca

SEQ ID NO: 88 - DNA, 1352 bp, Oryza sativa
aaagtgccatatttgtcatcatatttgttttctgtaaagtgccatatttgtcatctcttgaaaattcaaccctaacaggtggaccagaccacaccaaaggacaaatatatacatacatgcttttctaaaatacaattgtggattaaccgctacacagcaccactcacaccgtatatgcaggtaaaggaatgcaccttccgttaaccacgctagagaaaaattatcagaactacaagatacttctctattgaaaggacacaggtatgaaaatgcgagaaaatataaatgacgcatataagttttgtttaattttaaatcacacatataataaatgacgaaccaaataatgtgtattaaaacaatattgcattattacaatttccttaagaatatatattaatgaatgttaatttgttgtttgccacaagtgactggctgtttatgatttcccatgaatttacaggaacaattaataaccaccttaatttttgacagaagttaataatatgtcaggcatgtaccatgtaagaatcaaatcacgcaaaaaaacaccttcaaaccaaaaatttgagatgtcttttgaagttattctgatgttcatgttgctaatgtataattttggaccaagagttgaggaaccgcaaaatcagattttcccagggtatcttacttgaatgatgaactgcttcttcttttcactgataataacttcgtaaaacttagagaaaaaaaaacactgatagaattagctctatgcataatgcatggctgtctctagatgcattatcaatccttttaaataagcacctcaaaagaaatgatggataattagctccaaaaaaacaaaaatgtttactctcccatatacaaaccgtcatctttcgtgcacatttggactggactatttttttttcgcggcaaatccatatcactgccggttggtggactaaacctggaatgacaaacctccagggatatttttgtgtcaactccaactccatcactgcgccacatgcagcctgccgtttcgaaaactggcagggatctcttgttgtgtaccagtgaaaataaaggggaaaaaaatgttggatggctaatgcaagtgggggcaaaagaattagacatctgaccttgtttggttgtgacattattccatacttcttctaaatgctatagccagggaatgcaaacgagaaaatcgatgaagctctgagttggcctcatgaactagagtgctaccactacgctataaatacagctagccatacaccatctctgatcatcgacaccaagcaaagaatcaaacaattcctagttccactttgccagtcctaccaaagcacattttagcc

SEQ ID NO: 89 - DNA, 1656 bp, Oryza sativa
ccatctagaaaccatcgagtatatctaccgcaacaattagaaaagaaaattttcccctgcttgtgagccattgtagctttggtcacattatttgactcttatttgccctcgaatgctataccaggaggttcagttcagaagtcatcctcattttatcactgtaattggggtatcttgagttcttaatccattttagagtacagatttgtgatccaatttagatgtacacgtgctttagtaaaaaaaaaacagaagcaggtaggaggaggcaacaccgcaacaggtagcagaacagatcacacaacaaaacatccacgaattgcaagctgggaattcagaccgtgacgcaggccgtgacgccgccgctgcggtcaaggatgagagaacaatctggaactgcaagcacacaccaaaattaagtactaaaattcttgactgccggagagatagggtgagtgcgtttggtaatcgtgtgtgcacgtttgatgaagcatatcatatccttgtcgaaaagaacaaagaaaattaaggaagcacccgcctactctgtattagcggacgcaatatcacagcaagaaacaatctgtatgtaaattttctcctagcaggtaaaaaactgatactcgctccgtccctaaatatttaacgctatttattttttaaacatgtttgatcgttcgtcttattcaaaaaatttaagtaattattaattattttcctatcatttgattcattattaaatatactactactactcaagtttttaataatattcacaaaagtttttgaataagacgaacggttaaacatatttacaaaagtaaacggcgttaaatatttagggacggagggagtacatgacttacttatcttttgctaggaatagagagtaggcagaaaatatataatgtaaatcatagcgtgaatttaaaaactatagtaggtttttttttaaagaaaaattgagaaattcccaagggtcttccggctagctccacaagatggtgggctagatgacctaggttcaaagcctcaccccttctaattatttgatattaggtcattccctaatattcgagttttttttaaagaaaaatcatctgtgatttattcatgtagtaccttatagtgacgtagataaatcttaaaattttattttcttattattctacgatagttgtaaagtttgcaacaaatatttttcccaccattgcatataggtaataatcacttcatgaggtgttaaattctggctattatccttggtgtaaaataattttggaagcacatagtatgtgttatgtacattttgtattgcctcgaaccatctagtacaacatcctaacaaaaaaatatataacgaattttagttatgcatttgtattgtcaaaatccaaatgcatatatgtatatgccaaaataggccacacccatgaaccaggaagcctctttttggaataatcaacggactcataacagtcactgctgacgtaagccacacacatctccatcgcctataaaagctcacctgaacaacctccaaatcacacacaaaacgcagaagcagctagcttcactgatccacattactaccagcttcttcttccaatcttcttctccgacgagcagctagccagctagcggcgccggccggca

SEQ ID NO: 90 - DNA, 1751 bp, Oryza sativa
cctctgagttagaggaggagaagcctagcataaatctaggcgttgatctaacgatacaaaatcgataagtttttcatctttaaatttatatttaatgcatttaacaaatccgattacaaaggggatgatttttggctaagtcttctgtactcatactattagctggaaaataaaatacgacgaaaatcacttaaattaattctgaaattcatttttttctgtggcttataagctgaaatagggcccatccttgtcttgtgaaaaacgacacagggcccggccatcctggcatttgtggcctcagcccatatgcaaagcatacattccactccaacaagctgctacgtcaactgcaagtggtcaagcctattttaataccacacatataaaccagtagcaaacctgcattttcttctctcttttttttagagcctttttggattagtgtcaaaacggtgccctaccaatattttgatagtttgaatagtgtatatggtggttttggcttgaagccaaatcattggcattgccaatgtctagtttgatattttctatactctactcaaagcttgttctaaattggtctccaaccaaatacaactctaccctaccaaaaaattagtagtgccaaaacttgcctaggttttagcactaccaatattttggtaggtattaaaccaaacaaacccttaatccaacatacaagcaggtatcccggaatatgttcaggctccagaaccatcctgttcaatttgtacgtaataattacgttccaaaacaacattcctgtacgctttggccaatgacgcccaaaagagccaaaaacgccgcccggtcaacctaccggccaatgctgcggagaacatgcatctttcgtaaccttcacgtacattacgctacgtatgtgtataattaatttttttttagatatataactccctacattttgtatctccatgatcacccaagaattcctcctcatcacacagagataataataatattttaatattactaccaacacatacacagctaaccaatcaagcaggaacagccccaaatcattgcaagctcccaagcacatgggacgttattcccttgacgaattcacatatatctctttgattaatctattgtcttaatgcattcccttcccatatcgtcgtacctagctaattagacgtgtagattatgtactacatttatacatgaatcatgttactatcccctctccaatgaactggcctcggcgccgtcctttactctctgttttttctaaaataaaaaaatgttttttttttgggtattgactagctcattgctggctagctgagccgcgcgtcgtcgccacaacgcgtacgtatgaggaggcatacatgtgcgattttgttattggagctgattttgcattagagccggaaggtaagcacatgcctaagctaagtacgcagggaaaatacactggaaaaacgtacaggttgtcacctgtgcgtcactagtcatgcacacgtctctataaatacactgagctctgcgaggacatgctcatccacagccagccatctagatcagcagctagcagcagcatcagcagatcgagcagcaaaacattctttgcttaatttggttgttgatttgtgtgtcgtggccaggtacgtactccggtcgccggcgtcgtgtcgcgtcgcgtgcacggcggcgccattgattgatcgatcgatcgatgtcgtgtttttttgcaatttgcaggt

SEQ ID NO: 91 - DNA, 2229 bp, Oryza sativa
caaaccaaacgagatatataatcttttctaggagtactttatatactccctccatttcaaaatgtttaacaccgttgactttttagcacatgtttgaccgttcgtcttattcaaaaaaaatttgtgaaatatgtaaaattatatgtgcacatgaaagtatatttaacaataaatcaaatgatatgaaaagaataaataattacttaaatttttaaataagatgaatggtcaaacatatactaaaaagtaacaataaatcaaatgatatgaaaagaataaataattacttaaatttttaaataagatgaatggtcaaacatatatcaaaaatttaacgatgtcaaatattttaaaacggaggaagtatatatcaacagcgagacatatatactactagctatatacgaatgaatatatgcagagatcaacaagagggcatatatatgcatagtcgtagtaggtagccatcgatcgatcagtctctctcatatcaggtaaggttaagttgcaggcagggcagggtatgtaggcccggccagggattcctacgtacatactacgtaacgtacgcatgcatcttattcatatatgtacactcctattactacacatgcacctccggaaattaactactgagatagatagctagcaccaagccaccaactaccacagactttttaaataatggactgtctattacacatctcatataaaatatctcttaaaatcttataatttttaaaccgttaaatcacattaagattcgtgcagacactcaaaccctaatctattcctcctctttttcgttttccctcagcaccacaccccagaacctttcgataccctactcggttaatctccgttgtcgctgcctcctttgccgccatcacctaatgccccgtcgcctagcctcgccggttggttggaaggtggttgacggtgttggcgagacccacctgactagcctcctctccccctctacttctcctcaagttgtcggtggtaaggtgccccgcctcatcgtccttcacatcactatcagctccccgccacctgatccgctcctatcggccatcaccgcctaacccgccgctccattttgcccctctgccaacatcggcccaccgttgactgttgaccctacgtctttggtagtggcaacgccaccttgccgcctgccagttccacctcctacgtccataattctaagcccaaccatcttctccttcttctccctccctcttccccacccataccccaatacggaaccttaatgtgtagaccctctgctagatttaattttagggtgctatcgacactgaagaagaagagtaggtcgtcagagttgaaggaaaaggacctaaatcataaatcacaaagcatatttgttgatctaaattctataaaatttagggatgggttttctttcgattgaaggttggcaattccgtacgttttaacaatcacatctacacacatcttgttgcagtcgcatcttctgctggggcacgcatgcatcgcaatattatagttgccttgccggcagcctttgcaacctataactgcatgcctgccatccatctacttccactgtaaacgtacgtttcatttctcggacgacatctcaactaacgaaagggattatgtgttctactgtgctagctggagtagctgctagatcgaccagaaaaagtgtagggaagcagccacgcacatgcattttctgcgtagtacgtacgtactactagctatgttatcttagtttgcactgttcgtcatctttattaattaattcctgcaaaggaatcggaaaacttaattatgtatgtaatctctcccggccccacctaattttttctgatgctagctactatggtagtttgcatgtatgcaacgtgaaaaataacagcgtgtagggtaactttccatgacacagctgcagagctgctagcgtcgacagagctaattaattaagagttcaattagctgggctgtcatggaatgggcgcacacaagtgtatatataagaagcagcttccatccatctatctctcacactgatccaggctctctccactactatttgcaagttaagttagtgcactgaaggagagcctgagatacgtcgtcgtacacacttgattaattacaggtactgtctcatgcatctcatcgatcgatcgatcatatcgctagctagcttgctttaatttaattactcctgcaggt

SEQ ID NO: 92 - DNA, 3126 bp, Oryza sativa
actttggctgtgctaatcattggcaactccgaaggtgaaatctcgttgtcaaatgacaaaagtgatttaagtgatcaatcattctactcgtgtgtgcaggcagctagcagaaggccaccggccacatgcactgatgcaccatccagtttctttaattcttagcatctctccaagcagcctgcagttagcagagtgtactggggacaaaagaatcggagttagatagcacaggttaattagcttccagaaactggaaaaaagaaacggagcaagtaagctagctagctaactaaaacagttcagttcactgagatgatgagtcgccattgctgctgtttctgaactgtctagtgtctactagttgcccatgtggccatgagagatgagccccccgatgaaagctggtgtttgtttctgcttctgcctacctgctatgccttttctgaacaaaatgataccacttcaactacctaacagtgtgtccactggtccatccatctacgagttgttgcaggcttagccggcgacccggcgtctctcgtgcgtgcggatgattgatcaattccagcgcggcgacgccgacgccgccgccggatgttcgatcgatcgcttctcatatcatcgtgtattcgtggtgatcgctgatcagagctctctttcctgttagttgcacatgtgcattccggctctgacgactcaaaatgcatcgtcagctgctttgagttttcagtttgttcgtttctcctttttgttggtgactggtcgtctcgcttgattcaacctagctagctatagctttcaggtgtaaacttgagctggatcggagcactctctgattggattttcagctcgccggttcactgtgttcatttatttcaggatttgaaaaaaaggggatagttagctaggaatggctcagctgacggacaacgtgttgtggactcatcgatcaggcctgtcgcgtgcaatgaacgatacgtaagttaattcagtacataagcgtagagtttgtactttttgcctactactacttcggttctgctcatgattcagggttcaggacatcgtctggatttctacattcaaacaggcaaatataaccagtacttagtgtagacggattctaatcacccagtggacattcacttgtttattatatgtcaactaaatagttataaaaaatttttaattttttttgacaagatagattaatatgtaatatatcattccacaaacatgcaagttgaaatttaatttctataagtcgtaacaaaaacaacaaatttaactgcaaatatacatatactaatttgagttttatttgttatttttcttttacaatttgtagaagttaaattttaagttgcatgttacgtttgtggagtgatatattacatattgatttatcttaccaattttttcataattatttagatgacatgcattaaacagtgcacatccattcgggtgattaaaagagtttccccataataatcctaactctcgttctagaacccaaatattaaacttatagcatatttgtcatttttcactccttaaatggatgttccctcgtttctcgcatgctatctaaatagttatattttttaaataataataataataataataataataataataataataatatagatcaatatgtatatcattctacaacatgtaggtttaagttcaagtcatttttttcataattgtttatacgatataaataaaacaaggggacatctgaacaaaagagaactatctcagaagaaagcagcattttttttaactcccattctagtactgaatcttgaacccaaataggtaaagaggttaaacggcacatttgccatttttttactacattttcacataactaacttatcagaagcgcaggatggtacatgttctccctccgtttcaaaatatttgacaccgttgactttttagcacatatttgaccgttcatcttatttaaaaatttttgtgaaatatgtaaaactatttgtgtacatgaaagtatatttaacaataaatcaaatgatgtgaaaagaataaataattactttaattttttgaataaaacgaatgattaaacacgtactaaaaagtcaactgtatcaaacattttaaaacggagagagtagttaattaaggtgtgaaatggtattgtttcctcgggcattaatctttgtcagttgctacagcctgcaacagtgcacatggcttgaaccgatggttacaggccacagaaaaaaaaaagggaatccaagtactttgtctgatcatcacctctaaagttaatcaacatgacagcatgtggataatctcccacgtcctctcagttgcggttcagaagttcacagcgagaaataaattactcttcaaattgaccaactttgctttagattccatgcaaagcacacgcctacaggatcgatcaagcagcagagacatctcgcactgattttgtcagtacaacgtacaaaaatacgttctcgatcgacgaattcttctcctcatctctttcgctgctgctgctggcctgctgctaacaaccagtaggtgctgcattcttgactcctgcccggcgcccgcgatgatcagaactccacaaccaactgtttccgtagccgattttaaaaaagtttttgcagtacaattggtacttcgttcttggatggcaattagtatctgaatgcgtacctgctacaagaaagattcggcaaagcgaaaacaagaacagctcattgtgccatttgcataatttgcatgcgtgattcttcagaaagtgcagctagcaggtagctaagccagcctaccacatgggggaatagggacaggatgggccaggacggcgccgcgctataaatctcgcgccaattcttgcccgcaaattcagcagcagcagctaccagcaacaagcattctcttctagccttagcctagctagctagctttgttgtctagctctgatcgaggttggtggtgatcaggtaagctgtgcatgcagtgtccattctccattgcgtcgcggctgctggtgtatagtagtagtgttcgatcaggtcgcctgacaacctcgtcgccggccggcgtcgactgtctgtgcatgcaggt

SEQ ID NO: 93 - DNA, 3118 bp, Oryza sativa
gaacggttttattaagaaaaggcaatgtggttacctaagggctgacaacatagttgattagttattgacgcaatagcccattagccctagaactagaaggccagcctatagcggccagcccacacctgtcttgtttatgacattatattatatcttttatgactaccatggtttttcttaccaacactaaattgcatcctctcctattctcactaggaagcagtgtaaaaagttttcatagcaaaaactaaagtttgcttcttatagtcgtcacttgtcatgacaatgttagattgatctgaattttacagttatctatacactaacaccaaggaagatgtgaattctacagttatctatacccgttgcaatatgtatgccctactatacattactataagtcaaatgcatcatacttcctccgtctcattttaaatgcaaccataagtttccacgctcaactttgatcgtccatcttatttgaaaattttttataattagtacttttgttgttatgagatgataaaacatgaatagtactttactcgtgacttatgttttaaattttttttaaaacaatttcaattaagacggactgttaaagttaggcgcgaaaaaccatggctgcacttaaaatgagacggatggagtaatattatactacctctgttgttttttttctaattgacgttggttagttaaaaaaatagggagtataatttactaatcatttagacagatgaattgtgtaggataaacctgatcattgagtccagtgagcagagtcgcatccatcaattccaacacgttgattcgtttagaccgacagtggcaaactaacagctacttacatgaatgcatgaacattgaacagtacaataaggctacatagtacaatacaatgtatatacgttgtcgctgctgacataagtggcaaggcaaagaacctgtgttcataggataataaatcccacagctatttgatcaagacgtgtcaaggtagttagcctcaatgatttttctaaatggtcctagcttaaccgatttttggacatatcgaagataatatcaacaacactgtgtaaatagttactatgtctacgtataatgtggaaaaaatggtagttggtactagtagacaacaaccaaatagtgttggtgttttggtttacttaatttcttttagtgcataactaccatgtcccttttgtttcaaatccttccatagtctgagtgcttttcaaagcatttcgtcattaaatgcaaagaaactctggctggtaataaccaatagacattgaggtaacagctcacctttgctggtccatatatatctatctgaactattgcttcaatttgcaaaaattcaattccaagtgtgttagttttcccctattttcttctgaaagatgtctgctggtcctgtcacatgtatggtccatccatgcaactcagttcaagtgctatatactgctttgaaagtctacttacaaagcacactgctactgtgtaattctttaatccgagaatcaaacttttgcacaggaaaaggaactgtgctccttctgaaaacattgcattcttctagtagttcaaacttcaaatgatatggtcattcagtcattcagtgcgatagaagtcaattctgaagcaaaagtcttgtgctttgactactgcaaatgatcctactggcagatgaaaccaagggagctgtcatatcagcagggtcagagagccaataactaatctgaagctaattgactttgggagatagcattagcagcagtgcaaacaattaaacatgccagtgcgagaaaactggcgtgagatccaataattgcggccattgaccataccacattacagacaattcactattgtcgttctctgaacaatacctgtagtatctattatcttttcttttcttttcttctgtcaggcaatcagtcaccaacaggcagcatctaacagtaacggtaacagtaaagttagtaagacaggcaccgacaatatagttatacacagcctaaatcagaattttaaaacttaattttacagttgattggaggttttttcattgtagtttatttttaagtactagcttttaaatcgctaagttcgtaaatataaaagttttgcatataaattatttttggttgcttgtttattttttcacgcttatcagtcataagcaaacttacgagagtggaatgcttcagcaacaattacaagcaagaacagaaatcccggcaagcttcgcgttcgattgactgcatcttttcctgaaagtgaacatgcagtgcatggagtttcttgcaggatcaggatgcagcattgcattgcagccgaccctgtcgtggagtaatcattagcagtagaaatgaaggcagcgacccaacccaaatccaactctagatttcctcctcttatcctttctgttcttgaattgatcttgttaagctcatcttctcatccgctaatgacagtgatggtaagcaaagcgccatgagcattgggaccggatttgcaacgcgagatgattagggttcatggtgttccccacatgacagcgtgcttggatcaaccgcttttgtcttctccagctcagctcgtcttctccgcttcatccatctattataaaactgaaactctgatgagtttttcattgcttccttgtctattgtttctctgggatttgtctgtctgttgatctcactggatttggaaacgccgtgttcttggatactcgtttttcgttttttttttagttgtgctgctacctaattgttgaaatattgaattgaaggtttgaaggttgtggcattctgttgatttggattaataatagaattgggttttggagtattttatactcgtcgtgtacggatatatttgcccattcctctgaattttaaaaagaattttgcttttgagtaaaatattggcatagttgacgacttttaaaagcgaattgagcactatataaacaaccacatcccgcaggtgagtcactcgcagctactagtagtttttttcgtttgatttttttttagatttgaggatgttggattggtttgggtcgaattcgtttgatggatttgagcgggggcgtgcaggt

SEQ ID NO: 94 - DNA, 2310 bp, Oryza sativa
ttcaaaaaaaagttatatagaaaaatgaacaaaataaaagttgtaaatcttgataagttatacaactttgttgttgacaatttttccatttaaaatcacttactaccaaaaaaattattttgactaataaaataaattattatactgcatttaattattttgctataggtcaactcacaaatataaacatttcgtatgaaaaatgaaaaaaaaaatactaagtcatcttaatcgggcattagtatagagcccatcatagatgacgggcctagttgttaccaattgagatatggtcgcacagacatgttagatggtcataaaactcatactcgtcatggatgacacctatcagtgacgggccgtagtttagacccgattgagataacattctaaatctcaatcgggcaataagttggcgcccgtcacagatgatggaagcaacttgggccccatttgagatgtggaatgttatctcaatcgggcctaaactacgacccgtcactaatatgtgtcatccgtgatgggcctgagtttatgccatctaacatgtctgcgcgaccatatctcaatcgggcgcttttttggctcgattgaaatgacttccttgtgatggcccgctaattccaggcatgttagagaccgtcacacaggagagaggatctatactagtgattggtcagaccggagttatgcgagtagtcagaccagccaactcaatggtttgactgaccgactctaaggtatcggtttcgggttgtttatttggatactcgtgattaattcatatttatgacttctagatgatcaatgtacatatgtaatactgttctatgctaatattgagtcaagtttaagagaacttgtgctcggatatgatttcttatttgattcatgtgttggtgtgccatgatgccttagaagagtattatcggtgatgtatcaggagtcaacttgggagaaaatgacgtccagacgatcgggatattatacgagatgctaaggctaaaaaacaatgcacgtaatcgatggagattccatgcggcataagaaacatgtatctagcaaattcatgtatagggagattgtgtgtgtatagaatgagttgtgaatttggtaatatcacgaggtcaattggatgaggtaaaacatgtatctccatgacacaaaatcaaacggataaatattataattgattgtcgatgaaacatcgggttgtacgtggtgaaacaacaaaattagtgattaaaacatttcaaagatttgtgaaacaaaataaaattcctattgaaacatttcactctcgtatatgaaacaatgatgcgattcctattgaaacatttcactctcgtatatgaaacaatgatgcgaacaacttttaaaatgttacaatcatgctccaaactcacaccttgtttaacatgtgatcatacaaagtaactagcaaattctcccaaataggagtatgataaagaaacaaaagctcaatttgaatcctacctacccaacccaaacaaatgtcgccttccccaaacaaaacgcatcccaaactagagtgcttgacctcacccggttttccctcgcaccgttacagcgattcgtcaactcatttcgtccacacgcgcttagcgggacccctccgcagccaaacggcgcagtcccccgcccgagtcgccatgcacgttaagtttgacctgccaacctctcgaaaccttataaaaggacacctcggctcgatccattgctcgatccatctcaagtctcaagcttaattaagctcgtaatctcatataacctctttttggacgaagcttgccactcgatcgtgttaaccactcgagcttgagcgagcgagctagctttgctctgacgagaaaacaggtgagttaatttgtcaacgcaatttcttctcgtagaagttttaggtaattaagttggtacatgtcggtgttcggtactagggttttaattccatactgcaatttggccagctggttcgataattaatgaagtgggaactaggagcaagtagagtagtgtagcggtagattaataagcgggtcgcaagtcaaaagtgtgttgacactttacatcagtgcaaacgtacctcaccctcaccgcccggccggaatacgatcattgacgaggcatcccatgagttattaaaatccttagctgttaggctcataatgcatagtagatgtttctgaatcctataaaaacttatctgaaaaaaaaaatcctatgttaatctaatcgattaattatgtgctggttcggtcgtgatgttgacgtaactatacacatgcaggt

SEQ ID NO: 95 - DNA, 1323 bp, Arabidopsis thaliana
cgaagatttgattgcctagctagaaagtattcagaaaaaagtgtcgaatgctctggaatatgttgtaatgggcatagataaaattttggatcttcaaaatgaaatagtttctataactttttatagtttaatggtttgtgatcaacagttgtgttacacatctaaactcgtttattttcagcaaaaaaaatctaaactcgtttattacatgttgtaacccaaaaaaataaaaaaatgaaaaaccgtcgcaagtgatatcgacctttcgcctactttcgcactcatgttggtgtcttgacaaaacccactgtggacgcatataaccaccggaggagtacaggccaaaatttctattattaatatgagcttagttagataatccaagtctagtaaaagatttgaataaaagcaagttgtgtccacaaaactaaaataaaccaaatccaaagtcagtcacatttggaattatgttgtaatgacagtcactgctttgtcatcatctcaccttttaaggcattgaccttttgtttttgttcgacaaaaaccattttcgcattcatcactttcgaataaaattatataacagtctaagtaacaaactcatattaggataccaatttgaatttgacttttattggatatcgtagctaacgaaaaaaattcttaatcattagacgattaagattatcgataactcaaatgtttttttttctaagttatggtcgaaatattggtatacatcattcaattttaaagcataactagctagtgataatgactactacatacgccataagatgaaccagaatcctatgataatgatatatccaaaaaaaaactgaattcaaattgttgaattatctgagtttaactttttatcacacacaaacaacaactgaaacatatggaacaagaaaaagaacactttattttcttgaaaactctgtaaacttaagaattagtttatttaaaaattaaaccaaagaagcgttttaaatgaactaaaatccacatttatacagctcaatctgtacttctttttttctaaatttagcaacagaaaaataccaaattgaacaccttcacatataaaataaaagataaacatgaattgttttgtaagaaatttgataaccccacatgcatttggtcattatttaacaacaaaaagtagactcaattttaatagtatatctttagatcgaatcagcataatgacgtatctttctgacgtatagcttttgtcttggttgttctttacacatctataataaccaaacctgtgtgaacaagacctaaaacagagagaaattcacaacaaaa

SEQ ID NO: 96 - DNA, 1407 bp, Arabidopsis thaliana
cgataagccttgcaccttcatttattcctttcagttcaatatagtagttgggctattataatcgcaacacctggcccataggccatatgtcttcttcttttttttcttctaggccatatgtctacgattgaccaaaagtaaaataaagtaaatgttgtttacggattagattggtgtgaaggagctgtgagcctatggcatatgtaccacgttaagatttaagaaaatctggaataaaaaatgcaactagatattatgttatacaaatctaggccagagtcggttgttttcggaccgttctgccgtgaaactgtgttccttaacattttgatttaacgtttccatatttctgacattccaataaattattgataactagtaattaattaaaacggtttactctgaactctgaagtgtgtgaggtcatcaaatgtcaacggctacaatttcattagttacgtgcgtgactgcgtgttgacatattaagcagacaaaattataattacgttagatcattagcaattagatacagtcacgaatgcattgaatgtaagttgattataaaactttaaagcggactaattgtatttaaaaagagagctatagttagattggaacttggaaccatcgatatgattttatccgttaaataatttgagaaaatgtttaatgctgacaaataattgaacaatttgtcaacatattgcaagtttgcaacaacgaaaaacgataaattaacacctaataagtcagttgtacaaatatacctacaccaaacatgcatttacatatttgttcaaatatttttctttgaaaggagtgttgcaccccccataaaataaaacgtcaatattctttcctgttttgtttctttcggtaattagagaaactgattgtgaattctttaccaacaaacaaacaaatttacctagcaaagttttaatacttcaacaagcttacatgatattaaatcatttgtctcgaaattccgtgttatgatgcactaatgtcaatcaaactattaataaaaacaaatatcataacaggtaacggctactattaataactcaattatcatcgagatacgcatatagatagacacataatcaaatactaatattttctctataaacgtatgaatataatgttctgcaataacagataggcatctttgaataagcagacagagtttccgatgattagtaaattactttctgcaaacaaacctacgcgtttttatgactcccaaacgagttgactcaggattatgatcgcattacaatatcatagttatcatacacatattaaaaaattaaaatggacctacctcatcctcattaatcatcaccacacttatataaaccacttaacctaaagtttgtattcacataaaactttccagtacca

SEQ ID NO: 97 - DNA, 2512 bp, Arabidopsis thaliana
ttaatataagattaattaaccattacaatatgatataaagtaatgaataaaaatggagattatatagactctccagcttttaataataaaccagctagtttaacattttcttttaatgtgactaagaggtttaattacgacgatgtgagtgtgagggaatacgtgtttaaatcacacataagaaaaaagaaatcctatgtaattgtaatctcttaagaaaatttaaaataagctatatatgtgtaatactgaaattctcctactttagaatattttatggctgttaattatgaaaaaaaaaattggactataaacagaaacaattaattaatggatatatcaaaaaaaaaaaacgcaaacgtaaaatagaataacatttaggtcaataaaattaaccaattgagttaataatatatataacgtctaaatatgtacattagtttccaactcgaggcaatgaaagtttttttttttttaattcaataaaggatgtaatgaaaataaggatgattggttttcaagatgaagttaaatttcataaaatagtatatttggtatcacgagattttcgccaatttccggcagaaaactgggtgatgaatattagtctcatctcatattgtataatgtacatgtacgtgctcggtgatgggtgaaatttgtcctctccgtctctggtttttataactagaccgatattaattaaaattatgtgtcctgtataaagttatcgttcttaattatacttgaaatgtgaatattggttcctttctttatcttaatctataactcttaactgtaaagtgaaatttgaataagacttttacaaaaaacataaaatacaattagtgaagtcaatcaaagttggaacgattcagtttgaattcactgttcgaaatgaatctaatcattggtggaaaatgattaaagggataatagtaaggataattatatatcagtggttgactttgacaataaagaaaaagacaagtcaatgatattatgccttcgaagttgtctcttggtggtttcattaaacaagacatcctcattcctcagtcaaaattatatgctctttttttttttacccaccgactatctagtaaacattaaacaagacatcaagcttatctctttacagctttgataaattcgtaacagttttgacaaaaaaaaataaaaaatactagtttcatacggttttcattaattcgtaacagttttgtttttagttaatatttttcaaaatggtaatttctctgcagcacacttttaaagtcgaattcatttacaagttgtgatttagcggtcacacacgtttttccgtcatcagaaaattacacatacacatacatatgcatgccagctgcaagtatatcatataatgttatactgtattactttgtaactcgttaattactttaattaagggaactgaataactaactacagccatcactatacatattctctttgttttgtactttccccatgcagaaacctacttagtctattattgttcaatggttcaatttttttattttatttgtatggttcaaattttccatggcggtgaatactttaatgttttttatccttcaaagaataaatagtagtattatatattggctaatccaaaagagtagagaaacaaatataccattataatagacgatttgatcattttaaaaagtcaactatgcccgttacattttcatcatccctaacgttctgaccaaaactttgtaatctatattatcgccacgtaactcattgatctaatctatgaagcaatatttgaccctaataaaacatcactattgtggtaccaactggtatcggagattttactttttaagccaactgattaatattttgtcacgaataatttgcgtttatatttttcaaaatcacattttagtttttacaaaaaaaaaatcacattttagtgtttcttgaatagatttgatcacttttcttaatgtatcgttcatttttttccaattaatcaactgcaattttggccgatttagctacatgtcgaaaaacactaatcgatttttcttccagagttttaagatgctttacgtttataccattcaccttatattagttttcattttgttttcgaccaaaactattgatccaaattattatagctaggtacgatttgaacaaactttgattctcttaccaatcatctacaacttaaaaccaagatagcttcgctcaaaaagaaaaaaataatgacaacatgtataatacatatatgttcaatggaccgactcatctacactgttattcaaactaattattttataaatgattactaaatcagcttattaaattcccataatttctgcgtcgtgtgccgaagttgctcgttacaattgttattcccacaactttttttgcctatatatacaaaccctttaacatcaaactcaaaacacacaacaaacacaacttctacaagactcaaatagtttctatttaattactaaaaagaaaa

SEQ ID NO: 98 - DNA, 3000 bp, Arabidopsis thaliana
tatacccgaagatttgatgagtaaacacatgcataaaatattttttagctatatttggtatctttacgatgtttatatttttttctttttgataaagtgtttttgaaagatatgcctccaatttgatataattaataatataagatgagatgtagttttgagttttattcccccgaatggaatctctcttcctaacaacgtgtaaagtttgtaaactacgtgtattcacgtgttctttcacaatgcatgtttattatctttcttactatttttgtttctttctgacatttacgttttgttctgtcatatactcatataggctttaagttgggttaagatgttcaaataaatatacatgtaagatgaaactaaaacaaattacattaatatcagatttttattgttaaaggtttgaagatattttttcctgaaaactaaaaagtatgttcttaacaaaaaaaaaaaaaaaaaaaaaaaaaactaacaagtatgttgtttactgaaaagattagtgactgattttgtgagccttgacgataaaaatgtagaaaagtaaacacttcagcaagtggataatttataggtacctcatattctcgtcttttgatcaaacgtttcttttcctataaaaattaattacagataaaaagcgtcatatattaattaaccaatataatagcatatttattttggtgtattactttttctatcaattaaatatataaagggaaactatctcaaatctaaaagggaatccgatttaaagaagcttaacttcttctgttctcttttcataaaaaataaatataattacagatataaagtttaaacaatacaatagtatatttattttggcacattacttttttccattaaagaaattaaaggaagctatttcaaaactaaaaggaatccgatttaaagaagcttaacttcttctgttctctcgtttgcactttggctctacgtgatcaagaaaggtcggcaacttatactcagtctctggcaacctataagttgaatgacctgagatccggcgcagaacgttgaagaatctcactgtttgtaggcaaaatgtacctccttgtggatttgaggattgtttggtagacaactaattgagaatcaaaattggcaacaaaaaaaaaaatatggttaatccaaaatagtaattacgttatggaaatttggtaactgtcagagaaaacagactgtacctacattttggatcgtctttctccggaaacttaaacctttgtcgttgactctatccctcgttaacctggatccttgtggacagatcacgctaaatattcaccaaataacgatcaagtagtactcgtaaaaacaaggtgtgaatacatgagtgactttcatgtggaccgtatatgtaacctcaccataaataaaaaaaataaaaaaaaaaaaataaaatgaaaaagggtttaacgtccattcaccatatcagctatttttttgtttgtctgaaaatgtatgaccatatcattatgacattatctgctaattaaactgccttaataaattaacttcgtatatattttgctaatgtatctctaataattattcgttattatcaacttctttaaattttattatgaagatacgatatcaactctagttaaaattttcataacacagaagcgactctgatgtgatgggagtgaaaactaatacagctccacattctcacatgatgcataaacaatgctaaaacctcagataccataacttttcatggaattatgtttctcgattacaactatgtacccttccagatttcagaccaaattttaccatattttttctaataataaaagaaaactttatcttacatatatgcatttagccttctgtatacgcatttgcatgcatgcattaatgagtttcttctgatatacatgtgtgaggatttgtatatttaattcaaaaatatttgcataatggtcttactttttttttgttttttttctttttgacggcacttactttttatatattaaaagagttttttaacatgtgggttatgacaattttctcagtaactttaatttattttaatcacaaaaatatactatatagtggaatataatgttgtttatgctttattgtatgtgagagtctgggtctagtttatttattttaataaatgtgttacatctttctctttgcaatattcttgcttattgtttatggcgtcgatgttgtttcatggaccaactgaaatatataaaaggaataagcatctaatcgactaatcactttaaaatactccatatagtactaatcaaatacgtaccccataaaatagtccaatgattcaccacgaacttaaaaaaactgtataaatgggattcccacttttcctaaagccttgtgaccggctgtaccaatttaaaacgattttatgctataattcatttattgaatgttagggtaagatttgattaaaattaccctaaaatcatggtaaattttgatttaatttataacaataacgacacaaaattagagacaatgaaataaattttgatttttagtatgtggtggagatgtcaaacagaatctccattcaatttttttaaaaaaatacttatatacatataaattagggcatataattatgtataaatacatacaaatatgccttatatttatcttatattctagatatggggttttattccaaaaaattatattgtttgtatcattcactaatcataagtcttaaaatatattgcaggcagaaccgtctatcttagtttcaagtgttacggattgtttatcgctgggctttcgcggaggacgacaaaataacatatattaaaaatatatactcttaataatttttaaaatagggctgcaaatcgtttgagacaactctgaacacggtgcatgtatgatctcttctatataaagagatcttaatttgcacgtatatcatcatagaaaataaaacaattactacattttacaaaacaa

SEQ ID NO: 99 - DNA, 2750 bp, Arabidopsis thaliana
gaaaaaatggtccacacacatgtaaaatatcgtggacttacaaatgagactgagagcgtacgtacgtatagatgcatcagaattcatgtacaatatcaagagggcctctttcgatgacttcttatttgtctctgtttacaggaggacccctctactgctttttaccagtcaagactcttaatctcttagatcatagaatctatgaaaaaactgttgtcatgaaaacattttttttttcatttttgagttgaaatgtaaatgatgttttcattatggaaaatgaaaagctatttaaaaaatttaatgaataatatataagtttttttaggattataaaatactttatttttggttttggataatggttttctgttttataacaacaacttacatggatatagagtttttttcagatcaaaactagattagcccttttcagaccaagaagcaaaattggcaaaggaggcataatagttatgtgatattgactctcacgtcacggccgttgttgttcgctgtgaaaccaagtttgaagccagtggaaacctatagttcgtaaattgtgtgagtgatggaattggatgttctattccttatggtgaagtctatatttttgaaaacttgatctgaggctcttcgtttgcgcctgcactaagtctatatgtgtagaatgtagggtagataaaaaaatctaaattgaatgtgatgttcctttttctaaattaataatgtatacaatatctcatatatatatgaagagttttatgatgatgcgaaagcgagctagccgaaaaatagaactaacgtttcgtttatagtctgtagttagttaagtttgacgtaaacattgatcttgaaggcatggttcagaccactttttaaaatacctttaaagttactagtatagcggaaaaaaaaaatttgattaacatctggttcaaattataaaattaaacattttttatacagaaaaattggaaatagttttagataaaaaacaataccaattagcacatgaaaaatagcggctactgaataaaataagtaaaaacataagctcactcaagatcaaatccgatttgagttaaagtattgcgatagtcaagattagatttgatctgtgtatcgtggaatcatcctaaatgttataatttaagattacgaatcaaacaaacacatcttgaaaacaaccacatggctgttaaaaacaattagtattactattaactaacttcttacatcttgaaaaaacaaagagttccttcacatgaggccatgagcatgtgacatgcttgagttgactttgaaatttgaatggatagggccacgtcaacgcattcaaggatccttaattagcgcttaagtaaaacctctttatttgtcgtgactcgcgaaccccttttttattaacgttttagtcaacacaacatttcattaatgataattctactactattagtttgcaatgttaactaaactctttttacgtgagaaaacttaagattatcatttccagaccaccgcaagttccttgaaaagattgttatatatataacagctgcatatcttaatacggatttatgggctttaatttgaaatcaattgtatcaaataggtttgaaaaaaaaaatcgtatcacatacctttattttttgagtgtagtataagcaagcaatattgatgaatgcgtgagtctgcaaaatttaaccccaaaaaaaaagtaagcaacaatatatattcagcaatcatgttagaaagtattttaatcatgttgaactgaacgatctccgcgctaattagtattcctaagagacaccaatcagaaactattggatagttcgacggtttagaatttgtccagttgagaatggttttcaaactattttataaaatttttttagcgaatttctaaagttaagttgaccagcacatcttgtggttaaatgtttcactcgtcgttgaaaaagtcttttcaacaaaatcttactttctggatataattaatatcatatgtacaaaaattgattaatgggtcttaaactatttcatgtatttactatttagatagagacgtttaaaaaaaaactattttcgtgtctttactatttagatagagattacacgacatggaaataatagtacatggtcaagtttatatacggacgactctcatgaaatcctacaacaagaaaacaaagcaacatatagtataatgtgaaatatacactgttaagcaacatattacgtattatagttatttttatgttaatgacgtacaatgtacaaattctagtattcttcacctgaattatttgatgctaaactacgtacgtcgtggttattttcattgttctttaattagccatctcgaaatataattatttcaatgttacaagattttagtcgctctaataggatgtttatgaatttaaaccgacccaatccgacttgttttttcttctaaaaaatattatcttgaaaatgattttattaaattcgttttcgtcttagtctaattcagctataaagtataaacgttatgaccaagtccataatcaaatcatcatagtatttctccttaatcacaactacaagaaaaggaaatgggtcatgactttcttataaaacattaactaagatttgaccaaacataattttgtattatcaatattacaccataaatacggccacatatcctcctagtttcttcacacaactctcccctcaaaacattccatcaaaggaaaaaa

SEQ ID NO: 100 - DNA, 959 bp, Arabidopsis thaliana
agctaagctcatgacaaccacacacaaccatatgatttgagtaaccaactaggtgttgttatccgattgaaccgtgttttgcaaatttaattacatgtcaaaaaaatatgaatccgtatgaaatcttttaacgtaattgcatttttttgtcaacttaatggacggtaatccacaaaacaagctaaaacaaagtcaacattttcaagacatgacaaaaattgcctaactgtcattatccgccgattcgtttgactacgcaacatgccaacatgtaaattaatgtaggcatagtcgtcgaatcgtcattaagcaacgacggtcatattatggagaaacaaaataatcgtaattagtactacctctgtttcatattagttgtatttaaggtttttacacacaaattaagaaaacataaaaatctcatttaataaaaacatcgtttattataatcaattcaactaataaaaaaatagataattaaatataattggttaaaaaatattaaatacatttattatttgcatagaaatttaaaaacaacatttaaattgaaaaaaaataaaaacccttaaactacatttatatagaaacagagggagtattattaacgcaaccaaatttctaattagacttgactaatttaaggagtctcctttgatcacaatattttatcttcatataaagacatttcttcattgaaccaaaacaactattcaattccatcatgtgaaaaaataattgtaattagttgtaaactcaaccaccttatcttatcttataaaattcatactagtgtctaagcacgttacttccatattatattgtcatcgactaatcaaatgaatcttgttttgacaatcctttataagtatataaacacattttcttcattgacgctaaaaagaaaaaaaacacaaaactcatccatttggcttctctcttgcatattcgctgcaagg

SEQ ID NO: 101 - DNA, 1650 bp, Oryza sativa
cctctgagttagaggaggagaagcctagcataaatctaggcgttgatctaacgatacaaaatcgataagtttttcatctttaaatttatatttaatgcatttaacaaatccgattacaaaggggatgatttttggctaagtcttctgtactcatactattagctggaaaataaaatacgacgaaaatcacttaaattaattctgaaattcatttttttctgtggcttataagctgaaatagggcccatccttgtcttgtgaaaaacgacacagggcccggccatcctggcatttgtggcctcagcccatatgcaaagcatacattccactccaacaagctgctacgtcaactgcaagtggtcaagcctattttaataccacacatataaaccagtagcaaacctgcattttcttctctcttttttttagagcctttttggattagtgtcaaaacggtgccctaccaatattttgatagtttgaatagtgtatatggtggttttggcttgaagccaaatcattggcattgccaatgtctagtttgatattttctatactctactcaaagcttgttctaaattggtctccaaccaaatacaactctaccctaccaaaaaattagtagtgccaaaacttgcctaggttttagcactaccaatattttggtaggtattaaaccaaacaaacccttaatccaacatacaagcaggtatcccggaatatgttcaggctccagaaccatcctgttcaatttgtacgtaataattacgttccaaaacaacattcctgtacgctttggccaatgacgcccaaaagagccaaaaacgccgcccggtcaacctaccggccaatgctgcggagaacatgcatctttcgtaaccttcacgtacattacgctacgtatgtgtataattaatttttttttagatatataactccctacattttgtatctccatgatcacccaagaattcctcctcatcacacagagataataataatattttaatattactaccaacacatacacagctaaccaatcaagcaggaacagccccaaatcattgcaagctcccaagcacatgggacgttattcccttgacgaattcacatatatctctttgattaatctattgtcttaatgcattcccttcccatatcgtcgtacctagctaattagacgtgtagattatgtactacatttatacatgaatcatgttactatcccctctccaatgaactggcctcggcgccgtcctttactctctgttttttctaaaataaaaaaatgttttttttttgggtattgactagctcattgctggctagctgagccgcgcgtcgtcgccacaacgcgtacgtatgaggaggcatacatgtgcgattttgttattggagctgattttgcattagagccggaaggtaagcacatgcctaagctaagtacgcagggaaaatacactggaaaaacgtacaggttgtcacctgtgcgtcactagtcatgcacacgtctctataaatacactgagctctgcgaggacatgctcatccacagccagccatctagatcagcagctagcagcagcatcagcagatcgagcagcaaaacattctttgcttaatttggttgttgatttgtgtgtcgtggcc

SEQ ID NO: 102 - DNA, 2149 bp, Oryza sativa
caaaccaaacgagatatataatcttttctaggagtactttatatactccctccatttcaaaatgtttaacaccgttgactttttagcacatgtttgaccgttcgtcttattcaaaaaaaatttgtgaaatatgtaaaattatatgtgcacatgaaagtatatttaacaataaatcaaatgatatgaaaagaataaataattacttaaatttttaaataagatgaatggtcaaacatatactaaaaagtaacaataaatcaaatgatatgaaaagaataaataattacttaaatttttaaataagatgaatggtcaaacatatatcaaaaatttaacgatgtcaaatattttaaaacggaggaagtatatatcaacagcgagacatatatactactagctatatacgaatgaatatatgcagagatcaacaagagggcatatatatgcatagtcgtagtaggtagccatcgatcgatcagtctctctcatatcaggtaaggttaagttgcaggcagggcagggtatgtaggcccggccagggattcctacgtacatactacgtaacgtacgcatgcatcttattcatatatgtacactcctattactacacatgcacctccggaaattaactactgagatagatagctagcaccaagccaccaactaccacagactttttaaataatggactgtctattacacatctcatataaaatatctcttaaaatcttataatttttaaaccgttaaatcacattaagattcgtgcagacactcaaaccctaatctattcctcctctttttcgttttccctcagcaccacaccccagaacctttcgataccctactcggttaatctccgttgtcgctgcctcctttgccgccatcacctaatgccccgtcgcctagcctcgccggttggttggaaggtggttgacggtgttggcgagacccacctgactagcctcctctccccctctacttctcctcaagttgtcggtggtaaggtgccccgcctcatcgtccttcacatcactatcagctccccgccacctgatccgctcctatcggccatcaccgcctaacccgccgctccattttgcccctctgccaacatcggcccaccgttgactgttgaccctacgtctttggtagtggcaacgccaccttgccgcctgccagttccacctcctacgtccataattctaagcccaaccatcttctccttcttctccctccctcttccccacccataccccaatacggaaccttaatgtgtagaccctctgctagatttaattttagggtgctatcgacactgaagaagaagagtaggtcgtcagagttgaaggaaaaggacctaaatcataaatcacaaagcatatttgttgatctaaattctataaaatttagggatgggttttctttcgattgaaggttggcaattccgtacgttttaacaatcacatctacacacatcttgttgcagtcgcatcttctgctggggcacgcatgcatcgcaatattatagttgccttgccggcagcctttgcaacctataactgcatgcctgccatccatctacttccactgtaaacgtacgtttcatttctcggacgacatctcaactaacgaaagggattatgtgttctactgtgctagctggagtagctgctagatcgaccagaaaaagtgtagggaagcagccacgcacatgcattttctgcgtagtacgtacgtactactagctatgttatcttagtttgcactgttcgtcatctttattaattaattcctgcaaaggaatcggaaaacttaattatgtatgtaatctctcccggccccacctaattttttctgatgctagctactatggtagtttgcatgtatgcaacgtgaaaaataacagcgtgtagggtaactttccatgacacagctgcagagctgctagcgtcgacagagctaattaattaagagttcaattagctgggctgtcatggaatgggcgcacacaagtgtatatataagaagcagcttccatccatctatctctcacactgatccaggctctctccactactatttgcaagttaagttagtgcactgaaggagagcctgagatacgtcgtcgtacacacttgattaatta

SEQ ID NO: 103 - DNA, 3000 bp, Oryza sativa
actttggctgtgctaatcattggcaactccgaaggtgaaatctcgttgtcaaatgacaaaagtgatttaagtgatcaatcattctactcgtgtgtgcaggcagctagcagaaggccaccggccacatgcactgatgcaccatccagtttctttaattcttagcatctctccaagcagcctgcagttagcagagtgtactggggacaaaagaatcggagttagatagcacaggttaattagcttccagaaactggaaaaaagaaacggagcaagtaagctagctagctaactaaaacagttcagttcactgagatgatgagtcgccattgctgctgtttctgaactgtctagtgtctactagttgcccatgtggccatgagagatgagccccccgatgaaagctggtgtttgtttctgcttctgcctacctgctatgccttttctgaacaaaatgataccacttcaactacctaacagtgtgtccactggtccatccatctacgagttgttgcaggcttagccggcgacccggcgtctctcgtgcgtgcggatgattgatcaattccagcgcggcgacgccgacgccgccgccggatgttcgatcgatcgcttctcatatcatcgtgtattcgtggtgatcgctgatcagagctctctttcctgttagttgcacatgtgcattccggctctgacgactcaaaatgcatcgtcagctgctttgagttttcagtttgttcgtttctcctttttgttggtgactggtcgtctcgcttgattcaacctagctagctatagctttcaggtgtaaacttgagctggatcggagcactctctgattggattttcagctcgccggttcactgtgttcatttatttcaggatttgaaaaaaaggggatagttagctaggaatggctcagctgacggacaacgtgttgtggactcatcgatcaggcctgtcgcgtgcaatgaacgatacgtaagttaattcagtacataagcgtagagtttgtactttttgcctactactacttcggttctgctcatgattcagggttcaggacatcgtctggatttctacattcaaacaggcaaatataaccagtacttagtgtagacggattctaatcacccagtggacattcacttgtttattatatgtcaactaaatagttataaaaaatttttaattttttttgacaagatagattaatatgtaatatatcattccacaaacatgcaagttgaaatttaatttctataagtcgtaacaaaaacaacaaatttaactgcaaatatacatatactaatttgagttttatttgttatttttcttttacaatttgtagaagttaaattttaagttgcatgttacgtttgtggagtgatatattacatattgatttatcttaccaattttttcataattatttagatgacatgcattaaacagtgcacatccattcgggtgattaaaagagtttccccataataatcctaactctcgttctagaacccaaatattaaacttatagcatatttgtcatttttcactccttaaatggatgttccctcgtttctcgcatgctatctaaatagttatattttttaaataataataataataataataataataataataataataatatagatcaatatgtatatcattctacaacatgtaggtttaagttcaagtcatttttttcataattgtttatacgatataaataaaacaaggggacatctgaacaaaagagaactatctcagaagaaagcagcattttttttaactcccattctagtactgaatcttgaacccaaataggtaaagaggttaaacggcacatttgccatttttttactacattttcacataactaacttatcagaagcgcaggatggtacatgttctccctccgtttcaaaatatttgacaccgttgactttttagcacatatttgaccgttcatcttatttaaaaatttttgtgaaatatgtaaaactatttgtgtacatgaaagtatatttaacaataaatcaaatgatgtgaaaagaataaataattactttaattttttgaataaaacgaatgattaaacacgtactaaaaagtcaactgtatcaaacattttaaaacggagagagtagttaattaaggtgtgaaatggtattgtttcctcgggcattaatctttgtcagttgctacagcctgcaacagtgcacatggcttgaaccgatggttacaggccacagaaaaaaaaaagggaatccaagtactttgtctgatcatcacctctaaagttaatcaacatgacagcatgtggataatctcccacgtcctctcagttgcggttcagaagttcacagcgagaaataaattactcttcaaattgaccaactttgctttagattccatgcaaagcacacgcctacaggatcgatcaagcagcagagacatctcgcactgattttgtcagtacaacgtacaaaaatacgttctcgatcgacgaattcttctcctcatctctttcgctgctgctgctggcctgctgctaacaaccagtaggtgctgcattcttgactcctgcccggcgcccgcgatgatcagaactccacaaccaactgtttccgtagccgattttaaaaaagtttttgcagtacaattggtacttcgttcttggatggcaattagtatctgaatgcgtacctgctacaagaaagattcggcaaagcgaaaacaagaacagctcattgtgccatttgcataatttgcatgcgtgattcttcagaaagtgcagctagcaggtagctaagccagcctaccacatgggggaatagggacaggatgggccaggacggcgccgcgctataaatctcgcgccaattcttgcccgcaaattcagcagcagcagctaccagcaacaagcattctcttctagccttagcctagctagctagctttgttgtctagctctgatcgaggttggtggtgatc

SEQ ID NO: 104 - DNA, 3000 bp, Oryza sativa
gaacggttttattaagaaaaggcaatgtggttacctaagggctgacaacatagttgattagttattgacgcaatagcccattagccctagaactagaaggccagcctatagcggccagcccacacctgtcttgtttatgacattatattatatcttttatgactaccatggtttttcttaccaacactaaattgcatcctctcctattctcactaggaagcagtgtaaaaagttttcatagcaaaaactaaagtttgcttcttatagtcgtcacttgtcatgacaatgttagattgatctgaattttacagttatctatacactaacaccaaggaagatgtgaattctacagttatctatacccgttgcaatatgtatgccctactatacattactataagtcaaatgcatcatacttcctccgtctcattttaaatgcaaccataagtttccacgctcaactttgatcgtccatcttatttgaaaattttttataattagtacttttgttgttatgagatgataaaacatgaatagtactttactcgtgacttatgttttaaattttttttaaaacaatttcaattaagacggactgttaaagttaggcgcgaaaaaccatggctgcacttaaaatgagacggatggagtaatattatactacctctgttgttttttttctaattgacgttggttagttaaaaaaatagggagtataatttactaatcatttagacagatgaattgtgtaggataaacctgatcattgagtccagtgagcagagtcgcatccatcaattccaacacgttgattcgtttagaccgacagtggcaaactaacagctacttacatgaatgcatgaacattgaacagtacaataaggctacatagtacaatacaatgtatatacgttgtcgctgctgacataagtggcaaggcaaagaacctgtgttcataggataataaatcccacagctatttgatcaagacgtgtcaaggtagttagcctcaatgatttttctaaatggtcctagcttaaccgatttttggacatatcgaagataatatcaacaacactgtgtaaatagttactatgtctacgtataatgtggaaaaaatggtagttggtactagtagacaacaaccaaatagtgttggtgttttggtttacttaatttcttttagtgcataactaccatgtcccttttgtttcaaatccttccatagtctgagtgcttttcaaagcatttcgtcattaaatgcaaagaaactctggctggtaataaccaatagacattgaggtaacagctcacctttgctggtccatatatatctatctgaactattgcttcaatttgcaaaaattcaattccaagtgtgttagttttcccctattttcttctgaaagatgtctgctggtcctgtcacatgtatggtccatccatgcaactcagttcaagtgctatatactgctttgaaagtctacttacaaagcacactgctactgtgtaattctttaatccgagaatcaaacttttgcacaggaaaaggaactgtgctccttctgaaaacattgcattcttctagtagttcaaacttcaaatgatatggtcattcagtcattcagtgcgatagaagtcaattctgaagcaaaagtcttgtgctttgactactgcaaatgatcctactggcagatgaaaccaagggagctgtcatatcagcagggtcagagagccaataactaatctgaagctaattgactttgggagatagcattagcagcagtgcaaacaattaaacatgccagtgcgagaaaactggcgtgagatccaataattgcggccattgaccataccacattacagacaattcactattgtcgttctctgaacaatacctgtagtatctattatcttttcttttcttttcttctgtcaggcaatcagtcaccaacaggcagcatctaacagtaacggtaacagtaaagttagtaagacaggcaccgacaatatagttatacacagcctaaatcagaattttaaaacttaattttacagttgattggaggttttttcattgtagtttatttttaagtactagcttttaaatcgctaagttcgtaaatataaaagttttgcatataaattatttttggttgcttgtttattttttcacgcttatcagtcataagcaaacttacgagagtggaatgcttcagcaacaattacaagcaagaacagaaatcccggcaagcttcgcgttcgattgactgcatcttttcctgaaagtgaacatgcagtgcatggagtttcttgcaggatcaggatgcagcattgcattgcagccgaccctgtcgtggagtaatcattagcagtagaaatgaaggcagcgacccaacccaaatccaactctagatttcctcctcttatcctttctgttcttgaattgatcttgttaagctcatcttctcatccgctaatgacagtgatggtaagcaaagcgccatgagcattgggaccggatttgcaacgcgagatgattagggttcatggtgttccccacatgacagcgtgcttggatcaaccgcttttgtcttctccagctcagctcgtcttctccgcttcatccatctattataaaactgaaactctgatgagtttttcattgcttccttgtctattgtttctctgggatttgtctgtctgttgatctcactggatttggaaacgccgtgttcttggatactcgtttttcgttttttttttagttgtgctgctacctaattgttgaaatattgaattgaaggtttgaaggttgtggcattctgttgatttggattaataatagaattgggttttggagtattttatactcgtcgtgtacggatatatttgcccattcctctgaattttaaaaagaattttgcttttgagtaaaatattggcatagttgacgacttttaaaagcgaattgagcactatataaacaaccacatcccg

SEQ ID NO: 105 - DNA, 1875 bp, Oryza sativa
ttcaaaaaaaagttatatagaaaaatgaacaaaataaaagttgtaaatcttgataagttatacaactttgttgttgacaatttttccatttaaaatcacttactaccaaaaaaattattttgactaataaaataaattattatactgcatttaattattttgctataggtcaactcacaaatataaacatttcgtatgaaaaatgaaaaaaaaaatactaagtcatcttaatcgggcattagtatagagcccatcatagatgacgggcctagttgttaccaattgagatatggtcgcacagacatgttagatggtcataaaactcatactcgtcatggatgacacctatcagtgacgggccgtagtttagacccgattgagataacattctaaatctcaatcgggcaataagttggcgcccgtcacagatgatggaagcaacttgggccccatttgagatgtggaatgttatctcaatcgggcctaaactacgacccgtcactaatatgtgtcatccgtgatgggcctgagtttatgccatctaacatgtctgcgcgaccatatctcaatcgggcgcttttttggctcgattgaaatgacttccttgtgatggcccgctaattccaggcatgttagagaccgtcacacaggagagaggatctatactagtgattggtcagaccggagttatgcgagtagtcagaccagccaactcaatggtttgactgaccgactctaaggtatcggtttcgggttgtttatttggatactcgtgattaattcatatttatgacttctagatgatcaatgtacatatgtaatactgttctatgctaatattgagtcaagtttaagagaacttgtgctcggatatgatttcttatttgattcatgtgttggtgtgccatgatgccttagaagagtattatcggtgatgtatcaggagtcaacttgggagaaaatgacgtccagacgatcgggatattatacgagatgctaaggctaaaaaacaatgcacgtaatcgatggagattccatgcggcataagaaacatgtatctagcaaattcatgtatagggagattgtgtgtgtatagaatgagttgtgaatttggtaatatcacgaggtcaattggatgaggtaaaacatgtatctccatgacacaaaatcaaacggataaatattataattgattgtcgatgaaacatcgggttgtacgtggtgaaacaacaaaattagtgattaaaacatttcaaagatttgtgaaacaaaataaaattcctattgaaacatttcactctcgtatatgaaacaatgatgcgattcctattgaaacatttcactctcgtatatgaaacaatgatgcgaacaacttttaaaatgttacaatcatgctccaaactcacaccttgtttaacatgtgatcatacaaagtaactagcaaattctcccaaataggagtatgataaagaaacaaaagctcaatttgaatcctacctacccaacccaaacaaatgtcgccttccccaaacaaaacgcatcccaaactagagtgcttgacctcacccggttttccctcgcaccgttacagcgattcgtcaactcatttcgtccacacgcgcttagcgggacccctccgcagccaaacggcgcagtcccccgcccgagtcgccatgcacgttaagtttgacctgccaacctctcgaaaccttataaaaggacacctcggctcgatccattgctcgatccatctcaagtctcaagcttaattaagctcgtaatctcatataacctctttttggacgaagcttgccactcgatcgtgttaaccactcgagcttgagcgagcgagctagctttgctctgacgagaaaa

(19) United States
(12) Patent Application Publication
(10) Pub. No.: US 2014/0230093 A1
(43) Pub. Date: Aug. 14, 2014

(54) REGULATORY POLYNUCLEOTIDES AND USES THEREOF

(71) Applicant: MONSANTO TECHNOLOGY LLC, St. Louis, MO (US)

(72) Inventors: Tedd D. Elich, Durham, NC (US); Philip N. Benfey, Chapel Hill, NC (US); Ian Davis, Durham, NC (US); David A. Orlando, Somerville, MA (US)

(21) Appl. No.: 14/232,895

(22) PCT Filed: Jul. 18, 2012
(86) PCT No.: PCT/US2012/047117

§ 371 Date: Apr. 23, 2014

Related U.S. Application Data
(60) Provisional application No. 61/509,395, filed on Jul. 19, 2011.

(51) Int. Cl. C12N 15/82

(52) U.S. Cl. CPC C12N 15/8227; C12N 15/8241
USPC 800/287; 536/24.1; 435/419; 800/298; 800/320.3; 800/320.1; 800/320.2; 800/320; 800/312; 800/314; 800/306; 800/317.2; 435/29

(57) ABSTRACT

The present disclosure provides compositions and methods for regulating expression of transcribable polynucleotides in plant cells, plant tissues, and plants. Compositions include regulatory polynucleotide molecules capable of providing expression in plant tissues and plants. Methods for expressing polynucleotides in a plant cell, plant tissue, or plants using the regulatory polynucleotide molecules disclosed herein are also provided.

### RELATED APPLICATIONS

This application claims the benefit of U.S. Provisional Patent Application No. 61/509,395 filed Jul. 19, 2011; which is hereby incorporated by reference.

### SEQUENCE LISTING

The instant application contains a Sequence Listing which has been submitted in ASCII format via EFS-Web and is hereby incorporated by reference in its entirety. Said ASCII copy, created on Jul. 10, 2012, is named 13904-19.txt and is 221,860 bytes in size.

### FIELD

The present invention relates to polynucleotide molecules for regulating expression of transcribable polynucleotides in cells (including plant tissues and plants) and uses thereof.

### BACKGROUND

The development of transgenic plants having agronomically desirable characteristics often depends on the ability to control the spatial and temporal expression of the polynucleotide responsible for the desired trait. The control of the expression is largely dependent on the availability and use of regulatory control sequences that are responsible for the expression of the operably linked polynucleotide. Where expression in specific tissues or organs is desired, tissue-preferred regulatory elements may be used. Where expression in response to a stimulus is desired, inducible regulatory polynucleotides are the regulatory element of choice. In contrast, where continuous expression is desired throughout the cells of a plant, constitutive regulatory polynucleotides are utilized.

The proper regulatory elements typically must be present and be in the proper location with respect to the polynucleotide in order to obtain expression of the newly inserted transcribable polynucleotide in the plant cell. These regulatory elements may include a promoter region, various cis-elements, regulatory introns, a 5′ non-translated leader sequence and a 3′ transcription termination/polyadenylation sequence.

Since the patterns of expression of transcribable polynucleotides introduced into a plant are controlled using regulatory elements, there is an ongoing interest in the isolation and identification of novel regulatory elements which are capable of controlling expression of such transcribable polynucleotides.

### SUMMARY

In one aspect, an isolated regulatory polynucleotide is provided that comprises a polynucleotide molecule selected from the group consisting of: (a) a polynucleotide molecule comprising a nucleic acid molecule having a sequence selected from the group consisting of SEQ ID NOS: 1-105 that is capable of regulating transcription of an operably linked transcribable polynucleotide molecule; (b) a polynucleotide molecule having at least about 70% sequence identity to a sequence selected from the group consisting of SEQ ID NOS: 1-105 that is capable of regulating transcription of an operably linked transcribable polynucleotide molecule; and (c) a fragment of the polynucleotide molecule of (a) or (b) capable of regulating transcription of an operably linked transcribable polynucleotide molecule. In some aspects, the isolated regulatory polynucleotide is capable of regulating tissue-specific transcription. The isolated regulatory polynucleotide may comprise an intron.

In another aspect, a recombinant polynucleotide construct is provided comprising a regulatory polynucleotide described herein operably linked to a heterologous transcribable polynucleotide molecule. The transcribable polynucleotide molecule may encode a protein of agronomic interest.

In other aspects, such a recombinant polynucleotide construct is used to provide a transgenic host cell comprising the recombinant polynucleotide construct and to provide a transgenic plant stably transformed with the recombinant polynucleotide construct. Seed produced by such transgenic plants are also provided.

In a further aspect, a chimeric polynucleotide molecule is provided that comprises:

(1) a first polynucleotide molecule selected from the group consisting of

(a) a polynucleotide molecule comprising a nucleic acid molecule having a sequence selected from the group consisting of SEQ ID NOS: 1-105 that is capable of regulating transcription of an operably linked transcribable polynucleotide molecule;

(b) a polynucleotide molecule having at least about 70% sequence identity to a sequence selected from the group consisting of SEQ ID NOS:1-105 that is capable of regulating transcription of an operably linked transcribable polynucleotide molecule; and

(c) a fragment of the polynucleotide molecule of (a) or (b) capable of regulating transcription of an operably linked transcribable polynucleotide molecule, and

(2) a second polynucleotide molecule capable of regulating transcription of an operably linked polynucleotide molecule, wherein the first polynucleotide molecule is operably linked to the second polynucleotide molecule.

In yet a further aspect, an isolated polynucleotide molecule is provided that comprises a regulatory element derived from SEQ ID NOS: 1-105, wherein the regulatory element is capable of regulating transcription of an operably linked transcribable polynucleotide molecule.

In another aspect, a method of directing expression of a transcribable polynucleotide molecule in a host cell is provided that comprises:

(a) introducing the recombinant nucleic acid construct described herein into a host cell to produce a transgenic host cell; and

(b) selecting a transgenic host cell exhibiting expression of the transcribable polynucleotide molecule.

In a further aspect, a method of directing expression of a transcribable polynucleotide molecule in a plant is provided that comprises:

(a) introducing the recombinant nucleic acid construct described herein into a plant cell;

(b) regenerating a plant from the plant cell; and

(c) selecting a transgenic plant exhibiting expression of the transcribable polynucleotide molecule.

### BRIEF DESCRIPTION OF THE DRAWINGS

FIGS. 1-16, 32-66, 71-82, and 190-195 each provide the nucleotide sequence of a regulatory polynucleotide corresponding to the Arabidopsis gene having the accession number specified in the Figure. Where the regulatory polynucleotide has been modified to include the first intron from the coding sequence of the specified gene attached at the 3′ end of the 5′ UTR, the Figure indicates the gene accession number followed by the indicia “+intron”.

FIGS. 17-31, 67-70, 83-94, and 196-200 each provide the nucleotide sequence of a regulatory polynucleotide of a rice ortholog having the identified accession number specified in the Figure. Where the regulatory polynucleotide has been modified to include the first intron from the coding sequence of the specified gene attached at the 3′ end of the 5′ UTR, the Figure indicates the gene accession number followed by the indicia “+intron”.

FIGS. 95A-D through 158A-D illustrate the expression data of the underlying Arabidopsis genes that correspond to the regulatory polynucleotides of FIGS. 1-16, 32-66, and 71-82. FIGS. 95A-95D provide a schematic representation of the endogenous expression data for the Arabidopsis gene having the accession number specified in the Figure. FIG. 95A provides the expression values of this gene in different cell types which were sorted on the basis of expressing the indicated GFP markers. FIG. 95B provides the expression values of this gene from root sections along the longitudinal axis of the root. FIG. 95C provides the developmental specific expression of the gene. FIG. 95D provides the expression of the gene in response to various abiotic stresses. FIGS. 96-158 provide the endogenous expression data for the identified genes in the same format as FIGS. 95A-D.

FIGS. 159 through 189 show expression data for some of the underlying rice genes that correspond to the regulatory polynucleotides of FIGS. 17-31, 67-70, and 83-94. Expression data for the underlying rice genes is shown where available. Also, when more than one set of expression data was available, the further data may also be shown. FIG. 159 provides a schematic representation of the endogenous expression data for the rice ortholog having the specified accession number. The black bars represent expression data obtained from root tissue while the hatched bars represent expression data from above-ground plant tissue. FIGS. 160-189 provide the endogenous expression data for the identified genes in the same format as FIG. 159.

FIG. 200 depicts an image of the individual T2 seedling root for SEQ ID NO: 9, maturation zone.

FIGS. 201A-B depict images of the individual T2 seedling root for SEQ ID NO: 1; (A) meristematic zone; (B) elongation zone.

FIG. 202 depicts an image of the individual T2 seedling root for SEQ ID NO: 33, maturation zone.

FIG. 203 depicts an image of the individual T2 seedling root for SEQ ID NO: 36, meristematic zone.

FIGS. 204A-B depict images of the individual T2 seedling root for SEQ ID NO: 54; (A) meristematic zone; (B) elongation zone.

FIGS. 205A-B depict images of the individual T2 seedling root for SEQ ID NO: 55; (A) meristematic zone; (B) elongation zone.

### DETAILED DESCRIPTION

The present disclosure relates to regulatory polynucleotides that are capable of regulating expression of a transcribable polynucleotide in a host cell. In some embodiments, the regulatory polynucleotides are capable of regulating expression of a transcribable polynucleotide in a plant cell, plant tissue, plant, or plant seed. In other embodiments, the regulatory polynucleotides are capable of providing for tissue-specific expression of an operably linked polynucleotide in plants and plant tissues.

The present disclosure also provides recombinant constructs comprising such regulatory polynucleotides, as well as transgenic host cells, and organisms containing such recombinant constructs. Also provided are methods of directing expression of a transcribable polynucleotide in a host cell or organism.

Prior to describing this invention in further detail, however, the following terms will first be defined.

### DEFINITIONS

As used herein, the phrase “polynucleotide molecule” refers to a single- or double-stranded DNA or RNA of any origin (e.g., genomic or synthetic origin), i.e., a polymer of deoxyribonucleotide or ribonucleotide bases, respectively, read from the 5′ (upstream) end to the 3′ (downstream) end.

As used herein, the phrase “polynucleotide sequence” refers to the sequence of a polynucleotide molecule. The nomenclature for DNA bases as set forth at 37 CFR §1.822 is used.

As used herein, the term “transcribable polynucleotide molecule” refers to any polynucleotide molecule capable of being transcribed into a RNA molecule including, but not limited to, protein coding sequences (e.g., transgenes) and functional RNA sequences (e.g., a molecule useful for gene suppression).

As used herein, the terms “regulatory element” and “regulatory polynucleotide” refer to polynucleotide molecules having regulatory activity (i.e., one that has the ability to affect the transcription of an operably linked transcribable polynucleotide molecule). The terms refer to a polynucleotide molecule containing one or more elements such as core promoter regions, cis-elements, leaders or UTRs, enhancers, introns, and transcription termination regions, all of which have regulatory activity and may play a role in the overall expression of nucleic acid molecules in living cells. The “regulatory elements” determine if, when, and at what level a particular polynucleotide is transcribed. The regulatory elements may interact with regulatory proteins or other proteins or be involved in nucleotide interactions, for example, to provide proper folding of a regulatory polynucleotide.

As used herein, the terms “core promoter” and “minimal promoter” refer to a minimal region of a regulatory polynucleotide required to properly initiate transcription. A core promoter typically contains the transcription start site (TSS), a binding site for RNA polymerase, and general transcription factor binding sites. Core promoters can include promoters produced through the manipulation of known core promoters to produce artificial, chimeric, or hybrid promoters, and can be used in combination with other regulatory elements, such as cis-elements, enhancers, or introns, for example, by adding a heterologous regulatory element to an active core promoter with its own partial or complete regulatory elements.

As used herein, the term “cis-element” refers to a cis-acting transcriptional regulatory element that confers an aspect of the overall control of the expression of an operably linked transcribable polynucleotide. A cis-element may function to bind transcription factors, which are trans-acting protein factors that regulate transcription. Some cis-elements bind more than one transcription factor, and transcription factors may interact with different affinities with more than one cis-element. Cis-elements can confer or modulate expression, and can be identified by a number of techniques, including deletion analysis (i.e., deleting one or more nucleotides from the 5′ end or internal to a promoter), DNA binding protein analysis using DNase I footprinting, methylation interference, electrophoresis mobility-shift assays, in vivo genomic footprinting by ligation-mediated PCR, and other conventional assays; or by DNA sequence similarity analysis with known cis-element motifs by conventional DNA sequence comparison methods. The fine structure of a cis-element can be further studied by mutagenesis (or substitution) of one or more nucleotides or by other conventional methods. Cis-elements can be obtained by chemical synthesis or by isolation from regulatory polynucleotides that include such elements, and they can be synthesized with additional flanking nucleotides that contain useful restriction enzyme sites to facilitate subsequence manipulation.

As used herein, the term “enhancer” refers to a transcriptional regulatory element, typically 100-200 base pairs in length, which strongly activates transcription, for example, through the binding of one or more transcription factors. Enhancers can be identified and studied by methods such as those described above for cis-elements. Enhancer sequences can be obtained by chemical synthesis or by isolation from regulatory elements that include such elements, and they can be synthesized with additional flanking nucleotides that contain useful restriction enzyme sites to facilitate subsequence manipulation.

As used herein, the term “intron” refers to a polynucleotide molecule that may be isolated or identified from the intervening sequence of a genomic copy of a transcribed polynucleotide which is spliced out during mRNA processing prior to translation. Introns may themselves contain sub-elements such as cis-elements or enhancer domains that affect the transcription of operably linked polynucleotide molecules. Some introns are capable of increasing gene expression through a mechanism known as intron mediated enhancement (IME). IME, as distinguished from the effects of enhancers, is based on introns residing in the transcribed region of a polynucleotide. In general, IME is mediated by the first intron of a gene, which can reside in either the 5′-UTR sequence of a gene or between the first and second protein coding (CDS) exons of a gene. Without being limited by theory, because IME may be particularly important in highly expressed, constitutive genes, it may also play a role in the expression of genes expressed in a tissue-specific manner.

As used herein, the terms “leader” or “5′-UTR” refer to a polynucleotide sequence between the transcription and translation start sites of a gene. 5′-UTRs may themselves contain sub-elements such as cis-elements, enhancer domains, or introns that affect the transcription of operably linked polynucleotide molecules.

As used herein, the term “ortholog” refers to a polynucleotide from a different species that encodes a similar protein that performs the same biological function. For example, the ubiquitin genes from, for example, Arabidopsis and rice, are orthologs. Orthologs may also exhibit similar tissue expression patterns (for example, tissue-specific expression in plant tissues). Typically, orthologous nucleotide sequences are characterized by significant sequence similarity. A nucleotide sequence of an ortholog in one species (for example, Arabidopsis) can be used to isolate the nucleotide sequence of the ortholog in another species (for example, rice) using standard molecular biology techniques.

The term “expression” or “gene expression” means the transcription of an operably linked polynucleotide. The term “expression” or “gene expression” in particular refers to the transcription of an operably linked polynucleotide into structural RNA (rRNA, tRNA) or mRNA with or without subsequent translation of the latter into a protein. The process includes transcription of DNA and processing of the resulting mRNA product.

“Tissue-specific expression” refers to the transcription of a polynucleotide at higher levels in preferred tissues/developmental zones at all stages of a plant's lifecycle or at higher levels in preferred tissues/developmental zones at preferred stages of a plant's lifecycle. “Tissue-specific plant regulatory polynucleotides” and “tissue-specific regulatory polynucleotides” are regulatory polynucleotides that have regulatory activity in particular preferred tissues/developmental zones of a plant throughout a plant's lifecycle or at preferred stages of a plant's lifecycle. It is understood that for the terms “tissue-specific expression” and “tissue-specific plant regulatory polynucleotide” that some expression or activity can exist outside of the targeted plant tissues/developmental zones and plant lifecycle stages, but that expression in the preferred tissues/developmental zones during the preferred plant lifecycle stage(s) is selectively enhanced as compared to other non-preferred tissues and as compared to tissues/developmental zones (both preferred and non-preferred) during the non-preferred plant lifecycle stages. It is understood that the terms “plant lifecycle” and “stage of a plant's lifecycle” refer to a stage of the whole plant in its lifecycle (e.g., germinating seed, seedling, vegetative stage, reproductive stage, etc.) and that the term “developmental zone” refers to a region of cells in a plant sharing a common developmental stage, most commonly in the root of a plant (e.g., the meristematic, elongation, and maturation zones of the root).

With respect to the “developmental zones” of roots, the different cell types of the root arise from the quiescent centre (QC), where initial cells that surround a mitotically less active stem cell niche divide. Cell types are constrained within cell files, so that each new cell division successively displaces an older cell distal to the quiescent centre. Cells undergo division, elongation, and differentiation when they enter the meristematic, elongation, and maturation zones, respectively, along the longitudinal axis. Because cells are constrained within these files and new cells are born at the root apex, a cell's developmental time line can be tracked along the root's longitudinal axis.

“Root-specific expression” refers to the transcription of a polynucleotide at higher levels in at least one root tissue/developmental zone as compared to non-root tissues at some or all stages of a plant's lifecycle. “Root-specific plant regulatory polynucleotides” and “root-specific regulatory polynucleotides” are regulatory polynucleotides that have regulatory activity in at least one root tissue/developmental zone of a plant at some or all stages of a plant's lifecycle. It is understood that for the terms “root-specific expression” and “root-specific plant regulatory polynucleotide” that some expression or activity can exist outside of the targeted root tissue(s)/developmental zone(s) and stage(s) of a plant's lifecycle, but that expression in at least one root tissue/developmental zone during the preferred plant lifecycle stage(s) is selectively enhanced as compared to non-root tissues and as compared to tissues/developmental zones (both root and non-root) during any non-preferred plant lifecycle stages (i.e., different root-specific regulatory polynucleotides may regulate tissue-specific expression in different root tissues/developmental zones). It is understood that “root-specific regulatory polynucleotides” may have expression patterns differing from one another (i.e., differing in expression level, root tissue(s)/developmental zone(s), and/or preferred stages of a plant's lifecycle).

As used herein, the term “chimeric” refers to the product of the fusion of portions of two or more different polynucleotide molecules. As used herein, the term “chimeric regulatory polynucleotide” refers to a regulatory polynucleotide produced through the manipulation of known promoters or other polynucleotide molecules, such as cis-elements. Such chimeric regulatory polynucleotides may combine enhancer domains that can confer or modulate expression from one or more regulatory polynucleotides, for example, by fusing a heterologous enhancer domain from a first regulatory polynucleotide to a promoter element (e.g. a core promoter) from a second regulatory polynucleotide with its own partial or complete regulatory elements.

As used herein, the term “operably linked” refers to a first polynucleotide molecule, such as a core promoter, connected with a second polynucleotide molecule, such as a transcribable polynucleotide (e.g., a polynucleotide encoding a protein of interest), where the polynucleotide molecules are so arranged that the first polynucleotide molecule affects the transcription of the second polynucleotide molecule. The two polynucleotide molecules may be part of a single contiguous polynucleotide molecule and may be adjacent. For example, a promoter is operably linked to a polynucleotide encoding a protein of interest if the promoter modulates transcription of the polynucleotide of interest in a cell.

An “isolated” or “purified” polynucleotide or polypeptide molecule, refers to a molecule that is not in its native environment such as, for example, a molecule not normally found in the genome of a particular host cell, or a DNA not normally found in the host genome in an identical context, or any two sequences adjacent to each other that are not normally or naturally adjacent to each other.

### Regulatory Polynucleotide Molecules

The regulatory polynucleotide molecules described herein were discovered using bioinformatic screening techniques of databases containing expression and sequence data for genes in various plant species. Such bioinformatic techniques are described in more detail in the Examples set forth below.

In one embodiment, isolated regulatory polynucleotide molecules are provided. The regulatory polynucleotides provided herein include polynucleotide molecules having transcription regulatory activity in host cells, such as plant cells. In some embodiments, the regulatory polynucleotides are capable of regulating tissue-specific transcription of an operably linked transcribable polynucleotide molecule in transgenic plants and plant tissues. In some embodiments, the regulatory polynucleotides are capable of regulating root-specific transcription of an operably linked transcribable polynucleotide molecule in transgenic plants and plant tissues.

The isolated regulatory polynucleotide molecules comprise a polynucleotide molecule selected from the group consisting of a) a polynucleotide molecule comprising a nucleic acid molecule having a sequence selected from the group consisting of SEQ ID NOs: 1-105 that is capable of regulating transcription of an operably linked transcribable polynucleotide molecule; b) a polynucleotide molecule having at least about 70% sequence identity to the sequence of SEQ ID NOs: 1-105 that is capable of regulating transcription of an operably linked transcribable polynucleotide molecule; and c) a fragment of the polynucleotide molecule of a) or b) capable of regulating transcription of an operably linked transcribable polynucleotide molecule. Such fragments can be a UTR, a core promoter, an intron, an enhancer, a cis-element, or any other regulatory element.

Thus, the regulatory polynucleotide molecules include those molecules having sequences provided in SEQ ID NO: 1 through SEQ ID NO: 105. These polynucleotide molecules are capable of affecting the expression of an operably linked transcribable polynucleotide molecule in plant cells and plant tissues and therefore can regulate expression in transgenic plants. The present disclosure also provides methods of modifying, producing, and using such regulatory polynucleotides. Also included are compositions, transformed host cells, transgenic plants, and seeds containing the regulatory polynucleotides, and methods for preparing and using such regulatory polynucleotides.

The disclosed regulatory polynucleotides are capable of providing for expression of operably linked transcribable polynucleotides in any cell type, including, but not limited to plant cells. For example, the regulatory polynucleotides may be capable of providing for the expression of operably linked heterologous transcribable polynucleotides in plants and plant cells. In one embodiment, the regulatory polynucleotides are capable of directing tissue-specific expression in a transgenic plant, plant tissue(s), or plant cell(s).

In one embodiment, the regulatory polynucleotides may comprise multiple regulatory elements, each of which confers a different aspect to the overall control of the expression of an operably linked transcribable polynucleotide. In another embodiment, regulatory elements may be derived from the polynucleotide molecules of SEQ ID NOs: 1-105. Thus, regulatory elements of the disclosed regulatory polynucleotides are also provided.

The disclosed polynucleotides include, but are not limited to, nucleic acid molecules that are between about 0.1 Kb and about 5 Kb, between about 0.1 Kb and about 4 Kb, between about 0.1 Kb and about 3 Kb, and between about 0.1 Kb and about 2 Kb, about 0.25 Kb and about 2 Kb, or between about 0.10 Kb and about 1.0 Kb.

The regulatory polynucleotides as provided herein also include fragments of SEQ ID NOs: 1-105. The fragment polynucleotides include those polynucleotides that comprise at least 50, at least 75, at least 100, at least 125, at least 150, at least 175, or at least 200 contiguous nucleotide bases where the fragment's complete sequence in its entirety is identical to a contiguous fragment of the referenced polynucleotide molecule. In some embodiments, the fragments contain one or more regulatory elements capable of regulating the transcription of an operably linked polynucleotide. Such fragments may include regulatory elements such as introns, enhancers, core promoters, leaders, and the like.

Thus also provided are regulatory elements derived from the polynucleotides having the sequences of SEQ ID NOs: 1-105. In some embodiments, the regulatory elements are capable of regulating transcription of operably linked transcribable polynucleotides in plants and plant tissues. The regulatory elements that may be derived from the polynucleotides of SEQ ID NOs: 1-105 include, but are not limited to introns, enhancers, leaders, and the like. In addition, the regulatory elements may be used in recombinant constructs for the expression of operably linked transcribable polynucleotides of interest.

The present disclosure also includes regulatory polynucleotides that are substantially homologous to SEQ ID NOs: 1-105. As used herein, the phrase “substantially homologous” refers to polynucleotide molecules that generally demonstrate a substantial percent sequence identity with the regulatory polynucleotides provided herein. Substantially homologous polynucleotide molecules include polynucleotide molecules that function in plants and plant cells to direct transcription and have at least about 70% sequence identity, at least about 80% sequence identity, at least about 90% sequence identity, or even greater sequence identity, specifically including about 73%, 75%, 78%, 83%, 85%, 88%, 92%, 94%, 95%, 96%, 97%, 98%, 99% or greater sequence identity with the regulatory polynucleotide molecules provided in SEQ ID NOs: 1-105. Polynucleotide molecules that are capable of regulating transcription of operably linked transcribable polynucleotide molecules and are substantially homologous to the polynucleotide sequences of the regulatory polynucleotides provided herein are encompassed herein.

As used herein, the “percent sequence identity” is determined by comparing two optimally aligned sequences over a comparison window, where the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, divided by the number of matched positions by the total number of positions in the window of comparison and multiplying the result by 100 to yield the percentage of sequence identity. Alignment for the purposes of determining the percentage identity can be achieved in various ways that are within the skill in the art, for example, using publicly available computer software such as BLAST. Those skilled in the art can determine appropriate parameters for measuring alignment, including any algorithms needed to achieve optimal alignment over the full length of the sequences being compared.

Additional regulatory polynucleotides substantially homologous to those identified herein may be identified by a variety of methods. For example, cDNA libraries may be constructed using cells or tissues of interest and screened to identify genes having an expression pattern similar to that of the regulatory elements described herein. The cDNA sequence for the identified gene may then be used to isolate the gene's regulatory sequences for further characterization. Alternately, transcriptional profiling or electronic northern techniques may be used to identify genes having an expression pattern similar to that of the regulatory polynucleotides described herein. Once these genes have been identified, their regulatory polynucleotides may be isolated for further characterization. The electronic northern technique refers to a computer-based sequence analysis which allows sequences from multiple cDNA libraries to be compared electronically based on parameters the researcher identifies including abundance in EST populations in multiple cDNA libraries, or exclusively to EST sets from one or combinations of libraries. The transcriptional profiling technique is a high-throughput method used for the systematic monitoring of expression profiles for thousands of genes. This DNA chip-based technology arrays thousands of oligonucleotides on a support surface. These arrays are simultaneously hybridized to a population of labeled cDNA or cRNA probes prepared from RNA samples of different cell or tissue types, allowing direct comparative analysis of expression. This approach may be used for the isolation of regulatory sequences such as promoters associated with those sequences.

In some embodiments, substantially homologous polynucleotide molecules may be identified when they specifically hybridize to form a duplex molecule under certain conditions. Under these conditions, referred to as stringency conditions, one polynucleotide molecule can be used as a probe or primer to identify other polynucleotide molecules that share homology. Accordingly, the nucleotide sequences of the present invention may be used for their ability to selectively form duplex molecules with complementary stretches of polynucleotide molecule fragments. Substantially homologous polynucleotide molecules may also be determined by computer programs that align polynucleotide sequences and estimate the ability of polynucleotide molecules to form duplex molecules under certain stringency conditions or show sequence identity with a reference sequence.

In some embodiments, the regulatory polynucleotides disclosed herein can be modified from their wild-type sequences to create regulatory polynucleotides that have variations in the polynucleotide sequence. The polynucleotide sequences of the regulatory elements of SEQ ID NOs: 1-105 may be modified or altered. One method of alteration of a polynucleotide sequence includes the use of polymerase chain reactions (PCR) to modify selected nucleotides or regions of sequences. These methods are well known to those of skill in the art. Sequences can be modified, for example, by insertion, deletion, or replacement of template sequences in a PCR-based DNA modification approach. In the context of the present invention, a “variant” is a regulatory polynucleotide containing changes in which one or more nucleotides of an original regulatory polynucleotide is deleted, added, and/or substituted. In one example, a variant regulatory polynucleotide substantially maintains its regulatory function. For example, one or more base pairs may be deleted from the 5′ or 3′ end of a regulatory polynucleotide to produce a “truncated” polynucleotide. One or more base pairs can also be inserted, deleted, or substituted internally to a regulatory polynucleotide. Variant regulatory polynucleotides can be produced, for example, by standard DNA mutagenesis techniques or by chemically synthesizing the variant regulatory polynucleotide or a portion thereof.

The methods and compositions provided for herein may be used for the efficient expression of transgenes in plants. The regulatory polynucleotide molecules useful for directing expression (including tissue-specific expression) of transcribable polynucleotides, may provide enhancement of expression (including enhancement of tissue-specific expression) (e.g., through the use of IME with the introns of the regulatory polynucleotides disclosed herein), and/or may provide for increased levels of expression of transcribable polynucleotides operably linked to a regulatory polynucleotide described herein. In addition, the introns identified in the regulatory polynucleotide molecules provided herein may also be included in conjunction with any other plant promoter (or plant regulatory polynucleotide) for the enhancement of the expression of selected transcribable polynucleotides.

Also provided are chimeric regulatory polynucleotide molecules. Such chimeric regulatory polynucleotides may contain one or more regulatory elements disclosed herein in operable combination with one or more additional regulatory elements. The one or more additional regulatory elements can be any additional regulatory elements from any source, including those disclosed herein, as well as those known in the art, for example, the actin 2 intron. In addition, the chimeric regulatory polynucleotide molecules may comprise any number of regulatory elements such as, for example, 2, 3, 4, 5, or more regulatory elements.

In some embodiments, the chimeric regulatory polynucleotides contain at least one core promoter molecule provided herein operably linked to one or more additional regulatory elements, such as one or more regulatory introns and/or enhancer elements. Alternatively, the chimeric regulatory polynucleotides may contain one or more regulatory elements as provided herein in combination with a minimal promoter sequence, for example, the CaMV 35S minimal promoter. Thus, the design, construction, and use of chimeric regulatory polynucleotides according to the methods disclosed herein for modulating the expression of operably linked transcribable polynucleotide molecules are also provided.

The chimeric regulatory polynucleotides as provided herein can be designed or engineered using any method. Many regulatory regions contain elements that activate, enhance, or define the strength and/or specificity of the regulatory region. Thus, for example, chimeric regulatory polynucleotides of the present invention may comprise core promoter elements containing the site of transcription initiation (e.g., RNA polymerase II binding site) combined with heterologous cis-elements located upstream of the transcription initiation site that modulate transcription levels. Thus, in one embodiment, a chimeric regulatory polynucleotide may be produced by fusing a core promoter fragment polynucleotide described herein to a cis-element from another regulatory polynucleotide; the resultant chimeric regulatory polynucleotide may cause an increase in expression of an operably linked transcribable polynucleotide molecule. Chimeric regulatory polynucleotides can be constructed such that regulatory polynucleotide fragments or elements are operably linked, for example, by placing such a fragment upstream of a minimal promoter. The core promoter regions, regulatory elements and fragments of the present invention can be used for the construction of such chimeric regulatory polynucleotides.

Thus, also provided are chimeric regulatory polynucleotide molecules comprising (1) a first polynucleotide molecule selected from the group consisting of a) a polynucleotide molecule comprising a nucleic acid molecule having the sequence of SEQ ID NOs: 1-105 that is capable of regulating transcription of an operably linked transcribable polynucleotide molecule; b) a polynucleotide molecule having at least about 70% sequence identity to the sequence of SEQ ID NOs: 1-105 that is capable of regulating transcription of an operably linked transcribable polynucleotide molecule; and c) a fragment of the polynucleotide molecule of a) or b) capable of regulating transcription of an operably linked transcribable polynucleotide molecule, and (2) a second polynucleotide molecule capable of regulating transcription of an operably linked polynucleotide molecule, wherein the first polynucleotide molecule is operably linked to the second polynucleotide molecule. The chimeric regulatory polynucleotide molecules may further comprise at least a third, fourth, fifth, or more additional polynucleotide molecules capable of regulating transcription of an operably linked polynucleotide, where the at least a third, fourth, fifth, or more additional polynucleotide molecules is/are operably linked to the first and second polynucleotide molecules.

The first and second polynucleotide molecules may be any combination of regulatory elements, including those provided herein. In one embodiment, the first polynucleotide comprises at least a core promoter element and the second polynucleotide comprises at least one additional regulatory element, including, but not limited to, an enhancer, an intron, and a leader molecule.

Methods for construction of chimeric and variant regulatory polynucleotides include, but are not limited to, combining elements of different regulatory polynucleotides or duplicating portions or regions of a regulatory polynucleotide. Those of skill in the art are familiar with the standard resource materials that describe specific conditions and procedures for the construction, manipulation, and isolation of macromolecules (e.g., polynucleotide molecules, plasmids, etc.), as well as the generation of recombinant organisms and the screening and isolation of polynucleotide molecules.

Thus, also provided are novel methods and compositions for the efficient expression of transcribable polynucleotides in plants through the use of the regulatory polynucleotides described herein. The regulatory polynucleotides described herein include tissue-specific promoters which may find wide utility in directing the expression of potentially any polynucleotide which one desires to have expressed preferentially in specific parts of a plant (or preferentially in specific parts of a plant during preferred stages of the plant lifecycle). The regulatory elements disclosed herein may be used as promoters within expression constructs in order to increase the level of expression of transcribable polynucleotides operably linked to any one of the disclosed regulatory polynucleotides. Alternatively, the regulatory elements disclosed herein may be included in expression constructs in conjunction with any other plant promoter for the enhancement of the expression of one or more selected polynucleotides.

In some embodiments, the regulatory polynucleotides are capable of regulating tissue-specific transcription of an operably linked transcribable polynucleotide molecule in at least one root tissue and/or developmental zone of transgenic plants. For example, some root-tissue-specific polynucleotides regulate expression in the following tissues/developmental zones:
(1) All or substantially all root tissues in all root developmental zones (“broad root”);
(2) Epidermal, vascular, cortex, and phloem tissues, in all root developmental zones;
(3) Pericycle, endodermis, and cortex tissues, in all root developmental zones;
(4) Epidermal tissue, including root hairs, in all root developmental zones
(5) Meristem and root cap tissues in the root meristematic and elongation zones.
   In some embodiments, regulatory polynucleotides having the tissue-specific patterns above may exhibit at least about 4-fold, 5-fold, 6-fold, 7-fold, 8-fold, 9-fold, or 10-fold greater expression in root tissue than in seed tissues, may exhibit at least about 2-fold, 2.5-fold, or 3-fold greater expression in target tissues/zones than in non-target tissues/zones, may exhibit GC-RMA expression levels above about 4 units in all target tissues and zones, and/or may be minimally responsive to abiotic stress. In other embodiments, the regulatory polynucleotides having the tissue-specific patterns above (i.e., patterns 1-5) may exhibit one or more of the following:
(1) for pattern 1, the regulatory polynucleotides may exhibit (a) 10-fold greater expression in root tissues than in seed tissues under normal conditions, (b) GC-RMA expression levels above 4 in all tissue and developmental zones, and/or (c) 10-fold greater expression in root tissues than in shoot tissues across all abiotic stress conditions.
(2) for pattern 2, the regulatory polynucleotides may exhibit (a) 10-fold greater expression in root tissues than in seed tissues under normal conditions, (b) GC-RMA expression levels above 4 in one or more tissues marked by SUC2, S32, CORTEX, APL, and/or S18, (c) 3.5-fold greater expression in one or more tissues marked by SUC2, S32, CORTEX, APL, and/or S18 than in other root tissues; and/or (d) exhibited 3-fold greater expression in root tissues than in shoot tissues across all abiotic stress conditions.
(3) for pattern 3, the regulatory polynucleotides may exhibit (a) 7.5-fold greater expression in root tissues than in seed tissues under normal conditions, (b) GC-RMA expression levels above 4 in one or more tissues marked by S17, J2661, J0571, J0121, and/or CORTEX, (c) 3.25-fold greater expression in one or more tissues marked by S17, J2661, J0571, J0121, and/or CORTEX than in other root tissues, (d) greater expression in root tissues than in shoot tissues across all abiotic stress conditions, (e) 10-fold greater expression in root tissues than in seed and shoot tissues under normal conditions, (f); GC-RMA expression levels above 5 in one or more tissues marked by S17, J2661, J0571, J0121, and/or CORTEX, (g) 6-fold greater expression in one or more tissues marked by S17, J2661, J0571, J0121, and/or CORTEX than in other root tissues, and/or (h); 1.8-fold greater expression in root tissues than in shoot tissues across all abiotic stress conditions.
(4) for pattern 4, the regulatory polynucleotides may exhibit (a) 10-fold greater expression in root tissues than in seed tissues under normal conditions, (b) GC-RMA expression levels above 4 in one or more tissues marked by GL2 and/or COBL9, (c) 2.8-fold greater expression in one or more tissues marked by GL2 and/or COBL9 than in other root tissues, and/or (d) 2-fold greater expression in root tissues than in shoot tissues across all abiotic stress conditions.
(5) for pattern 5, the regulatory polynucleotides may exhibit (a) 10-fold greater expression in root tissues than in seed tissues under normal conditions, (b) GC-RMA expression levels above 4 in one or more tissues marked by PET111 and/or LRC and above 3.6 in developmental zones 1-8, (c) 2.3-fold greater expression in those developmental zones than in zones 9-13, (d) and/or 2-fold greater expression in root tissues than in shoot tissues across all abiotic stress conditions.

### Recombinant Constructs

The disclosed regulatory polynucleotide molecules find use in the production of recombinant polynucleotide constructs, for example to express transcribable polynucleotides encoding proteins of interest in a host cell.

The recombinant constructs comprise (1) an isolated regulatory polynucleotide molecule comprising a polynucleotide molecule selected from the group consisting of a) a polynucleotide molecule comprising a nucleic acid molecule having the sequence of SEQ ID NOs: 1-105 that is capable of regulating transcription of an operably linked transcribable polynucleotide molecule; b) a polynucleotide molecule having at least about 70% sequence identity to the sequence of SEQ ID NOs: 1-105 that is capable of regulating transcription of an operably linked transcribable polynucleotide molecule; and c) a fragment of the polynucleotide molecule of a) or b) capable of regulating transcription of an operably linked transcribable polynucleotide molecule operably linked to (2) a transcribable polynucleotide molecule.

The constructs provided herein may contain any recombinant polynucleotide molecule having a combination of regulatory elements linked together in a functionally operative manner. For example, the constructs may contain a regulatory polynucleotide operably linked to a transcribable polynucleotide molecule operably linked to a 3′ transcription termination polynucleotide molecule. In addition, the constructs may include, but are not limited to, additional regulatory polynucleotide molecules from the 3′-untranslated region (3′ UTR) of plant genes (e.g., a 3′ UTR to increase mRNA stability, such as the PI-II termination region of potato or the octopine or nopaline synthase 3′ termination regions). Constructs may also include but are not limited to the 5′ untranslated regions (5′ UTR) of an mRNA polynucleotide molecule which can play an important role in translation initiation and can also be a regulatory component in a plant expression construct. For example, non-translated 5′ leader polynucleotide molecules derived from heat shock protein genes have been demonstrated to enhance expression in plants. These additional upstream and downstream regulatory polynucleotide molecules may be derived from a source that is native or heterologous with respect to the other elements present on the promoter construct.

Thus, constructs generally comprise regulatory polynucleotides such as those provided herein (including modified and chimeric regulatory polynucleotides), operatively linked to a transcribable polynucleotide molecule so as to direct transcription of the transcribable polynucleotide molecule at a desired level or in a desired tissue or developmental pattern upon introduction of the construct into a plant cell. In some cases, the transcribable polynucleotide molecule comprises a protein-coding region, and the promoter provides for transcription of a functional mRNA molecule that is translated and expressed as a protein product. Constructs may also be constructed for transcription of antisense RNA molecules or other similar inhibitory RNA in order to inhibit expression of a specific RNA molecule of interest in a target host cell.

Exemplary transcribable polynucleotide molecules for incorporation into the disclosed constructs include, for example, transcribable polynucleotides from a species other than the target species, or even transcribable polynucleotides that originate with or are present in the same species, but are incorporated into recipient cells by genetic engineering methods rather than classical reproduction or breeding techniques. Exogenous polynucleotide or regulatory element is intended to refer to any polynucleotide molecule or regulatory polynucleotide that is introduced into a recipient cell. The type of polynucleotide included in the exogenous polynucleotide can include polynucleotides that are already present in the plant cell, polynucleotides from another plant, polynucleotides from a different organism, or polynucleotides generated externally, such as a polynucleotide molecule containing an antisense message of a protein-encoding molecule, or a polynucleotide molecule encoding an artificial or modified version of a protein.

The disclosed regulatory polynucleotides can be incorporated into a construct using marker genes and can be tested in transient analyses that provide an indication of expression in stable plant systems. As used herein, the term “marker gene” refers to any transcribable polynucleotide molecule whose expression can be screened for or scored in some way.

Methods of testing for marker expression in transient assays are known to those of skill in the art. Transient expression of marker genes has been reported using a variety of plants, tissues, and DNA delivery systems. For example, types of transient analyses include but are not limited to direct DNA delivery via electroporation or particle bombardment of tissues in any transient plant assay using any plant species of interest. Such transient systems would include but are not limited to electroporation of protoplasts from a variety of tissue sources or particle bombardment of specific tissues of interest. Any transient expression system may be used to evaluate regulatory polynucleotides or regulatory polynucleotide fragments operably linked to any transcribable polynucleotide molecule including, but not limited to, selected reporter genes, marker genes, or polynucleotides encoding proteins of agronomic interest. Any plant tissue may be used in the transient expression systems and include but are not limited to leaf base tissues, callus, cotyledons, roots, endosperm, embryos, floral tissue, pollen, and epidermal tissue.

Any scorable or screenable marker can be used in a transient assay as provided herein. For example, markers for transient analyses of the regulatory polynucleotides or regulatory polynucleotide fragments of the present invention include GUS or GFP. The constructs containing the regulatory polynucleotides or regulatory polynucleotide fragments of the present invention operably linked to a marker are delivered to the tissues and the tissues are analyzed by the appropriate mechanism, depending on the marker. The quantitative or qualitative analyses are used as a tool to evaluate the potential expression profile of the promoters or promoter fragments when operatively linked to polynucleotides encoding proteins of agronomic interest in stable plants.

Thus, in one embodiment, a regulatory polynucleotide molecule, or a variant, or derivative thereof, capable of regulating transcription, is operably linked to a transcribable polynucleotide molecule that provides for a selectable, screenable, or scorable marker. Markers for use in the practice of the present invention include, but are not limited to, transcribable polynucleotide molecules encoding β-glucuronidase (GUS), green fluorescent protein (GFP), luciferase (LUC), proteins that confer antibiotic resistance, or proteins that confer herbicide tolerance. Useful antibiotic resistance markers, including those encoding proteins conferring resistance to kanamycin (nptII), hygromycin B (aph IV), streptomycin or spectinomycin (aad, spec/strep), and gentamycin (aac3 and aacC4), are known in the art. Herbicides for which transgenic plant tolerance has been demonstrated and for which the methods disclosed herein can be applied include, but are not limited to, glyphosate, glufosinate, sulfonylureas, imidazolinones, bromoxynil, delapon, cyclohezanedione, protoporphyrionogen oxidase inhibitors, and isoxasflutole herbicides. Polynucleotide molecules encoding proteins involved in herbicide tolerance are known in the art, and include, but are not limited to, a polynucleotide molecule encoding 5-enolpyruvylshikimate-3-phosphate synthase (EPSP synthase) for glyphosate tolerance; a polynucleotide molecule encoding bromoxynil nitrilase (Bxn) for Bromoxynil tolerance; a polynucleotide molecule encoding phytoene desaturase (crtI) for norflurazon tolerance; a polynucleotide molecule encoding acetohydroxyacid synthase (AHAS, aka ALS) for tolerance to sulfonylurea herbicides; and the bar gene for glufosinate and bialaphos tolerance.

The regulatory polynucleotide molecules can be operably linked to any transcribable polynucleotide molecule of interest. Such transcribable polynucleotide molecules include, for example, polynucleotide molecules encoding proteins of agronomic interest. Proteins of agronomic interest can be any protein desired to be expressed in a host cell, such as, for example, proteins that provide a desirable characteristic associated with plant morphology, physiology, growth and development, yield, nutritional content, disease or pest resistance, or environmental or chemical tolerance. The expression of a protein of agronomic interest is desirable in order to confer an agronomically important trait on the plant containing the polynucleotide molecule. Proteins of agronomic interest that provide a beneficial agronomic trait to crop plants include, but are not limited to for example, proteins conferring herbicide resistance, insect control, fungal disease resistance, virus resistance, nematode resistance, bacterial disease resistance, starch production, modified oils production, high oil production, modified fatty acid content, high protein production, fruit ripening, enhanced animal and human nutrition, biopolymers, environmental stress resistance, pharmaceutical peptides, improved processing traits, improved digestibility, low raffinose, industrial enzyme production, improved flavor, nitrogen fixation, hybrid seed production, and biofuel production. Some proteins of agronomic interest that provide a beneficial agronomic trait to crop plants may also cause non-beneficial or harmful side effects, for example, host plant toxicity, decreased nutrition or digestibility, or decreased yield. In such cases, tissue-specific regulatory polynucleotide molecules may be particularly useful for expressing proteins of agronomic interest, when it is desirable to limit expression of said protein to only the tissues/developmental zones or plant lifecycle stages where it is necessary to obtain the agronomically important trait.

In other embodiments, the transcribable polynucleotide molecules can affect an agronomically important trait by encoding an RNA molecule that causes the targeted inhibition, or substantial inhibition, of expression of an endogenous gene (e.g., via antisense, RNAi, and/or cosuppression-mediated mechanisms). The RNA could also be a catalytic RNA molecule (i.e., a ribozyme) engineered to cleave a desired endogenous RNA product. Thus, any polynucleotide molecule that encodes a protein or mRNA that expresses a phenotype or morphology change of interest is useful for the practice of the present invention.

The constructs of the present invention may be double Ti plasmid border DNA constructs that have the right border (RB) and left border (LB) regions of the Ti plasmid isolated from Agrobacterium tumefaciens comprising a transfer DNA (T-DNA), that along with transfer molecules provided by the Agrobacterium cells, permits the integration of the T-DNA into the genome of a plant cell. The constructs also may contain the plasmid backbone DNA segments that provide replication function and antibiotic selection in bacterial cells, for example, an E. coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker. For plant transformation, the host bacterial strain is often Agrobacterium tumefaciens ABI, C58, or LBA4404, however, other strains known to those skilled in the art of plant transformation can function in the present invention.

### Transgenic Cells, Host Cells, Plants and Plant Cells

The polynucleotides and constructs as provided herein can be used in the preparation of transgenic host cells, tissues, organs, and organisms. Thus, also provided are transgenic host cells, tissues, organs, and organisms that contain an introduced regulatory polynucleotide molecule as provided herein.

The transgenic host cells, tissues, organs, and organisms disclosed herein comprise a recombinant polynucleotide construct having (1) an isolated regulatory polynucleotide molecule comprising a polynucleotide molecule selected from the group consisting of a) a polynucleotide molecule comprising a nucleic acid molecule having the sequence of SEQ ID NOs: 1-105 that is capable of regulating transcription of an operably linked transcribable polynucleotide molecule; b) a polynucleotide molecule having at least about 70% sequence identity to the sequence of SEQ ID NOs: 1-105 that is capable of regulating transcription of an operably linked transcribable polynucleotide molecule; and c) a fragment of the polynucleotide molecule of a) or b) capable of regulating transcription of an operably linked transcribable polynucleotide molecule, operably linked to (2) a transcribable polynucleotide molecule.

A plant transformation construct containing a regulatory polynucleotide as provided herein may be introduced into plants by any plant transformation method. The polynucleotide molecules and constructs provided herein may be introduced into plant cells or plants to direct transient expression of operably linked transcribable polynucleotides or be stably integrated into the host cell genome. Methods and materials for transforming plants by introducing a plant expression construct into a plant genome in the practice of this invention can include any of the well-known and demonstrated methods including electroporation; microprojectile bombardment; Agrobacterium-mediated transformation; and protoplast transformation.

Plants and plant cells for use in the production of the transgenic plants and plant cells include both monocotyledonous and dicotyledonous plants and plant cells. Methods for specifically transforming monocots and dicots are well known to those skilled in the art. Transformation and plant regeneration using these methods have been described for a number of crops including, but not limited to, soybean (Glycine max), Brassica sp., Arabidopsis thaliana, cotton (Gossypium hirsutum), peanut (Arachis hypogae), sunflower (Helianthus annuus), potato (Solanum tuberosum), tomato (Lycopersicon esculentum L.), rice, (Oryza sativa), corn (Zea mays), and alfalfa (Medicago sativa). It is apparent to those of skill in the art that a number of transformation methodologies can be used and modified for production of stable transgenic plants from any number of target crops of interest. Transgenic plants and plant cells include, but are not limited to, the above-identified plants as well as wheat, turf grass, millet, sorghum, switchgrass, miscanthus, sugarcane, and Bracypodium.

The transformed plants may be analyzed for the presence of the transcribable polynucleotides of interest and the expression level and/or profile conferred by the regulatory polynucleotides of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays.

The seeds of this invention can be harvested from fertile transgenic plants and be used to grow progeny generations of the transformed plants disclosed herein. The terms “seeds” and “kernels” are understood to be equivalent in meaning. In the context of the present invention, the seed refers to the mature ovule consisting of a seed coat, embryo, aleurone, and an endosperm.

Thus, also provided are methods for expressing transcribable polynucleotides in host cells, plant cells, and plants. In some embodiments, such methods comprise stably incorporating into the genome of a host cell, plant cell, or plant, a regulatory polynucleotide operably linked to a transcribable polynucleotide molecule of interest and regenerating a stably transformed plant that expresses the transcribable polynucleotide molecule. In other embodiments, such methods comprise the transient expression of a transcribable polynucleotide operably linked to a regulatory polynucleotide molecule provided herein in a host cell, plant cell, or plant.

Such methods of directing expression of a transcribable polynucleotide molecule in a host cell, such as a plant cell, include: A) introducing a recombinant nucleic acid construct into a host cell, the construct having (1) an isolated regulatory polynucleotide molecule comprising a polynucleotide molecule selected from the group consisting of a) a polynucleotide molecule comprising a nucleic acid molecule having the sequence of SEQ ID NOs: 1-105 that is capable of regulating transcription of an operably linked transcribable polynucleotide molecule; b) a polynucleotide molecule having at least about 70% sequence identity to the sequence of SEQ ID NOs: 1-105 that is capable of regulating transcription of an operably linked transcribable polynucleotide molecule; and c) a fragment of the polynucleotide molecule of a) or b) capable of regulating transcription of an operably linked transcribable polynucleotide molecule, operably linked to (2) a transcribable polynucleotide molecule; and B) selecting a transgenic host cell exhibiting expression of the transcribable polynucleotide molecule.

The articles “a” and “an” are used herein to refer to one or more than one (i.e., to at least one) of the grammatical object of the article. By way of example, “an element” means one or more elements.

As used herein, the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The following examples are offered by way of illustration and not by way of limitation.

### EXAMPLES

### Example 1

### Identification of Arabidopsis Tissue-Specific Regulatory Sequences

A bioinformatics approach was used to identify regulatory polynucleotides that have putative tissue-specific activity. The specificity of most plant regulatory polynucleotides (such as promoters) has been characterized at the organ level (i.e., roots, shoots, leaves, seeds) and not at the cell type/tissue level. The method used to identify the regulatory polynucleotides described herein was used to identify regulatory polynucleotides having specific patterns of expression activity at the cell type and/or tissue level.

Five patterns or categories of root tissue-specific activity were targeted:
(1) All or substantially all root tissues in all root developmental zones (“broad root”);
(2) Epidermal, vascular, cortex, and phloem tissues, in all root developmental zones;
(3) Pericycle, endodermis, and cortex tissues, in all root developmental zones;
(4) Epidermal tissue, including root hairs, in all root developmental zones;
(5) Meristem and root cap tissues in the root meristematic and elongation zones.
   For category 2, high expression in vascular tissue was prioritized, with reduced expression in non-target tissues as a secondary goal. For category 3, limiting significant expression to only the target tissues was prioritized.

Using existing microarray expression data, bioinformatics analysis methods were used to identify genes from this data collection that are highly expressed in target cell types and longitudinal zones of the Arabidopsis root and that are not expressed or expressed at lower levels in aerial tissue and non-target root tissues and root longitudinal zones.

Such existing data includes microarray expression profiles of all cell-types and developmental zones within Arabidopsis root tissue (Brady et al., Science, 318:801-806 (2007)). The radial dataset comprehensively profiles expression of 14 non-overlapping cell-types in the root, while the longitudinal data set profiles developmental zones by measuring expression in 13 longitudinal sections. This detailed expression profiling has mapped the spatiotemporal expression patterns of nearly all genes in the Arabidopsis root. To assess expression in aerial tissue and responsiveness to abiotic stress, the expression profiles of these candidates were also analyzed in the AtGenExpress Development and Abiotic Stress datasets (available on the World Wide Web at the site weigelworld.org/resources/microarray/AtGenExpress).

Each Arabidopsis gene was scored on multiple criteria, such as ratio of expression in root to expression in aerial tissues (shoots, flowers, and seeds); variation in expression under abiotic stresses; ratio of expression in target tissues/zones to expression in non-target tissues/zones; and absolute level of expression in target tissues/zones. High-dimensional visualizations of these score distributions were examined to determine appropriate cutoffs for each score component, and the genes meeting those cutoffs were prioritized manually. For pattern 1, selected genes exhibited 10-fold greater expression in root tissues than in seed tissues under normal conditions, had GC-RMA expression levels above 4 in all tissue and developmental zones, and exhibited 10-fold greater expression in root tissues than in shoot tissues across all abiotic stress conditions. For pattern 2, selected genes exhibited 10-fold greater expression in root tissues than in seed tissues under normal conditions; had GC-RMA expression levels above 4 in one or more tissues marked by SUC2, S32, CORTEX, APL, and/or S18; exhibited 3.5-fold greater expression in one or more tissues marked by SUC2, S32, CORTEX, APL, and/or S18 than in other root tissues; and exhibited 3-fold greater expression in root tissues than in shoot tissues across all abiotic stress conditions. For pattern 3, some selected genes exhibited 7.5-fold greater expression in root tissues than in seed tissues under normal conditions; had GC-RMA expression levels above 4 in one or more tissues marked by S17, J2661, J0571, J0121, and/or CORTEX; exhibited 3.25-fold greater expression in one or more tissues marked by S17, J2661, J0571, J0121, and/or CORTEX than in other root tissues; and exhibited greater expression in root tissues than in shoot tissues across all abiotic stress conditions. Other selected genes exhibited 10-fold greater expression in root tissues than in seed and shoot tissues under normal conditions; had GC-RMA expression levels above 5 in one or more tissues marked by S17, J2661, J0571, J0121, and/or CORTEX; exhibited 6-fold greater expression in one or more tissues marked by S17, J2661, J0571, J0121, and/or CORTEX than in other root tissues; and exhibited 1.8-fold greater expression in root tissues than in shoot tissues across all abiotic stress conditions. For pattern 4, selected genes exhibited 10-fold greater expression in root tissues than in seed tissues under normal conditions; had GC-RMA expression levels above 4 in one or more tissues marked by GL2 and/or COBL9; exhibited 2.8-fold greater expression in one or more tissues marked by GL2 and/or COBL9 than in other root tissues; and exhibited 2-fold greater expression in root tissues than in shoot tissues across all abiotic stress conditions. For pattern 5, selected genes exhibited 10-fold greater expression in root tissues than in seed tissues under normal conditions; had GC-RMA expression levels above 4 in one or more tissues marked by PET111 and/or LRC and above 3.6 in developmental zones 1-8; exhibited 2.3-fold greater expression in those developmental zones than in zones 9-13; and exhibited 2-fold greater expression in root tissues than in shoot tissues across all abiotic stress conditions.

To identify regulatory polynucleotide molecules responsible for driving tissue-specific expression of these candidate genes, upstream sequences of 1500 bp or less of the selected gene candidates were determined. Because transcription start sites are not always known, sequences upstream of the translation start site were used in all cases. Therefore, the selected regulatory polynucleotide molecules contain an endogenous 5′-UTR, and some of the endogenous 5′-UTRs may contain introns. The use of such introns in expression constructs containing these regulatory sequences may increase expression through IME. Without being limited by theory, because IME may be important for highly expressed constitutive genes, it is believed that IME may also play a role in the expression of genes expressed in a tissue-specific manner. To capture these regulatory molecules in genes that do not contain a 5′-UTR intron, chimeric regulatory polynucleotide molecules may be constructed wherein the first intron from the gene of interest is fused to the 3′-end of the 5′-UTR of the regulatory polynucleotide (which may be from the same or a different (e.g., exogenous) gene). To ensure efficient intron splicing, the introns in these chimeric molecules may be flanked by consensus splice sites.

Selected regulatory polynucleotides are listed in Table 1 below, with the corresponding tissue-specific category listed. Sequences including the regulatory polynucleotides plus the first intron from the coding region added at the 3′ end of the 5′ UTR are indicated by the corresponding gene accession number and the indicator “+intron”:

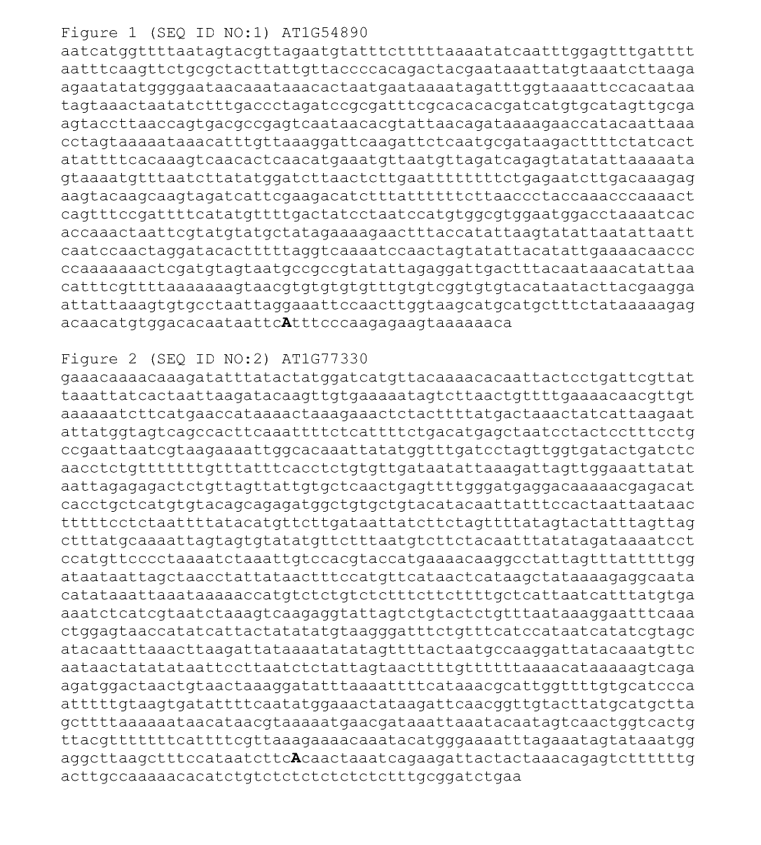

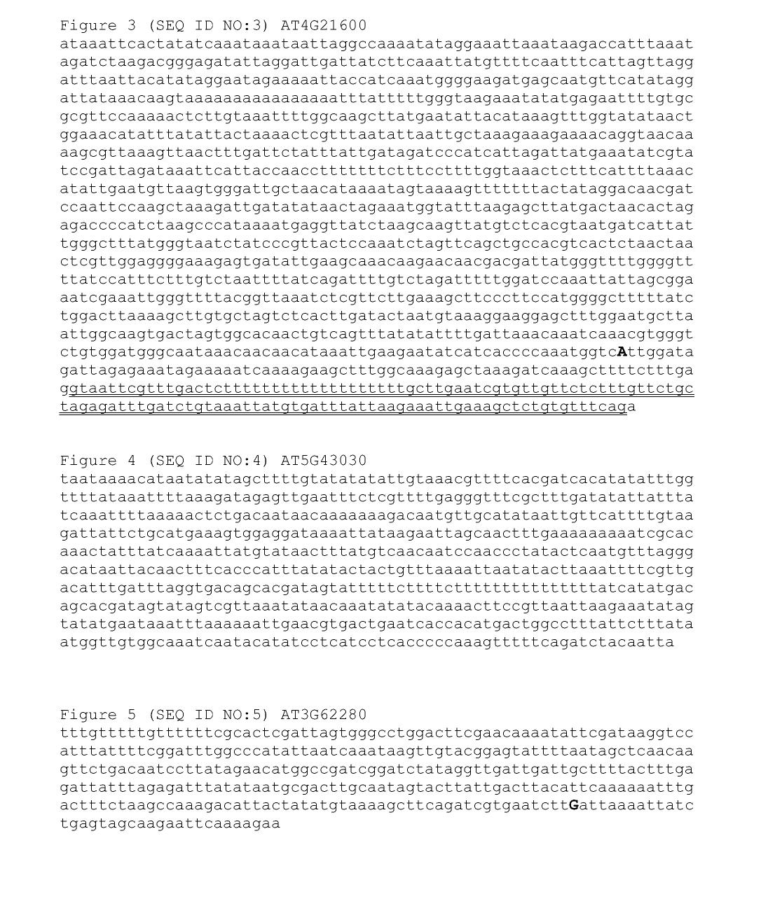

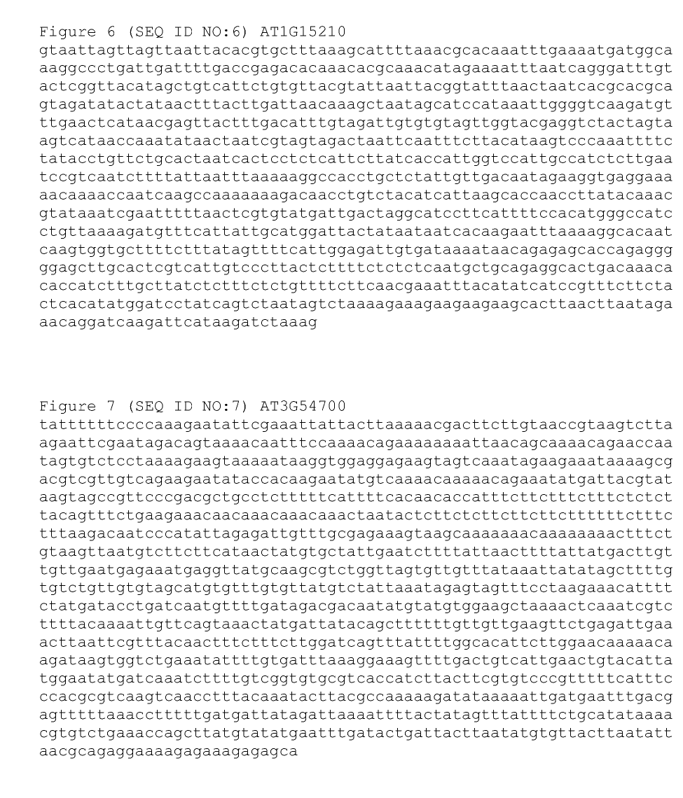

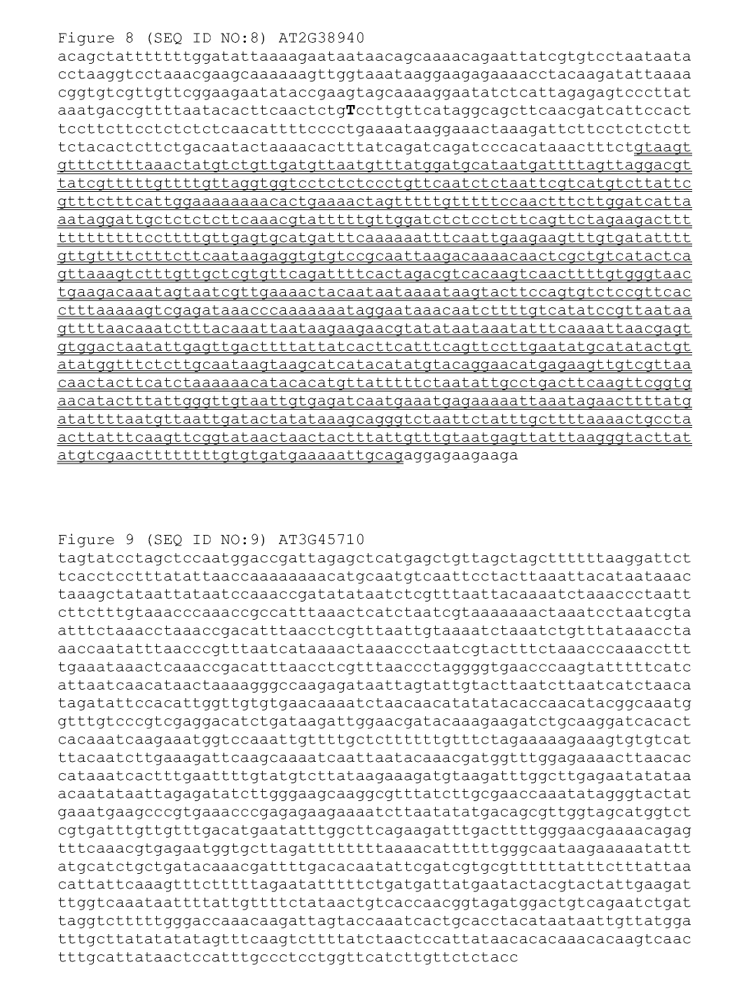

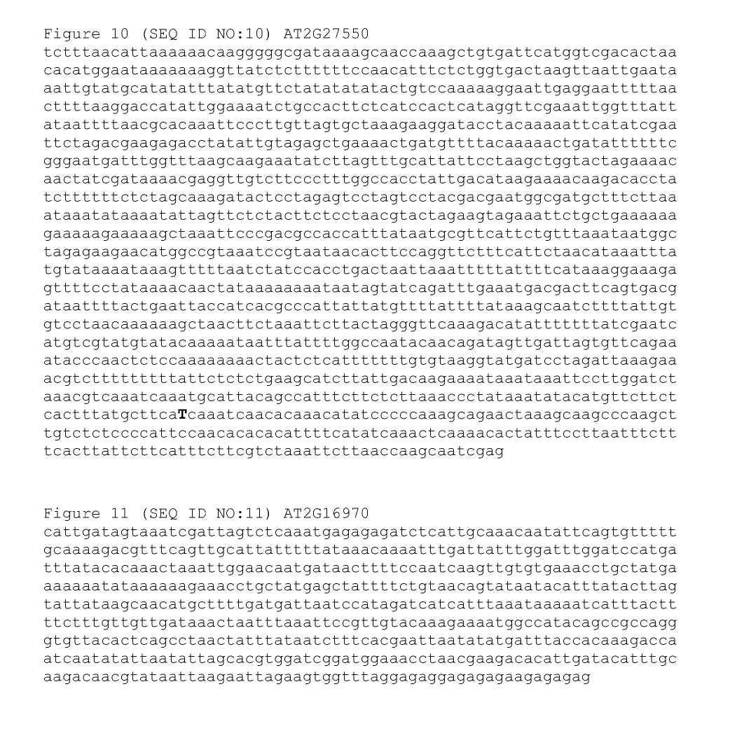

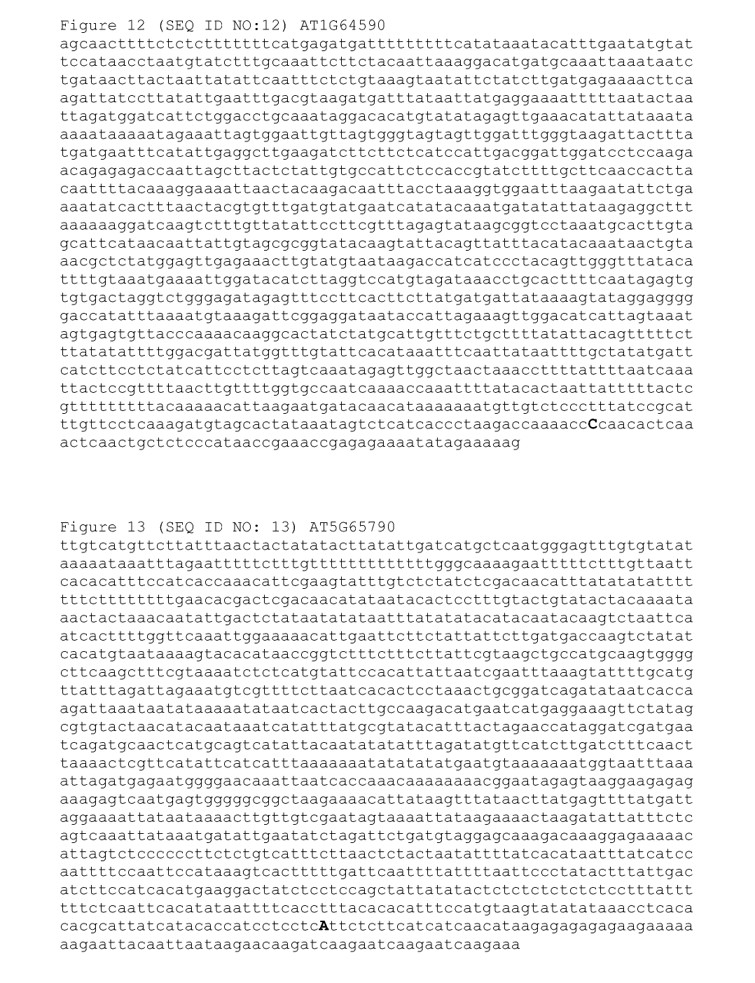

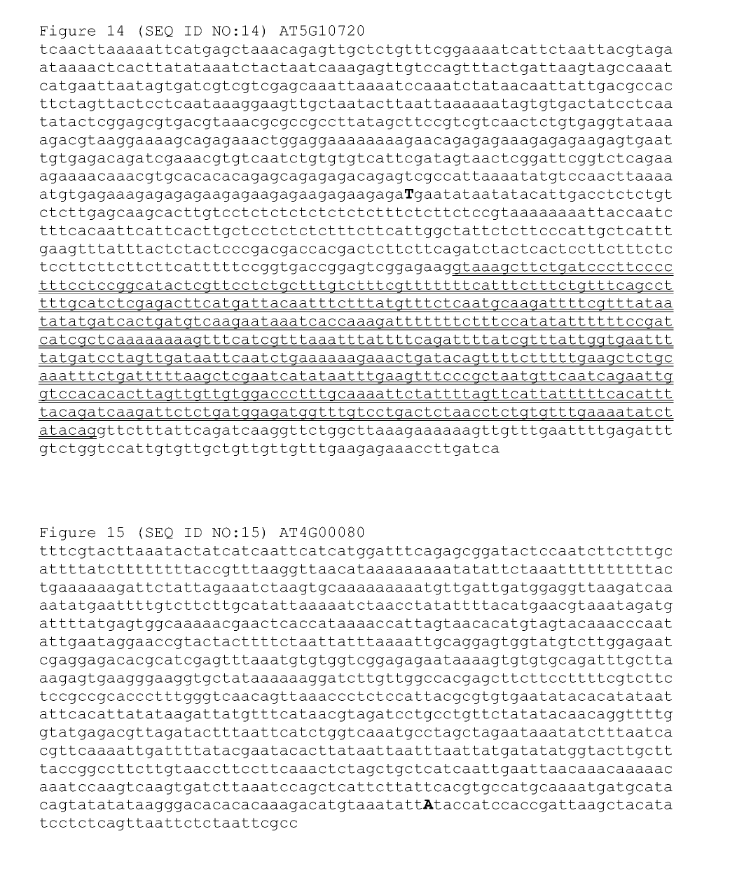

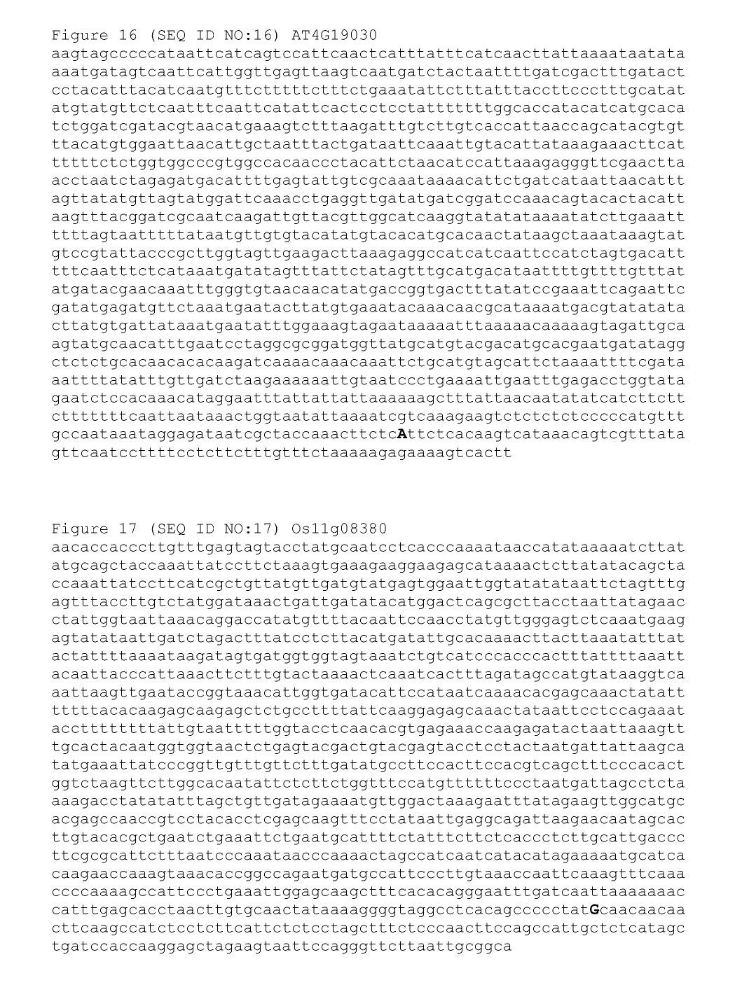

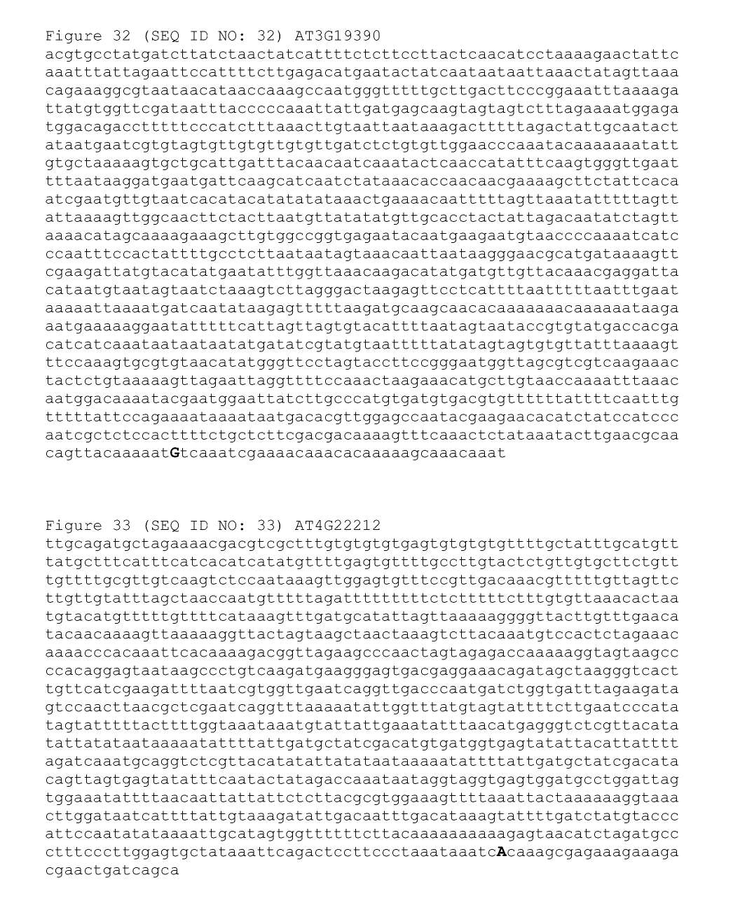

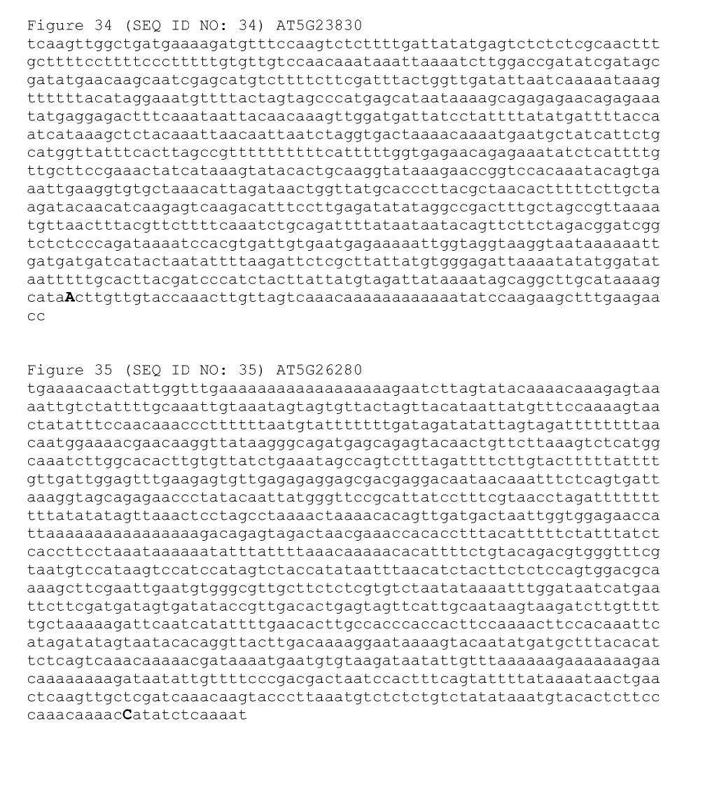

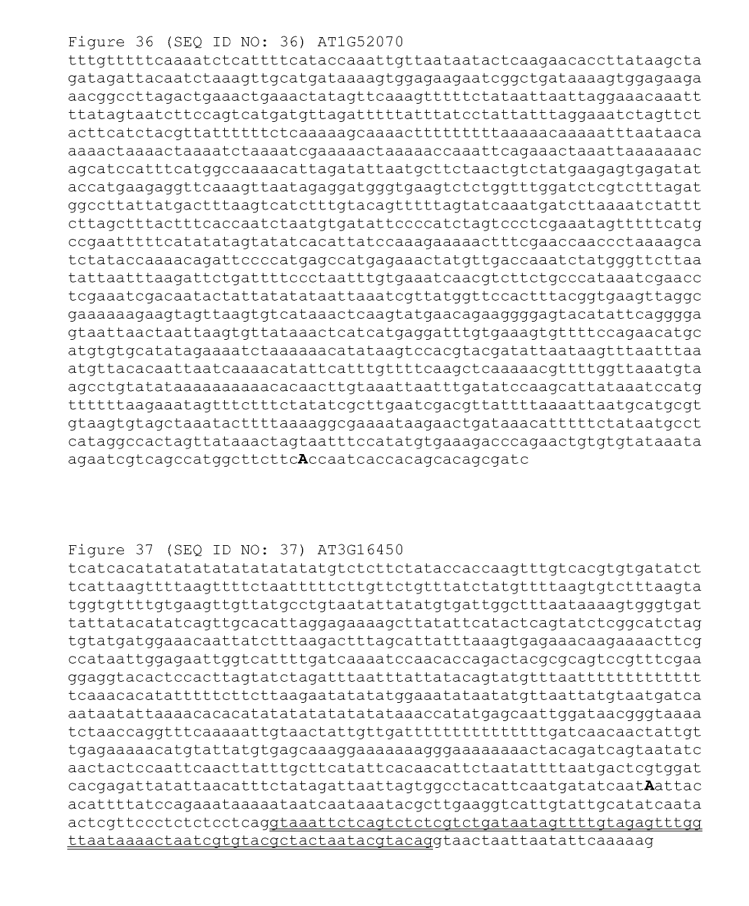

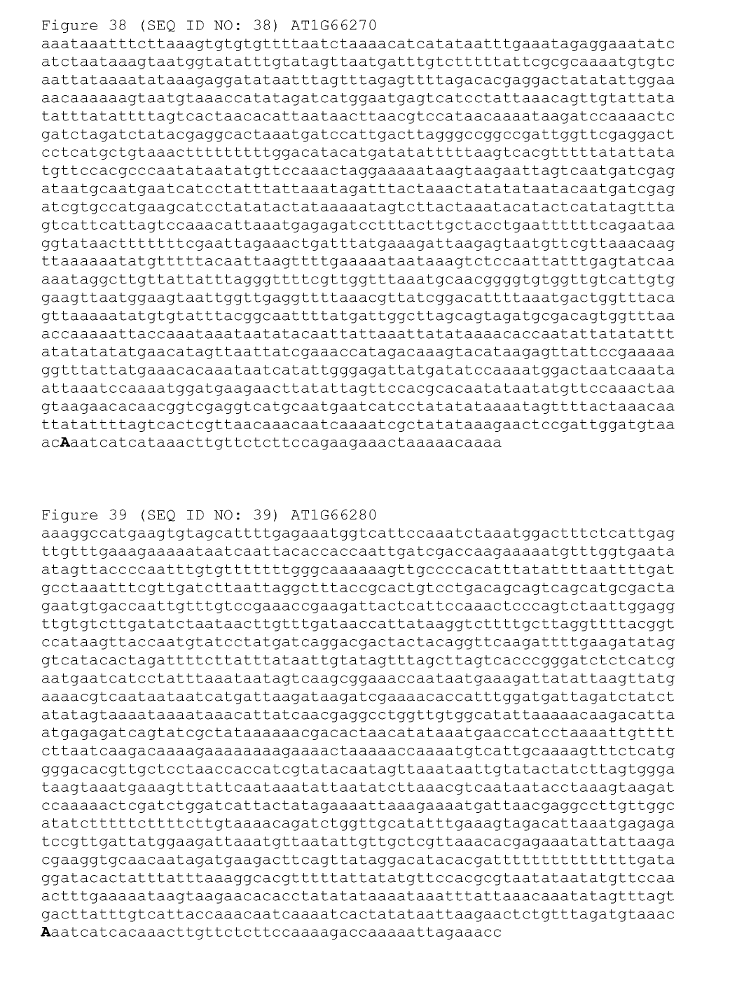

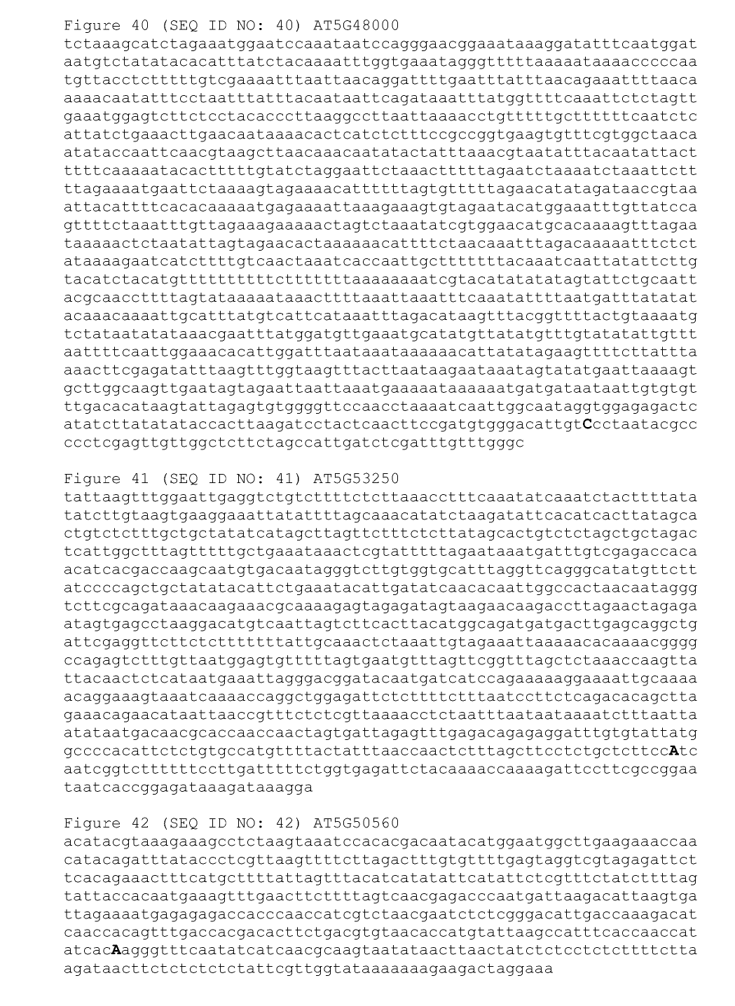

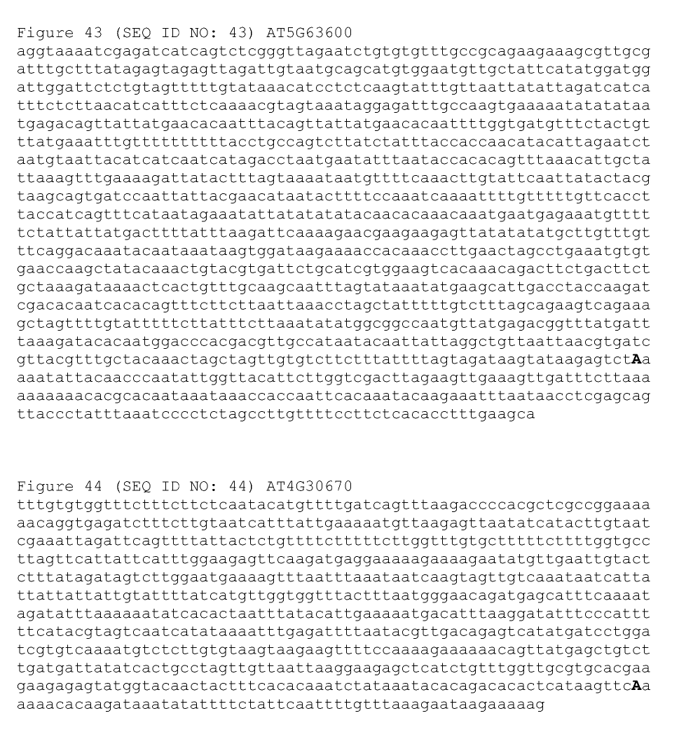

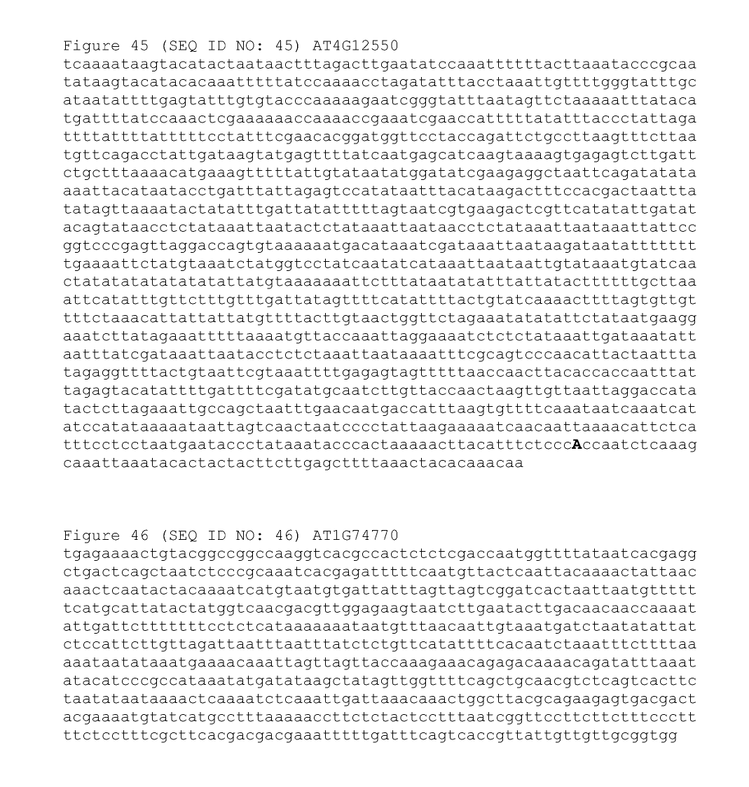

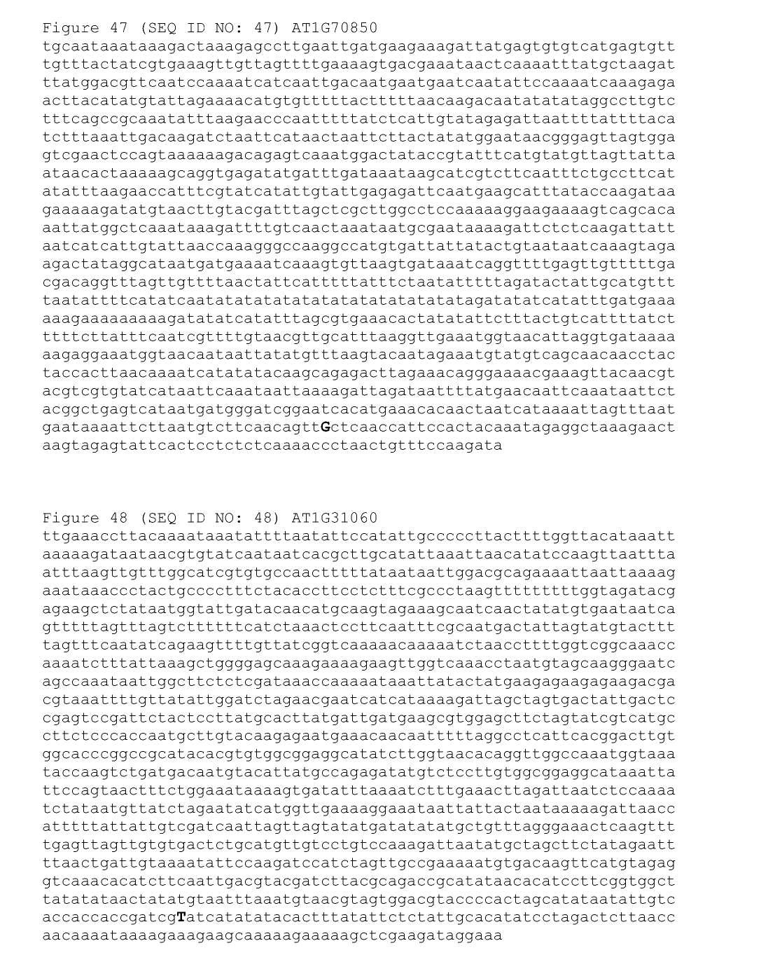

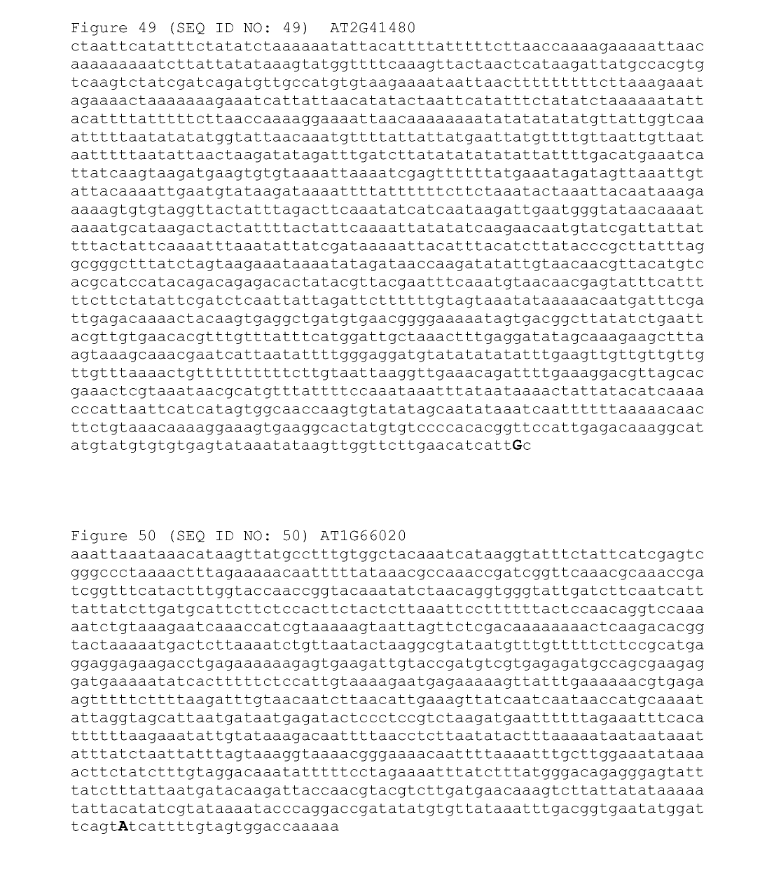

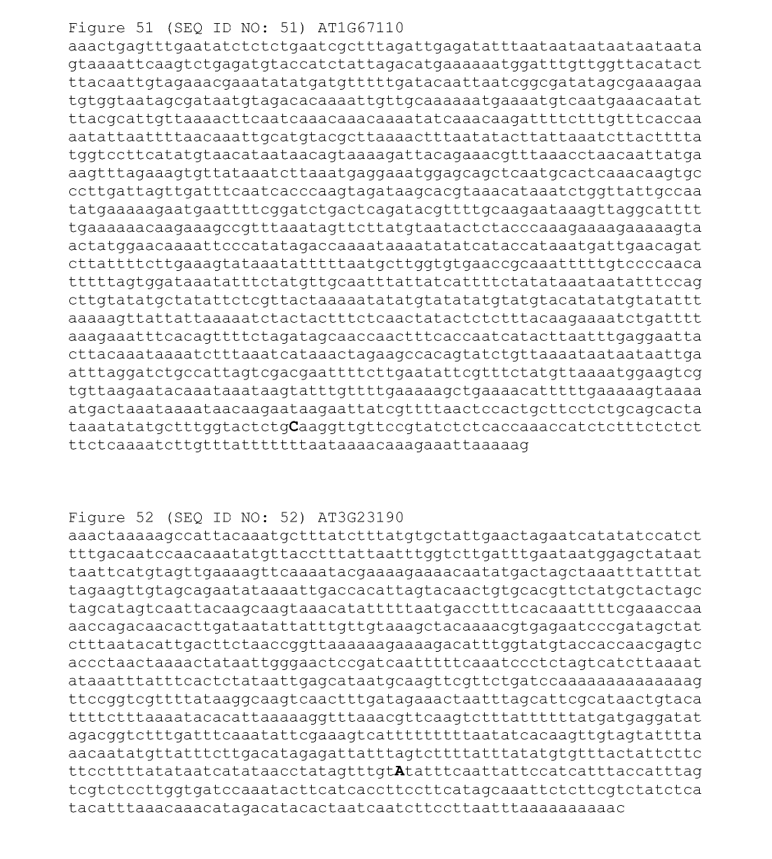

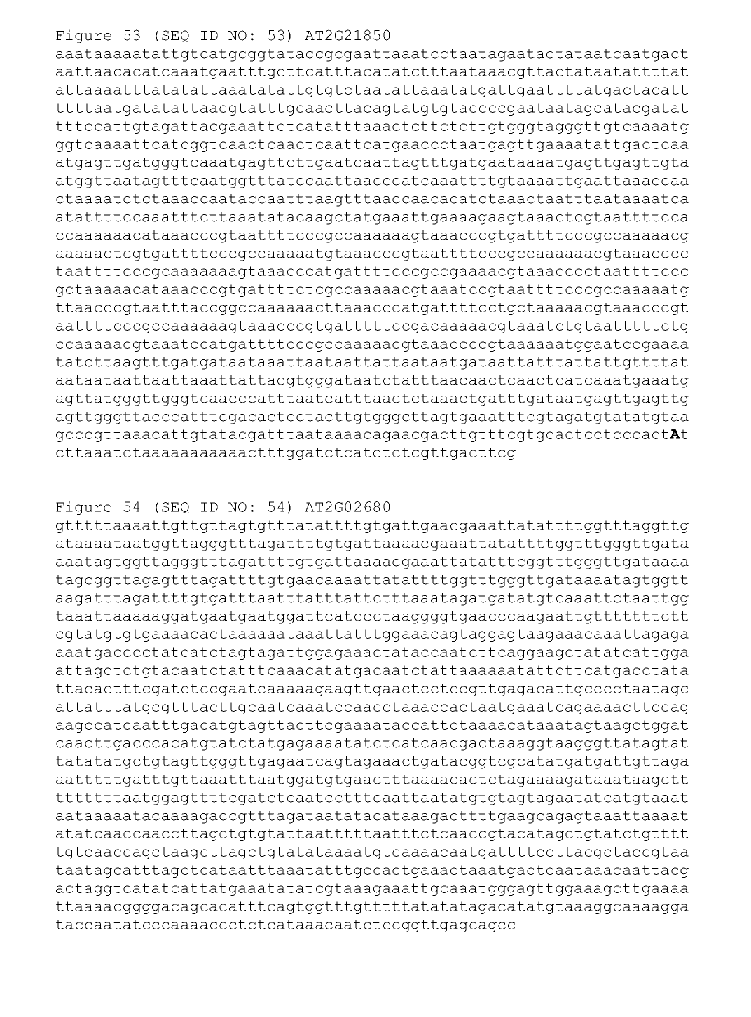

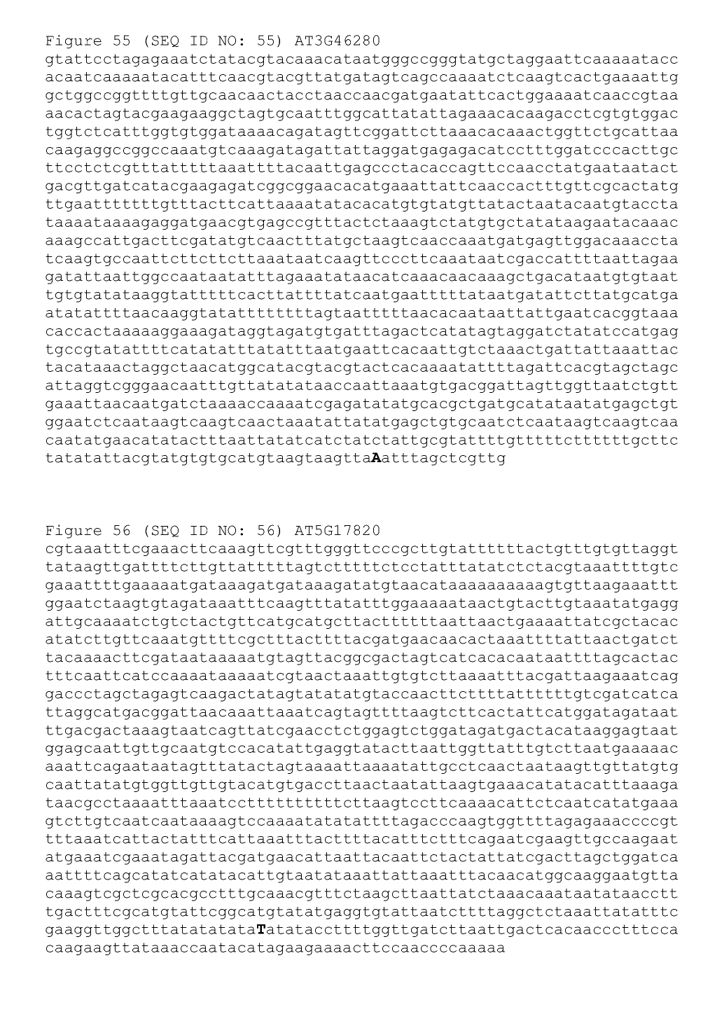

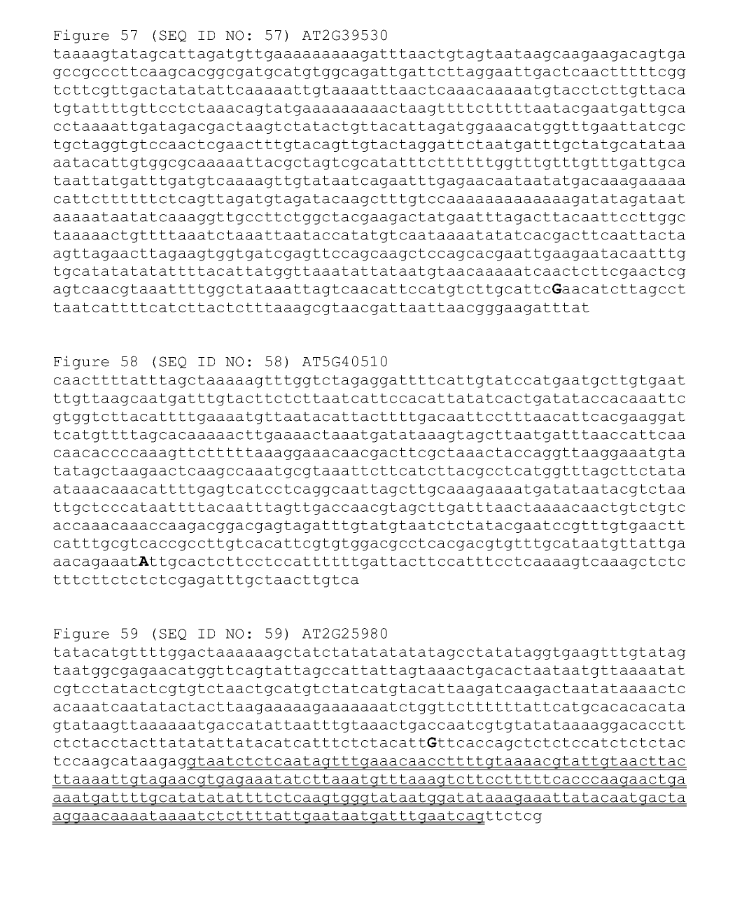

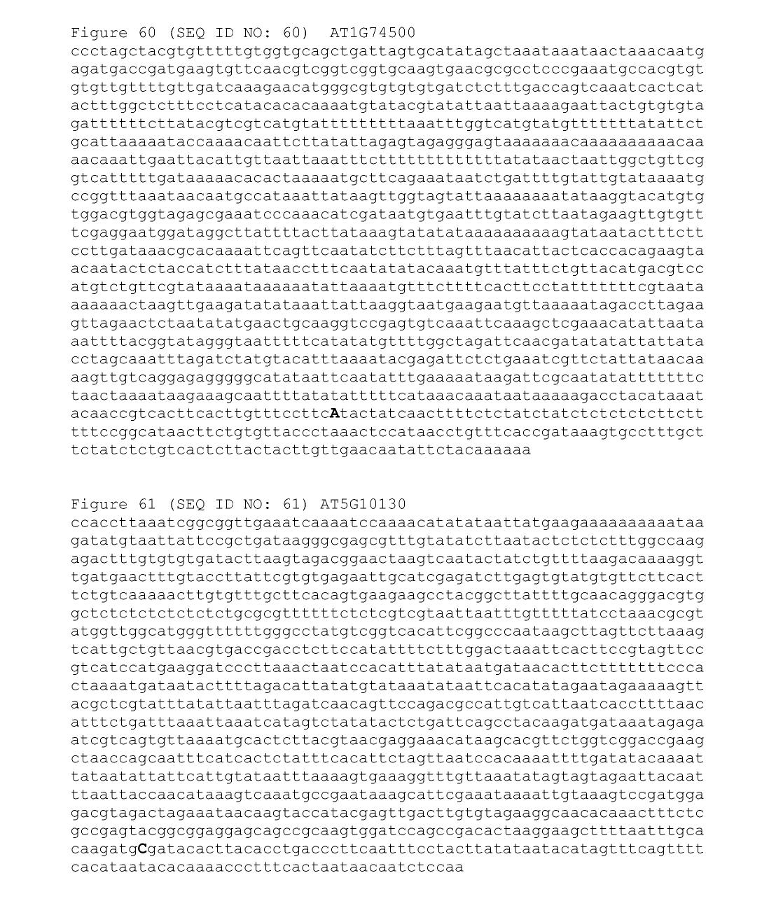

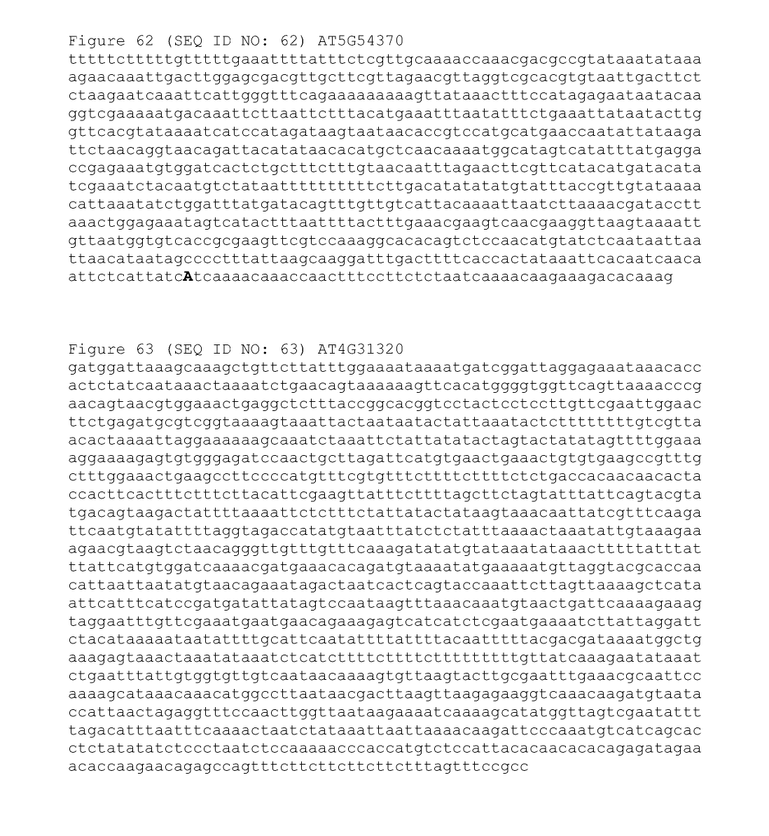

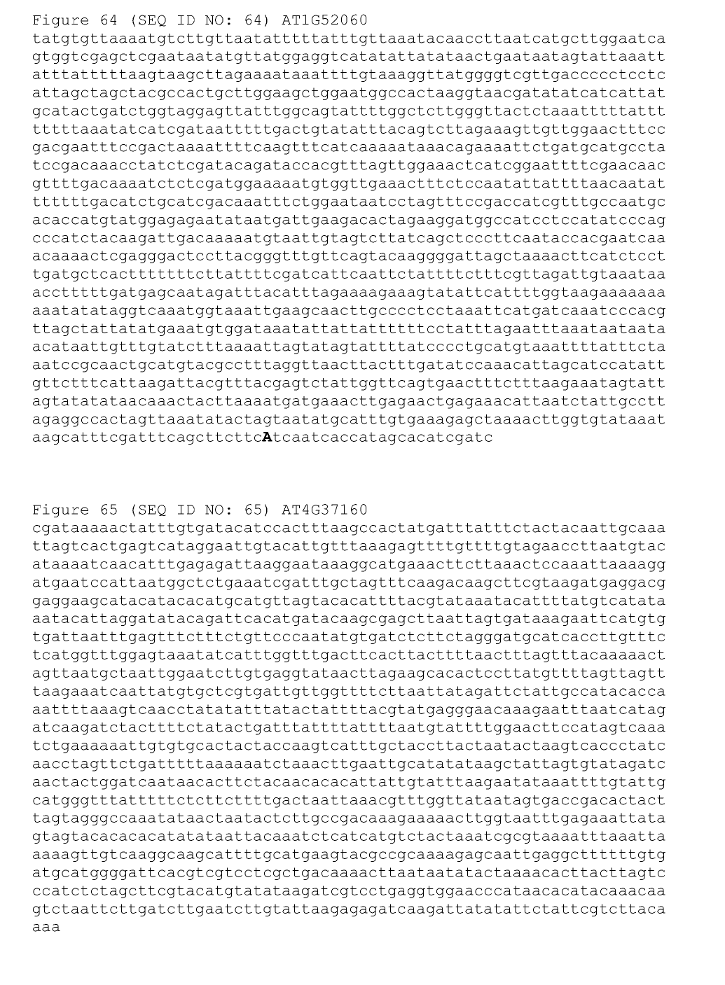

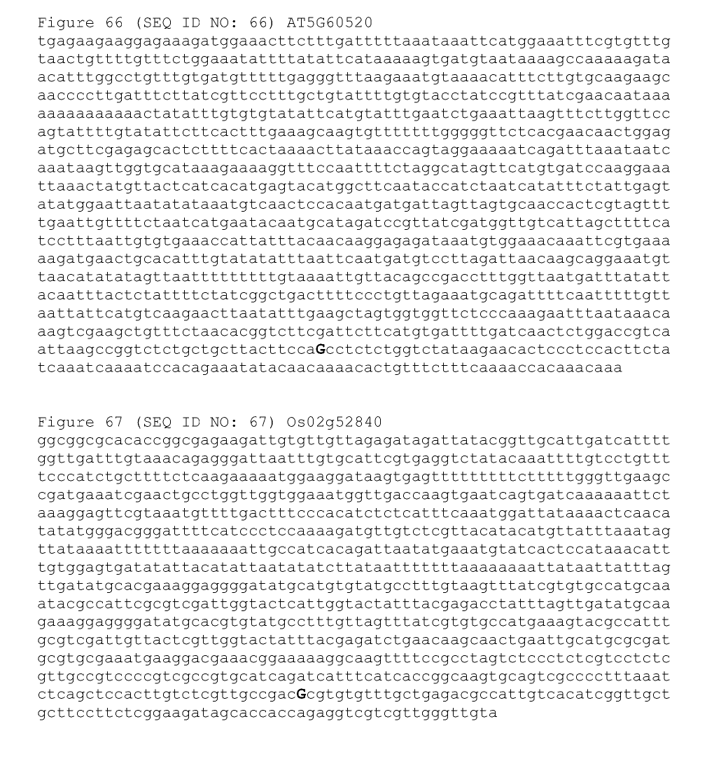

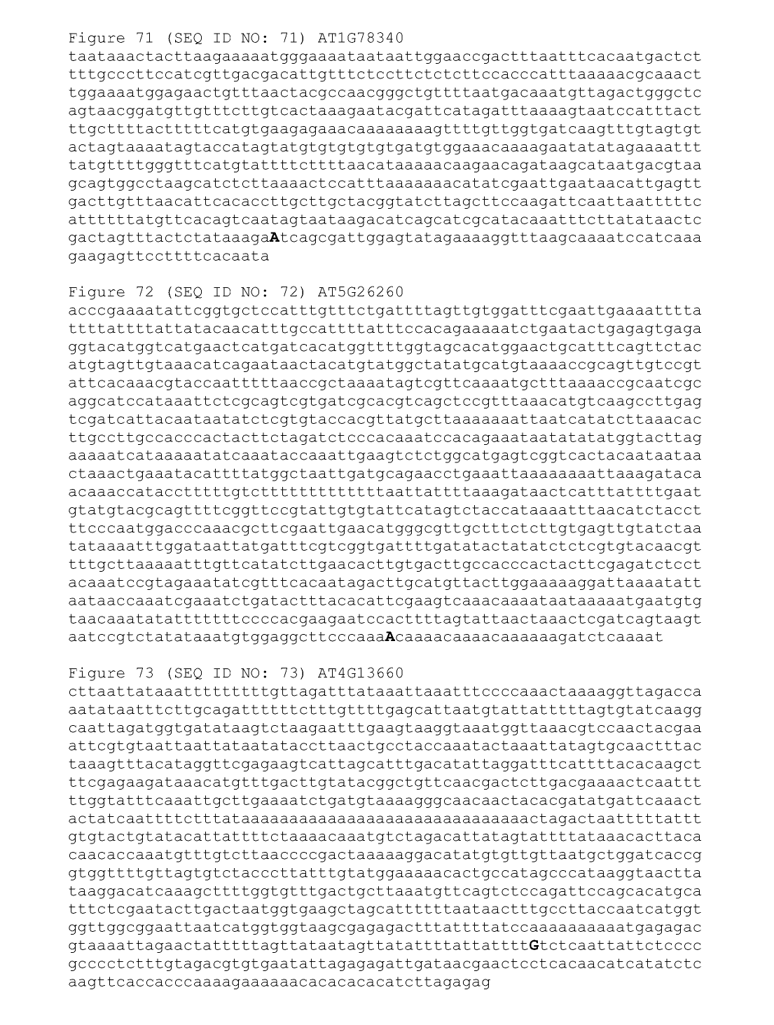

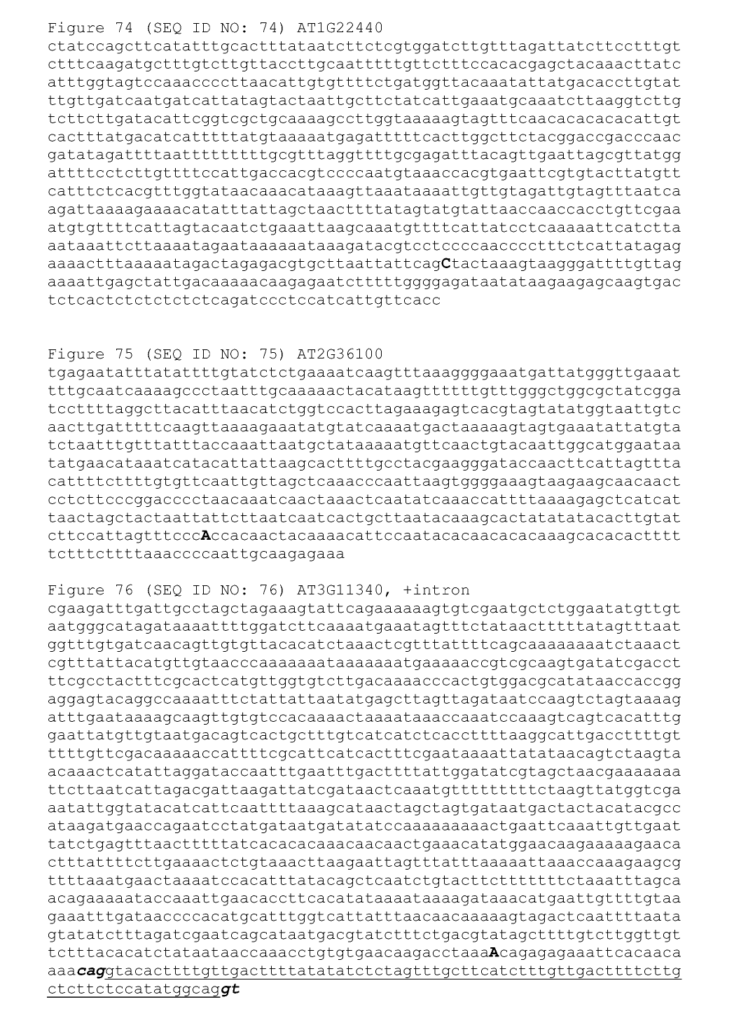

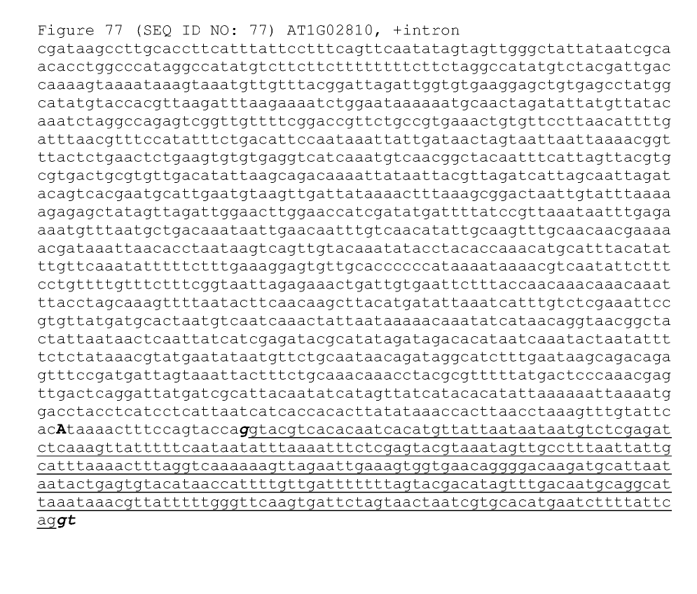

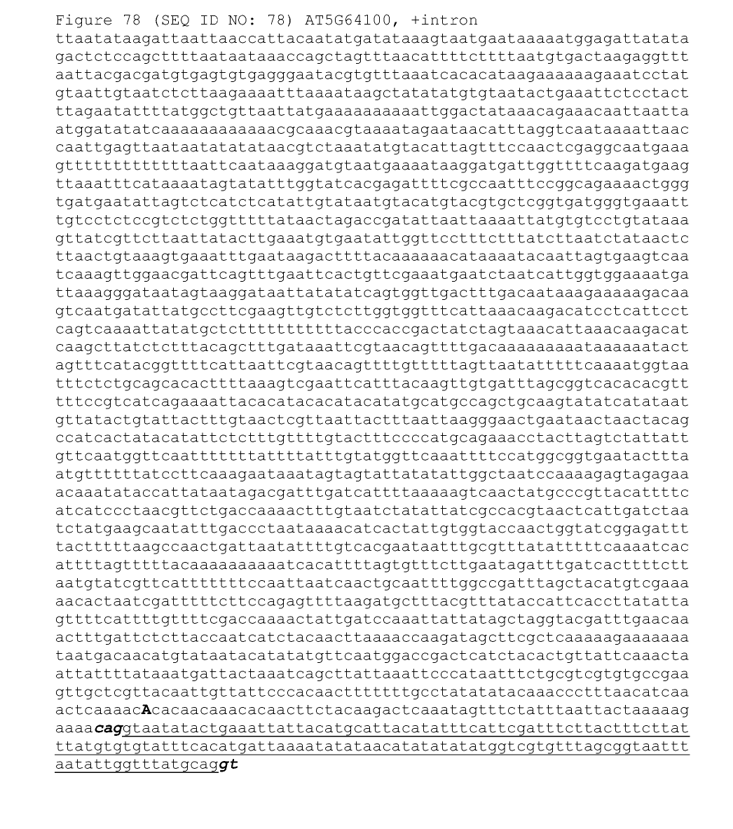

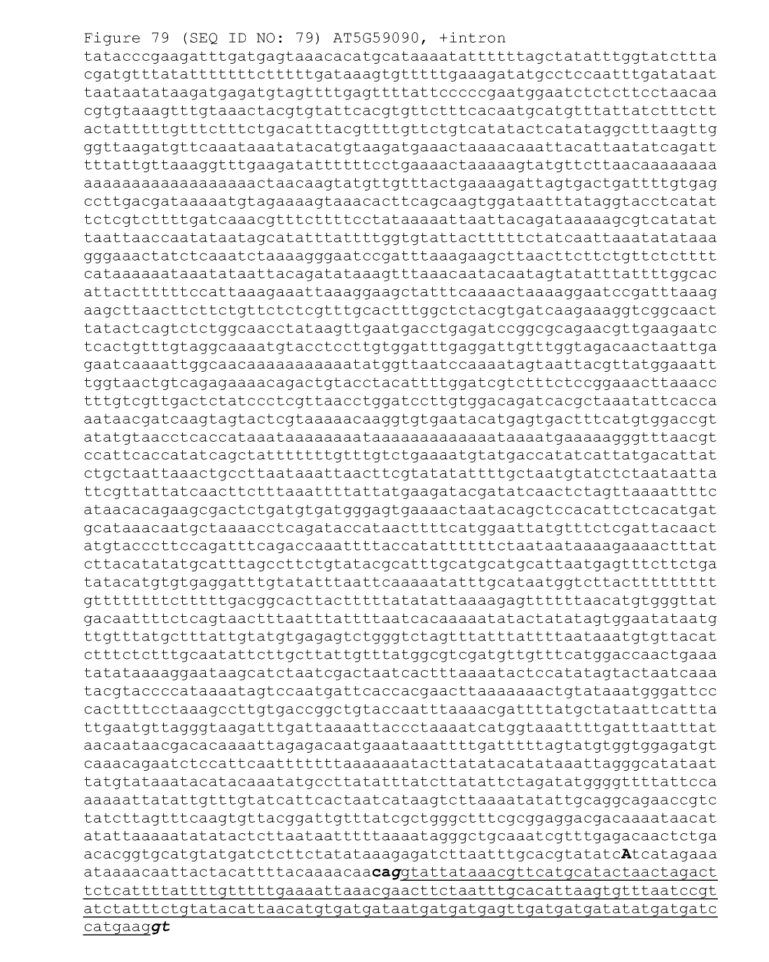

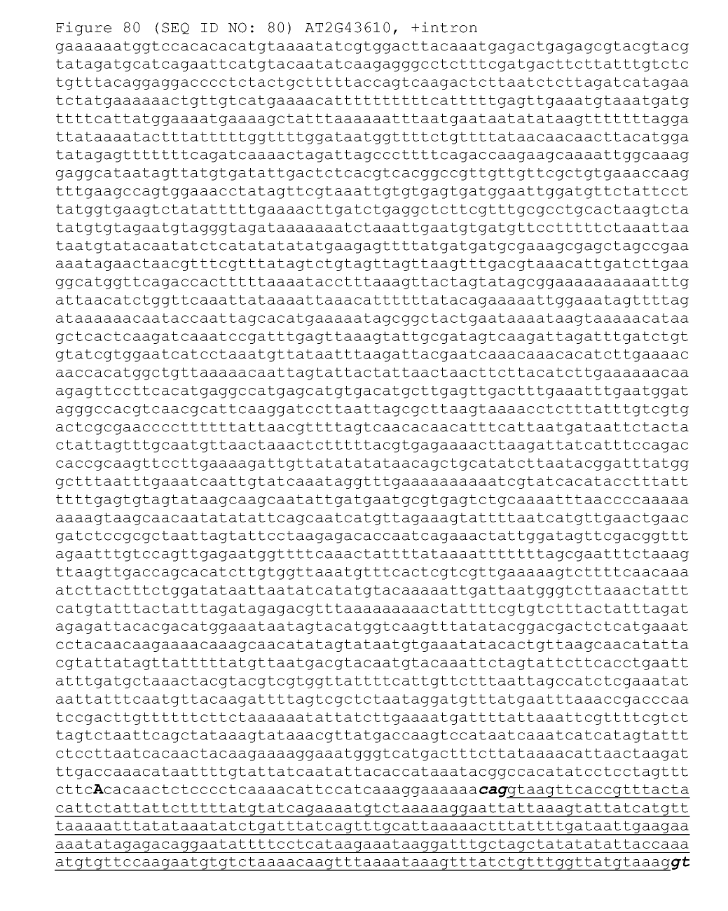

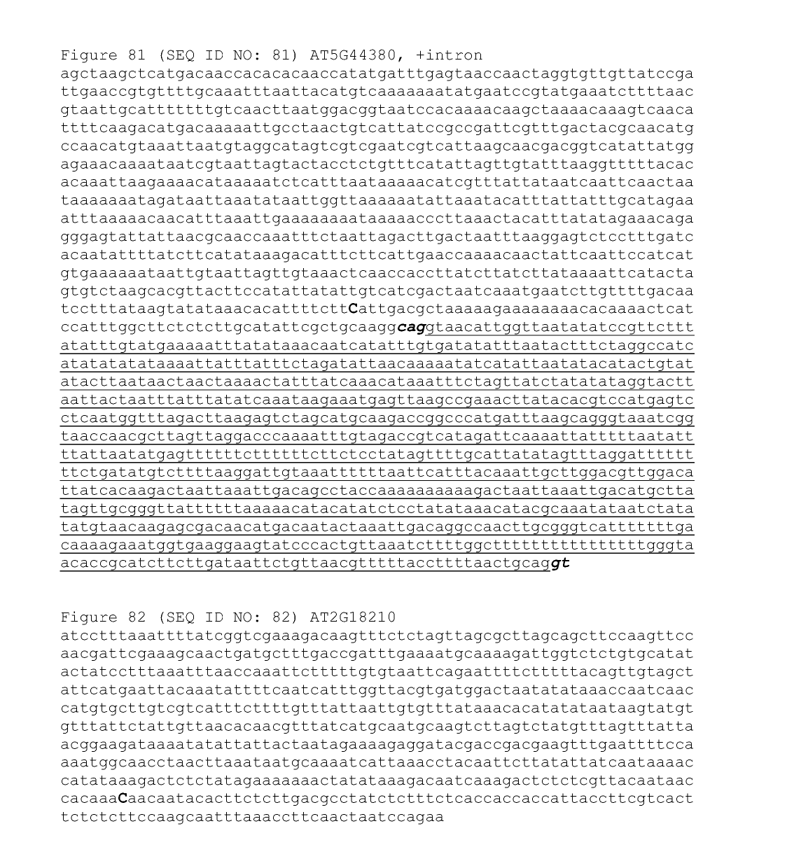

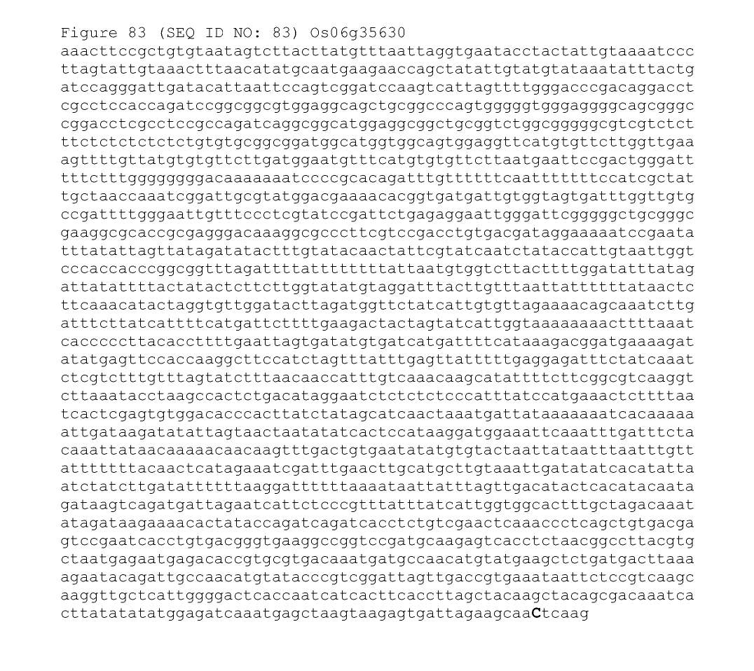

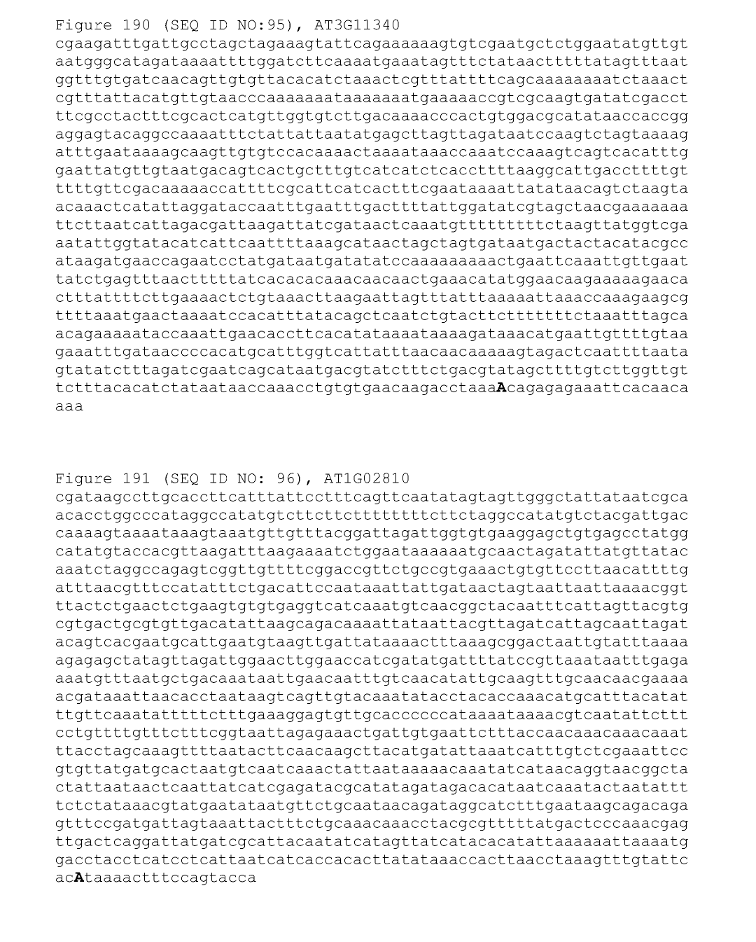

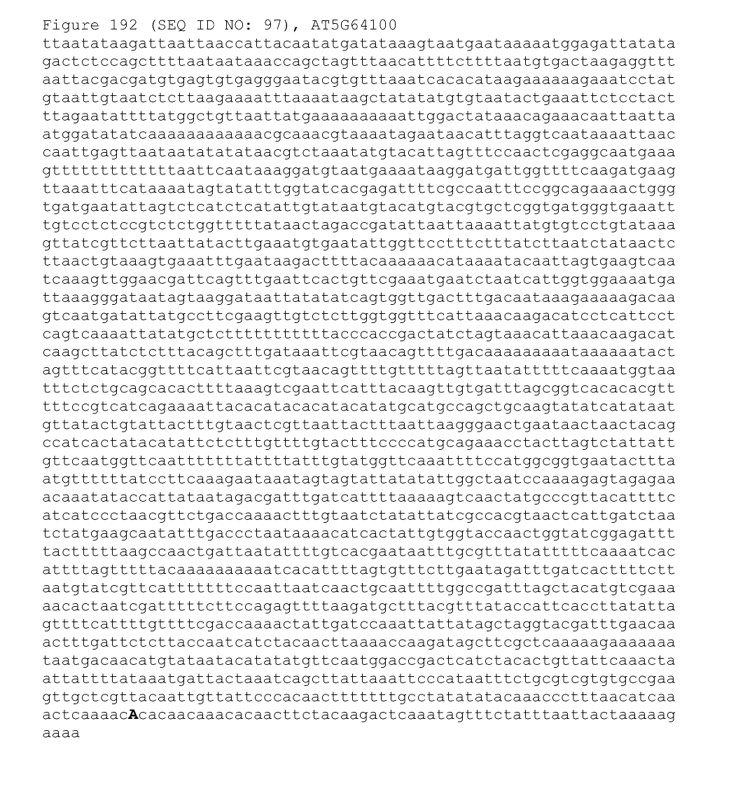

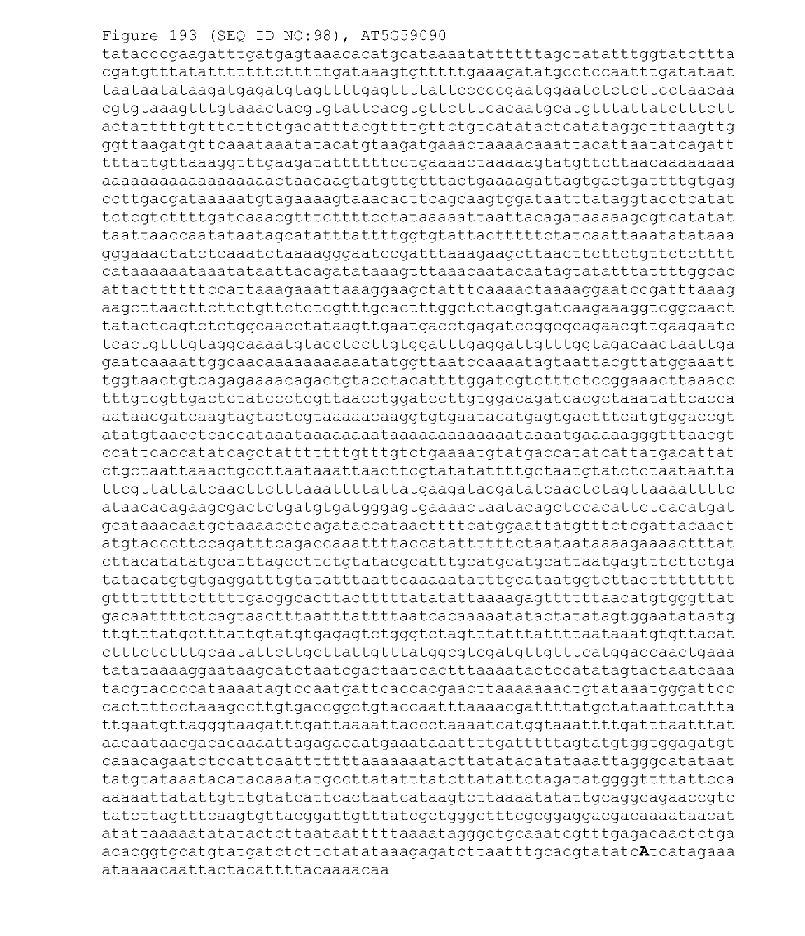

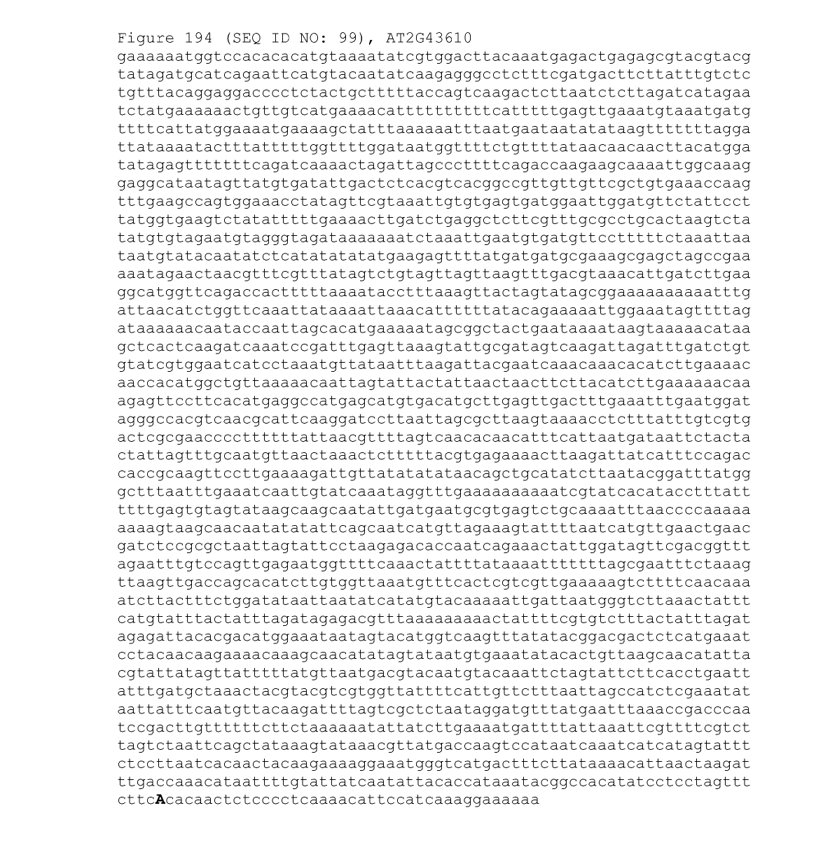

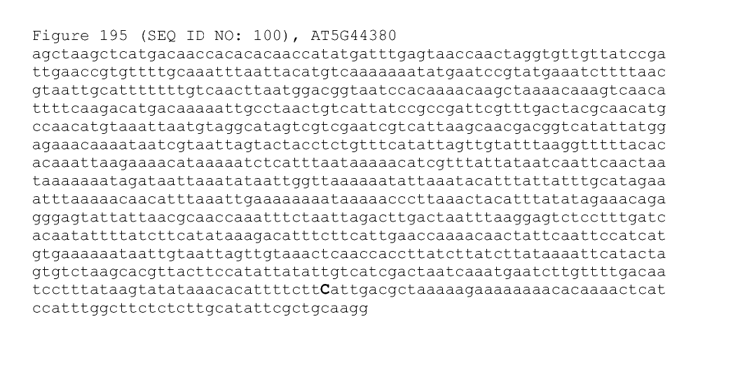

TABLE 1

| Fig- | SEQ | Corresponding | Tissue-Specific |
|---|---|---|---|
| ure | ID NO: | Gene Accession No. | Category |
| 1 | 1 | AT1G54890 | epidermis (including |
| | | | root hair), all root |
| | | | developmental zones |
| 2 | 2 | AT1G77330 | broad root |
| 3 | 3 | AT4G21600 | broad root |
| 4 | 4 | AT5G43030 | epidermis (including |
| | | | root hair), all root |
| | | | developmental zones |
| 5 | 5 | AT3G62280 | epidermis (including |
| | | | root hair), all root |
| | | | developmental zones |
| 6 | 6 | AT1G15210 | broad root |
| 7 | 7 | AT3G54700 | epidermal/vascular/ |
| | | | cortex/phloem, all root |
| | | | developmental zones |
| 8 | 8 | AT2G38940 | epidermal/vascular/ |
| | | | cortex/phloem, all root |
| | | | developmental zones |
| 9 | 9 | AT3G45710 | epidermal/vascular/ |
| | | | cortex/phloem, all root |
| | | | developmental zones |
| 10 | 10 | AT2G27550 | epidermal/vascular/ |
| | | | cortex/phloem, all root |
| | | | developmental zones |
| 11 | 11 | AT2G16970 | pericycle/endodermis/ |
| | | | cortex, all root |
| | | | developmental zones |
| 12 | 12 | AT1G64590 | pericycle/endodermis/ |
| | | | cortex, all root |
| | | | developmental zones |
| 13 | 13 | AT5G65790 | pericycle/endodermis/ |
| | | | cortex (all root |
| | | | developmental zones) |
| 14 | 14 | AT5G10720 | meristem/root cap, |
| | | | meristematic and |
| | | | elongation zones |
| 15 | 15 | AT4G00080 | meristem/root cap, |
| | | | meristematic and |
| | | | elongation zones |
| 16 | 16 | AT4G19030 | meristem/root cap, |
| | | | meristematic and |
| | | | elongation zones |
| 32 | 32 | AT3G19390 | broad root |
| 33 | 33 | AT4G22212 | broad root |
| 34 | 34 | AT5G23830 | broad root |
| 35 | 35 | AT5G26280 | broad root |
| 36 | 36 | AT1G52070 | broad root |
| 37 | 37 | AT3G16450 | broad root |
| 38 | 38 | AT1G66270 | broad root |
| 39 | 39 | AT1G66280 | broad root |
| 40 | 40 | AT5G48000 | epidermal/vascular/ |
| | | | cortex/phloem, all root |
| | | | developmental zones |
| 41 | 41 | AT5G53250 | epidermal/vascular/ |
| | | | cortex/phloem, all root |
| | | | developmental zones |
| 42 | 42 | AT5G50560 | epidermal/vascular/ |
| | | | cortex/phloem, all root |
| | | | developmental zones |
| 43 | 43 | AT5G63600 | epidermal/vascular/ |
| | | | cortex/phloem, all root |
| | | | developmental zones |
| 44 | 44 | AT4G30670 | epidermal/vascular/ |
| | | | cortex/phloem, all root |
| | | | developmental zones |
| 45 | 45 | AT4G12550 | epidermal/vascular/ |
| | | | cortex/phloem, all root |
| | | | developmental zones |
| 46 | 46 | AT1G74770 | epidermal/vascular/ |
| | | | cortex/phloem, all root |
| | | | developmental zones |
| 47 | 47 | AT1G70850 | epidermal/vascular/ |
| | | | cortex/phloem, all root |
| | | | developmental zones |
| 48 | 48 | AT1G31060 | pericycle/endodermis/ |
| | | | cortex, all root |
| | | | developmental zones |
| 49 | 49 | AT2G41480 | pericycle/endodermis/ |
| | | | cortex, all root |
| | | | developmental zones |
| 50 | 50 | AT1G66020 | pericycle/endodermis/ |
| | | | cortex, all root |
| | | | developmental zones |
| 51 | 51 | AT1G67110 | pericycle/endodermis/ |
| | | | cortex, all root |
| | | | developmental zones |
| 52 | 52 | AT3G23190 | epidermis (including |
| | | | root hair), all root |
| | | | developmental zones |
| 53 | 53 | AT2G21850 | epidermis (including |
| | | | root hair), all root |
| | | | developmental zones |
| 54 | 54 | AT2G02680 | epidermis (including |
| | | | root hair), all root |
| | | | developmental zones |
| 55 | 55 | AT3G46280 | epidermis (including |
| | | | root hair), all root |
| | | | developmental zones |
| 56 | 56 | AT5G17820 | epidermis (including |
| | | | root hair), all root |
| | | | developmental zones |
| 57 | 57 | AT2G39530 | epidermis (including |
| | | | root hair), all root |
| | | | developmental zones |
| 58 | 58 | AT5G40510 | epidermis (including |
| | | | root hair), all root |
| | | | developmental zones |
| 59 | 59 | AT2G25980 | meristem/root cap, |
| | | | meristematic and |
| | | | elongation zones |
| 60 | 60 | AT1G74500 | meristem/root cap, |
| | | | meristematic and |
| | | | elongation zones |
| 61 | 61 | AT5G10130 | meristem/root cap, |
| | | | meristematic and |
| | | | elongation zones |
| 62 | 62 | AT5G54370 | meristem/root cap, |
| | | | meristematic and |
| | | | elongation zones |
| 63 | 63 | AT4G31320 | meristem/root cap, |
| | | | meristematic and |
| | | | elongation zones |
| 64 | 64 | AT1G52060 | meristem/root cap, |
| | | | meristematic and |
| | | | elongation zones |
| 65 | 65 | AT4G37160 | meristem/root cap, |
| | | | meristematic and |
| | | | elongation zones |
| 66 | 66 | AT5G60520 | meristem/root cap, |
| | | | meristematic and |
| | | | elongation zones |
| 71 | 71 | AT1G78340 | broad root |
| 72 | 72 | AT5G26260 | broad root |
| 73 | 73 | AT4G13660 | broad root |
| 74 | 74 | AT1G22440 | broad root |
| 75 | 75 | AT2G36100 | broad root |
| 76 | 76 | AT3G11340 (+intron) | broad root |
| 77 | 77 | AT1G02810 (+intron) | broad root |
| 78 | 78 | AT5G64100 (+intron) | broad root |
| 79 | 79 | AT5G59090 (+intron) | broad root |
| 80 | 80 | AT2G43610 (+intron) | broad root |
| 81 | 81 | AT5G44380 (+intron) | broad root |
| 82 | 82 | AT2G18210 | broad root |
| 190 | 95 | AT3G11340 | broad root |
| 191 | 96 | AT1G02810 | broad root |
| 192 | 97 | AT5G64100 | broad root |
| 193 | 98 | AT5G59090 | broad root |
| 194 | 99 | AT2G43610 | broad root |
| 195 | 100 | AT5G44380 | broad root |

Where annotated, the nucleic acid sequences provided in FIGS. 1-16, 32-66, 71-82 and 190-195 indicate one transcription start site (Capital letter in bold), any endogenous 5′-UTR intron sequences (double underlining), any first intron added from the coding sequence (single underlining), and any added intron splice sequences (bold italics), although not all figures may be annotated. All Arabidopsis genome sequences and annotations (i.e. transcription start sites, translation start sites, and introns) are from The Arabidopsis Information Resource (TAIR, available on the worldwide web at the address Arabidopsis.org/index.jsp).

### Example 2

### Endogenous Expression of Candidate Arabidopsis Genes

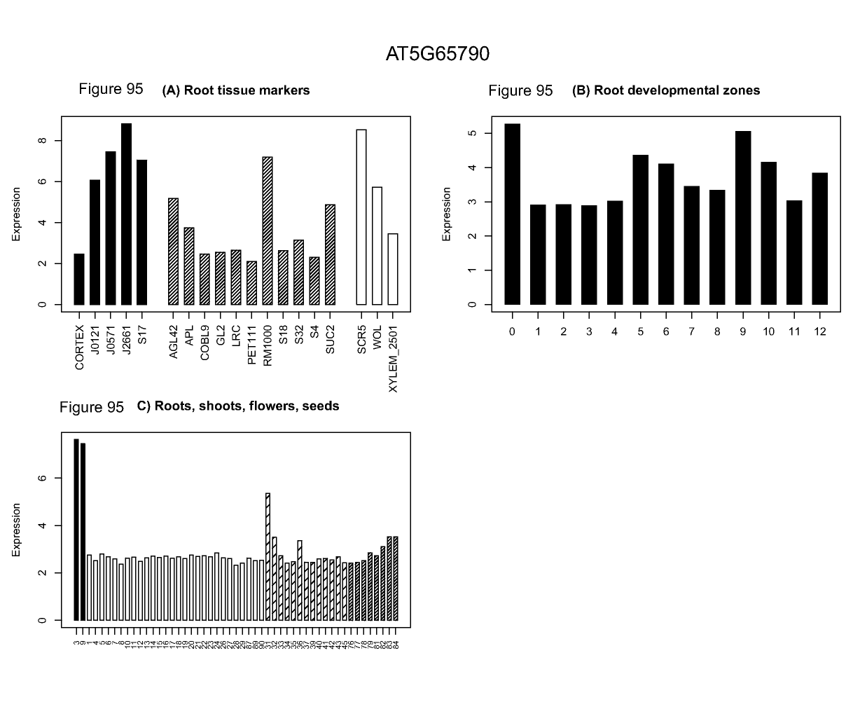

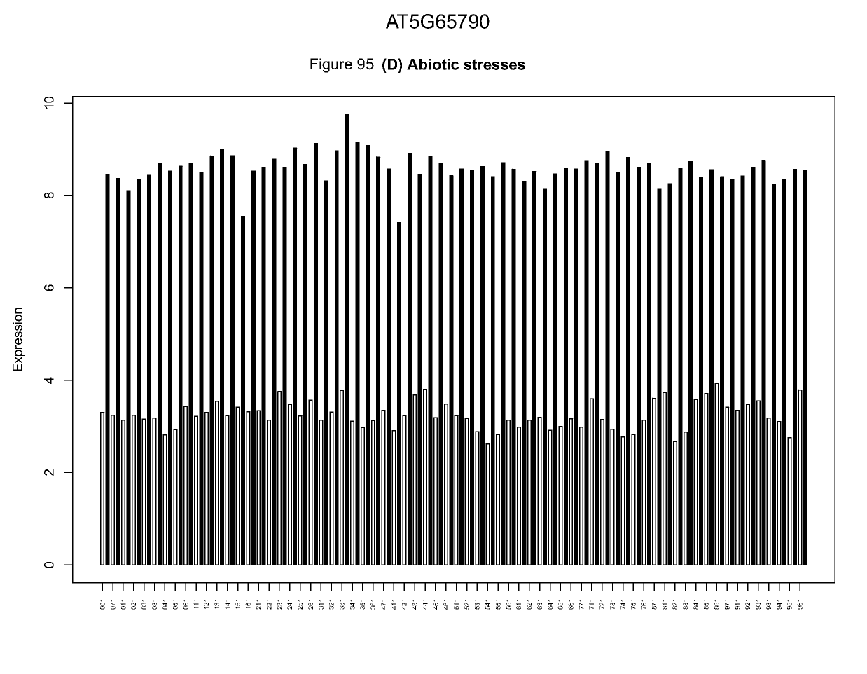

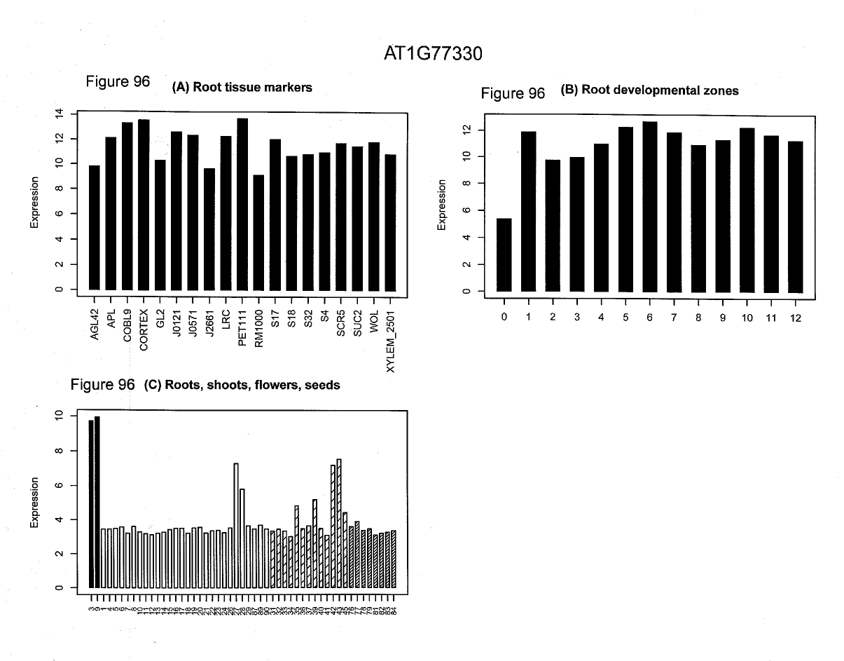

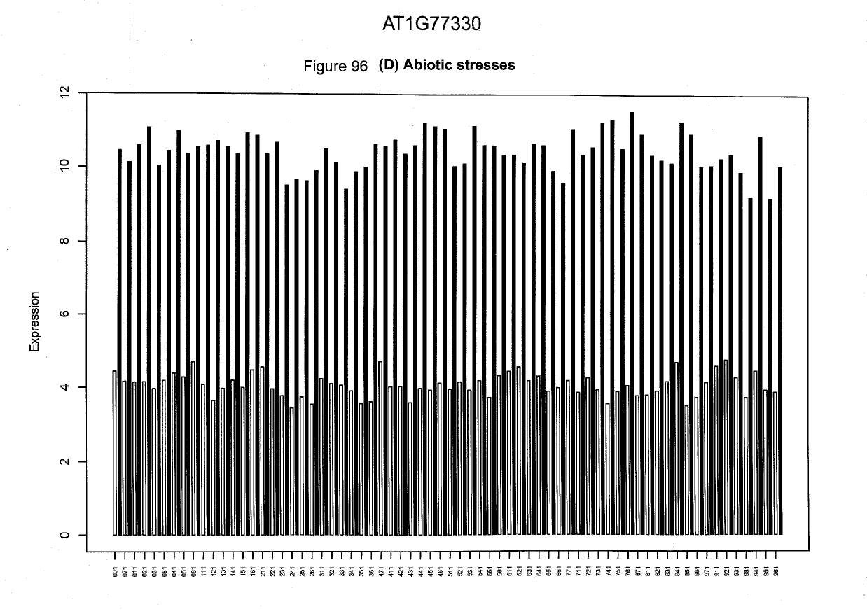

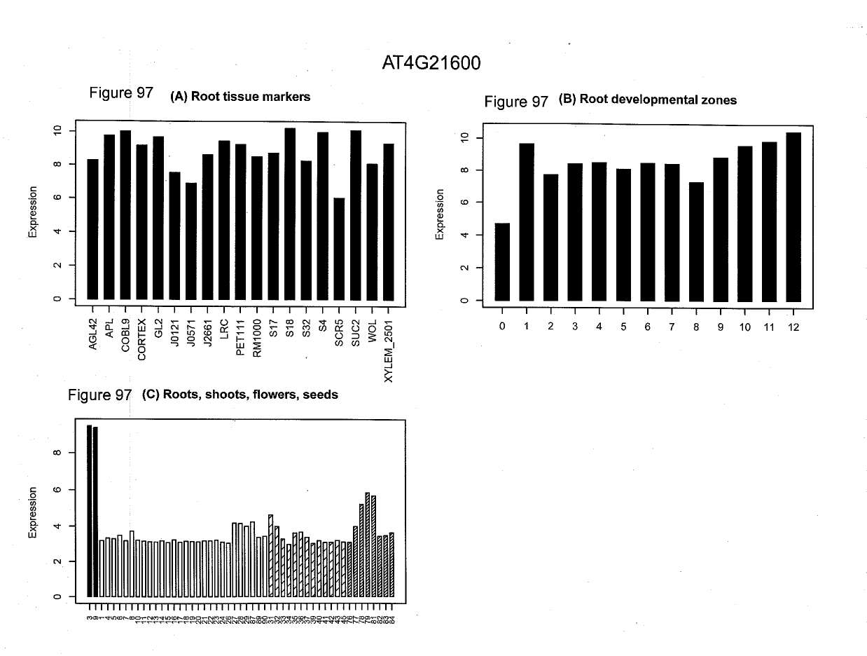

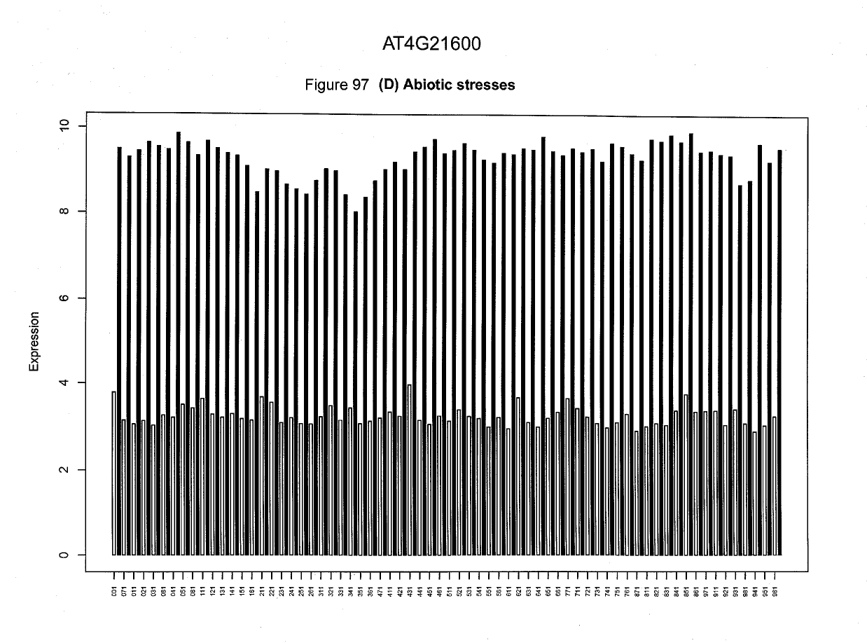

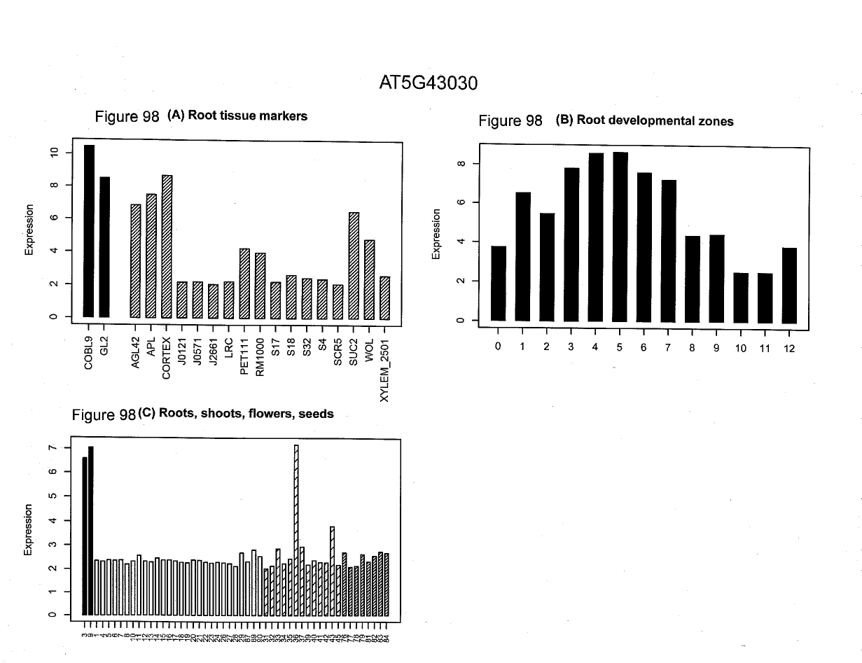

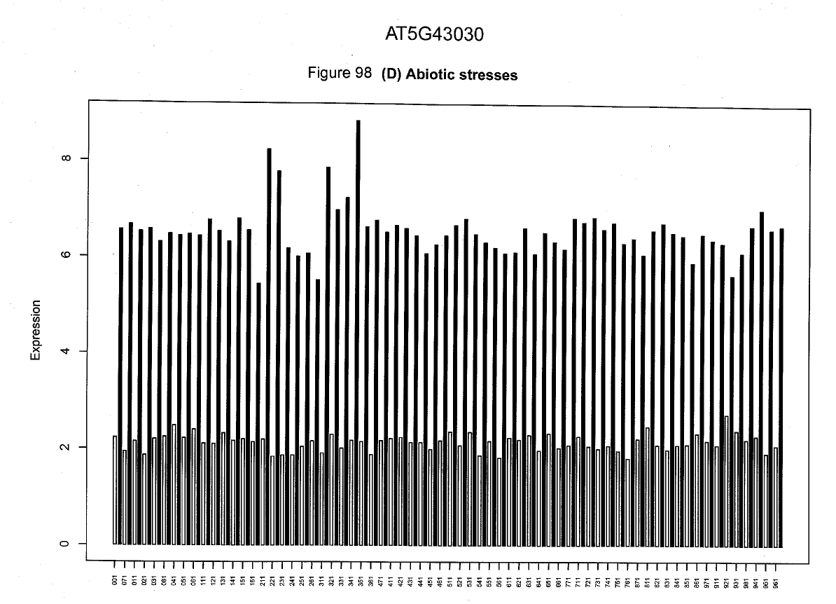

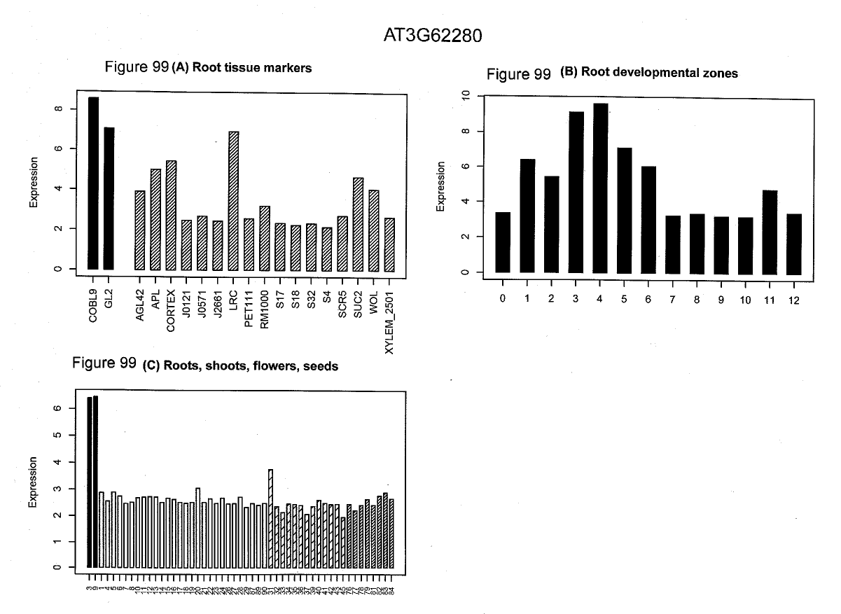

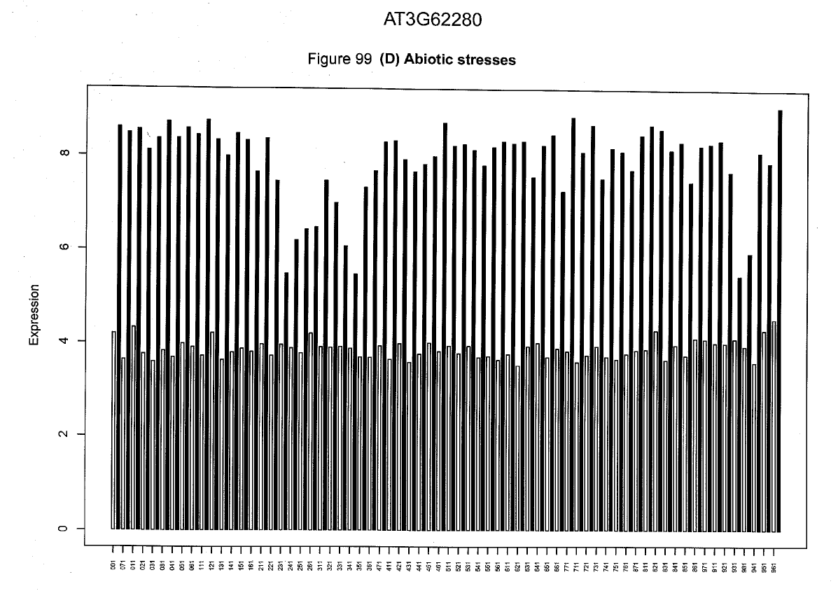

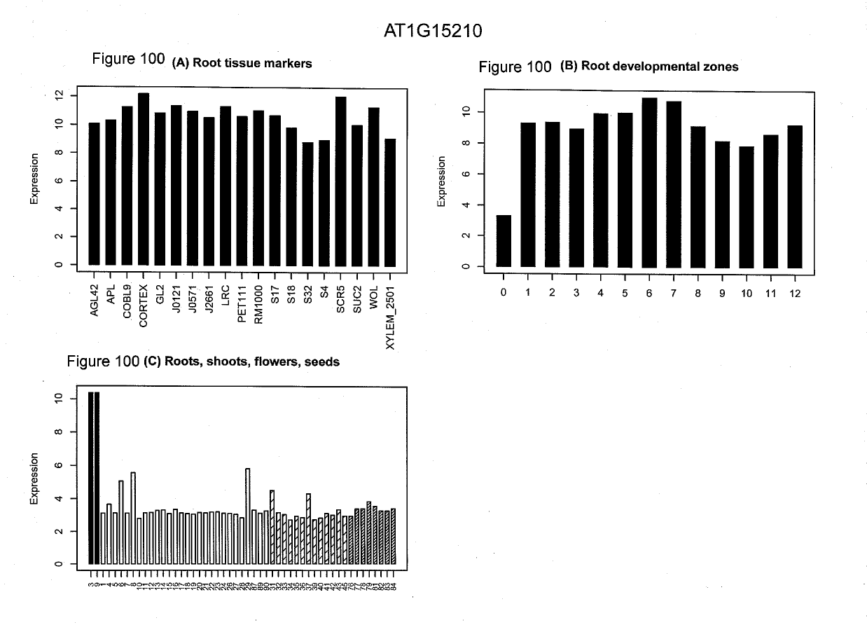

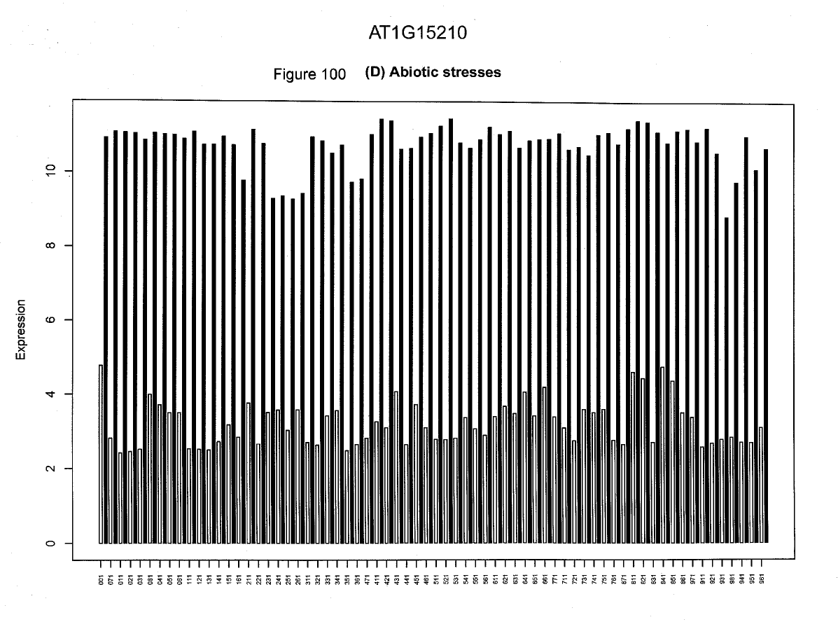

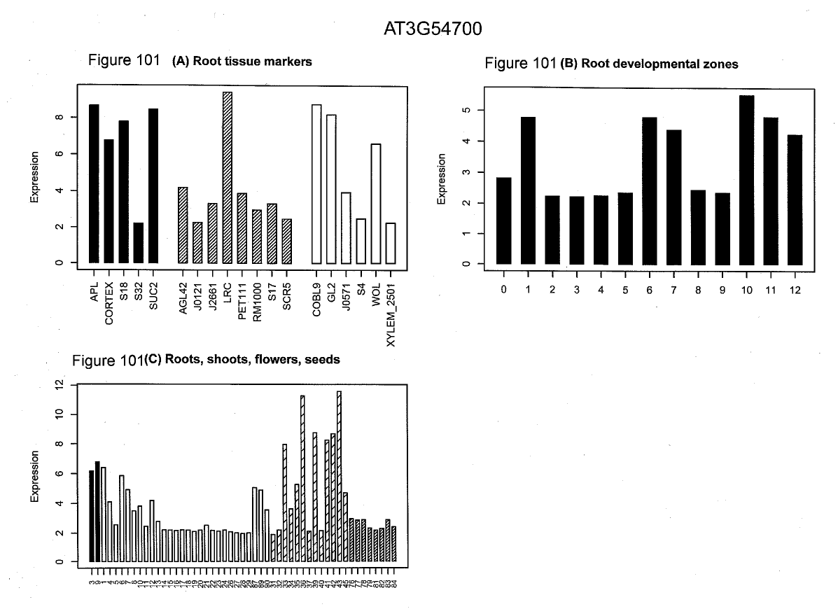

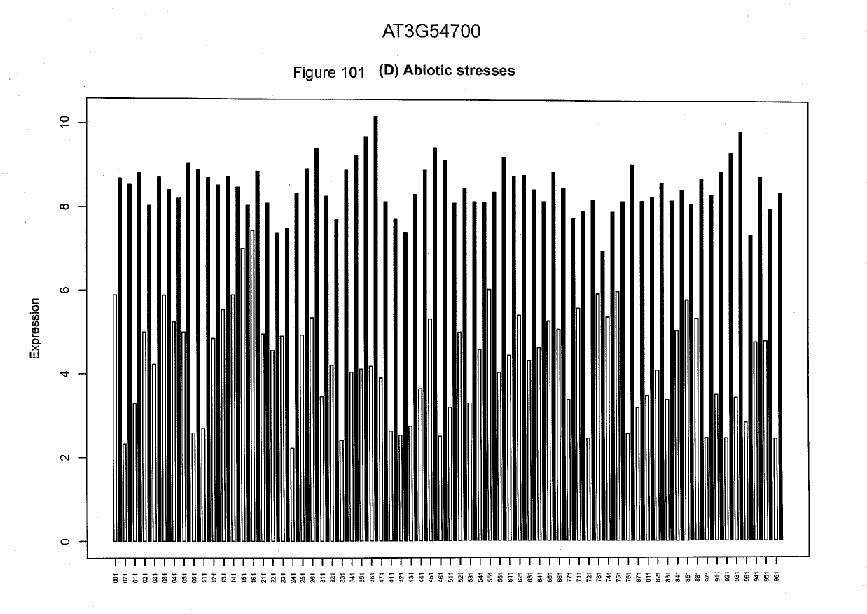

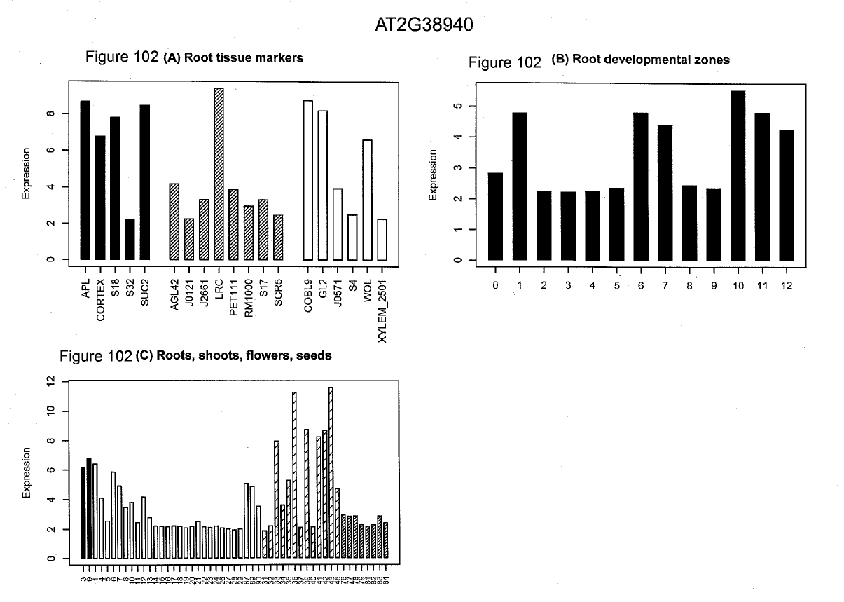

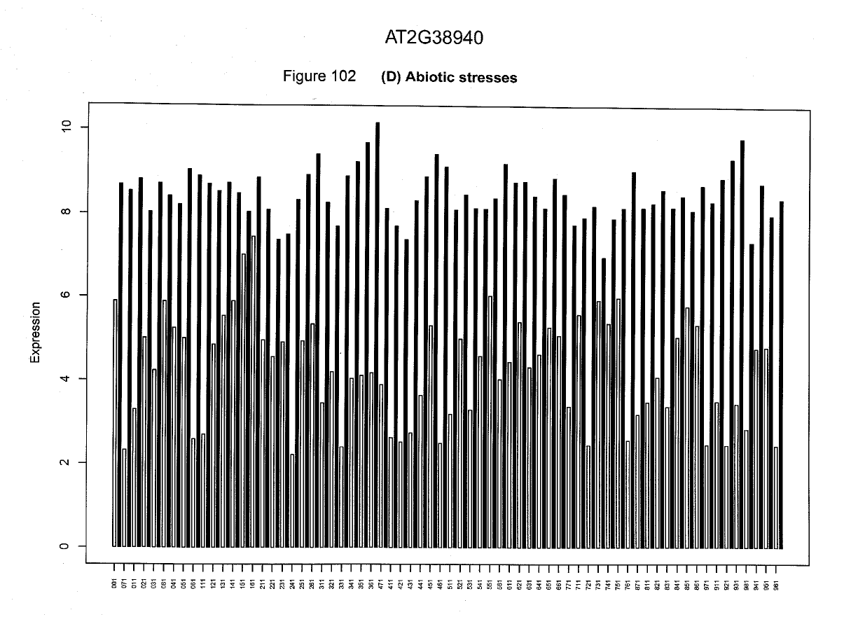

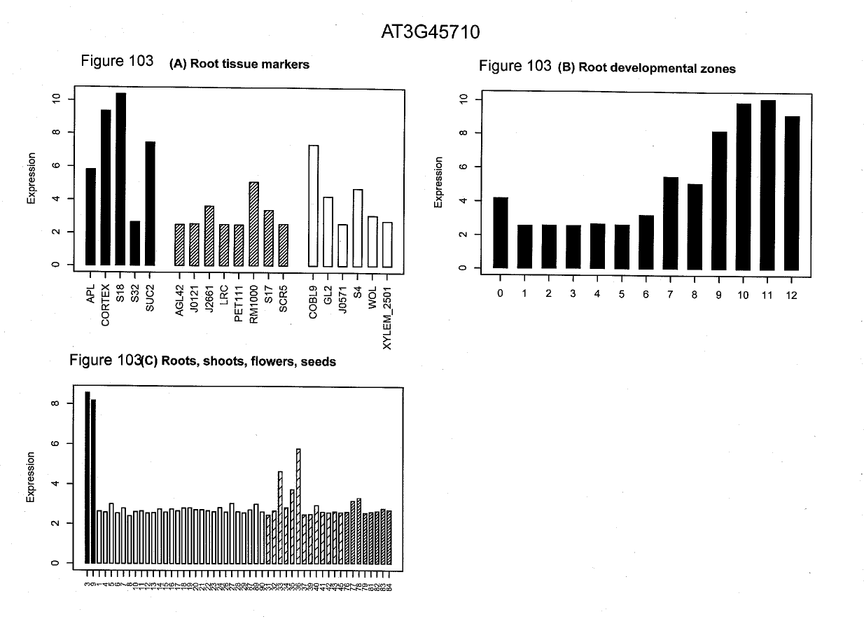

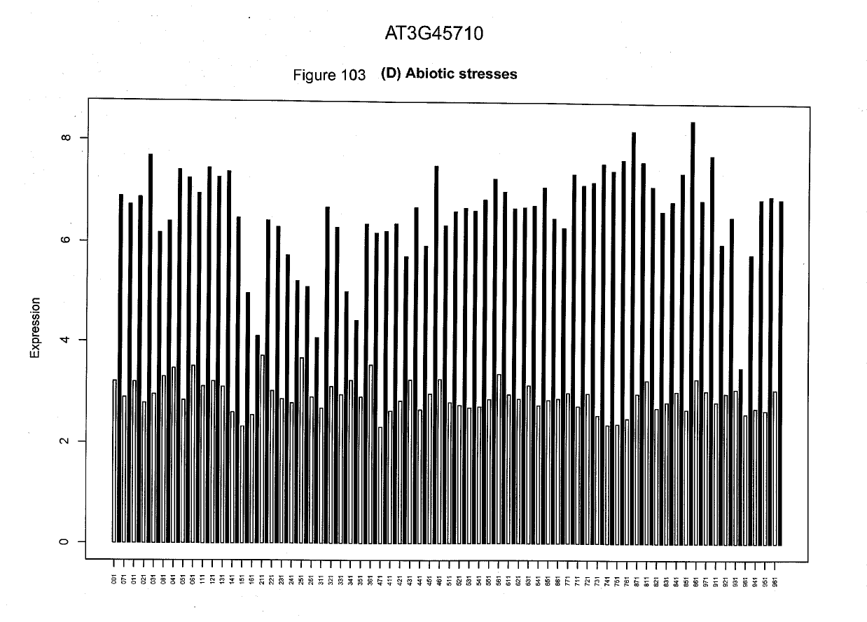

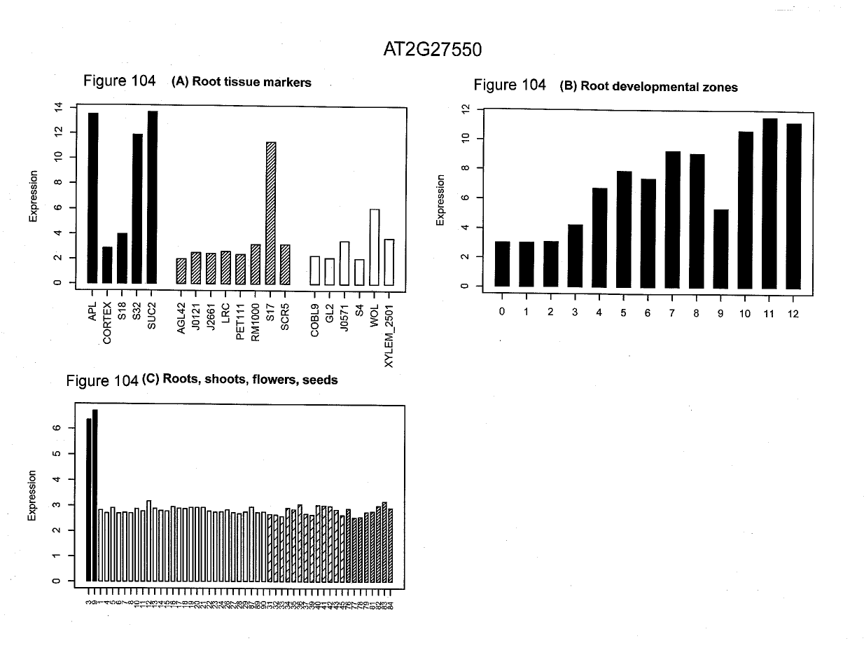

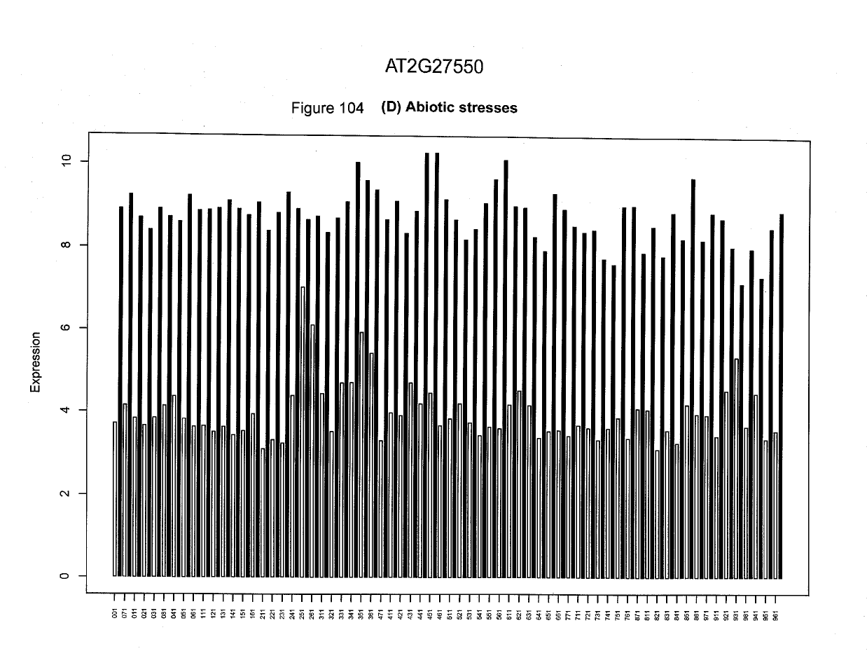

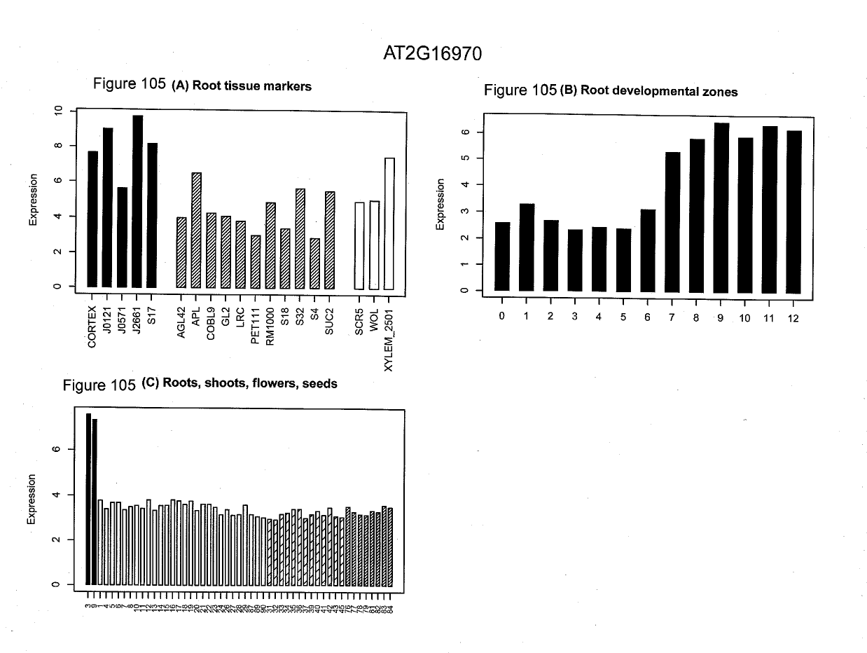

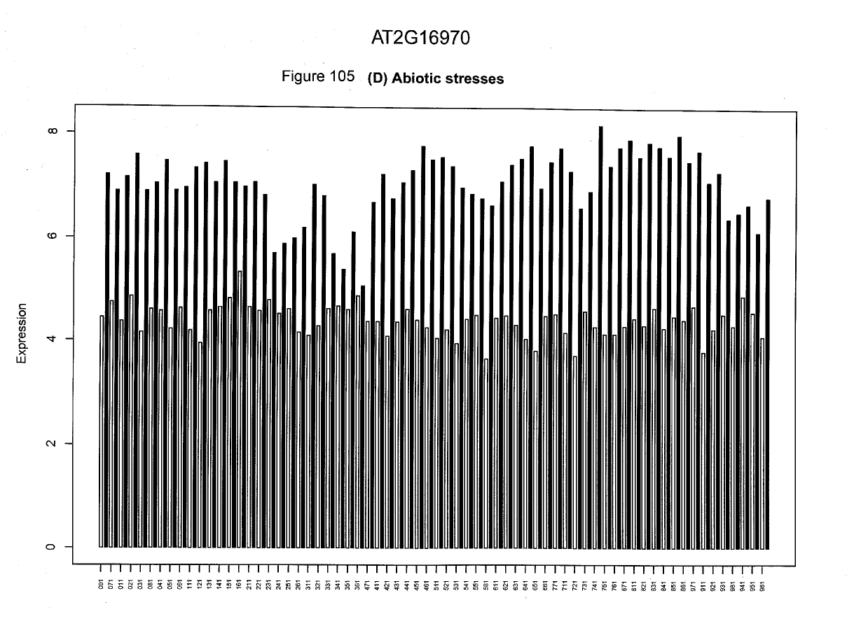

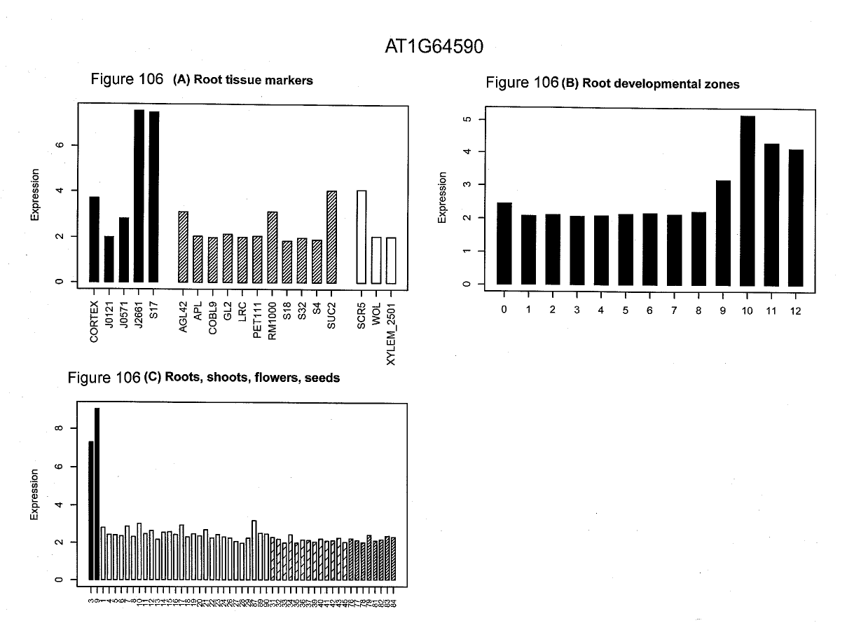

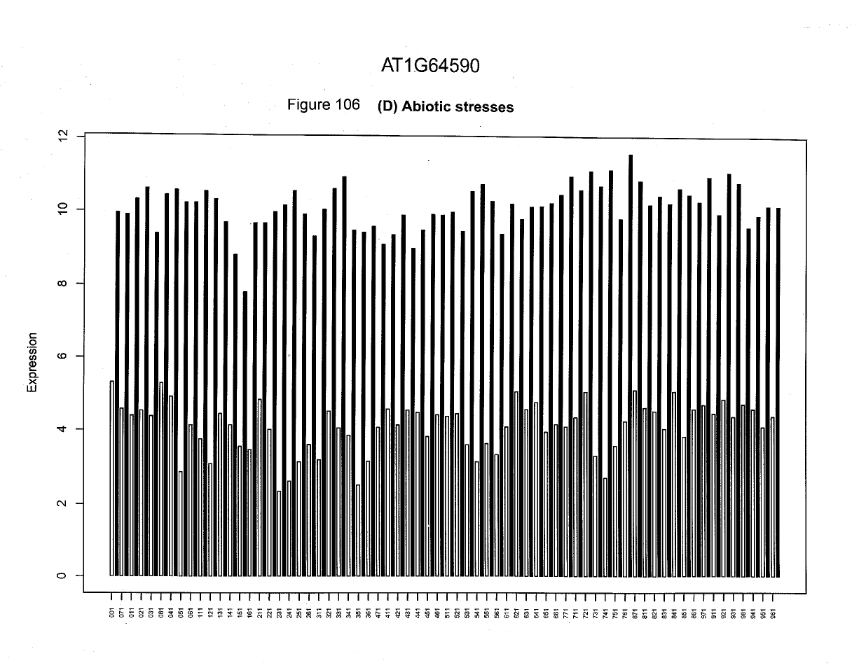

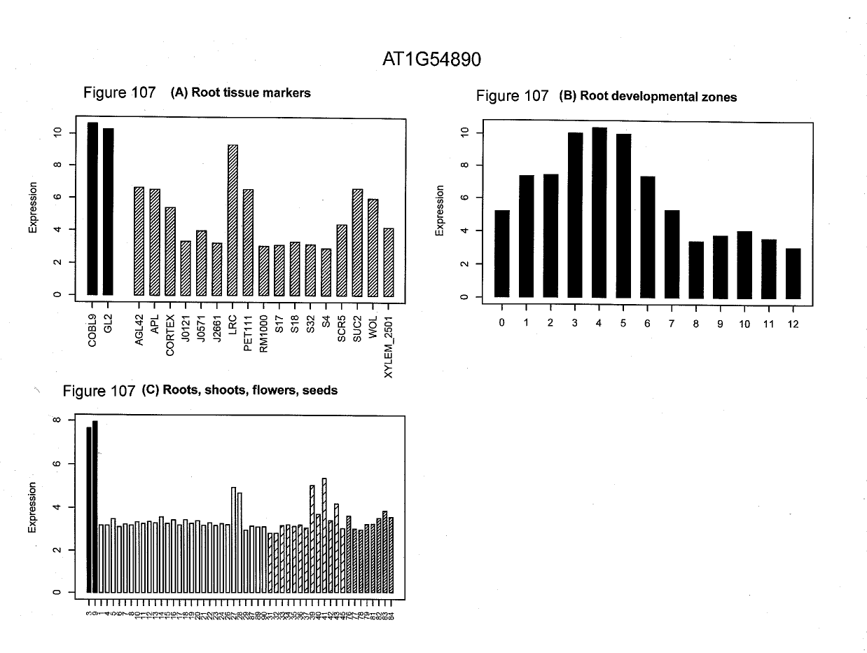

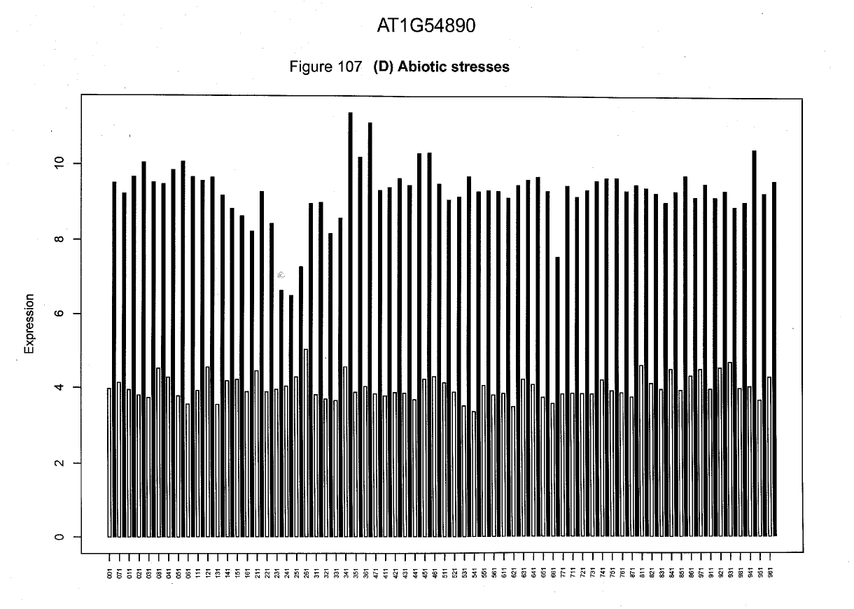

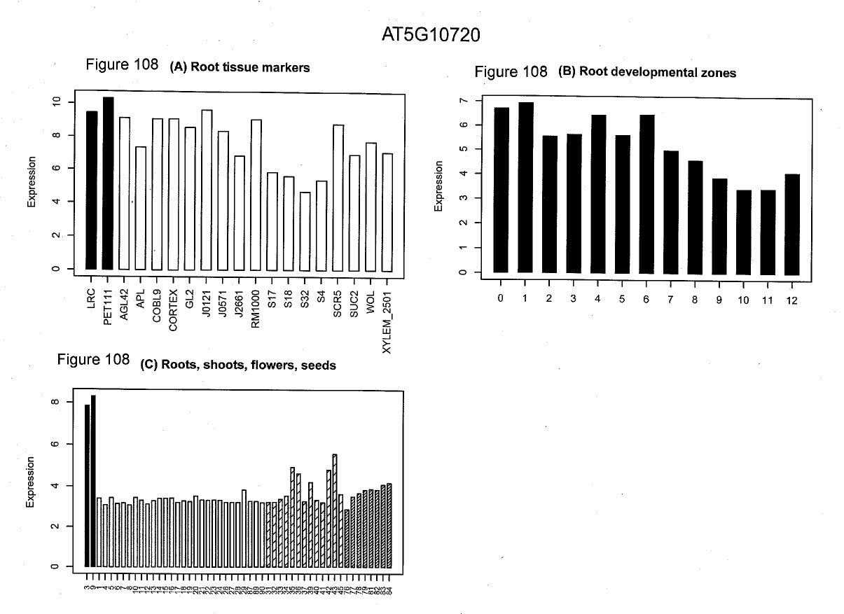

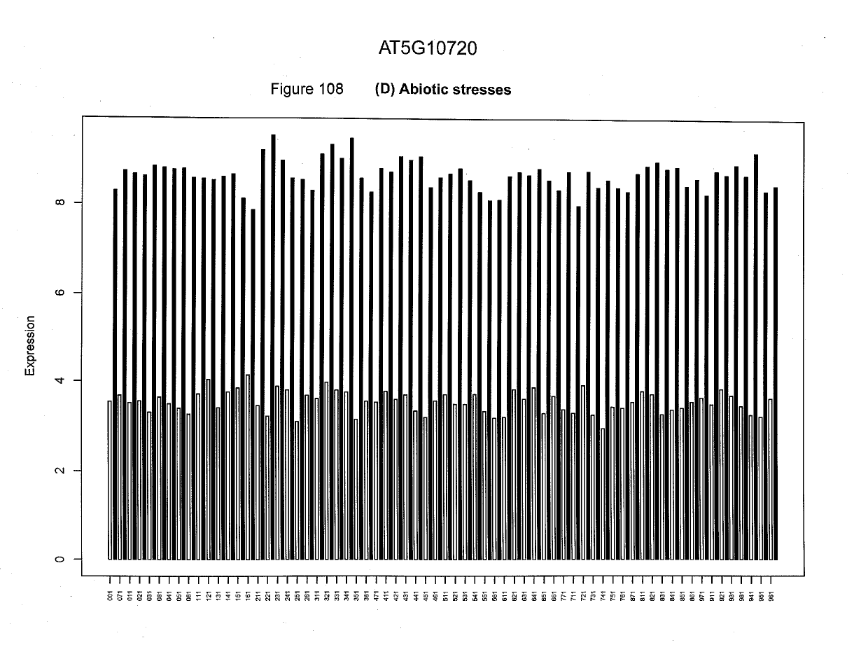

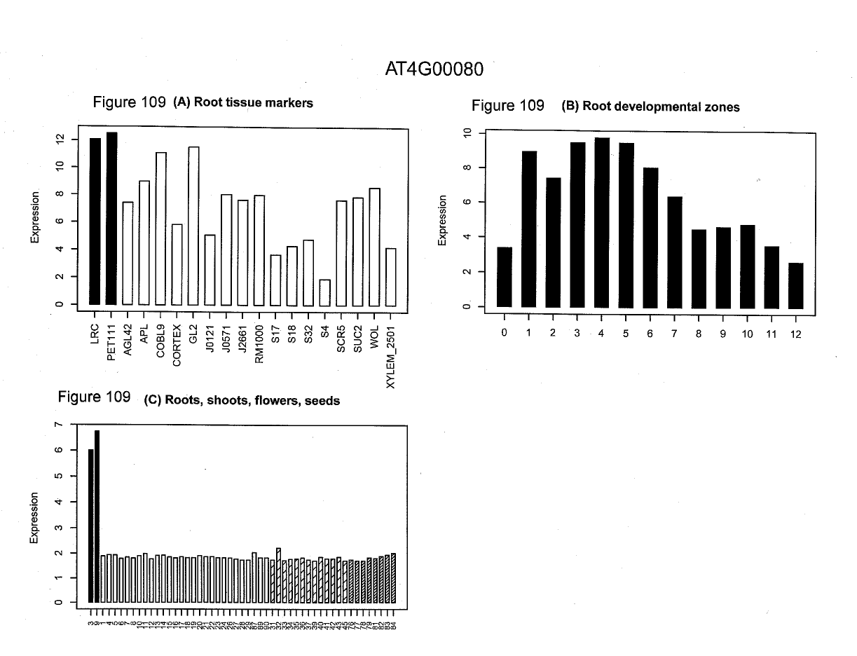

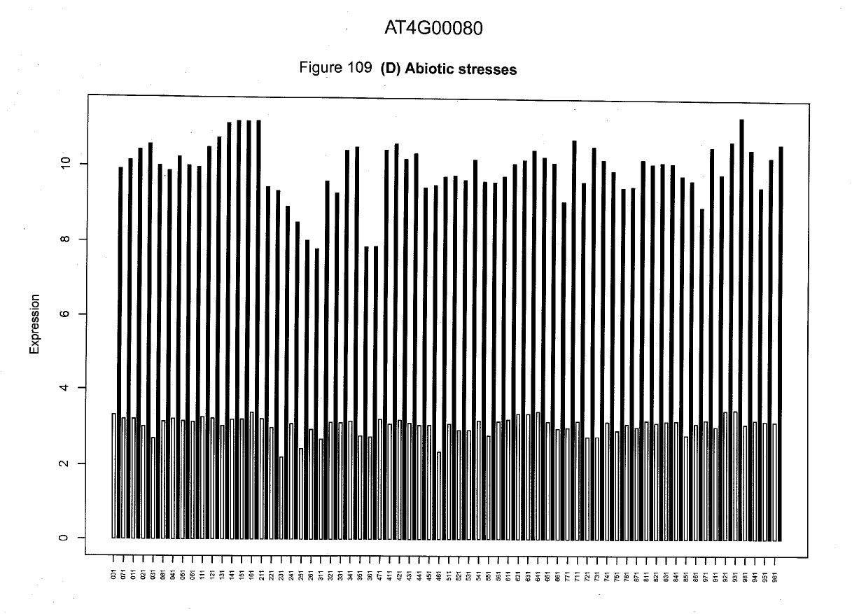

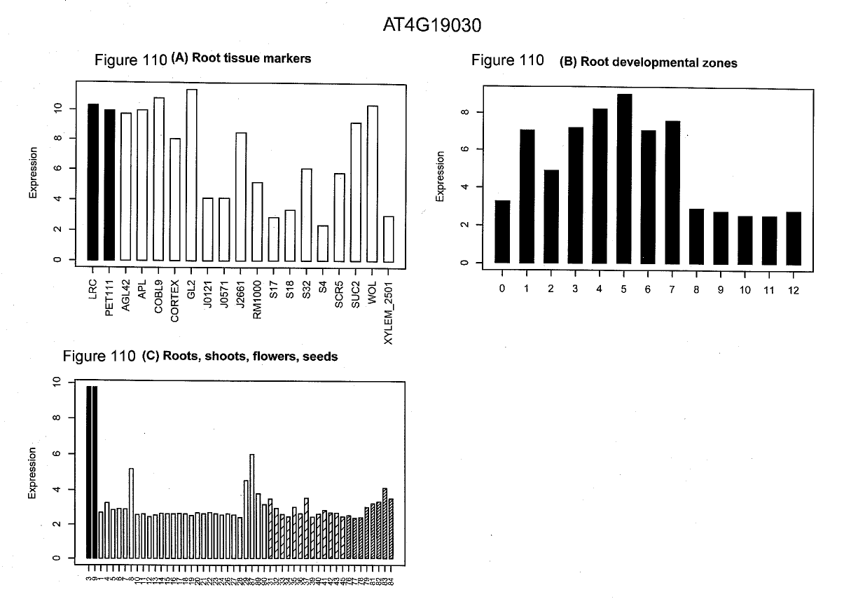

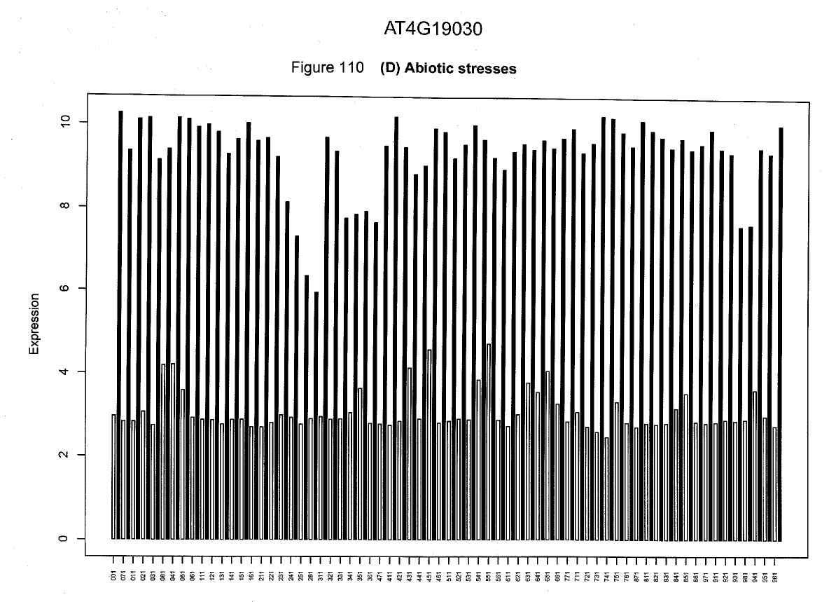

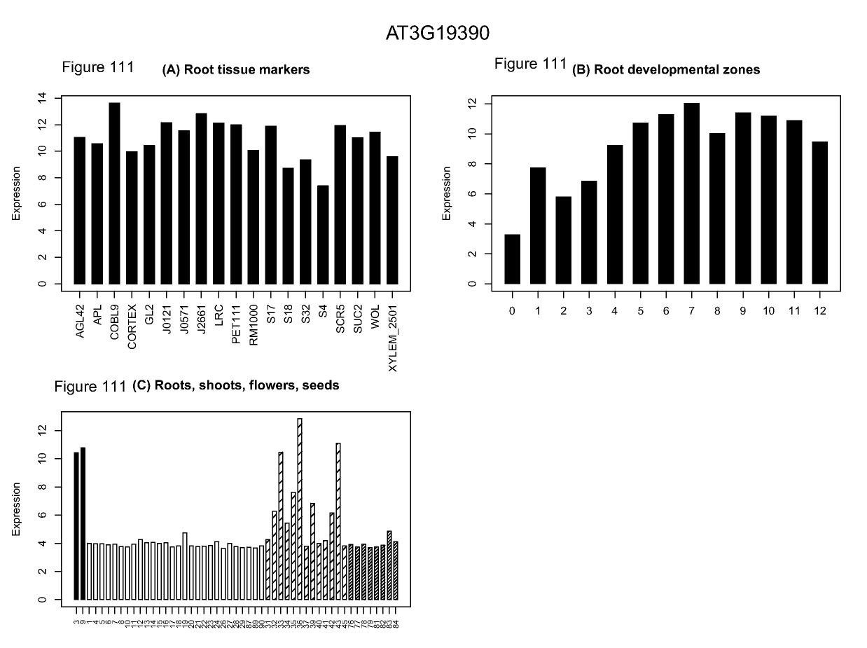

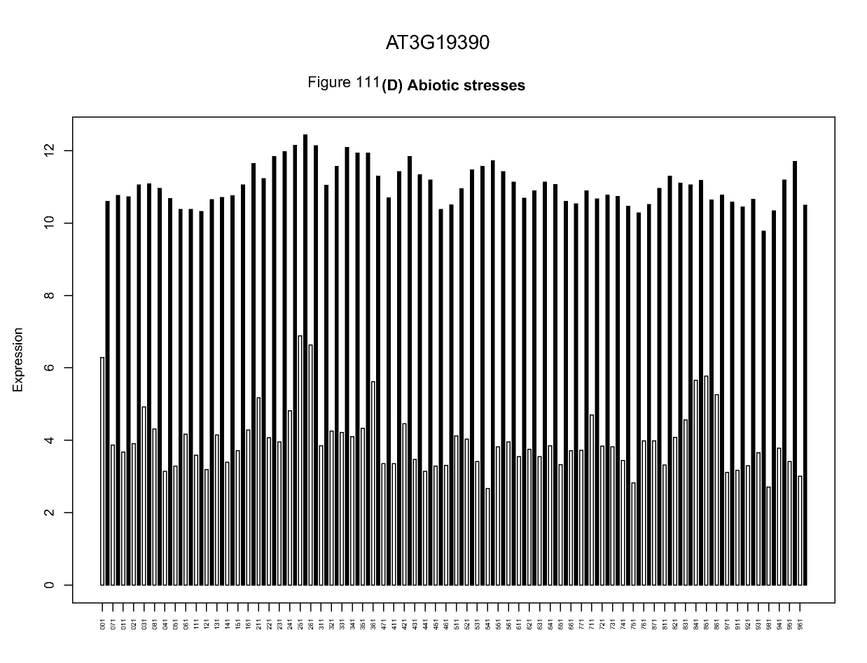

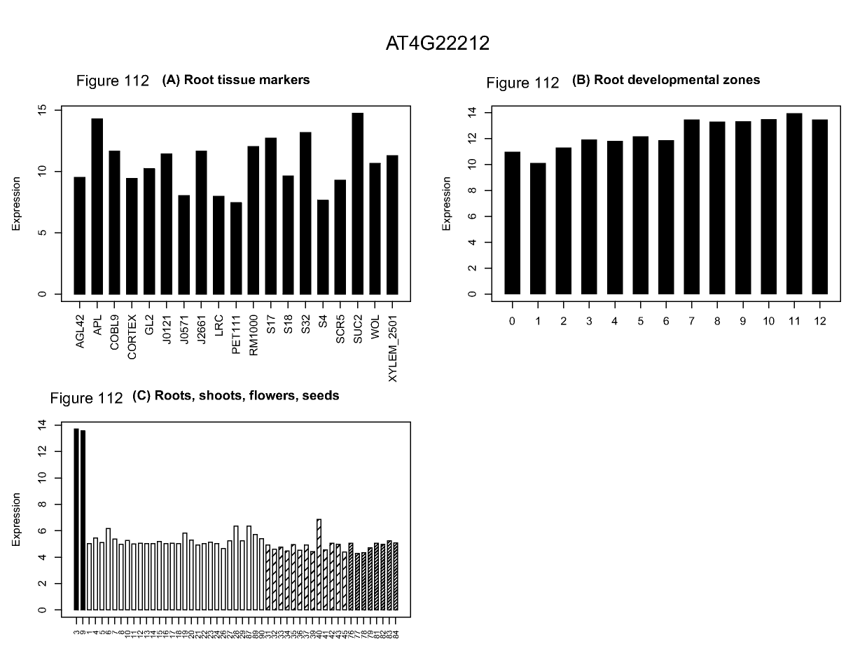

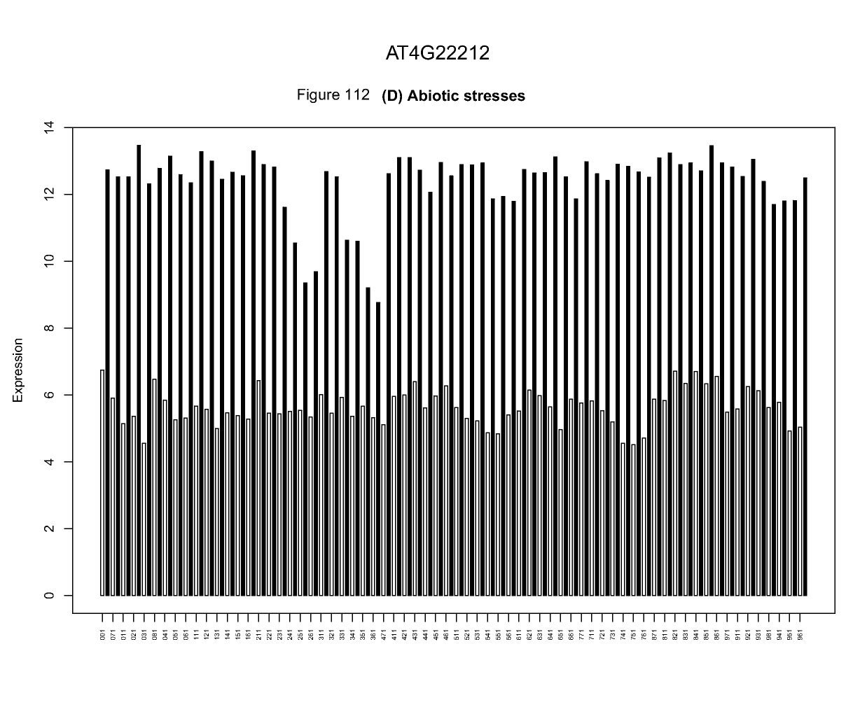

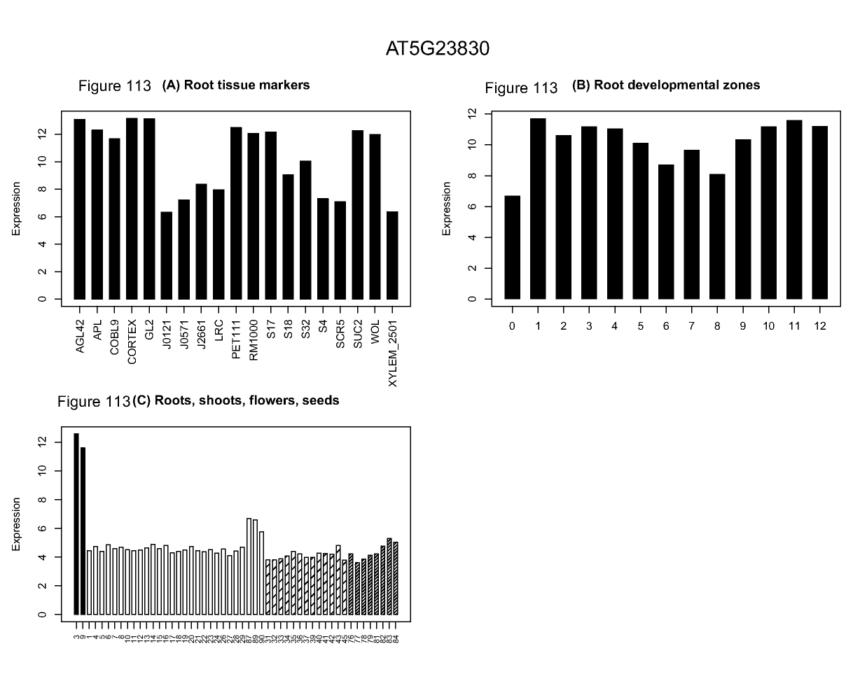

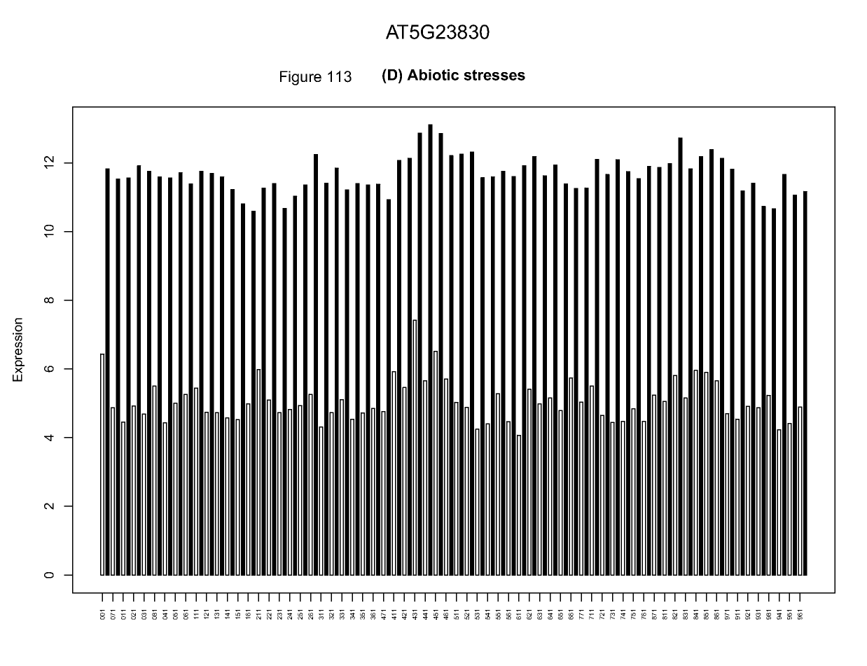

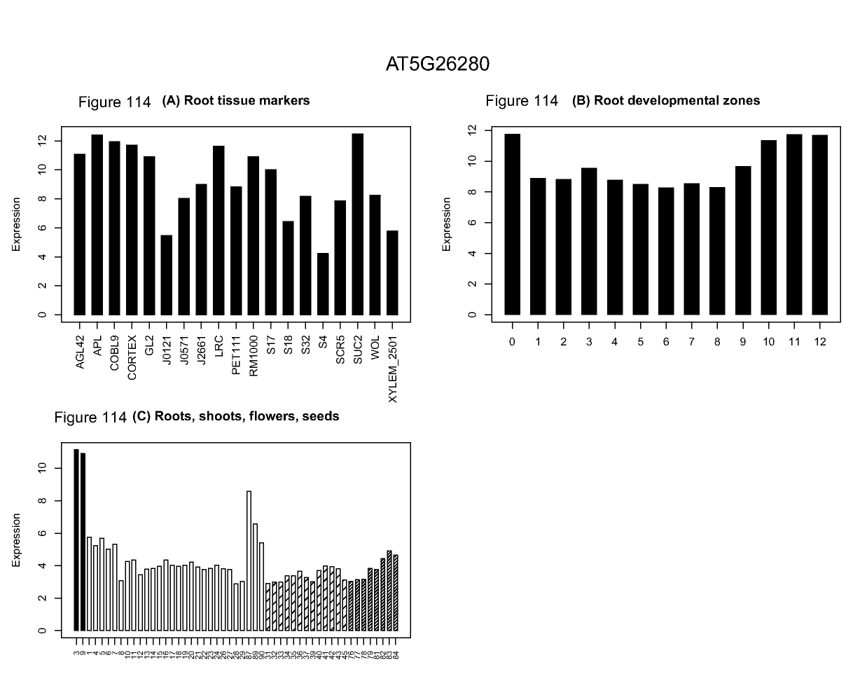

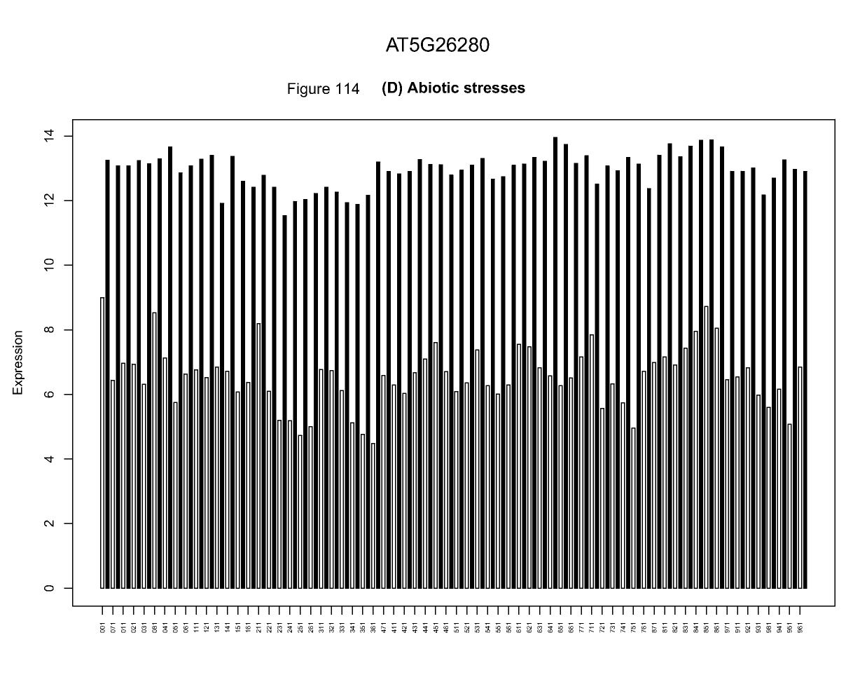

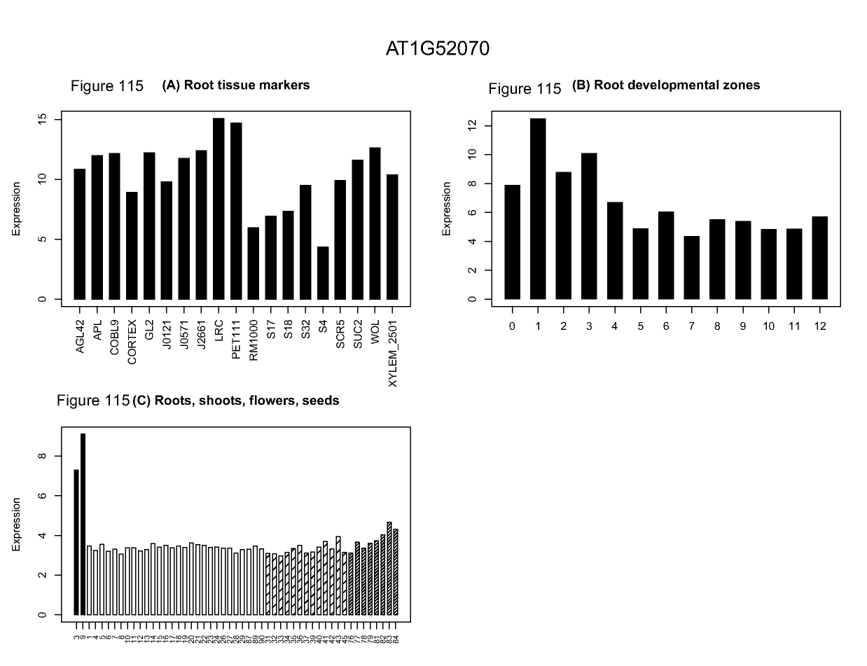

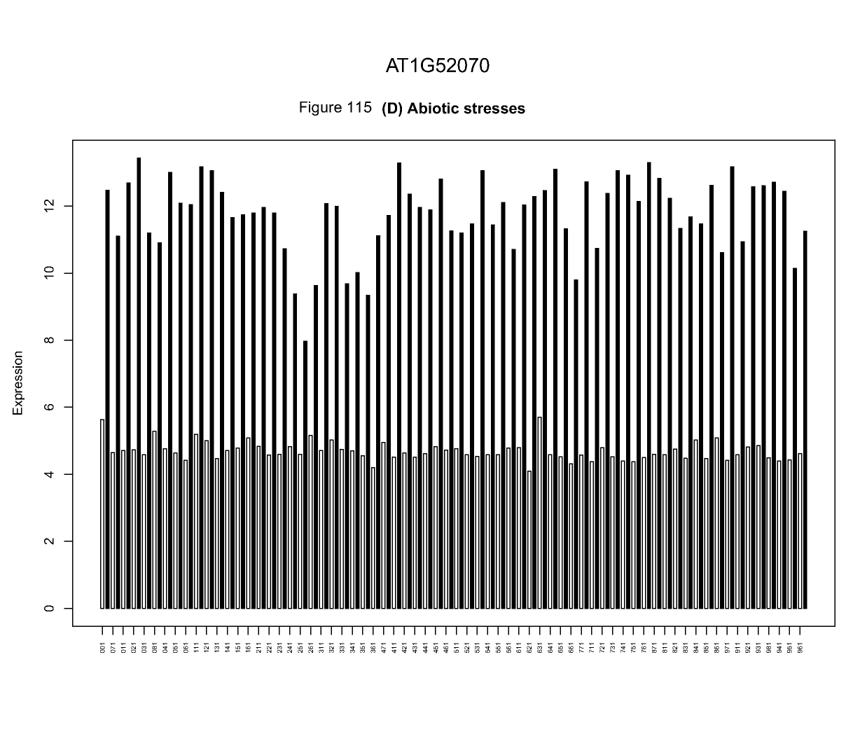

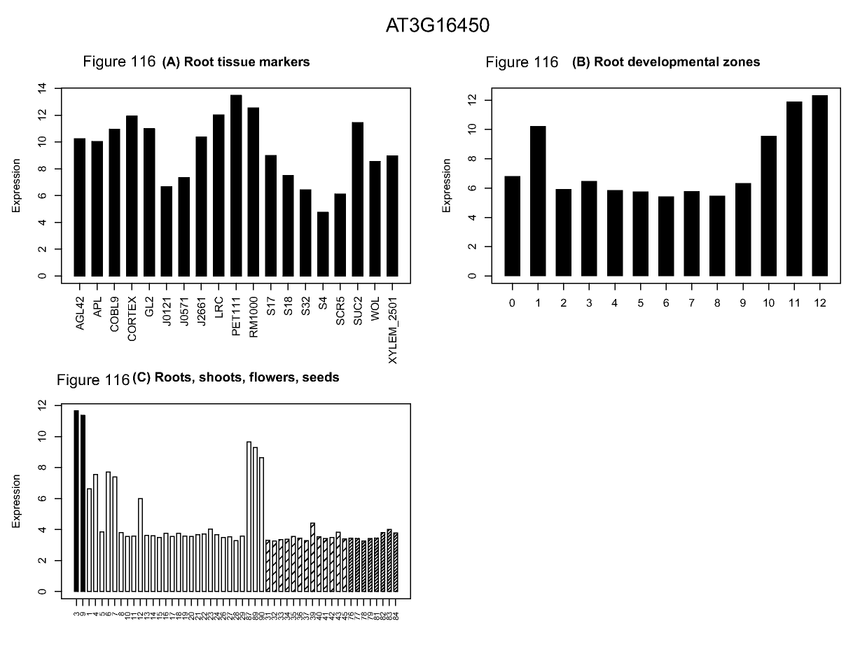

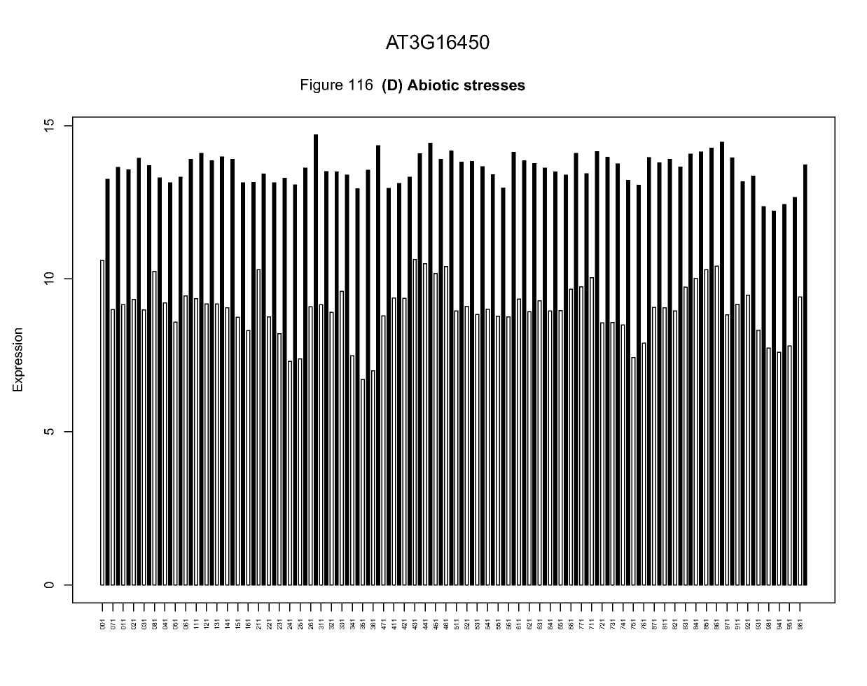

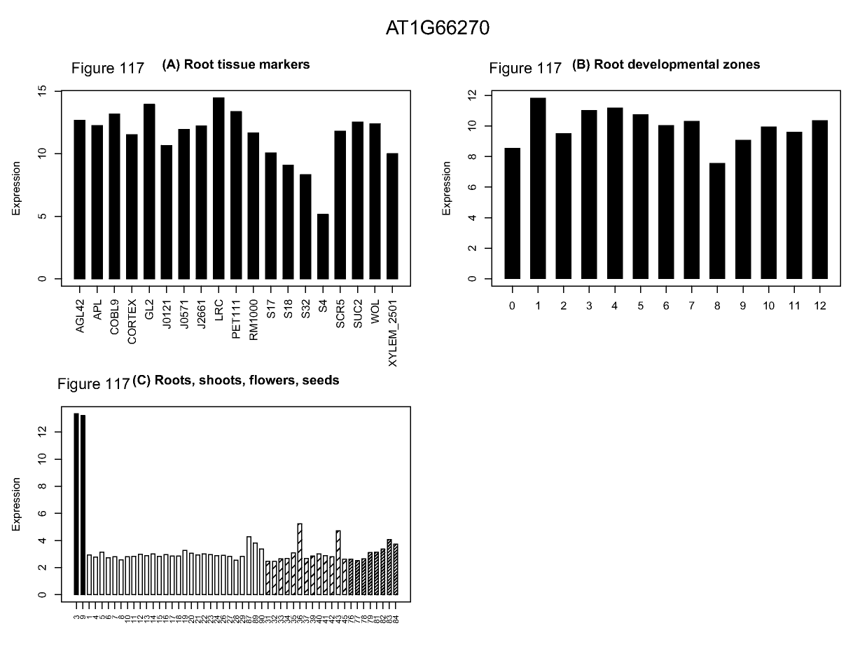

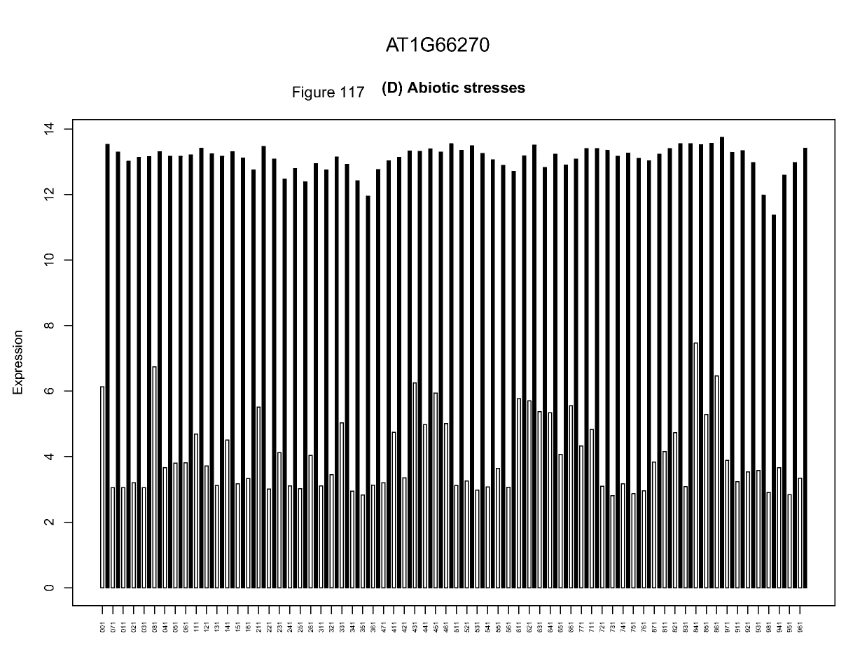

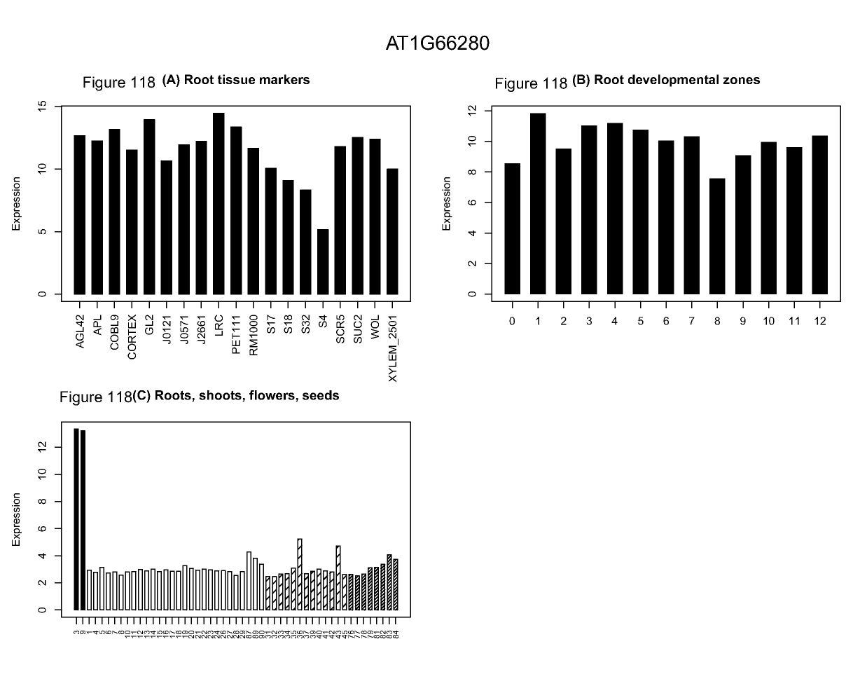

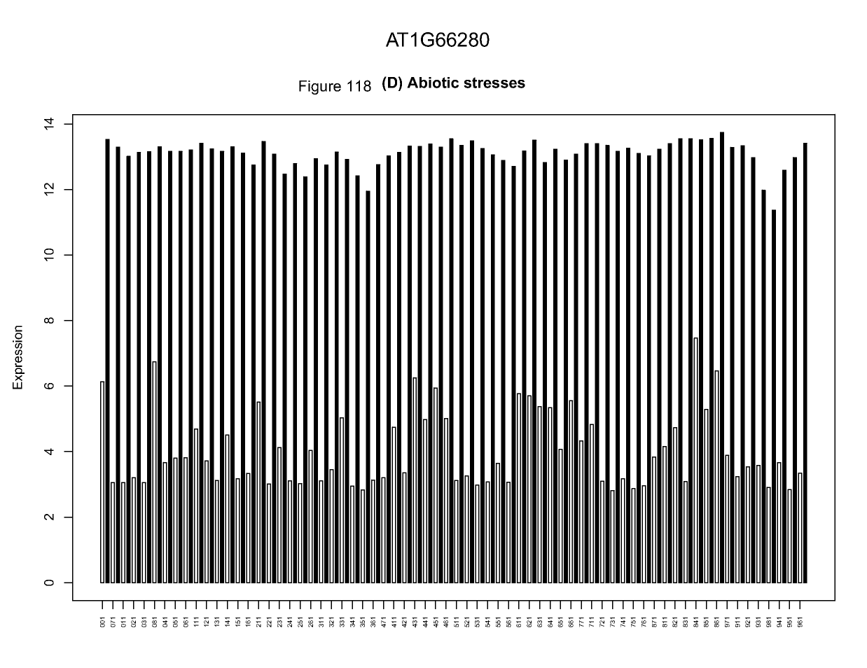

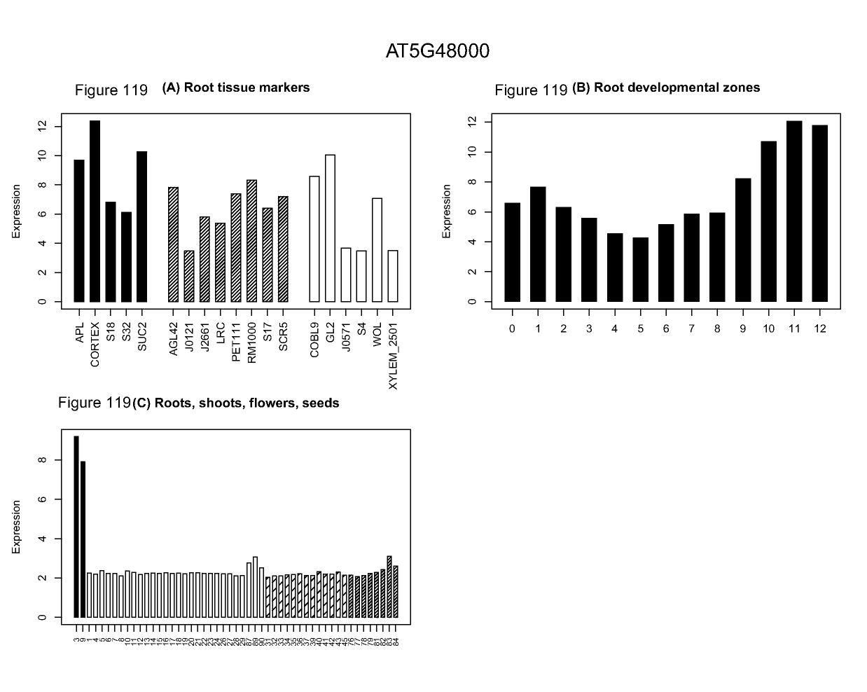

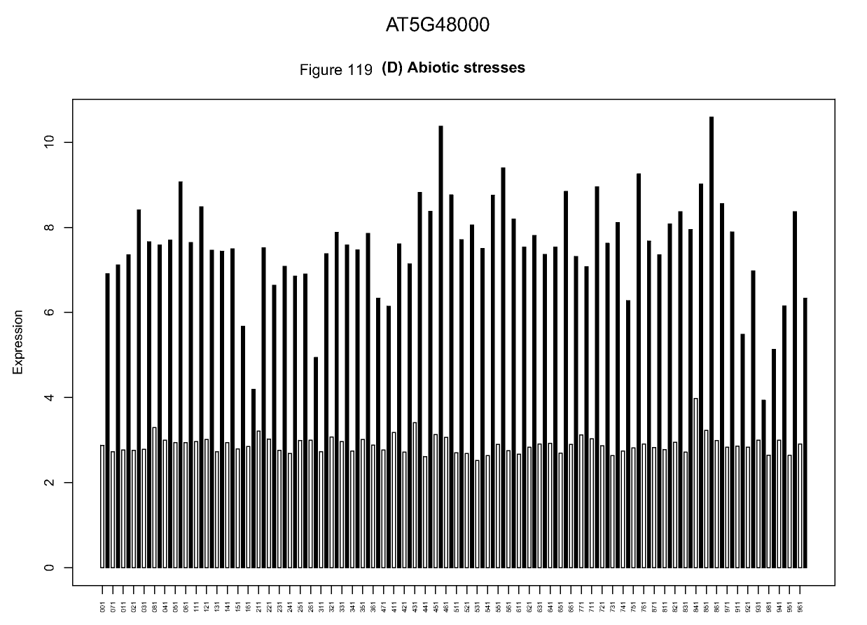

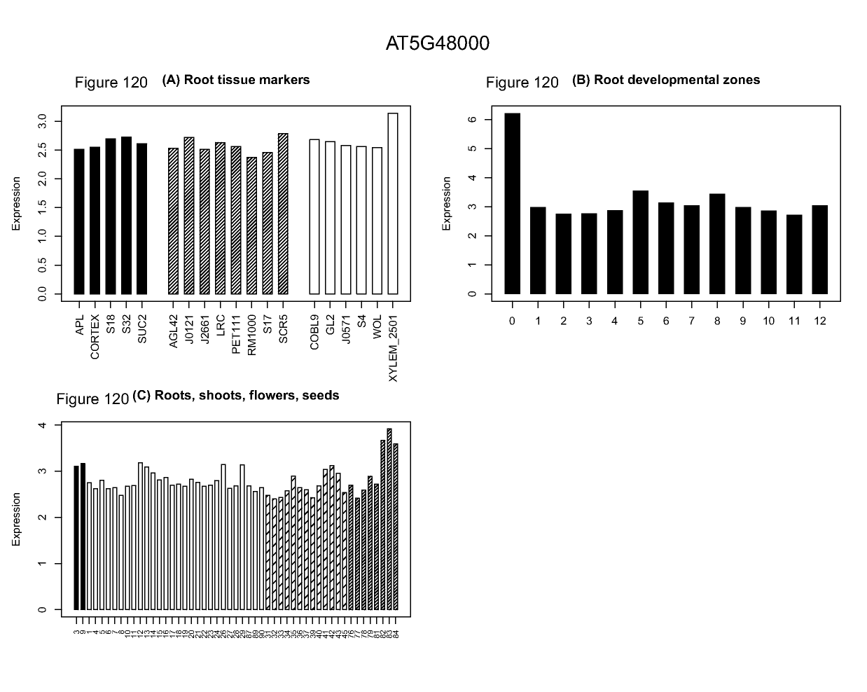

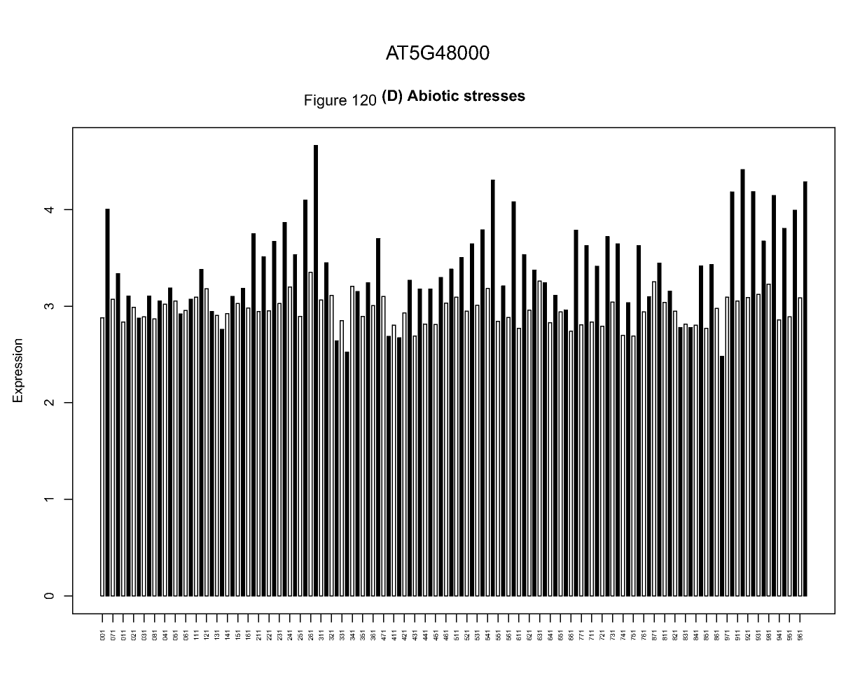

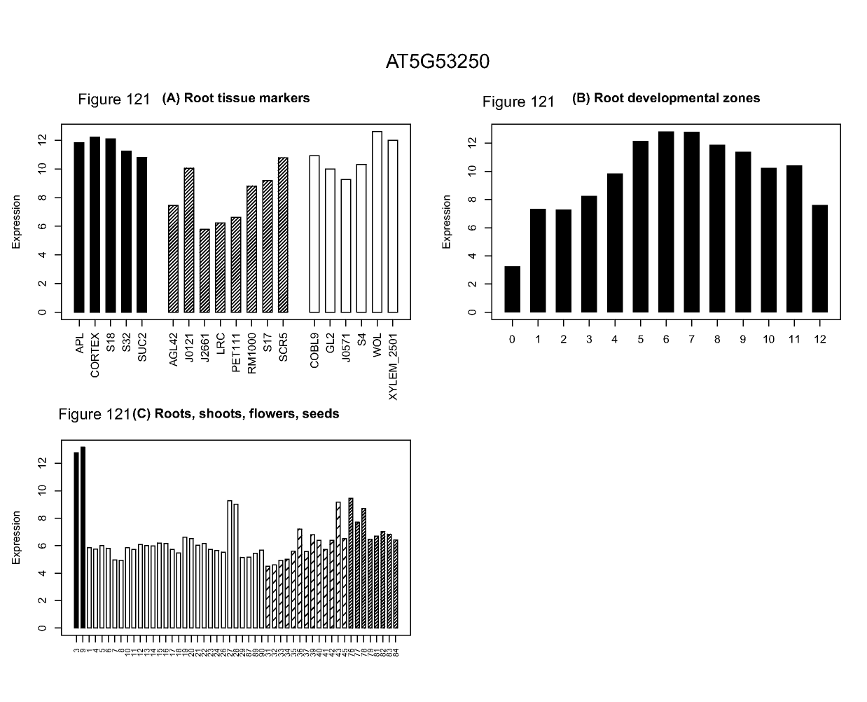

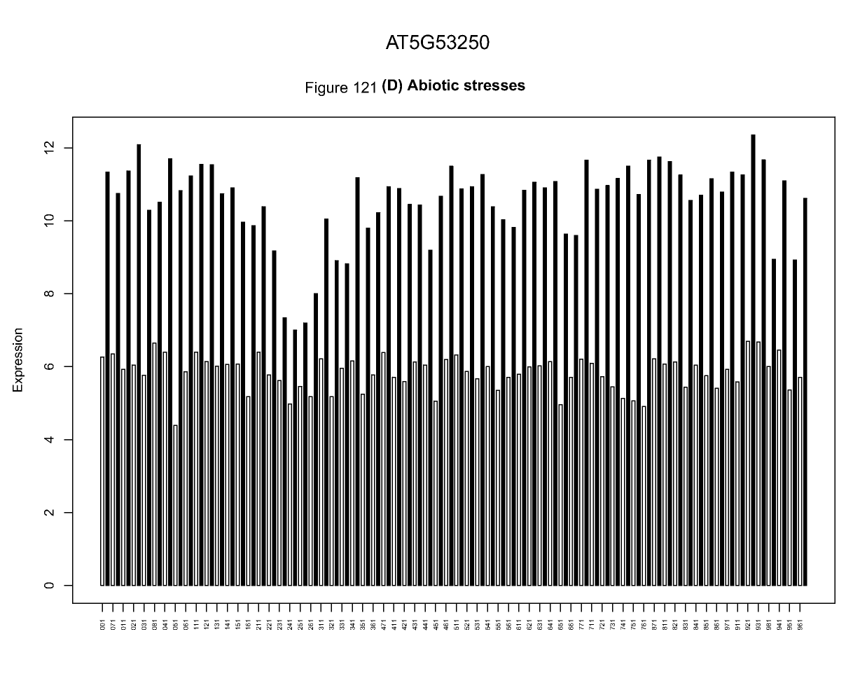

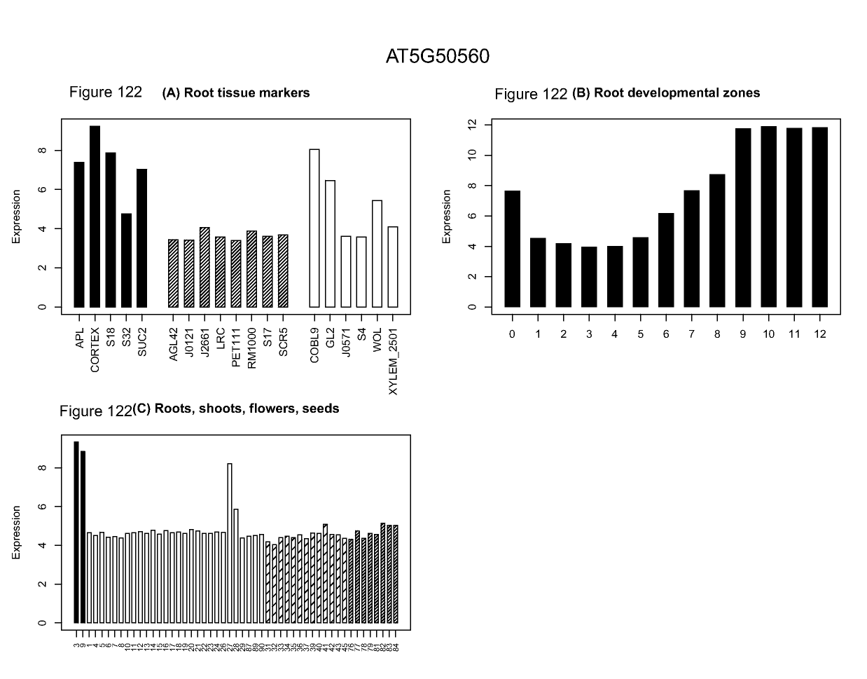

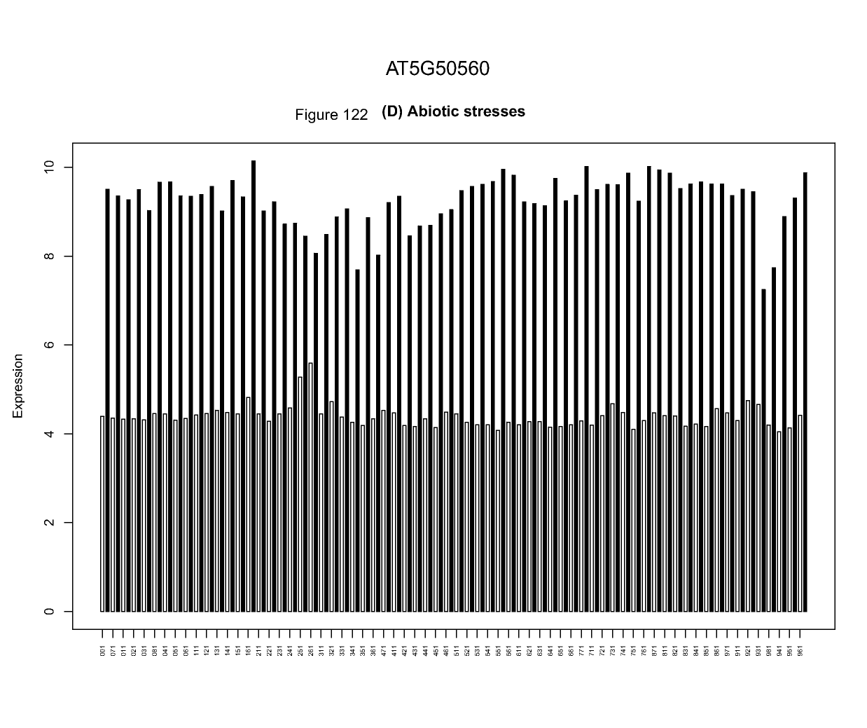

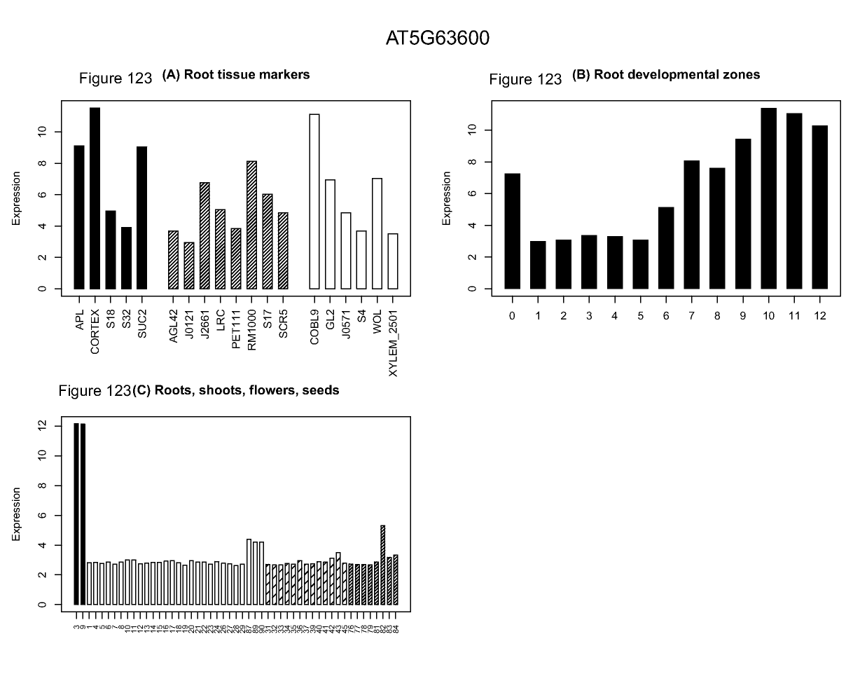

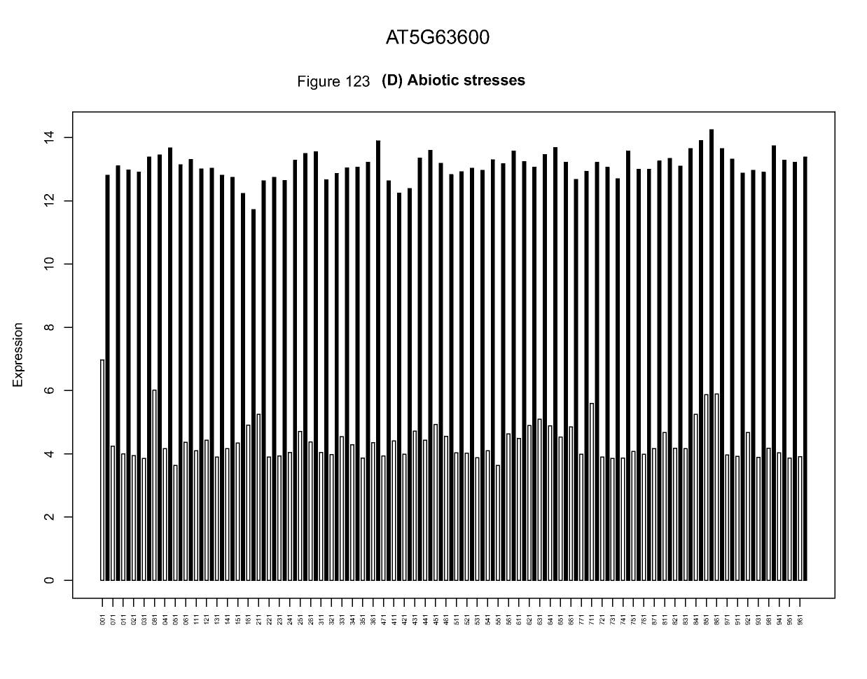

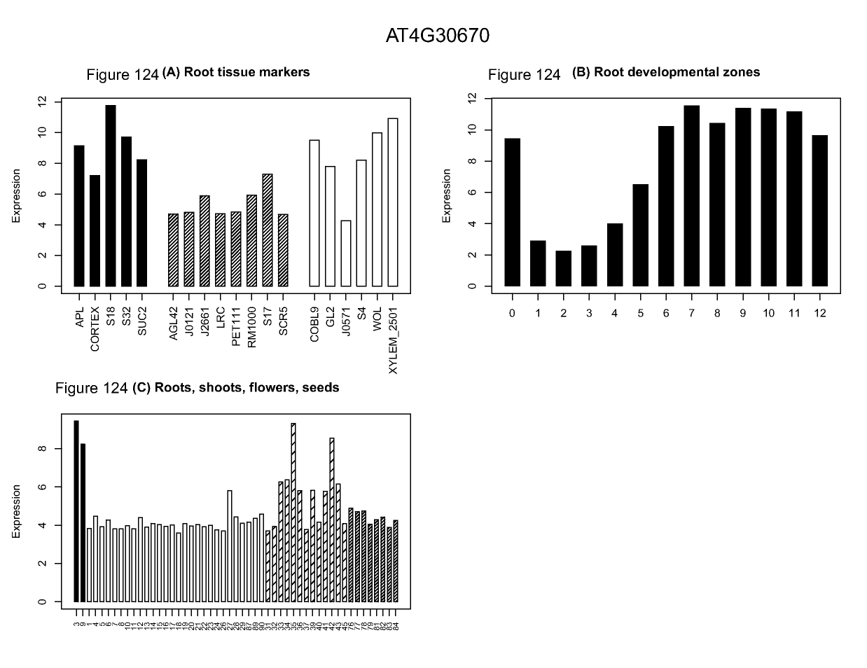

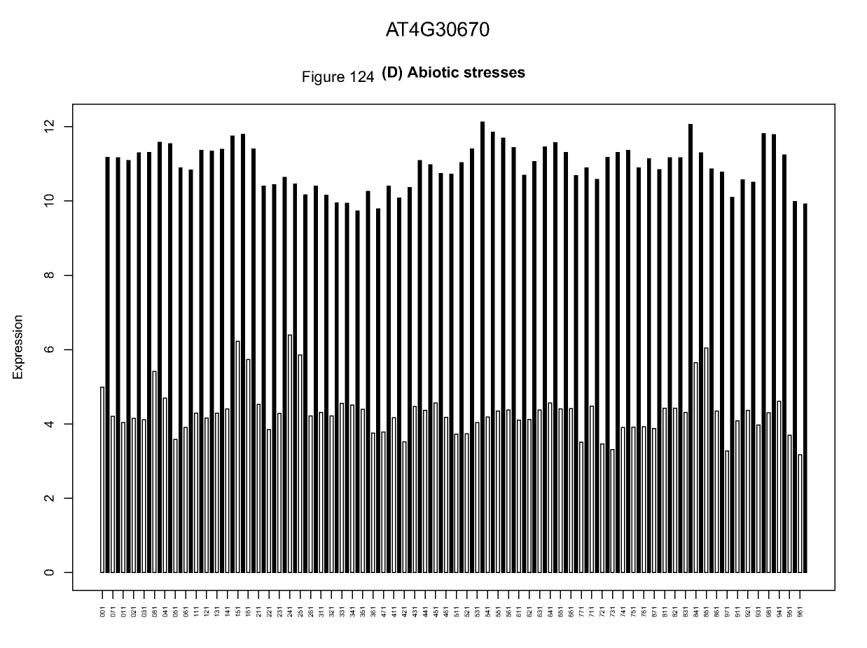

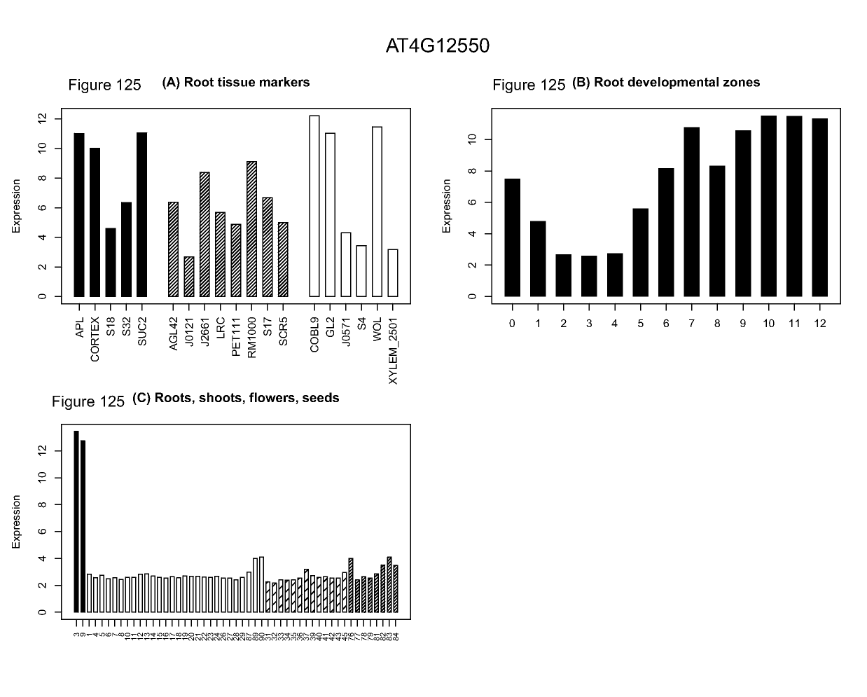

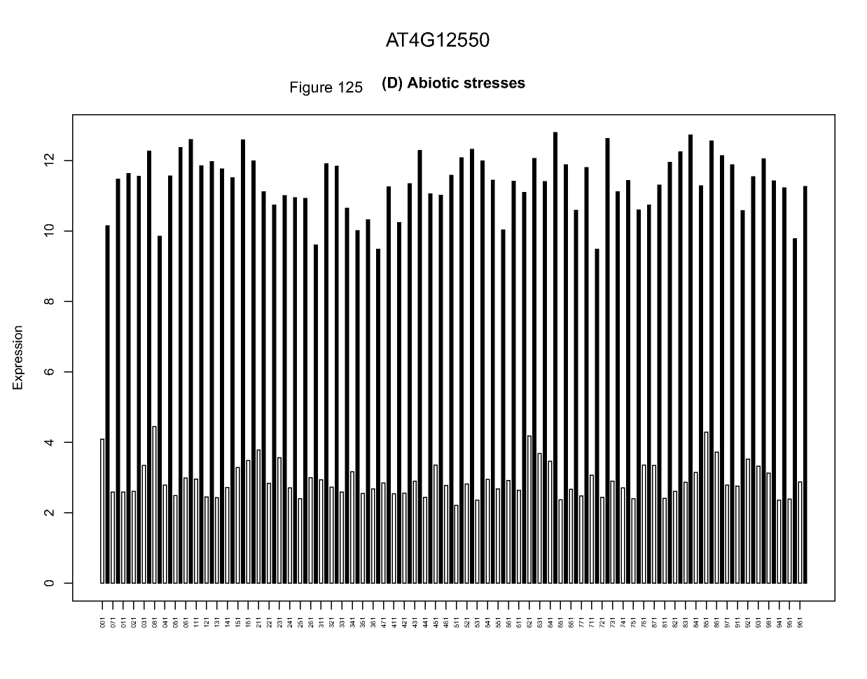

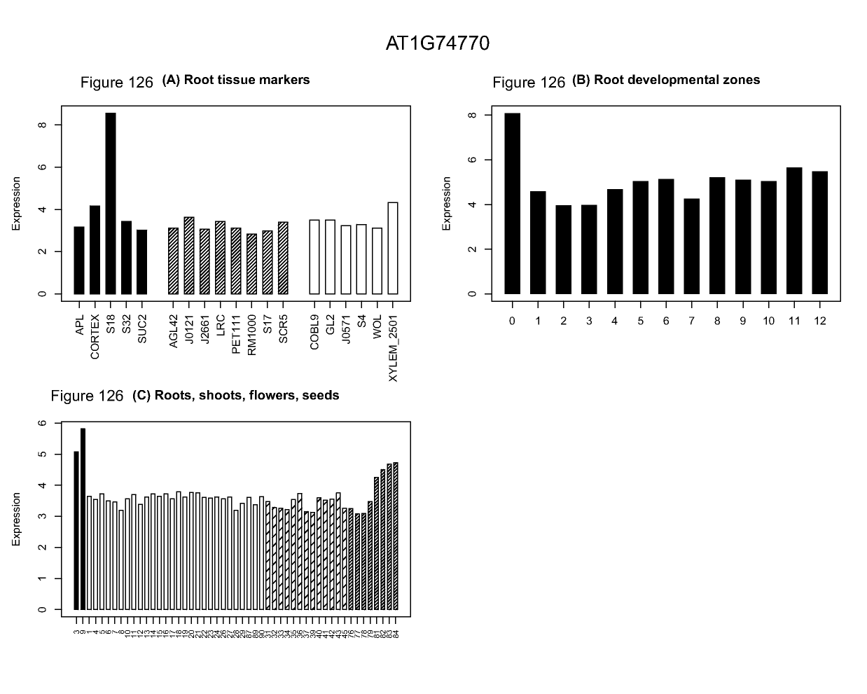

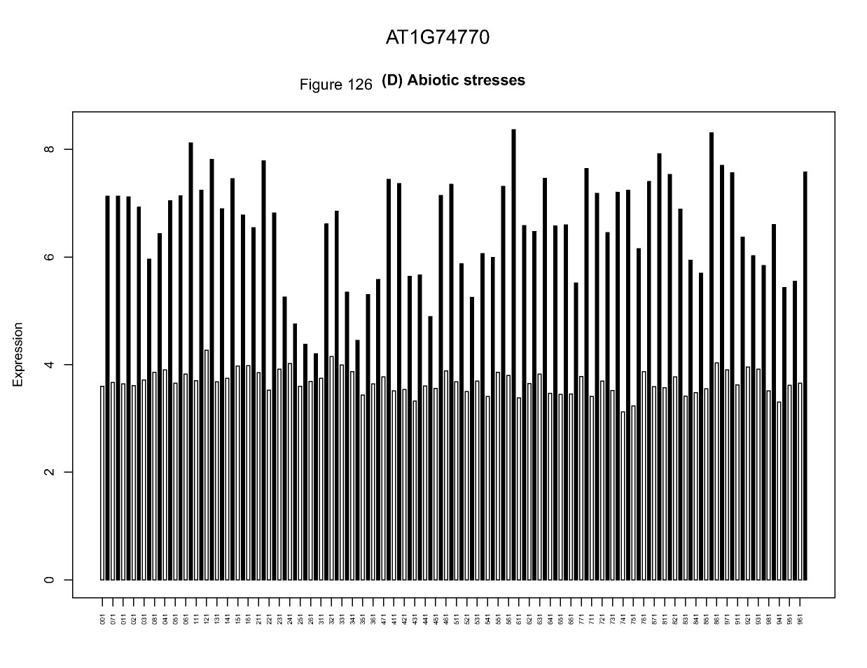

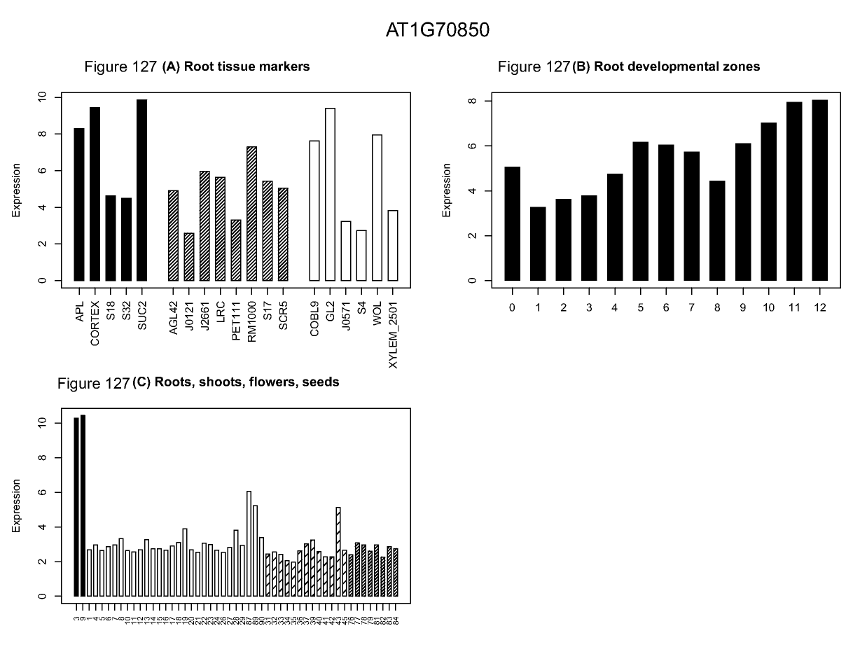

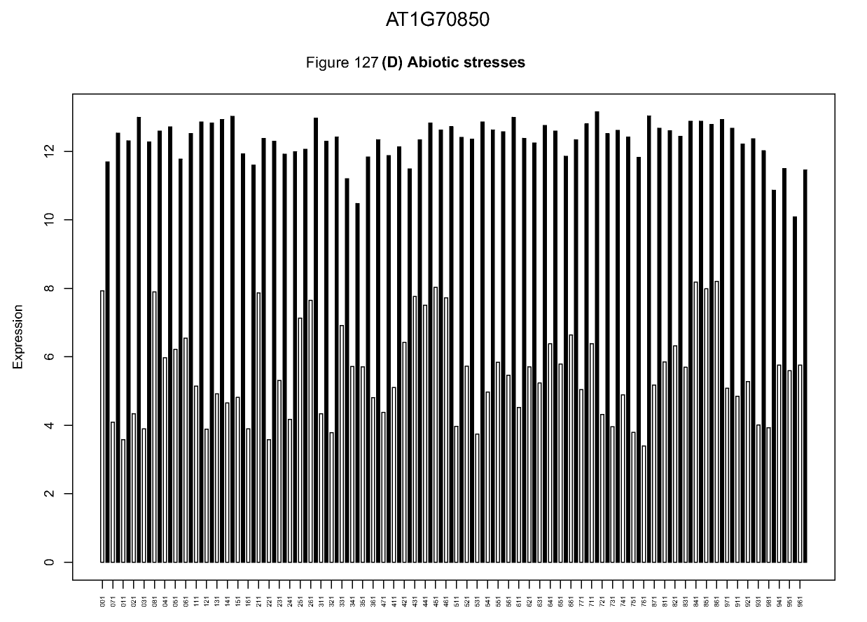

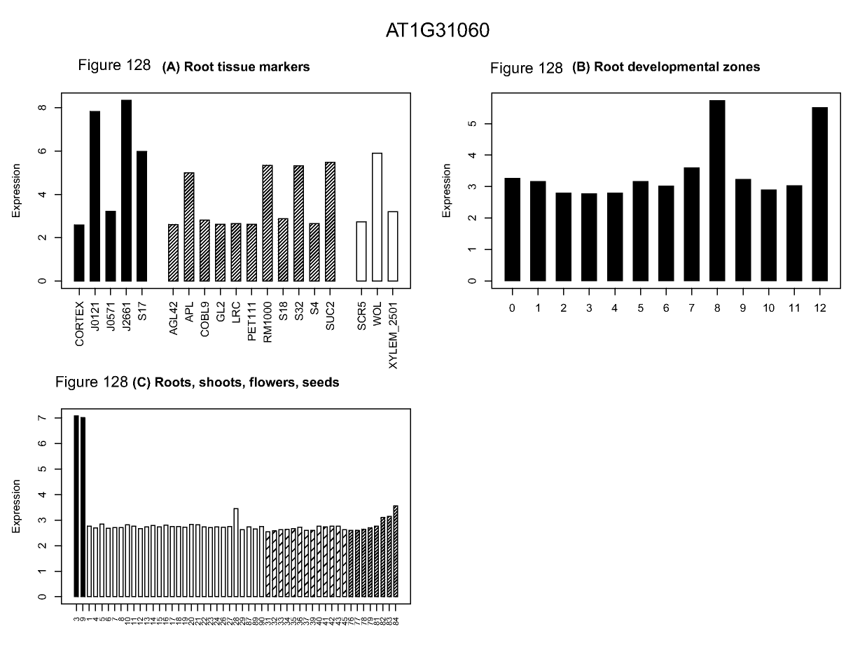

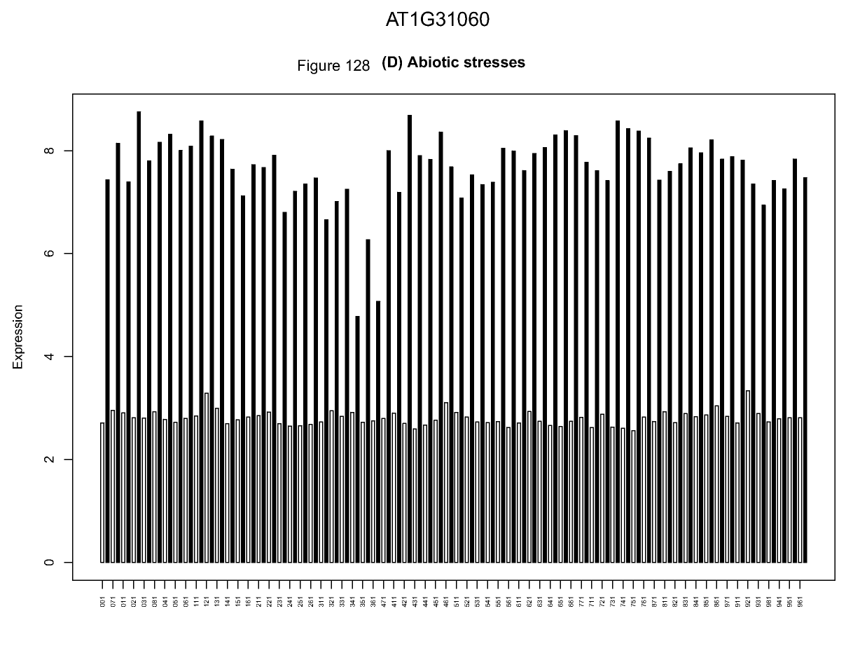

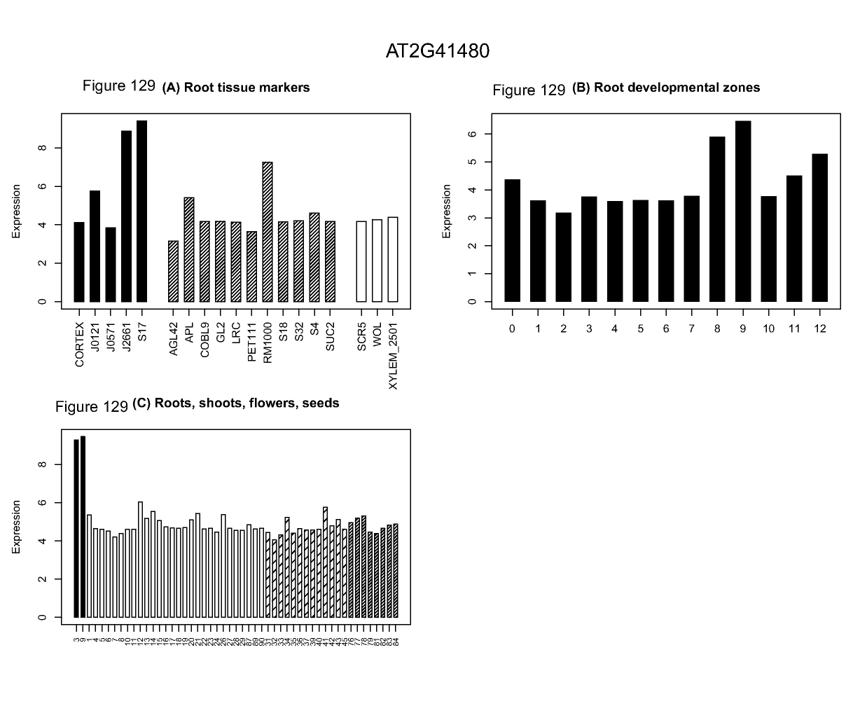

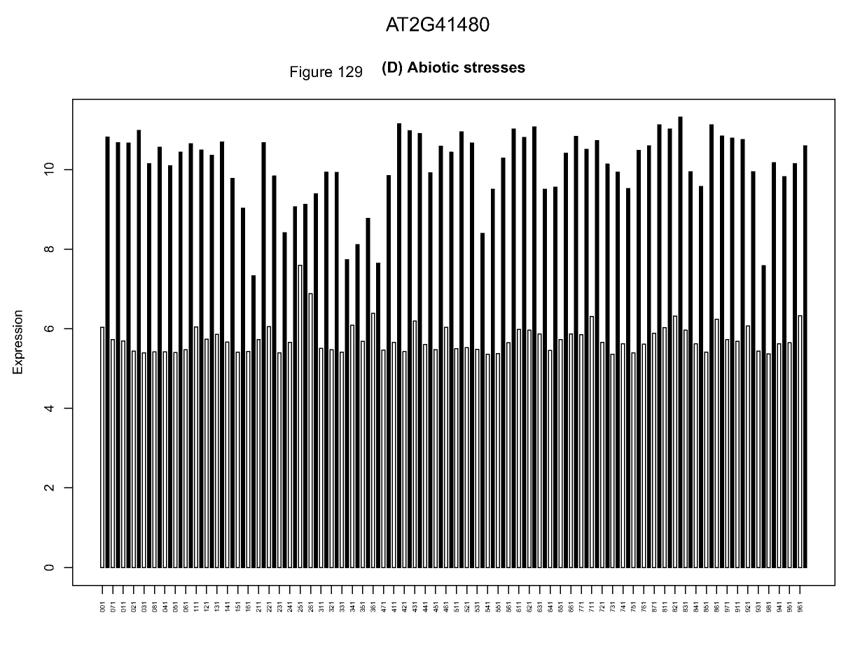

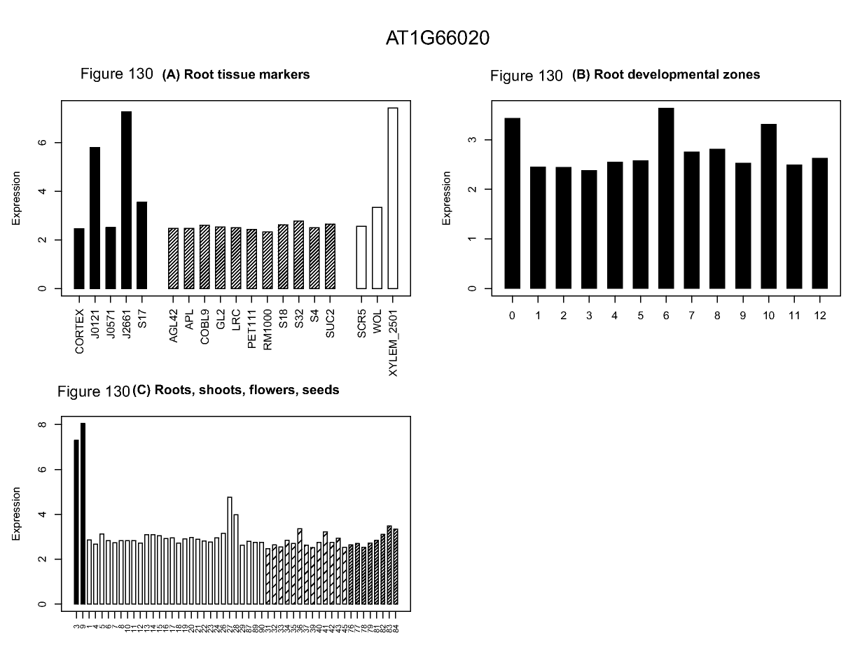

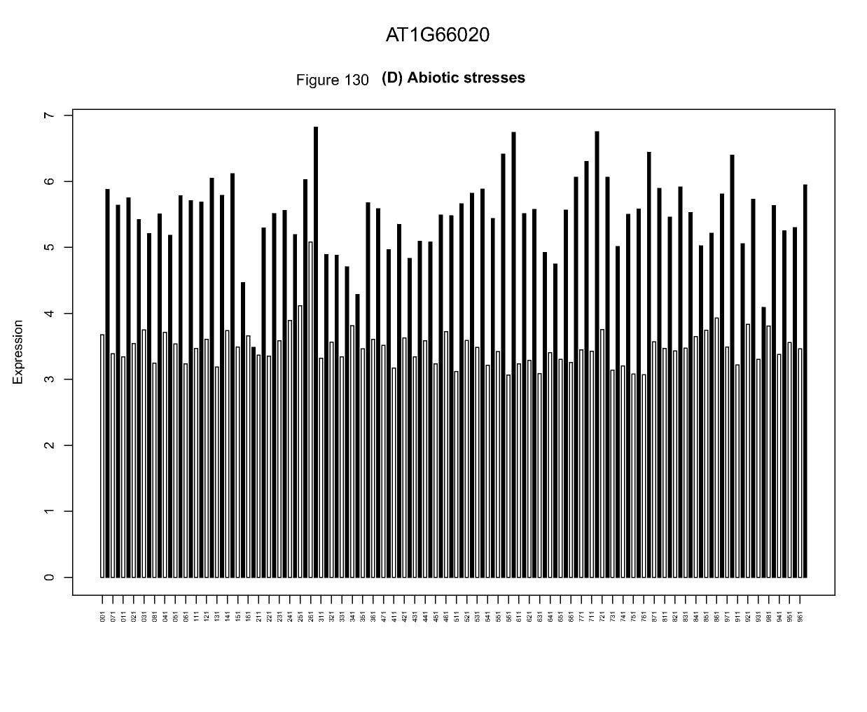

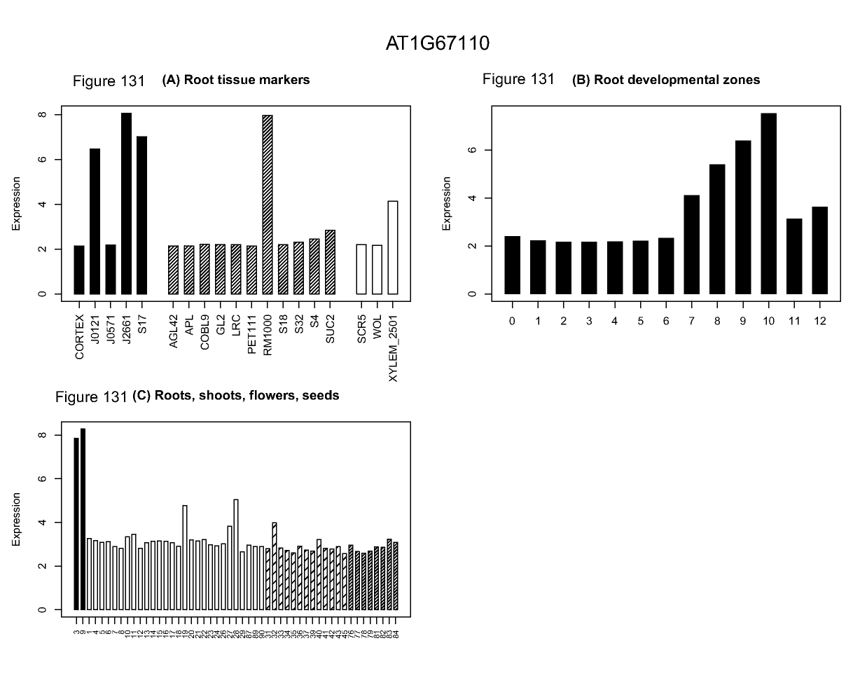

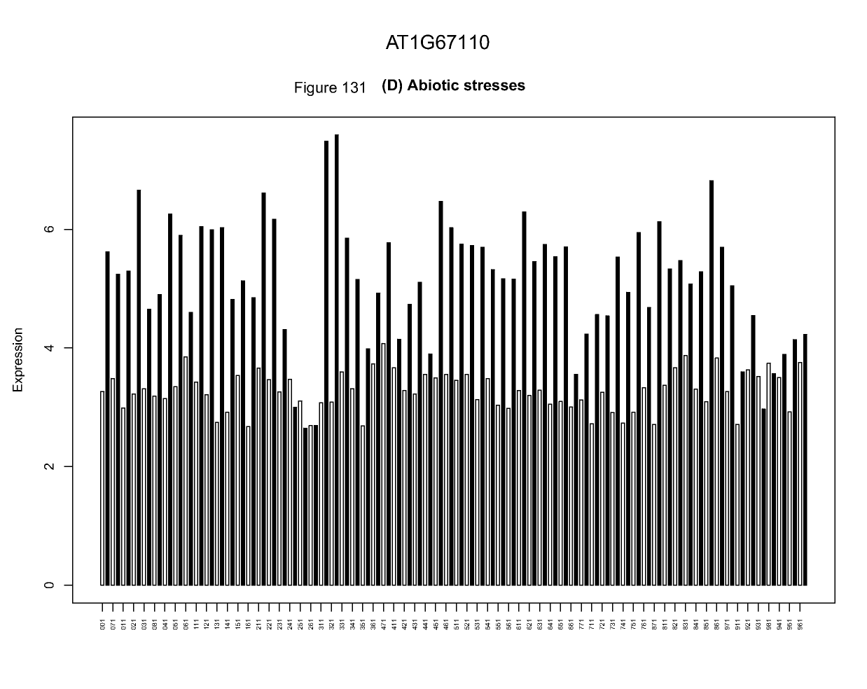

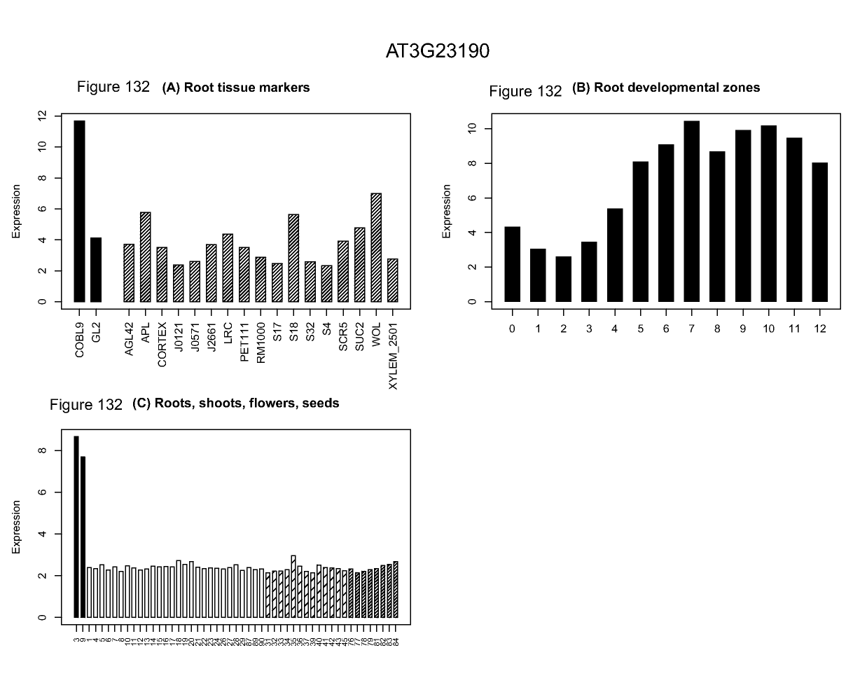

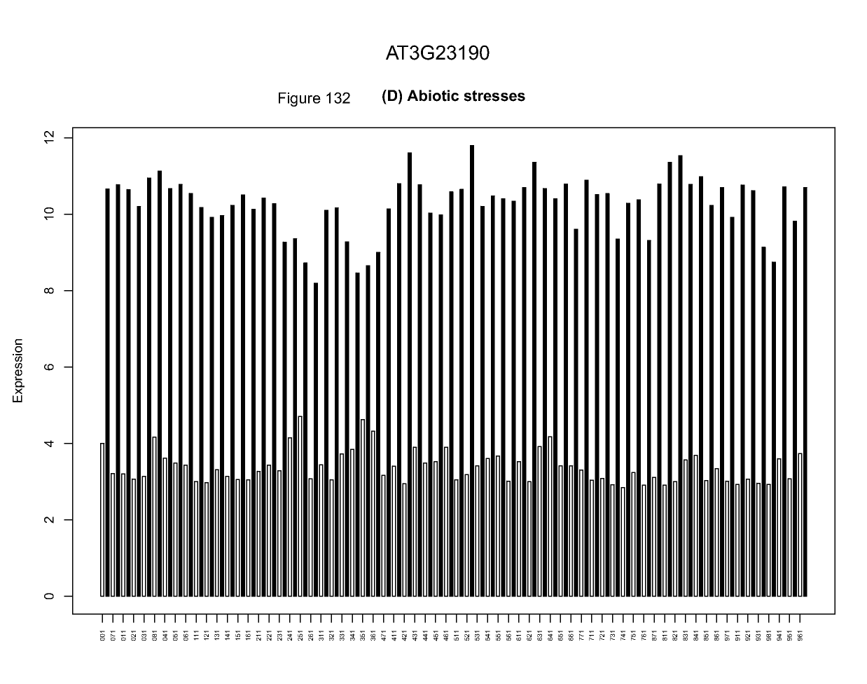

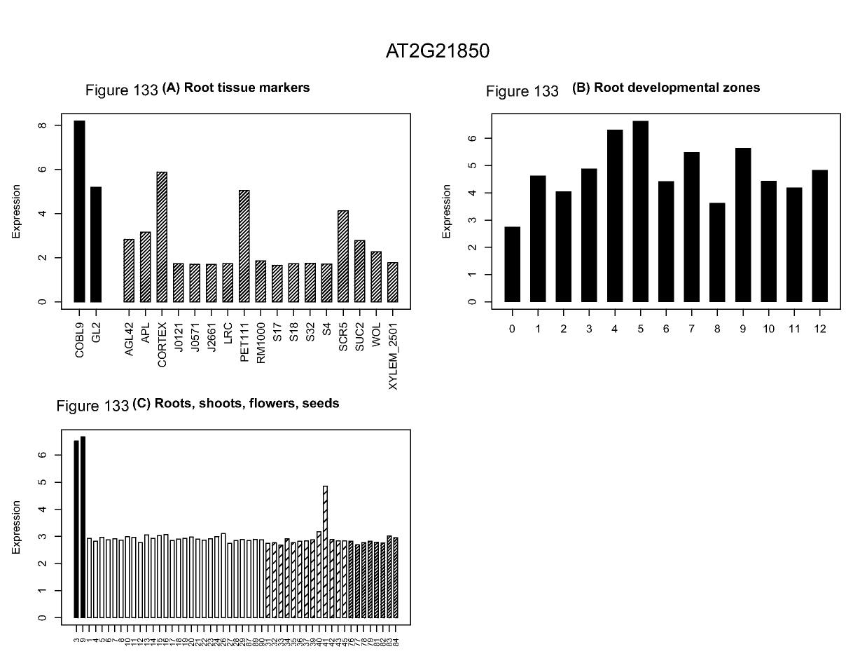

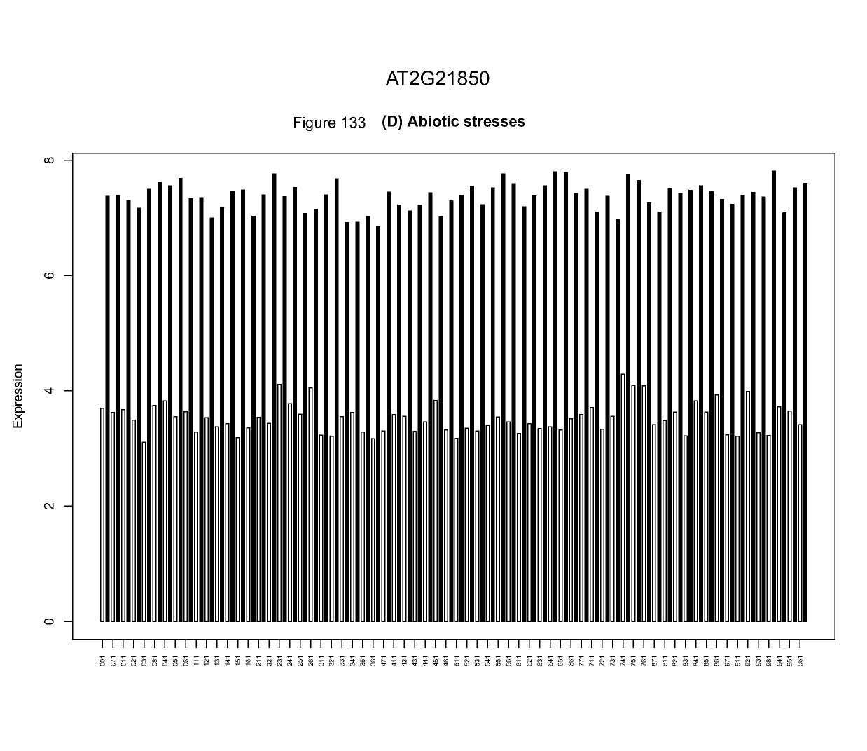

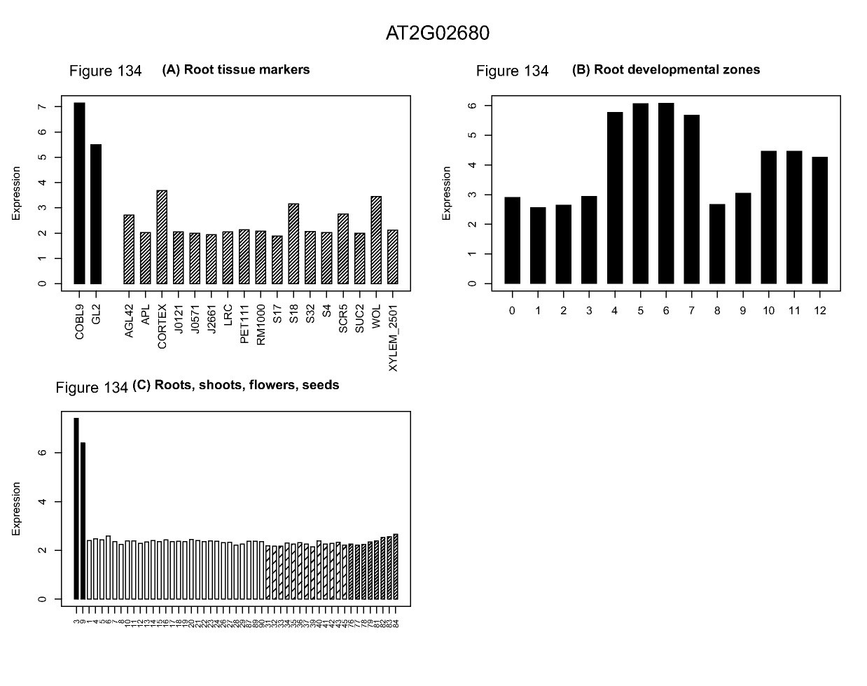

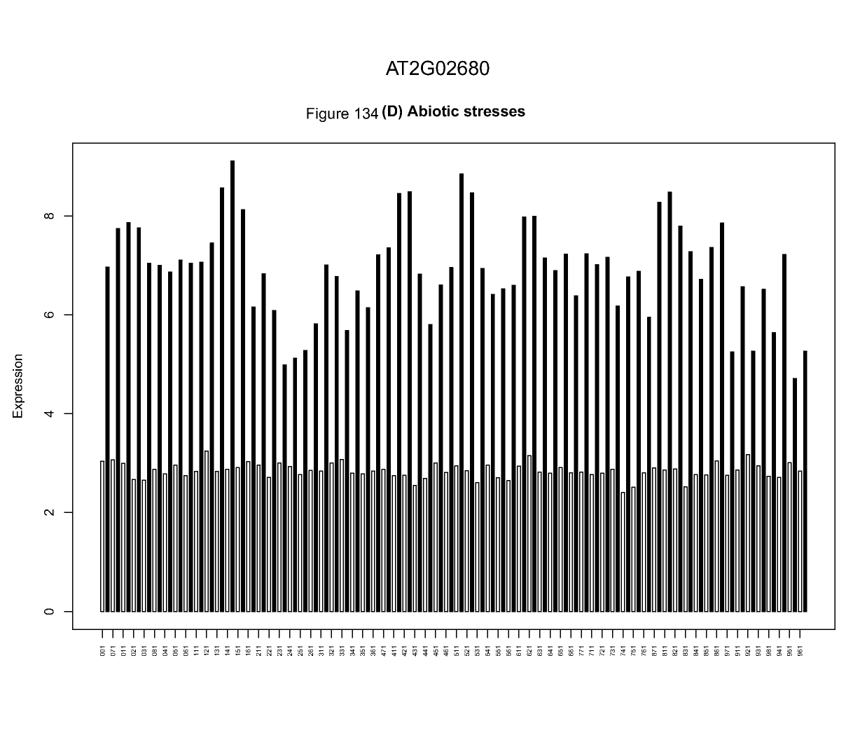

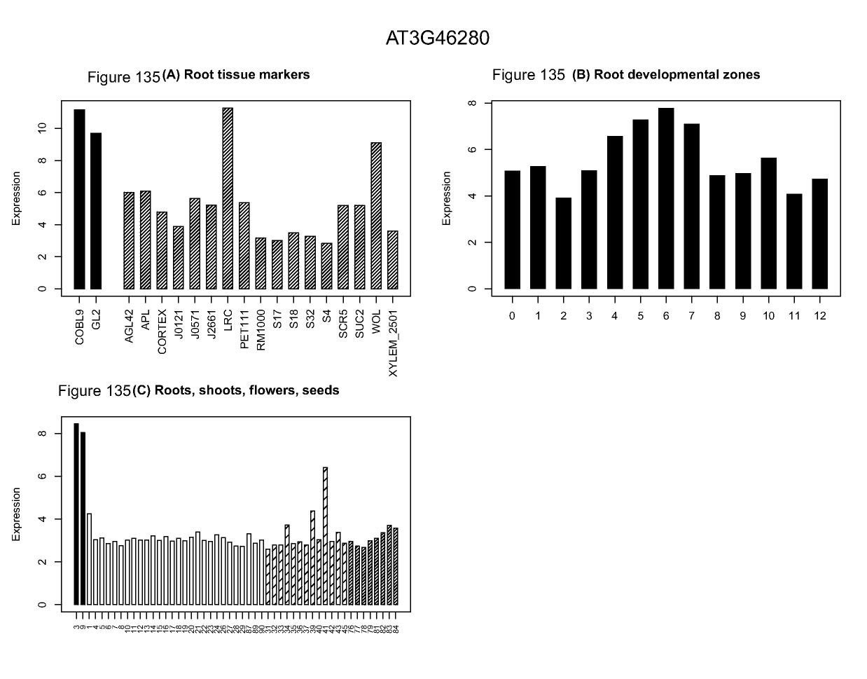

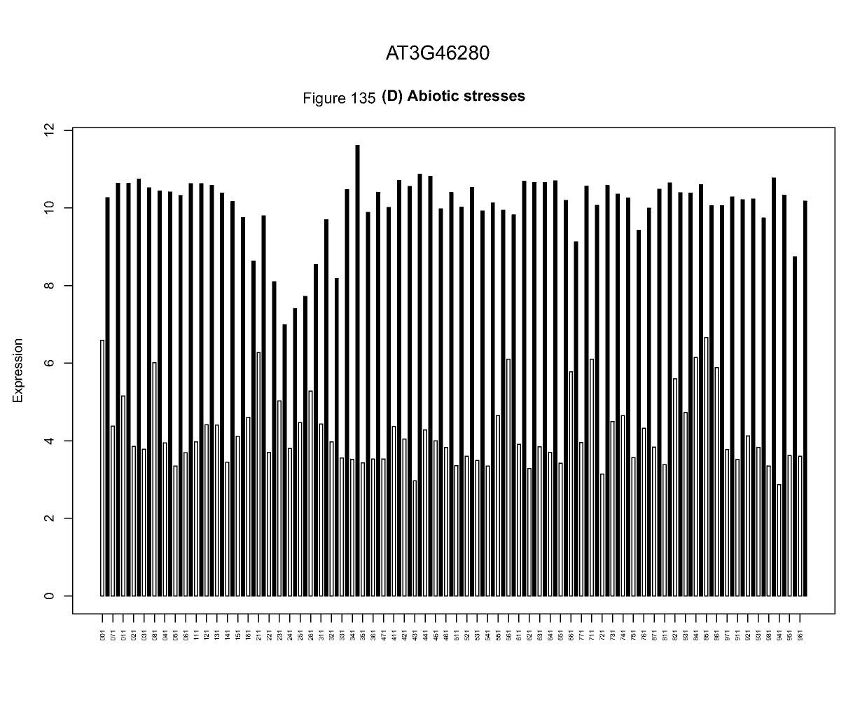

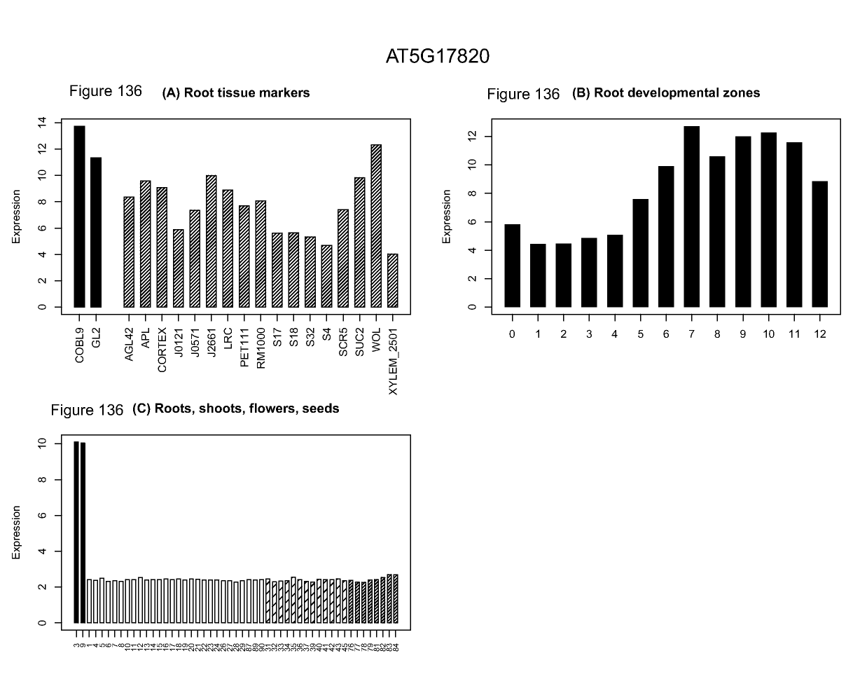

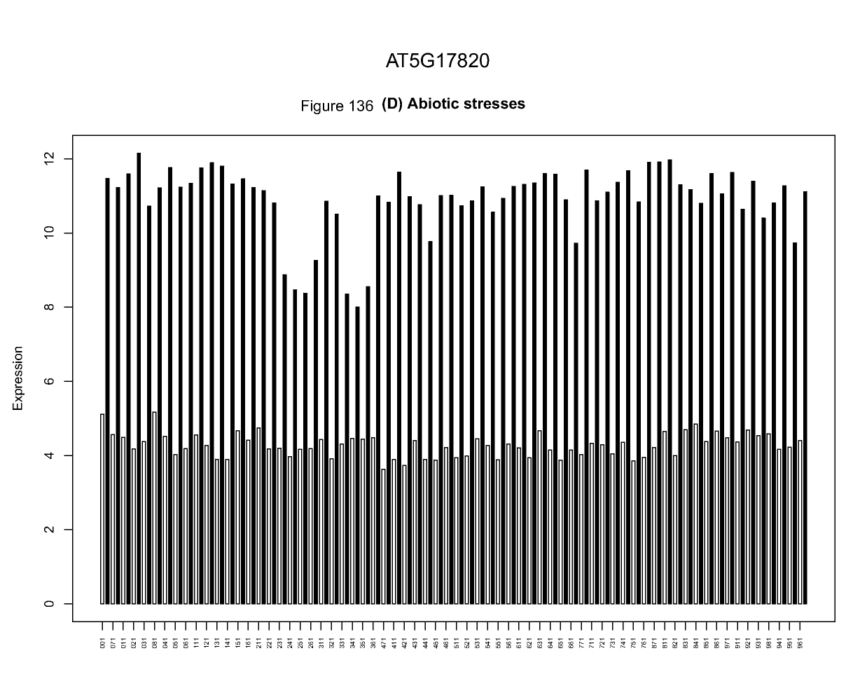

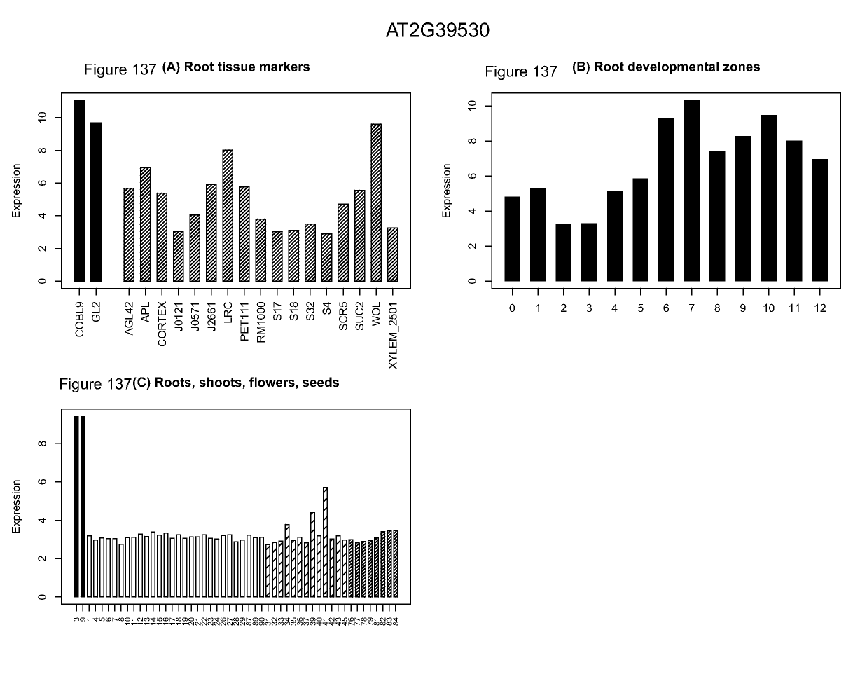

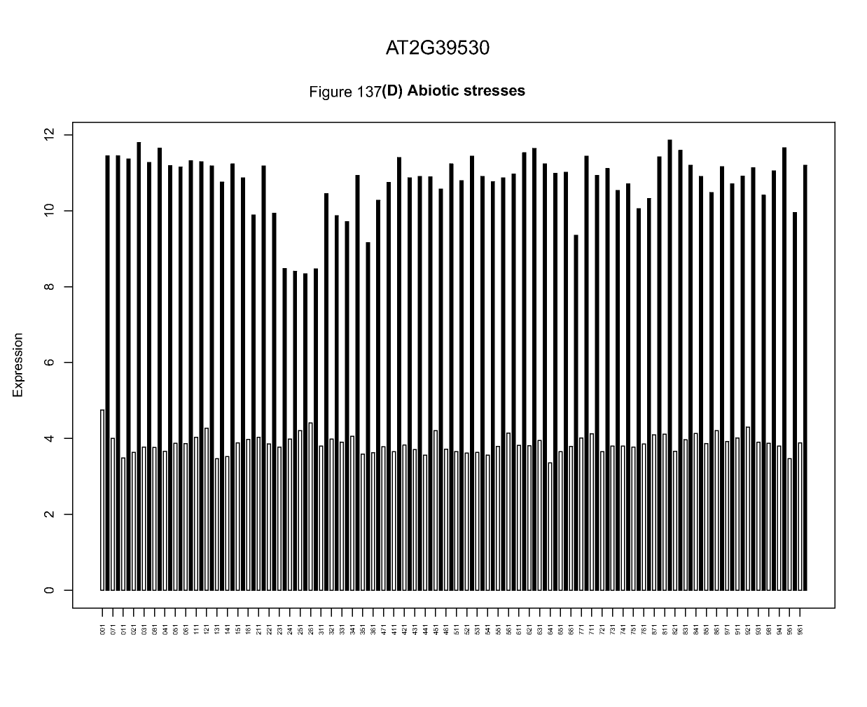

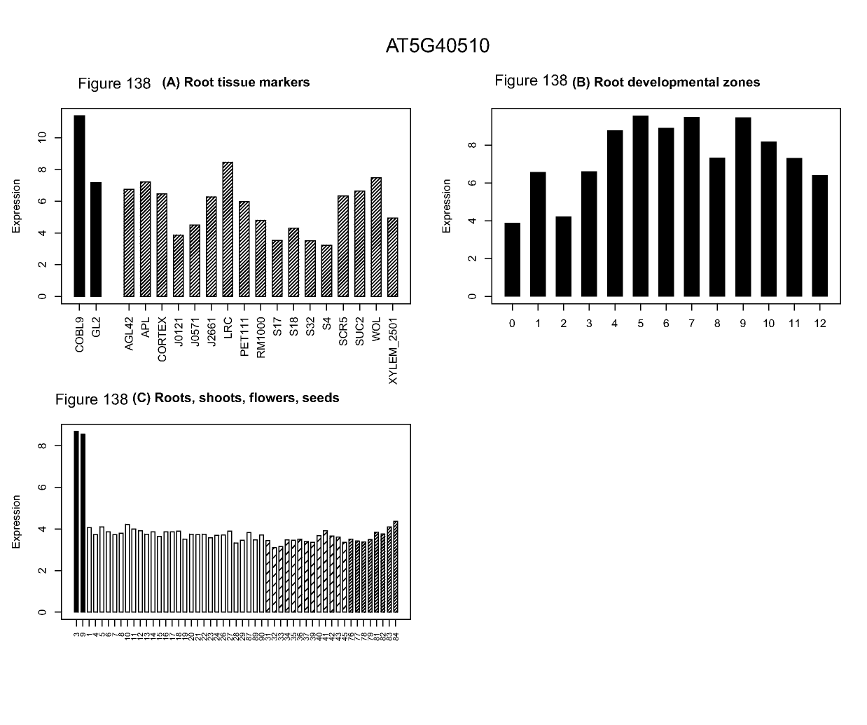

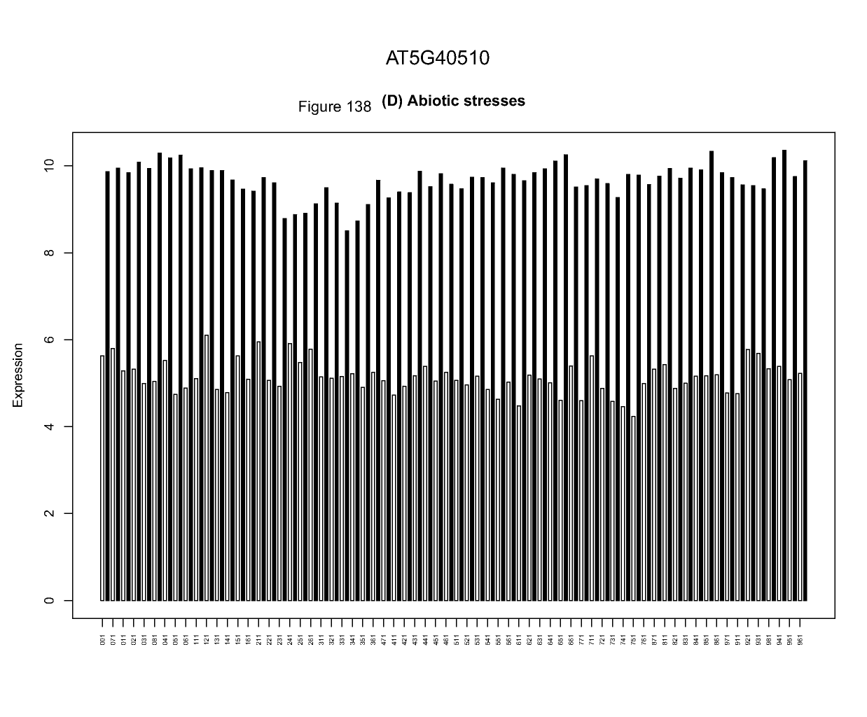

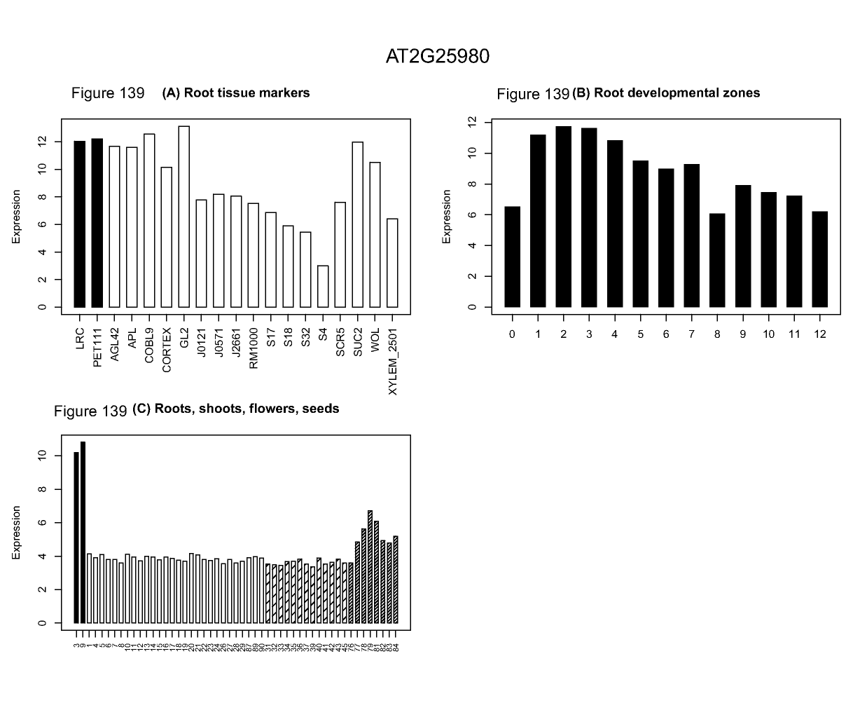

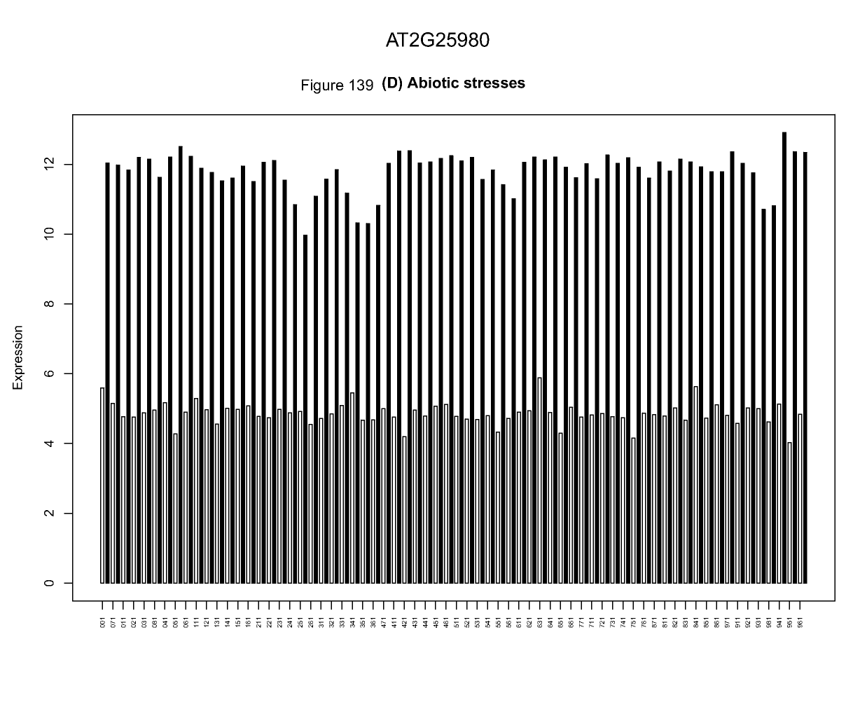

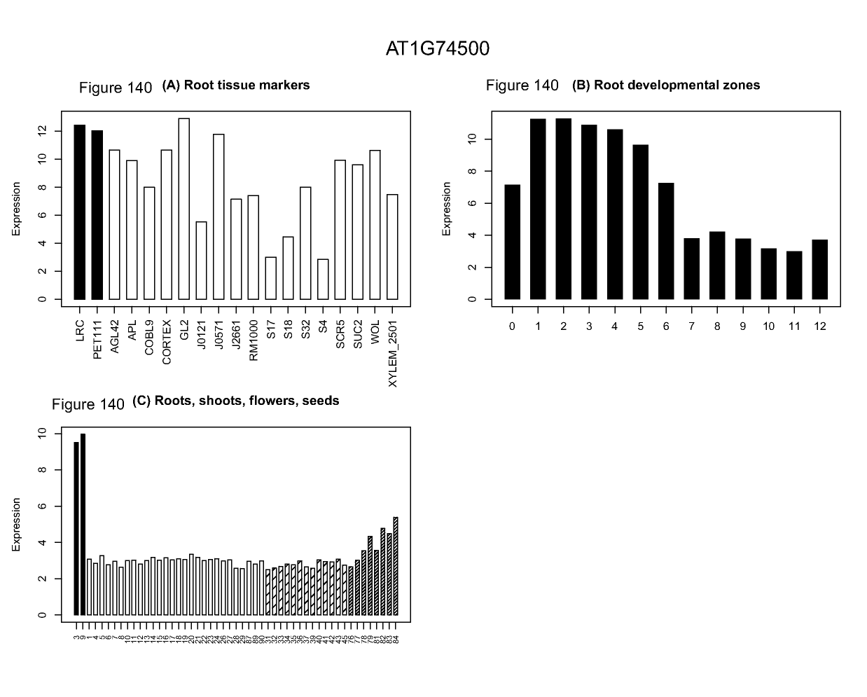

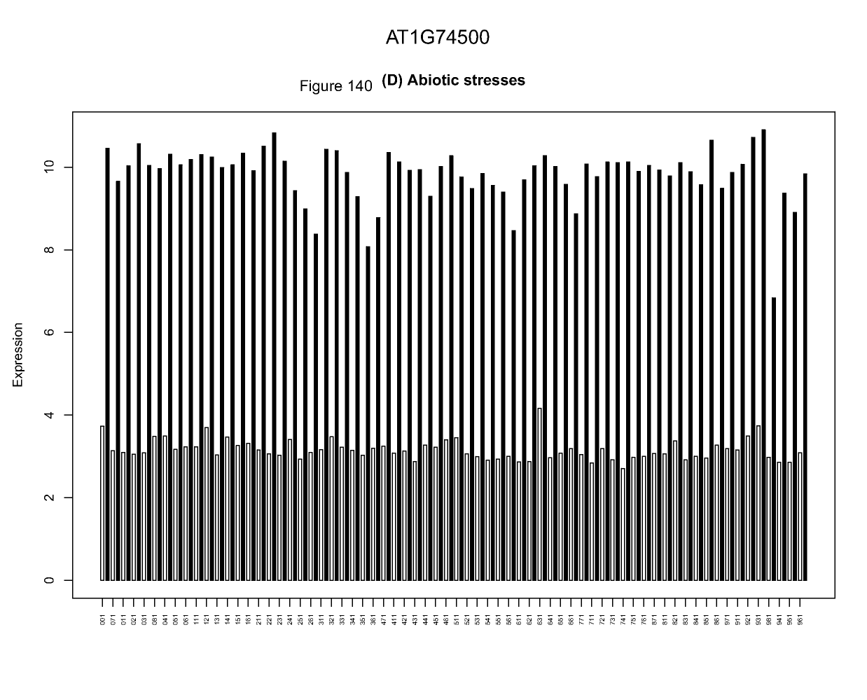

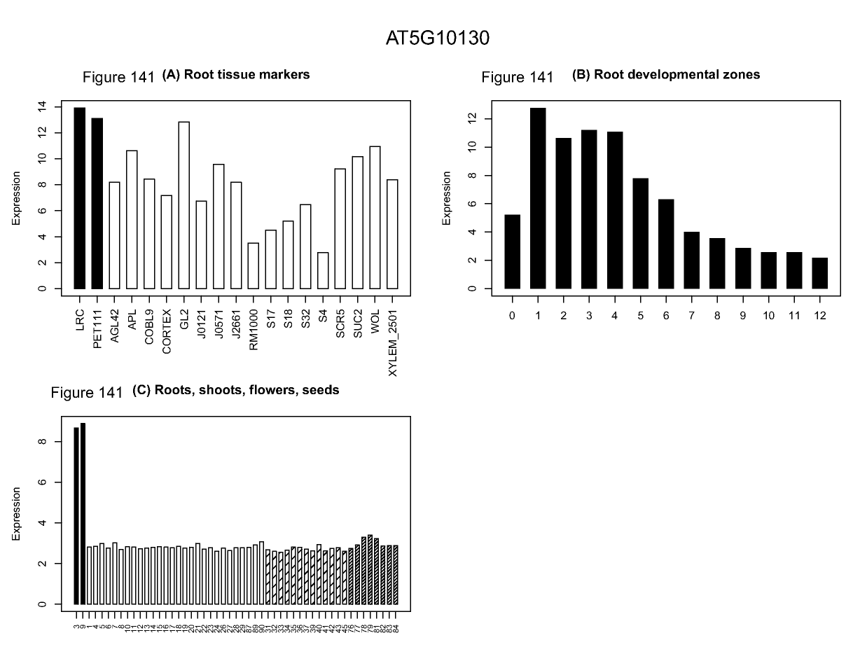

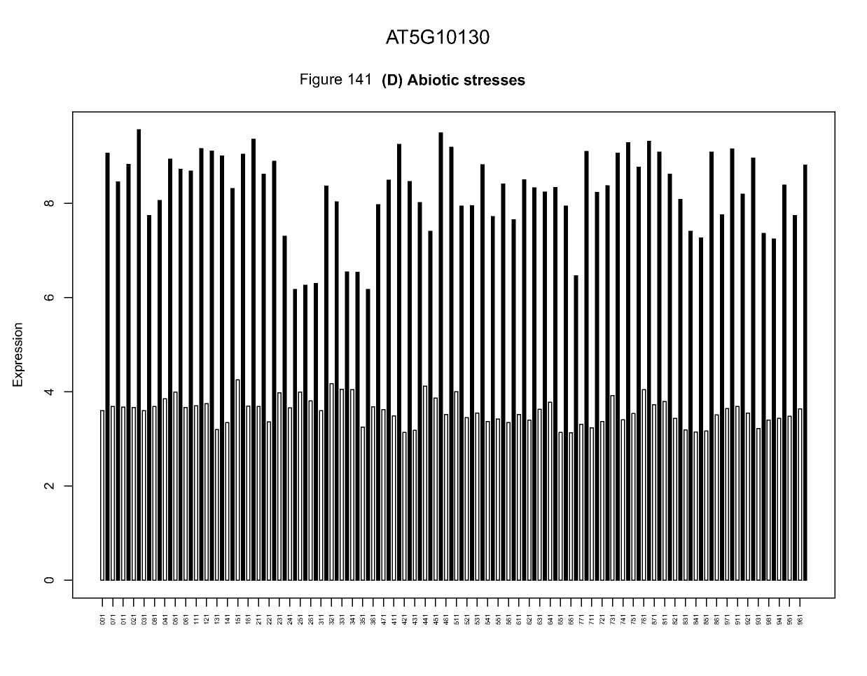

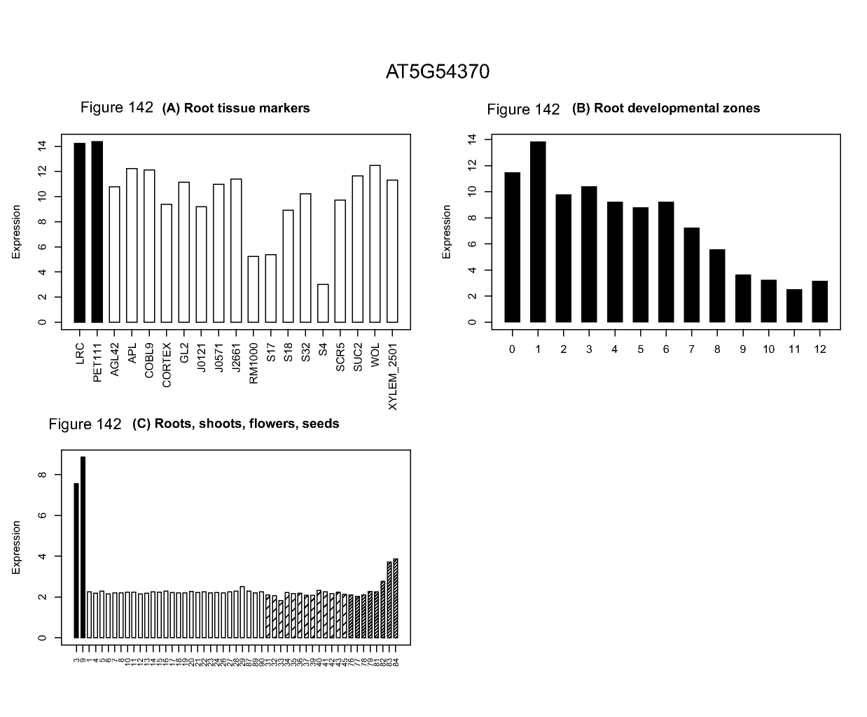

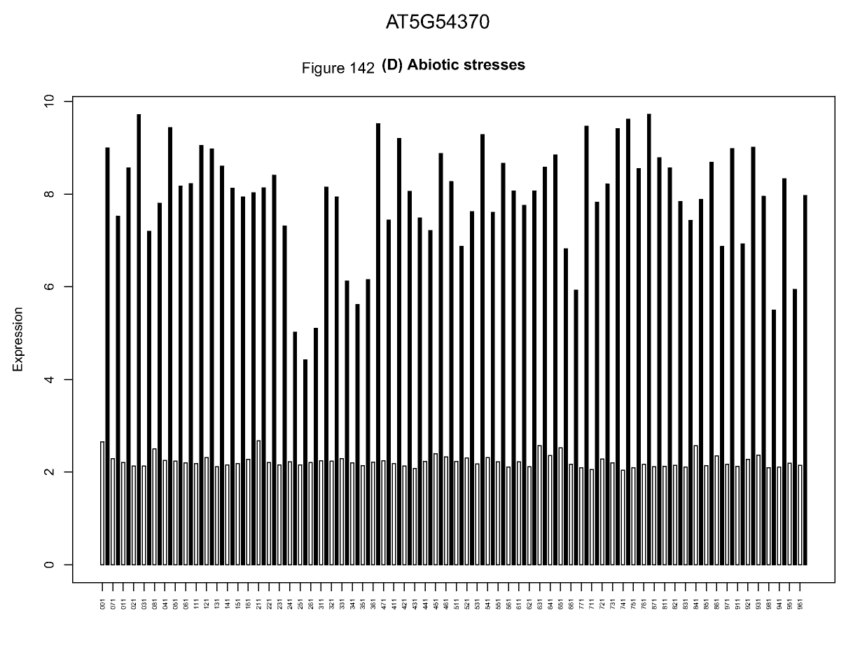

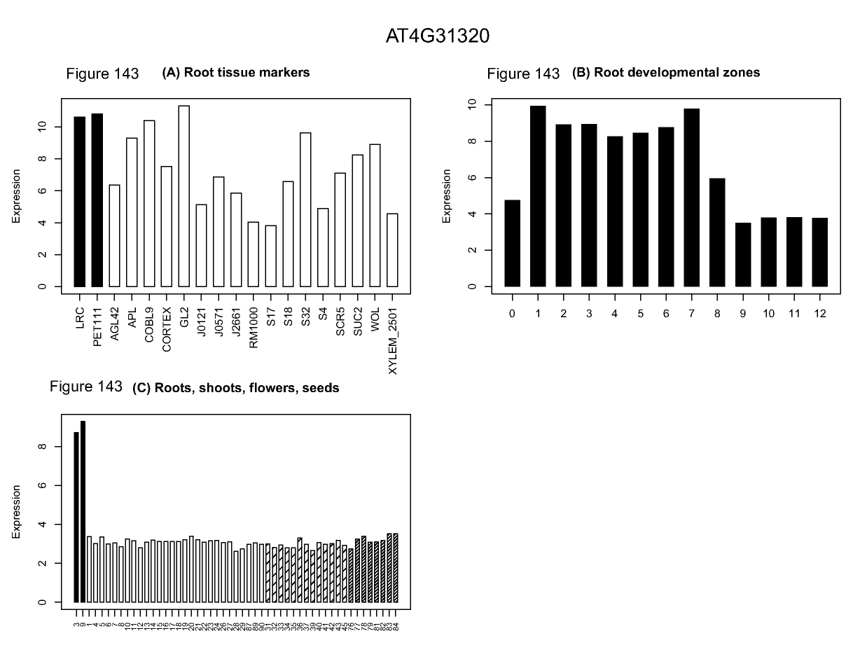

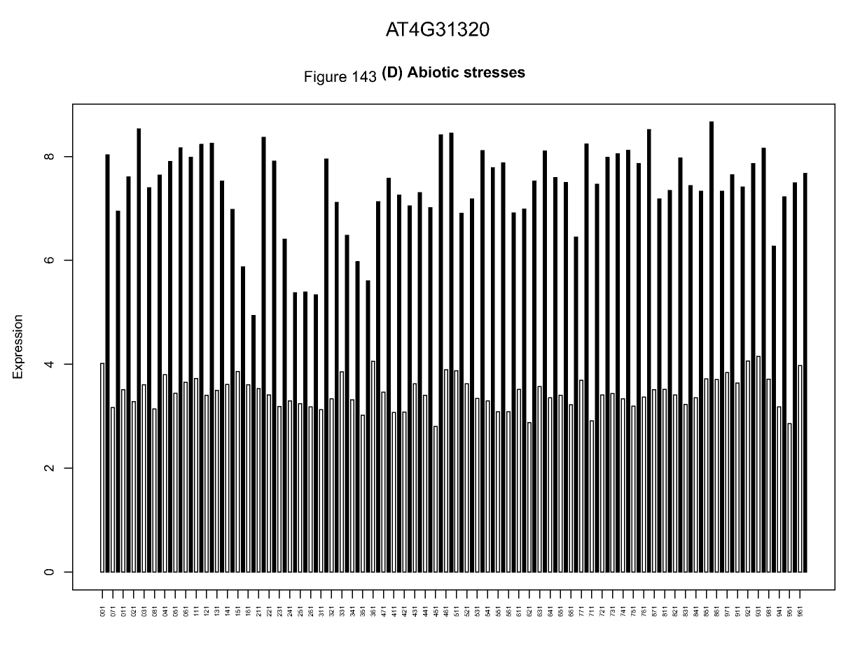

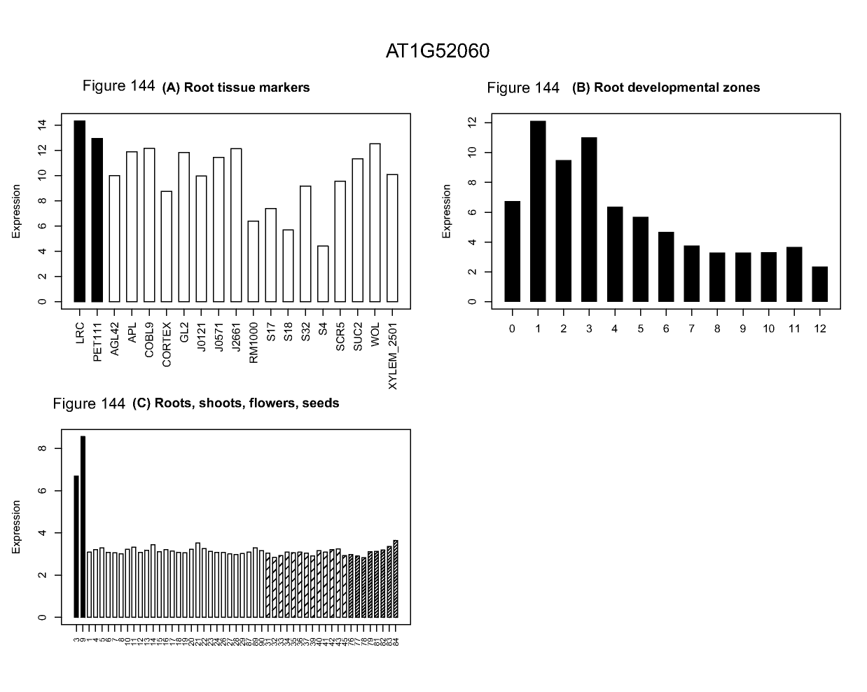

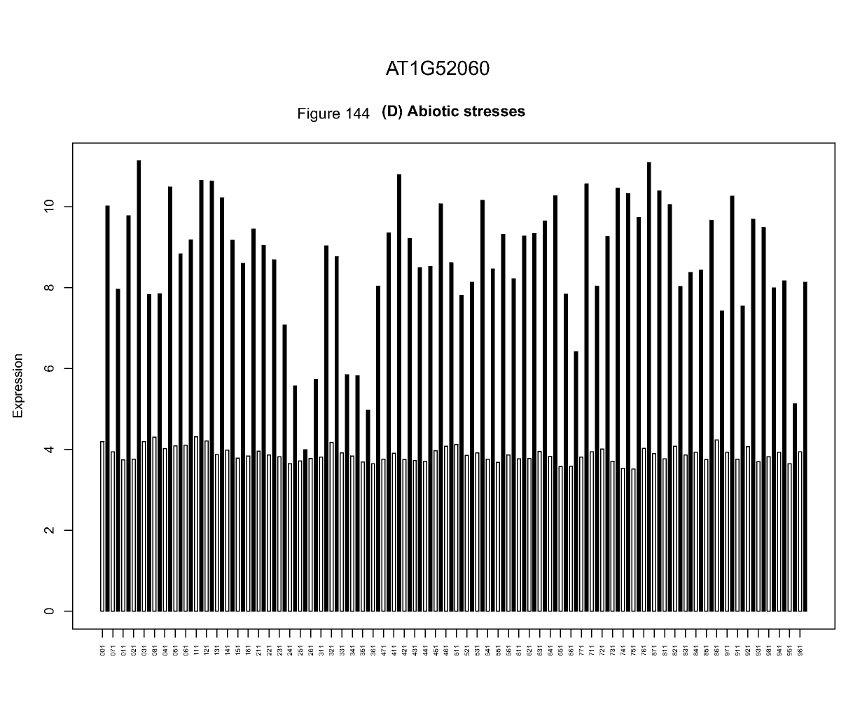

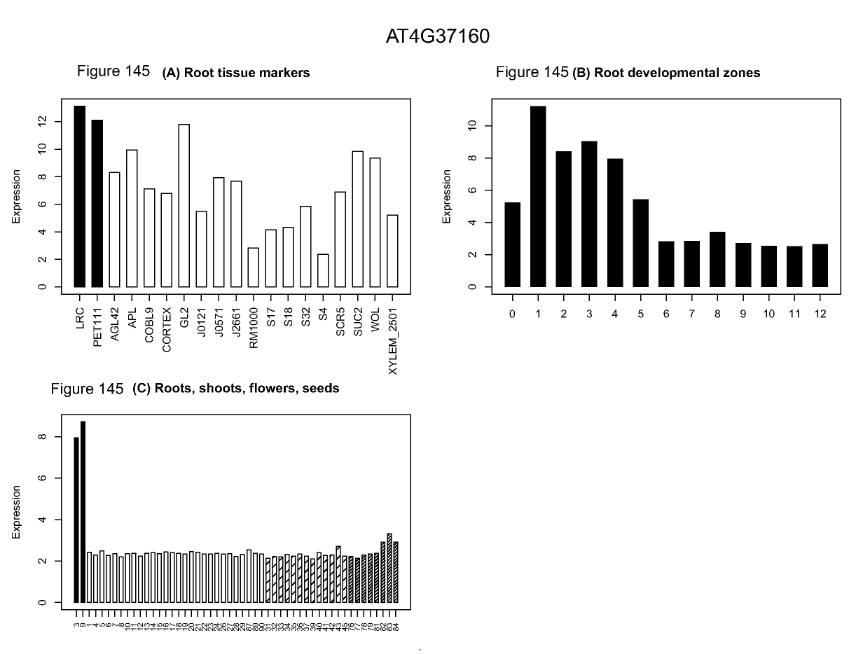

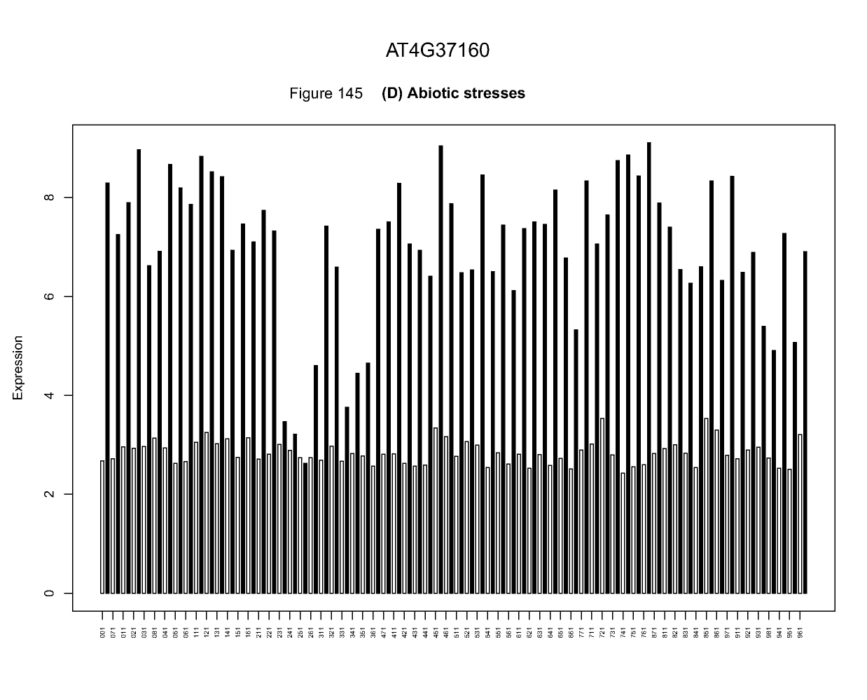

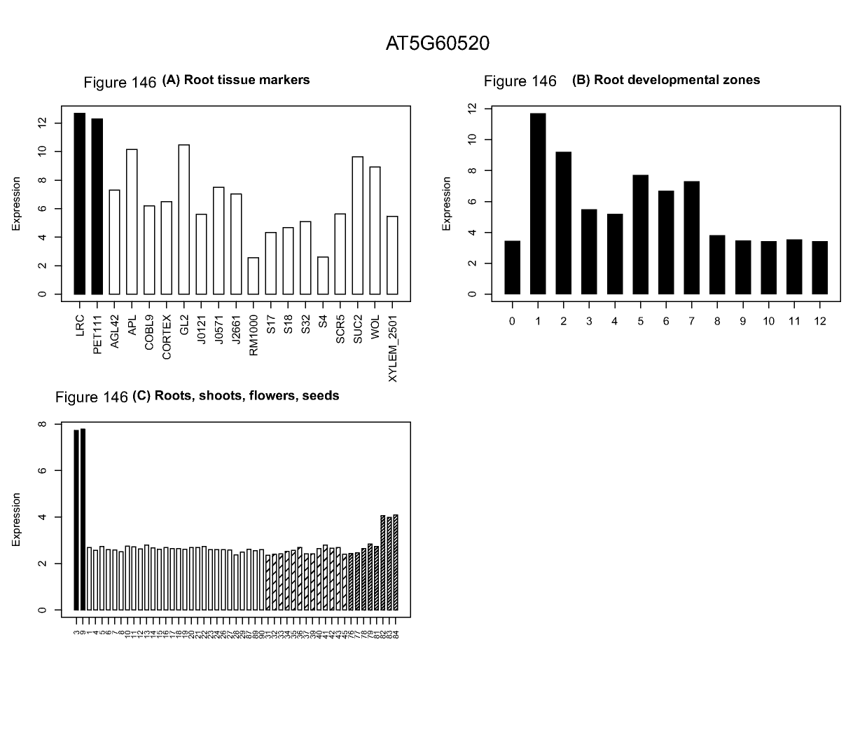

This example shows the endogenous expression data of the genes identified through the bioinformatics filtering of Example 1. Endogenous gene expression data for each gene corresponding to each of the identified Arabidopsis regulatory polynucleotides is provided in FIGS. 95A-D through 158A-D. All data shown in the figures are GC-RMA (GeneChip-RMA) normalized expression values (log 2 scale) from Affymetrix ATH1 microarrays which allow the detection of about 24,000 protein-encoding genes from Arabidopsis thaliana. For each gene, four plots labeled A-D are shown in the figures. Table 2 below shows the correspondence between the regulatory polynucleotides in Example 1 and the expression plots of FIGS. 95A-D through 158A-D. Also, when more than one set of expression data mapped to the same gene, the further data may also be shown.

TABLE 2

| Expression Figures | Regulatory Polynucleotide SEQ ID NOS |
|---|---|
| (Gene Accession No.) | (Corresponding Gene Accession No.) |
| 95A-D (AT5G65790) | 13 (AT5G65790) |
| 96A-D (AT1G77330) | 2 (AT1G77330) |
| 97A-D (AT4G21600) | 3 (AT4G21600) |
| 98A-D (AT5G43030) | 4 (AT5G43030) |
| 99A-D (AT3G62280) | 5 (AT3G62280) |
| 100A-D (AT1G15210) | 6 (AT1G15210) |
| 101A-D (AT3G54700) | 7 (AT3G54700) |
| 102A-D (AT2G38940) | 8 (AT2G38940) |
| 103A-D (AT3G45710) | 9 (AT3G45710) |
| 104A-D (AT2G27550) | 10 (AT2G27550) |
| 105A-D (AT2G16970) | 11 (AT2G16970) |
| 106A-D (AT1G64590) | 12 (AT1G64590) |
| 107A-D (AT1G54890) | 1 (AT1G54890) |
| 108A-D (AT5G10720) | 14 (AT5G10720) |
| 109A-D (AT4G00080) | 15 (AT4G00080) |
| 110A-D (AT4G19030) | 16 (AT4G19030) |
| 111A-D (AT3G19390) | 32 (AT3G19390) |
| 112A-D (AT4G22212) | 33 (AT4G22212) |
| 113A-D (AT5G23830) | 34 (AT5G23830) |
| 114A-D (AT5G26280) | 35 (AT5G26280) |
| 115A-D (AT1G52070) | 36 (AT1G52070) |
| 116A-D (AT3G16450) | 37 (AT3G16450) |
| 117A-D (AT1G66270) | 38 (AT1G66270) |
| 118A-D (AT1G66280) | 39 (AT1G66280) |
| 119A-D (AT5G48000) | 40 (AT5G48000) |
| 120A-D (AT5G48000) | 40 (AT5G48000) |
| 121A-D (AT5G53250) | 41 (AT5G53250) |
| 122A-D (AT5G50560) | 42 (AT5G50560) |
| 123A-D (AT5G63600) | 43 (AT5G63600) |
| 124A-D (AT4G30670) | 44 (AT4G30670) |
| 125A-D (AT4G12550) | 45 (AT4G12550) |
| 126A-D (AT1G74770) | 46 (AT1G74770) |
| 127A-D (AT1G70850) | 47 (AT1G70850) |
| 128A-D (AT1G31060) | 48 (AT1G31060) |
| 129A-D (AT2G41480) | 49 (AT2G41480) |
| 130A-D (AT1G66020) | 50 (AT1G66020) |
| 131A-D (AT1G67110) | 51 (AT1G67110) |
| 132A-D (AT3G23190) | 52 (AT3G23190) |
| 133A-D (AT2G21850) | 53 (AT2G21850) |
| 134A-D (AT2G02680) | 54 (AT2G02680) |
| 135A-D (AT3G46280) | 55 (AT3G46280) |
| 136A-D (AT5G17820) | 56 (AT5G17820) |
| 137A-D (AT2G39530) | 57 (AT2G39530) |
| 138A-D (AT5G40510) | 58 (AT5G40510) |
| 139A-D (AT2G25980) | 59 (AT2G25980) |
| 140A-D (AT1G74500) | 60 (AT1G74500) |
| 141A-D (AT5G10130) | 61 (AT5G10130) |
| 142A-D (AT5G54370) | 62 (AT5G54370) |
| 143A-D (AT4G31320) | 63 (AT4G31320) |
| 144A-D (AT1G52060) | 64 (AT1G52060) |
| 145A-D (AT4G37160) | 65 (AT4G37160) |
| 146A-D (AT5G60520) | 66 (AT5G60520) |
| 147A-D (AT1G78340) | 71 (AT1G78340) |
| 148A-D (AT5G26260) | 72 (AT5G26260) |
| 149A-D (AT4G13660) | 73 (AT4G13660) |
| 150A-D (AT1G22440) | 74 (AT1G22440) |
| 151A-D (AT2G36100) | 75 (AT2G36100) |
| 152A-D (AT3G11340) | 76 (AT3G11340 + intron) |
| 153A-D (AT1G02810) | 77 (AT1G02810 + intron) |
| 154A-D (AT5G64100) | 78 (AT5G64100 + intron) |
| 155A-D (AT5G59090) | 79 (AT5G59090 + intron) |
| 156A-D (AT2G43610) | 80 (AT2G43610 + intron) |
| 157A-D (AT5G44380) | 81 (AT5G44380 + intron) |
| 158A-D (AT2G18210) | 82 (AT2G18210) |

Plots A and B are derived from data published by Brady et al. (Science, 318:801-806 (2007)). Plot A in each figure shows expression values from cells sorted on the basis of expressing the indicated GFP marker. Table 3 contains a key showing the specific cell types in which each marker is expressed based on Brady et al. (Science, 318:801-806 (2007)). The table provides a description of cell types together with the associated markers. This table defines the relationship between cell-type and marker line, including which longitudinal sections of each cell-type are included. Lateral Root Primordia is included as a cell-type in this table, even though it may be a collection of multiple immature cell types. There are also no markers that differentiate between metaxylem and protoxylem or between metaphloem and protophloem, so those cell types are labeled Xylem and Phloem respectively. Together, these data provide expression information for virtually all cell-types found in the Arabidopsis root.

TABLE 3

| Cell Type | Markers | Longitudinal Section |
|---|---|---|
| Lateral root cap | LRC | 0-5 |
| Columella | PET111 | 0 |
| Quiescent centre | AGL42 | 1 |
| | RM1000 | 1 |
| | SCR5 | 1 |
| Hair cell | N/A | 1-6 |
| | COBL9 | 7-12 |
| Non-hair cell | GL2 | 1-12 |
| Cortex | J0571 | 1-12 |
| | CORTEX | 6-12 |
| Endodermis | J0571 | 1-12 |
| | SCR5 | 1-12 |
| Xylem pole pericycle | WOL | 1-8 |
| | J0121 | 8-12 |
| | J2661 | 12 |
| Phloem pole pericycle | WOL | 1-8 |
| | S17 | 7-12 |
| | J2661 | 12 |
| Phloem | APL | 3-12 |
| | S32 | 1-12 |
| | WOL | 1-8 |
| Phloem CCs | SUC2 | 9-12 |
| | WOL | 1-8 |
| Xylem | S4 | 1-6 |
| | S18 | 7-12 |
| | WOL | 1-8 |
| Lateral root primordial | RM1000 | 11 |
| Procambium | WOL | 1-8 |

Plot B in each figure shows expression values from root sections along the longitudinal axis. Different regions along this axis correspond to different developmental stages of root cell development. In particular, section 0 corresponds to the columella, sections 1-6 correspond to the meristematic zone, sections 7-8 correspond to the elongation zone, and sections 9-12 correspond to the maturation zone.

Plots C and D in each figure are derived from publically available expression data of the AtGeneExpress project (available on the World Wide Web at weigelworld.org/resources/microarray/AtGenExpress). Plot C shows developmental specific expression as described by Schmid et al. (Nat. Genet., 37: 501-506 (2005)). A key for the samples in this dataset is provided in Table 4. For ease of visualization, root expression values are indicated with black bars, shoot expression with white bars, flower expression with coarse hatched bars, and seed expression with fine hatched bars.

TABLE 4

| | | Experiment | | | | | | |
|---|---|---|---|---|---|---|---|---|
| No | Sample ID | Description | Genotype | Tissue | Age | | Photoperiod | Substrate |
| 1 | ATGE_1 | development | Wt | Cotyledons | 7 | days | continuous | Soil |
| | | baseline | | | | | light | |
| 2 | ATGE_2 | development | Wt | Hypocotyl | 7 | days | continuous | Soil |
| | | baseline | | | | | light | |
| 3 | ATGE_3 | development | Wt | Roots | 7 | days | continuous | Soil |
| | | baseline | | | | | light | |
| 4 | ATGE_4 | development | Wt | shoot apex, | 7 | days | continuous | Soil |
| | | baseline | | vegetative + | | | light | |
| | | | | young leaves | | | | |
| 5 | ATGE_5 | development | Wt | leaves 1 + 2 | 7 | days | continuous | Soil |
| | | baseline | | | | | light | |
| 6 | ATGE_6 | development | Wt | shoot apex, | 7 | days | continuous | Soil |
| | | baseline | | vegetative | | | light | |
| 7 | ATGE_7 | development | Wt | seedling, | 7 | days | continuous | Soil |
| | | baseline | | green parts | | | light | |
| 8 | ATGE_8 | development | Wt | shoot apex, | 14 | days | continuous | Soil |
| | | baseline | | transition | | | light | |
| | | | | (before bolting) | | | | |
| 9 | ATGE_9 | development | Wt | Roots | 17 | days | continuous | Soil |
| | | baseline | | | | | light | |
| 10 | ATGE_10 | development | Wt | rosette leaf #4, | 10 | days | continuous | Soil |
| | | baseline | | 1 cm long | | | light | |
| 11 | ATGE_11 | development | gl1-T | rosette leaf #4, | 10 | days | continuous | Soil |
| | | baseline | | 1 cm long | | | light | |
| 12 | ATGE_12 | development | Wt | rosette leaf # 2 | 17 | days | continuous | Soil |
| | | baseline | | | | | light | |
| 13 | ATGE_13 | development | Wt | rosette leaf # 4 | 17 | days | continuous | Soil |
| | | baseline | | | | | light | |
| 14 | ATGE_14 | development | Wt | rosette leaf # 6 | 17 | days | continuous | Soil |
| | | baseline | | | | | light | |
| 15 | ATGE_15 | development | Wt | rosette leaf # 8 | 17 | days | continuous | Soil |
| | | baseline | | | | | light | |
| 16 | ATGE_16 | development | Wt | rosette leaf # 10 | 17 | days | continuous | soil |
| | | baseline | | | | | light | |
| 17 | ATGE_17 | development | Wt | rosette leaf # 12 | 17 | days | continuous | soil |
| | | baseline | | | | | light | |
| 18 | ATGE_18 | development | gl1-T | rosette leaf # 12 | 17 | days | continuous | soil |
| | | baseline | | | | | light | |
| 19 | ATGE_19 | development | Wt | leaf 7, petiole | 17 | days | continuous | soil |
| | | baseline | | | | | light | |
| 20 | ATGE_20 | development | Wt | leaf 7, proximal | 17 | days | continuous | soil |
| | | baseline | | half | | | light | |
| 21 | ATGE_21 | development | Wt | leaf 7, distal | 17 | days | continuous | soil |
| | | baseline | | half | | | light | |
| 22 | ATGE_22 | development | Wt | developmental | 21 | days | continuous | soil |
| | | baseline | | drift, entire | | | light | |
| | | | | rosette after | | | | |
| | | | | transition to | | | | |
| | | | | flowering, but | | | | |
| | | | | before bolting | | | | |
| 23 | ATGE_23 | development | Wt | as above | 22 | days | continuous | soil |
| | | baseline | | | | | light | |
| 24 | ATGE_24 | development | Wt | as above | 23 | days | continuous | soil |
| | | baseline | | | | | light | |
| 25 | ATGE_25 | development | Wt | senescing leaves | 35 | days | continuous | soil |
| | | baseline | | | | | light | |
| 26 | ATGE_26 | development | Wt | cauline leaves | 21 | days | continuous | soil |
| | | baseline | | | | | light | |
| 27 | ATGE_27 | development | Wt | stem, 2nd | 21+ | days | continuous | soil |
| | | baseline | | internode | | | light | |
| 28 | ATGE_28 | development | Wt | 1st node | 21+ | days | continuous | soil |
| | | baseline | | | | | light | |
| 29 | ATGE_29 | development | Wt | shoot apex, | 21 | days | continuous | soil |
| | | baseline | | inflorescence | | | light | |
| | | | | (after bolting) | | | | |
| 30 | ATGE_31 | development | Wt | flowers stage 9 | 21+ | days | continuous | soil |
| | | baseline | | | | | light | |
| 31 | ATGE_32 | development | Wt | flowers stage | 21+ | days | continuous | soil |
| | | baseline | | 10/11 | | | light | |
| 32 | ATGE_33 | development | Wt | flowers stage | 21+ | days | continuous | soil |
| | | baseline | | 12 | | | light | |
| 33 | ATGE_34 | development | Wt | flowers stage | 21+ | days | continuous | soil |
| | | baseline | | 12, sepals | | | light | |
| 34 | ATGE_35 | development | Wt | flowers stage | 21+ | days | continuous | soil |
| | | baseline | | 12, petals | | | light | |
| 35 | ATGE_36 | development | Wt | flowers stage | 21+ | days | continuous | soil |
| | | baseline | | 12, stamens | | | light | |
| 36 | ATGE_37 | development | Wt | flowers stage | 21+ | days | continuous | soil |
| | | baseline | | 12, carpels | | | light | |
| 37 | ATGE_39 | development | Wt | flowers stage | 21+ | days | continuous | soil |
| | | baseline | | 15 | | | light | |
| 38 | ATGE_40 | development | Wt | flowers stage | 21+ | days | continuous | soil |
| | | baseline | | 15, pedicels | | | light | |
| 39 | ATGE_41 | development | Wt | flowers stage | 21+ | days | continuous | soil |
| | | baseline | | 15, sepals | | | light | |
| 40 | ATGE_42 | development | Wt | flowers stage | 21+ | days | continuous | soil |
| | | baseline | | 15, petals | | | light | |
| 41 | ATGE_43 | development | Wt | flowers stage | 21+ | days | continuous | soil |
| | | baseline | | 15, stamen | | | light | |
| 42 | ATGE_45 | development | Wt | flowers stage | 21+ | days | continuous | soil |
| | | baseline | | 15, carpels | | | light | |
| 43 | ATGE_46 | development | clv3-7 | shoot apex, | 21+ | days | continuous | soil |
| | | baseline | | inflorescence | | | light | |
| | | | | (after bolting) | | | | |
| 44 | ATGE_47 | development | lfy-12 | shoot apex, | 21+ | days | continuous | soil |
| | | baseline | | inflorescence | | | light | |
| | | | | (after bolting) | | | | |
| 45 | ATGE_48 | development | ap1-15 | shoot apex, | 21+ | days | continuous | soil |
| | | baseline | | inflorescence | | | light | |
| | | | | (after bolting) | | | | |
| 46 | ATGE_49 | development | ap2-6 | shoot apex, | 21+ | days | continuous | soil |
| | | baseline | | inflorescence | | | light | |
| | | | | (after bolting) | | | | |
| 47 | ATGE_50 | development | ap3-6 | shoot apex, | 21+ | days | continuous | soil |
| | | baseline | | inflorescence | | | light | |
| | | | | (after bolting) | | | | |
| 48 | ATGE_51 | development | ag-12 | shoot apex, | 21+ | days | continuous | soil |
| | | baseline | | inflorescence | | | light | |
| | | | | (after bolting) | | | | |
| 49 | ATGE_52 | development | ufo-1 | shoot apex, | 21+ | days | continuous | soil |
| | | baseline | | inflorescence | | | light | |
| | | | | (after bolting) | | | | |
| 50 | ATGE_53 | development | clv3-7 | flower stage | 21+ | days | continuous | soil |
| | | baseline | | 12; multi-carpel | | | light | |
| | | | | gynoeceum; enlarged | | | | |
| | | | | meristem; increased | | | | |
| | | | | organ number | | | | |
| 51 | ATGE_54 | development | lfy-12 | flower stage | 21+ | days | continuous | soil |
| | | baseline | | 12; shoot | | | light | |
| | | | | characteristics; | | | | |
| | | | | most organs | | | | |
| | | | | leaf-like | | | | |
| 52 | ATGE_55 | development | ap1-15 | flower stage | 21+ | days | continuous | soil |
| | | baseline | | 12; sepals | | | light | |
| | | | | replaced by | | | | |
| | | | | leaf-like | | | | |
| | | | | organs, petals | | | | |
| | | | | mostly lacking, | | | | |
| | | | | 2° flowers | | | | |
| 53 | ATGE_56 | development | ap2-6 | flower stage | 21+ | days | continuous | soil |
| | | baseline | | 12; no sepals | | | light | |
| | | | | or petals | | | | |
| 54 | ATGE_57 | development | ap3-6 | flower stage | 21+ | days | continuous | soil |
| | | baseline | | 12; no petals | | | light | |
| | | | | or stamens | | | | |
| 55 | ATGE_58 | development | ag-12 | flower stage | 21+ | days | continuous | soil |
| | | baseline | | 12; no stamens | | | light | |
| | | | | or carpels | | | | |
| 56 | ATGE_59 | development | ufo-1 | flower stage | 21+ | days | continuous | soil |
| | | baseline | | 12; filamentous | | | light | |
| | | | | organs in whorls | | | | |
| | | | | two and three | | | | |
| 57 | ATGE_73 | pollen | Wt | mature pollen | 6 | wk | continuous | soil |
| | | | | | | | light | |
| 58 | ATGE_76 | seed & | Wt | siliques, w/ | 8 | wk | long day | soil |
| | | silique | | seeds stage 3; | | | (16/8) | |
| | | development | | mid globular to | | | | |
| | | | | early heart | | | | |
| | | | | embryos | | | | |
| 59 | ATGE_77 | seed & | Wt | siliques, w/ | 8 | wk | long day | soil |
| | | silique | | seeds stage 4; | | | (16/8) | |
| | | development | | early to late | | | | |
| | | | | heart embryos | | | | |
| 60 | ATGE_78 | seed & | Wt | siliques, w/ | 8 | wk | long day | soil |
| | | silique | | seeds stage 5; | | | (16/8) | |
| | | development | | late heart to | | | | |
| | | | | mid torpedo | | | | |
| | | | | embryos | | | | |
| 61 | ATGE_79 | seed & | Wt | seeds, stage 6, | 8 | wk | long day | soil |
| | | silique | | w/o siliques; | | | (16/8) | |
| | | development | | mid to late | | | | |
| | | | | torpedo embryos | | | | |
| 62 | ATGE_81 | seed & | Wt | seeds, stage 7, | 8 | wk | long day | soil |
| | | silique | | w/o siliques; | | | (16/8) | |
| | | development | | late torpedo to | | | | |
| | | | | early walking- | | | | |
| | | | | stick embryos | | | | |
| 63 | ATGE_82 | seed & | Wt | seeds, stage 8, | 8 | wk | long day | soil |
| | | silique | | w/o siliques; | | | (16/8) | |
| | | development | | walking-stick | | | | |
| | | | | to early curled | | | | |
| | | | | cotyledons | | | | |
| | | | | embryos | | | | |
| 64 | ATGE_83 | seed & | Wt | seeds, stage 9, | 8 | wk | long day | soil |
| | | silique | | w/o siliques; | | | (16/8) | |
| | | development | | curled | | | | |
| | | | | cotyledons to | | | | |
| | | | | early green | | | | |
| | | | | cotyledons | | | | |
| | | | | embryos | | | | |
| 65 | ATGE_84 | seed & | Wt | seeds, stage 10, | 8 | wk | long day | soil |
| | | silique | | w/o siliques; | | | (16/8) | |
| | | development | | green | | | | |
| | | | | cotyledons | | | | |
| | | | | embryos | | | | |
| 66 | ATGE_87 | phase change | Wt | vegetative | 7 | days | short day | soil |
| | | | | rosette | | | (10/14) | |
| 67 | ATGE_89 | phase change | Wt | vegetative | 14 | days | short day | soil |
| | | | | rosette | | | (10/14) | |
| 68 | ATGE_90 | phase change | Wt | vegetative | 21 | days | short day | soil |
| | | | | rosette | | | (10/14) | |
| 69 | ATGE_91 | comparison | Wt | leaf | 15 | days | long day | 1x MS |
| | | with CAGE | | | | | (16/8) | agar, 1% |
| | | | | | | | | sucrose |
| 70 | ATGE_92 | comparison | Wt | flower | 28 | days | long day | soil |
| | | with CAGE | | | | | (16/8) | |
| 71 | ATGE_93 | comparison | Wt | root | 15 | days | long day | 1x MS |
| | | with CAGE | | | | | (16/8) | agar, 1% |
| | | | | | | | | sucrose |
| 72 | ATGE_94 | development | Wt | root | 8 | days | continuous | 1x MS |
| | | on MS agar | | | | | light | agar |
| 73 | ATGE_95 | development | Wt | root | 8 | days | continuous | 1x MS |
| | | on MS agar | | | | | light | agar, 1% |
| | | | | | | | | sucrose |
| 74 | ATGE_96 | development | Wt | seedling, green | 8 | days | continuous | 1x MS |
| | | on MS agar | | parts | | | light | agar |
| 75 | ATGE_97 | development | Wt | seedling, green | 8 | days | continuous | 1x MS |
| | | on MS agar | | parts | | | light | agar, 1% |
| | | | | | | | | sucrose |
| 76 | ATGE_98 | development | Wt | root | 21 | days | continuous | 1x MS |
| | | on MS agar | | | | | light | agar |
| 77 | ATGE_99 | development | Wt | root | 21 | days | continuous | 1x MS |
| | | on MS agar | | | | | light | agar, 1% |
| | | | | | | | | sucrose |
| 78 | ATGE_100 | development | Wt | seedling, green | 21 | days | continuous | 1x MS |
| | | on MS agar | | parts | | | light | agar |
| 79 | ATGE_101 | development | Wt | seedling, green | 21 | days | continuous | 1x MS |
| | | on MS agar | | parts | | | light | agar, 1% |
| | | | | | | | | sucrose |

Plot D in each figure shows expression in response to abiotic stress as described by Kilian et al. (Plant J., 50: 347-363 (2007)). The data are presented as expression values from pairs of shoots (white bars) and roots (black bars) per treatment. A key for the samples in this dataset is presented in Table 5. The table identifies the codes that are used along the x-axis in plot D in each figure. The codes are presented in 4 digit format, where the first digit represents the treatment (i.e., control=0, cold=1, osmotic stress=2, etc.), the second digit represents the time point, the third digit represents the tissue (1=shoot and 2=root), and the fourth digit represents the replication number. Since the figures provide the averages of the first and second replication, the last digit is not shown in the figures.

TABLE 5
Abiotic Stress Key

| | | Time | | Sam- |
|---|---|---|---|---|
| Code | Treatment | point | Organ | ple |
| 0011 | Control | 0 h | Shoots | 1 |
| 0012 | Control | 0 h | Shoots | 2 |
| 0021 | Control | 0 h | Roots | 1 |
| 0022 | Control | 0 h | Roots | 2 |
| 0711 | Control | 0.25 h | Shoots | 1 |
| 0712 | Control | 0.25 h | Shoots | 2 |
| 0721 | Control | 0.25 h | Roots | 1 |
| 0722 | Control | 0.25 h | Roots | 2 |
| 0111 | Control | 0.5 h | Shoots | 1 |
| 0112 | Control | 0.5 h | Shoots | 2 |
| 0121 | Control | 0.5 h | Roots | 1 |
| 0122 | Control | 0.5 h | Roots | 2 |
| 0211 | Control | 1.0 h | Shoots | 1 |
| 0212 | Control | 1.0 h | Shoots | 2 |
| 0221 | Control | 1.0 h | Roots | 1 |
| 0222 | Control | 1.0 h | Roots | 2 |
| 0311 | Control | 3.0 h | Shoots | 1 |
| 0312 | Control | 3.0 h | Shoots | 2 |
| 0321 | Control | 3.0 h | Roots | 1 |
| 0322 | Control | 3.0 h | Roots | 2 |
| 0811 | Control | 4.0 h | Shoots | 1 |
| 0812 | Control | 4.0 h | Shoots | 2 |
| 0821 | Control | 4.0 h | Roots | 1 |
| 0822 | Control | 4.0 h | Roots | 2 |
| 0411 | Control | 6.0 h | Shoots | 1 |
| 0412 | Control | 6.0 h | Shoots | 2 |
| 0421 | Control | 6.0 h | Roots | 1 |
| 0422 | Control | 6.0 h | Roots | 2 |
| 0511 | Control | 12.0 h | Shoots | 1 |
| 0512 | Control | 12.0 h | Shoots | 2 |
| 0521 | Control | 12.0 h | Roots | 1 |
| 0522 | Control | 12.0 h | Roots | 2 |
| 0611 | Control | 24.0 h | Shoots | 1 |
| 0612 | Control | 24.0 h | Shoots | 2 |
| 0621 | Control | 24.0 h | Roots | 1 |
| 0622 | Control | 24.0 h | Roots | 2 |
| 1111 | Cold (4° C.) | 0.5 h | Shoots | 1 |
| 1112 | Cold (4° C.) | 0.5 h | Shoots | 2 |
| 1121 | Cold (4° C.) | 0.5 h | Roots | 1 |
| 1122 | Cold (4° C.) | 0.5 h | Roots | 2 |
| 1211 | Cold (4° C.) | 1.0 h | Shoots | 1 |
| 1212 | Cold (4° C.) | 1.0 h | Shoots | 2 |
| 1221 | Cold (4° C.) | 1.0 h | Roots | 1 |
| 1222 | Cold (4° C.) | 1.0 h | Roots | 2 |
| 1311 | Cold (4° C.) | 3.0 h | Shoots | 1 |
| 1312 | Cold (4° C.) | 3.0 h | Shoots | 2 |
| 1321 | Cold (4° C.) | 3.0 h | Roots | 1 |
| 1322 | Cold (4° C.) | 3.0 h | Roots | 2 |
| 1411 | Cold (4° C.) | 6.0 h | Shoots | 1 |
| 1412 | Cold (4° C.) | 6.0 h | Shoots | 2 |
| 1421 | Cold (4° C.) | 6.0 h | Roots | 1 |
| 1422 | Cold (4° C.) | 6.0 h | Roots | 2 |
| 1511 | Cold (4° C.) | 12.0 h | Shoots | 1 |
| 1512 | Cold (4° C.) | 12.0 h | Shoots | 2 |
| 1521 | Cold (4° C.) | 12.0 h | Roots | 1 |
| 1522 | Cold (4° C.) | 12.0 h | Roots | 2 |
| 1611 | Cold (4° C.) | 24.0 h | Shoots | 1 |
| 1612 | Cold (4° C.) | 24.0 h | Shoots | 2 |
| 1621 | Cold (4° C.) | 24.0 h | Roots | 1 |
| 1622 | Cold (4° C.) | 24.0 h | Roots | 2 |
| 2111 | Osmotic stress | 0.5 h | Shoots | 1 |
| 2112 | Osmotic stress | 0.5 h | Shoots | 2 |
| 2121 | Osmotic stress | 0.5 h | Roots | 1 |
| 2122 | Osmotic stress | 0.5 h | Roots | 2 |
| 2211 | Osmotic stress | 1.0 h | Shoots | 1 |
| 2212 | Osmotic stress | 1.0 h | Shoots | 2 |
| 2221 | Osmotic stress | 1.0 h | Roots | 1 |
| 2222 | Osmotic stress | 1.0 h | Roots | 2 |
| 2311 | Osmotic stress | 3.0 h | Shoots | 1 |
| 2312 | Osmotic stress | 3.0 h | Shoots | 2 |
| 2321 | Osmotic stress | 3.0 h | Roots | 1 |
| 2322 | Osmotic stress | 3.0 h | Roots | 2 |
| 2411 | Osmotic stress | 6.0 h | Shoots | 1 |
| 2412 | Osmotic stress | 6.0 h | Shoots | 2 |
| 2421 | Osmotic stress | 6.0 h | Roots | 1 |
| 2422 | Osmotic stress | 6.0 h | Roots | 2 |
| 2511 | Osmotic stress | 12.0 h | Shoots | 1 |
| 2512 | Osmotic stress | 12.0 h | Shoots | 2 |
| 2521 | Osmotic stress | 12.0 h | Roots | 1 |
| 2522 | Osmotic stress | 12.0 h | Roots | 2 |
| 2611 | Osmotic stress | 24.0 h | Shoots | 1 |
| 2612 | Osmotic stress | 24.0 h | Shoots | 2 |
| 2621 | Osmotic stress | 24.0 h | Roots | 1 |
| 2622 | Osmotic stress | 24.0 h | Roots | 2 |
| 3111 | Salt stress | 0.5 h | Shoots | 1 |
| 3112 | Salt stress | 0.5 h | Shoots | 2 |
| 3121 | Salt stress | 0.5 h | Roots | 1 |
| 3122 | Salt stress | 0.5 h | Roots | 2 |
| 3211 | Salt stress | 1.0 h | Shoots | 1 |
| 3212 | Salt stress | 1.0 h | Shoots | 2 |
| 3221 | Salt stress | 1.0 h | Roots | 1 |
| 3222 | Salt stress | 1.0 h | Roots | 2 |
| 3311 | Salt stress | 3.0 h | Shoots | 1 |
| 3312 | Salt stress | 3.0 h | Shoots | 2 |
| 3321 | Salt stress | 3.0 h | Roots | 1 |
| 3322 | Salt stress | 3.0 h | Roots | 2 |
| 3411 | Salt stress | 6.0 h | Shoots | 1 |
| 3412 | Salt stress | 6.0 h | Shoots | 2 |
| 3421 | Salt stress | 6.0 h | Roots | 1 |
| 3422 | Salt stress | 6.0 h | Roots | 2 |
| 3511 | Salt stress | 12.0 h | Shoots | 1 |
| 3512 | Salt stress | 12.0 h | Shoots | 2 |
| 3521 | Salt stress | 12.0 h | Roots | 1 |
| 3522 | Salt stress | 12.0 h | Roots | 2 |
| 3611 | Salt stress | 24.0 h | Shoots | 1 |
| 3612 | Salt stress | 24.0 h | Shoots | 2 |
| 3621 | Salt stress | 24.0 h | Roots | 1 |
| 3622 | Salt stress | 24.0 h | Roots | 2 |
| 4711 | Drought stress | 0.25 h | Shoots | 1 |
| 4712 | Drought stress | 0.25 h | Shoots | 2 |
| 4721 | Drought stress | 0.25 h | Roots | 1 |
| 4722 | Drought stress | 0.25 h | Roots | 2 |
| 4111 | Drought stress | 0.5 h | Shoots | 1 |
| 4112 | Drought stress | 0.5 h | Shoots | 2 |
| 4121 | Drought stress | 0.5 h | Roots | 1 |
| 4122 | Drought stress | 0.5 h | Roots | 2 |
| 4211 | Drought stress | 1.0 h | Shoots | 1 |
| 4212 | Drought stress | 1.0 h | Shoots | 2 |
| 4221 | Drought stress | 1.0 h | Roots | 1 |
| 4222 | Drought stress | 1.0 h | Roots | 2 |
| 4311 | Drought stress | 3.0 h | Shoots | 1 |
| 4312 | Drought stress | 3.0 h | Shoots | 2 |
| 4321 | Drought stress | 3.0 h | Roots | 1 |
| 4322 | Drought stress | 3.0 h | Roots | 2 |
| 4411 | Drought stress | 6.0 h | Shoots | 1 |
| 4412 | Drought stress | 6.0 h | Shoots | 2 |
| 4421 | Drought stress | 6.0 h | Roots | 1 |
| 4422 | Drought stress | 6.0 h | Roots | 2 |
| 4511 | Drought stress | 12.0 h | Shoots | 1 |
| 4512 | Drought stress | 12.0 h | Shoots | 2 |
| 4521 | Drought stress | 12.0 h | Roots | 1 |
| 4522 | Drought stress | 12.0 h | Roots | 2 |
| 4611 | Drought stress | 24.0 h | Shoots | 1 |
| 4612 | Drought stress | 24.0 h | Shoots | 2 |
| 4621 | Drought stress | 24.0 h | Roots | 1 |
| 4622 | Drought stress | 24.0 h | Roots | 2 |
| 5111 | Genotoxic stress | 0.5 h | Shoots | 1 |
| 5112 | Genotoxic stress | 0.5 h | Shoots | 2 |
| 5121 | Genotoxic stress | 0.5 h | Roots | 1 |
| 5122 | Genotoxic stress | 0.5 h | Roots | 2 |
| 5211 | Genotoxic stress | 1.0 h | Shoots | 1 |
| 5212 | Genotoxic stress | 1.0 h | Shoots | 2 |
| 5221 | Genotoxic stress | 1.0 h | Roots | 1 |
| 5222 | Genotoxic stress | 1.0 h | Roots | 2 |
| 5311 | Genotoxic stress | 3.0 h | Shoots | 1 |
| 5312 | Genotoxic stress | 3.0 h | Shoots | 2 |
| 5321 | Genotoxic stress | 3.0 h | Roots | 1 |
| 5322 | Genotoxic stress | 3.0 h | Roots | 2 |
| 5411 | Genotoxic stress | 6.0 h | Shoots | 1 |
| 5412 | Genotoxic stress | 6.0 h | Shoots | 2 |
| 5421 | Genotoxic stress | 6.0 h | Roots | 1 |
| 5422 | Genotoxic stress | 6.0 h | Roots | 2 |
| 5511 | Genotoxic stress | 12.0 h | Shoots | 1 |
| 5512 | Genotoxic stress | 12.0 h | Shoots | 2 |
| 5521 | Genotoxic stress | 12.0 h | Roots | 1 |
| 5522 | Genotoxic stress | 12.0 h | Roots | 2 |
| 5611 | Genotoxic stress | 24.0 h | Shoots | 1 |
| 5612 | Genotoxic stress | 24.0 h | Shoots | 2 |
| 5621 | Genotoxic stress | 24.0 h | Roots | 1 |
| 5622 | Genotoxic stress | 24.0 h | Roots | 2 |
| 6111 | Oxidative stress | 0.5 h | Shoots | 1 |
| 6112 | Oxidative stress | 0.5 h | Shoots | 2 |
| 6124 | Oxidative stress | 0.5 h | Roots | 1 |
| 6122 | Oxidative stress | 0.5 h | Roots | 2 |
| 6211 | Oxidative stress | 1.0 h | Shoots | 1 |
| 6212 | Oxidative stress | 1.0 h | Shoots | 2 |
| 6223 | Oxidative stress | 1.0 h | Roots | 1 |
| 6224 | Oxidative stress | 1.0 h | Roots | 2 |
| 6311 | Oxidative stress | 3.0 h | Shoots | 1 |
| 6312 | Oxidative stress | 3.0 h | Shoots | 2 |
| 6323 | Oxidative stress | 3.0 h | Roots | 1 |
| 6322 | Oxidative stress | 3.0 h | Roots | 2 |
| 6411 | Oxidative stress | 6.0 h | Shoots | 1 |
| 6412 | Oxidative stress | 6.0 h | Shoots | 2 |
| 6421 | Oxidative stress | 6.0 h | Roots | 1 |
| 6422 | Oxidative stress | 6.0 h | Roots | 2 |
| 6511 | Oxidative stress | 12.0 h | Shoots | 1 |
| 6512 | Oxidative stress | 12.0 h | Shoots | 2 |
| 6523 | Oxidative stress | 12.0 h | Roots | 1 |
| 6524 | Oxidative stress | 12.0 h | Roots | 2 |
| 6611 | Oxidative stress | 24.0 h | Shoots | 1 |
| 6612 | Oxidative stress | 24.0 h | Shoots | 2 |
| 6621 | Oxidative stress | 24.0 h | Roots | 1 |
| 6622 | Oxidative stress | 24.0 h | Roots | 2 |
| 7711 | UV-B stress | 0.25 h | Shoots | 1 |
| 7712 | UV-B stress | 0.25 h | Shoots | 2 |
| 7721 | UV-B stress | 0.25 h | Roots | 1 |
| 7722 | UV-B stress | 0.25 h | Roots | 2 |
| 7111 | UV-B stress | 0.5 h | Shoots | 1 |
| 7112 | UV-B stress | 0.5 h | Shoots | 2 |
| 7121 | UV-B stress | 0.5 h | Roots | 1 |
| 7122 | UV-B stress | 0.5 h | Roots | 2 |
| 7211 | UV-B stress | 1.0 h | Shoots | 1 |
| 7212 | UV-B stress | 1.0 h | Shoots | 2 |
| 7221 | UV-B stress | 1.0 h | Roots | 1 |
| 7222 | UV-B stress | 1.0 h | Roots | 2 |
| 7311 | UV-B stress | 3.0 h | Shoots | 1 |
| 7312 | UV-B stress | 3.0 h | Shoots | 2 |
| 7321 | UV-B stress | 3.0 h | Roots | 1 |
| 7322 | UV-B stress | 3.0 h | Roots | 2 |
| 7411 | UV-B stress | 6.0 h | Shoots | 1 |
| 7412 | UV-B stress | 6.0 h | Shoots | 2 |
| 7421 | UV-B stress | 6.0 h | Roots | 1 |
| 7422 | UV-B stress | 6.0 h | Roots | 2 |
| 7511 | UV-B stress | 12.0 h | Shoots | 1 |
| 7512 | UV-B stress | 12.0 h | Shoots | 2 |
| 7521 | UV-B stress | 12.0 h | Roots | 1 |
| 7522 | UV-B stress | 12.0 h | Roots | 2 |
| 7611 | UV-B stress | 24.0 h | Shoots | 1 |
| 7612 | UV-B stress | 24.0 h | Shoots | 2 |
| 7621 | UV-B stress | 24.0 h | Roots | 1 |
| 7622 | UV-B stress | 24.0 h | Roots | 2 |
| 8715 | Wounding stress | 0.25 h | Shoots | 1 |
| 8712 | Wounding stress | 0.25 h | Shoots | 2 |
| 8723 | Wounding stress | 0.25 h | Roots | 1 |
| 8724 | Wounding stress | 0.25 h | Roots | 2 |
| 8111 | Wounding stress | 0.5 h | Shoots | 1 |
| 8112 | Wounding stress | 0.5 h | Shoots | 2 |
| 8124 | Wounding stress | 0.5 h | Roots | 1 |
| 8126 | Wounding stress | 0.5 h | Roots | 2 |
| 8211 | Wounding stress | 1.0 h | Shoots | 1 |
| 8214 | Wounding stress | 1.0 h | Shoots | 2 |
| 8224 | Wounding stress | 1.0 h | Roots | 1 |
| 8225 | Wounding stress | 1.0 h | Roots | 2 |
| 8313 | Wounding stress | 3.0 h | Shoots | 1 |
| 8314 | Wounding stress | 3.0 h | Shoots | 2 |
| 8324 | Wounding stress | 3.0 h | Roots | 1 |
| 8325 | Wounding stress | 3.0 h | Roots | 2 |
| 8411 | Wounding stress | 6.0 h | Shoots | 1 |
| 8412 | Wounding stress | 6.0 h | Shoots | 2 |
| 8423 | Wounding stress | 6.0 h | Roots | 1 |
| 8424 | Wounding stress | 6.0 h | Roots | 2 |
| 8511 | Wounding stress | 12.0 h | Shoots | 1 |
| 8512 | Wounding stress | 12.0 h | Shoots | 2 |
| 8524 | Wounding stress | 12.0 h | Roots | 1 |
| 8525 | Wounding stress | 12.0 h | Roots | 2 |
| 8611 | Wounding stress | 24.0 h | Shoots | 1 |
| 8612 | Wounding stress | 24.0 h | Shoots | 2 |
| 8624 | Wounding stress | 24.0 h | Roots | 1 |
| 8624_repl_8623 | Wounding stress | 24.0 h | Roots | 2 |
| 9711 | Heat stress | 0.25 h | Shoots | 1 |
| 9712 | Heat stress | 0.25 h | Shoots | 2 |
| 9721 | Heat stress | 0.25 h | Roots | 1 |
| 9722 | Heat stress | 0.25 h | Roots | 2 |
| 9111 | Heat stress | 0.5 h | Shoots | 1 |
| 9112 | Heat stress | 0.5 h | Shoots | 2 |
| 9121 | Heat stress | 0.5 h | Roots | 1 |
| 9122 | Heat stress | 0.5 h | Roots | 2 |
| 9211 | Heat stress | 1.0 h | Shoots | 1 |
| 9212 | Heat stress | 1.0 h | Shoots | 2 |
| 9221 | Heat stress | 1.0 h | Roots | 1 |
| 9222 | Heat stress | 1.0 h | Roots | 2 |
| 9311 | Heat stress | 3.0 h | Shoots | 1 |
| 9312 | Heat stress | 3.0 h | Shoots | 2 |
| 9321 | Heat stress | 3.0 h | Roots | 1 |
| 9322 | Heat stress | 3.0 h | Roots | 2 |
| 9811 | Heat stress | 4.0 h | Shoots | 1 |
| | (3 h) + 1 h | | | |
| 9812 | Heat stress | 4.0 h | Shoots | 2 |
| | (3 h) + 1 h | | | |
| 9821 | Heat stress | 4.0 h | Roots | 1 |
| | (3 h) + 1 h | | | |
| 9822 | Heat stress | 4.0 h | Roots | 2 |
| | (3 h) + 1 h | | | |
| 9411 | Heat stress | 6.0 h | Shoots | 1 |
| | (3 h) + 3 h | | | |
| 9412 | Heat stress | 6.0 h | Shoots | 2 |
| | (3 h) + 3 h | | | |
| 9421 | Heat stress | 6.0 h | Roots | 1 |
| | (3 h) + 3 h | | | |
| 9422 | Heat stress | 6.0 h | Roots | 2 |
| | (3 h) + 3 h | | | |
| 9511 | Heat stress | 12.0 h | Shoots | 1 |
| | (3 h) + 9 h | | | |
| 9512 | Heat stress | 12.0 h | Shoots | 2 |
| | (3 h) + 9 h | | | |
| 9521 | Heat stress | 12.0 h | Roots | 1 |
| | (3 h) + 9 h | | | |
| 9522 | Heat stress | 12.0 h | Roots | 2 |
| | (3 h) + 9 h | | | |
| 9611 | Heat stress | 24.0 h | Shoots | 1 |
| | (3 h) + 21 h | | | |
| 9612 | Heat stress | 24.0 h | Shoots | 2 |
| | (3 h) + 21 h | | | |
| 9621 | Heat stress | 24.0 h | Roots | 1 |
| | (3 h) + 21 h | | | |
| 9622 | Heat stress | 24.0 h | Roots | 2 |
| | (3 h) + 21 h | | | |
| C0_1 | Control | 0 h | Cell culture | 1 |
| C0_2 | Control | 0 h | Cell culture | 2 |
| C1_1 | Control | 3.0 h | Cell culture | 1 |
| C1_2 | Control | 3.0 h | Cell culture | 2 |
| C2_1 | Control | 6.0 h | Cell culture | 1 |
| C2_2 | Control | 6.0 h | Cell culture | 2 |
| C3_1 | Control | 12.0 h | Cell culture | 1 |
| C3_2 | Control | 12.0 h | Cell culture | 2 |
| C4_1 | Control | 24.0 h | Cell culture | 1 |
| C4_2 | Control | 24.0 h | Cell culture | 2 |
| C5_1 | Heat stress | 0.25 h | Cell culture | 1 |
| C5_2 | Heat stress | 0.25 h | Cell culture | 2 |
| C6_1 | Heat stress | 0.5 h | Cell culture | 1 |
| C6_2 | Heat stress | 0.5 h | Cell culture | 2 |
| C7_1 | Heat stress | 1.0 h | Cell culture | 1 |
| C7_2 | Heat stress | 1.0 h | Cell culture | 2 |
| C8_1 | Heat stress | 3.0 h | Cell culture | 1 |
| C8_2 | Heat stress | 3.0 h | Cell culture | 2 |
| C9_1 | Heat stress | 4.0 h | Cell culture | 1 |
| | (3 h) + 1 h | | | |
| C9_2 | Heat stress | 4.0 h | Cell culture | 2 |
| | (3 h) + 1 h | | | |
| C10_1 | Heat stress | 6.0 h | Cell culture | 1 |
| | (3 h) + 3 h | | | |
| C10_2 | Heat stress | 6.0 h | Cell culture | 2 |
| | (3 h) + 3 h | | | |
| C11_1 | Heat stress | 12.0 h | Cell culture | 1 |
| | (3 h) + 9 h | | | |
| C11_2 | Heat stress | 12.0 h | Cell culture | 2 |
| | (3 h) + 9 h | | | |
| C12_1 | Heat stress | 24.0 h | Cell culture | 1 |
| | (3 h) + 21 h | | | |
| C12_2 | Heat stress | 24.0 h | Cell culture | 2 |
| | (3 h) + 21 h | | | |

Treatment Codes
0 - Control plants, Group Kudla
The plants were treated like the treated plants; e.g.: Transfer of Magenta boxes out of the climate chamber. Opening of the boxes and lifting the raft as long as the treatments last. Then boxes were transferred back to the climate chamber.
1 - Cold stress (4° C.), Group Kudla
The Magenta boxes were placed on ice in the cold room (4° C.). The environmental light intensity was 20 μEinstein/cm2 sec. An extra light which was installed over the plants had 40 μEinstein/cm2 sec. The plants stayed there.
2 - Osmotic stress, Group Kudla
Mannitol was added to a concentration of 300 mM in the Media. To add Mannitol the raft was lifted out A magnetic stir bar and a stirrer were used to mix the media and the added Mannitol. After the rafts were put back in the boxes, they were transferred back to the climate chamber.
3 - Salt stress, Group Kudla
NaCl was added to a concentration of 150 mM in the Media. To add NaCl the raft was lifted out. A magnetic stir bar and a stirrer were used to mix the media and the added NaCl. After the rafts were put back in the boxes, they were transferred back to the climate chamber.
4 - Drought stress, Group Kudla
The plants were stressed by 15 min. dry air stream (clean bench) until 10% loss of fresh weight; then incubation in closed vessels in the climate chamber.
5 - Genotoxic stress, Group Puchta
Bleomycin + mitomycin (1.5 μg/ml bleomycin + 22 μg/ml mitomycin), were added to the indicated concentration in the Media. To add the reagents the raft was lifted out A magnetic stir bar and a stirrer were used to mix the media and the added reagents. After the rafts were put back in the boxes, they were transferred back to the climate chamber.
6 - Oxidative stress, Group Bartels
Methyl Viologen was added to a final concentration of 10 μM in the Media. To add the reagent the raft was lifted out A magnetic stir bar and a stirrer were used to mix the media and the added reagent. After the rafts were put back in the boxes, they were transferred back to the climate chamber.
7 - UV-B stress, Group Harter
15 min. 1.18 W/m2 Philips TL40W/12
8 - Wounding stress, Group Harter
Punctured with pins
9 - Beat stress, Group Nover/von Koskull-Döring
38° C., samples taken at 0.25, 0.5, 1.0, 3.0 h of hs and +1, +3, +9, +21 h recovery at 25° C.
C. - Beat stressed suspension culture, Group Nover/von Koskull-Döring
38° C., samples taken at 0.25, 0.5, 1.0, 3.0 h of hs and +1, +3, +9, +21 h recovery at 25° C.

### Example 3

### Testing Expression Using Identified Regulatory Polynucleotides

Regulatory polynucleotide molecules may be tested using transient expression assays using tissue bombardment and protoplast transfections following standard protocols. Reporter constructs including the respective candidate regulatory polynucleotide molecules linked to GUS are prepared and bombarded into Arabidopsis tissue obtained from different plant organs using a PDS-1000 Gene Gun (BioRad). GUS expression is assayed to confirm expression from the candidate promoters.

To further assess the candidate regulatory polynucleotide molecules in stable transformed plants, the candidate molecules are synthesized and cloned into commercially available constructs using the manufacturer's instructions. Regulatory polynucleotide:: GFP fusions are generated in a binary vector containing a selectable marker using commercially available vectors and methods, such as those previously described (J. Y. Lee et al., Proc Natl Acad Sci USA 103, 6055 (Apr. 11, 2006)). The final constructs are transferred to Agrobacterium for transformation into Columbia ecotype plants by the floral dip method (S. J. Clough, A. F. Bent, Plant J 16, 735 (December, 1998)). Transformed plants (T1) are selected by growth in the presence of the appropriate antibiotic or herbicide. Following selection, transformants are transferred to MS plates and allowed to recover.

For preliminary analysis, T1 root tips are excised, stained with propidium iodide and imaged for GFP fluorescence with a Zeiss 510 confocal microscope. Multiple T1 plants are analyzed per construct and multiple images along the longitudinal axis are taken in order to assess expression in the meristematic, elongation, and maturation zones of the root. In some cases expression may not be detectable as GFP fluorescence, but may detectable by qRT-PCR due to the higher sensitivity of the latter technique. Thus, qRT-PCR may also be used to detect the expression of GFP.

### Example 4

### Identification of Rice Regulatory Sequences

Several strategies were used to identify rice regulatory sequences.

In one strategy, aerial and root expression data of various rice genes was analyzed using two publically available rice Affymetrix datasets (Hirose et al. Plant Cell Physiol., 48: 523-539 (2007) and Jain et al. Plant Physiol., 143: 1467-1483 (2007)). The genes were filtered by requiring higher expression in root tissues than in most or all aerial tissues and agreement between the two data sets. This resulted in the identification of putative tissue-specific rice candidate genes.

In a second strategy, the Gramene.org database was queried to identify rice (Oryza sativa japonica) orthologs corresponding to Arabidopsis genes whose regulatory elements were identified as having tissue-specific activity (i.e., rice orthologs corresponding to Arabidopsis genes selected in Example 1 above or corresponding to Arabidopsis genes selected using methods described in Example 1 above but not listed in Example 1). In some cases, the Arabidopsis genes may lack a rice ortholog and in other cases the Arabidopsis genes may have more than one ortholog. As this strategy does not take any rice expression data into consideration, additional bioinformatics analyses (as described in the first strategy) were used to further identify rice orthologs that exhibit tissue-specific expression. In some cases where no rice expression data was available, the rice orthologs were chosen based on expression of the corresponding Arabidopsis orthologs.

To identify regulatory polynucleotide sequences responsible for driving tissue specific expression of all candidate rice genes, upstream sequences of 1500 bp or less of the selected gene candidates were determined. Because transcription start sites are not always known, sequences upstream of the translation start site were used in all cases. Therefore, the identified regulatory polynucleotides contain an endogenous 5′-UTR, and some of the endogenous 5′-UTRs may contain introns. The use of such introns in expression constructs containing these regulatory molecules may increase expression through IME. Without being limited by theory, because IME may be important for highly expressed constitutive genes, it is believed that IME may also play a role in the expression of genes expressed in a tissue-specific manner. In order to capture these regulatory sequences in genes that do not contain a 5′-UTR intron, chimeric regulatory polynucleotide molecules may be constructed wherein the first intron from the gene in question is fused to the 3′-end of the 5′-UTR of the regulatory polynucleotide (which may be from the same or a different (e.g. exogenous) gene). In order to ensure efficient intron splicing, the introns in these chimeric sequences may be flanked by consensus splice sites.

These strategies resulted in a list of rice regulatory sequences listed in Table 6, with the corresponding tissue-specific category (as explained in Example 1) listed (sequences including the regulatory polynucleotides plus the first intron from the coding region added at the 3′ end of the 5′ UTR are indicated by the corresponding gene accession number and the indicator “+intron”). Where there is a known Arabidopsis ortholog in Table 1, it is listed.

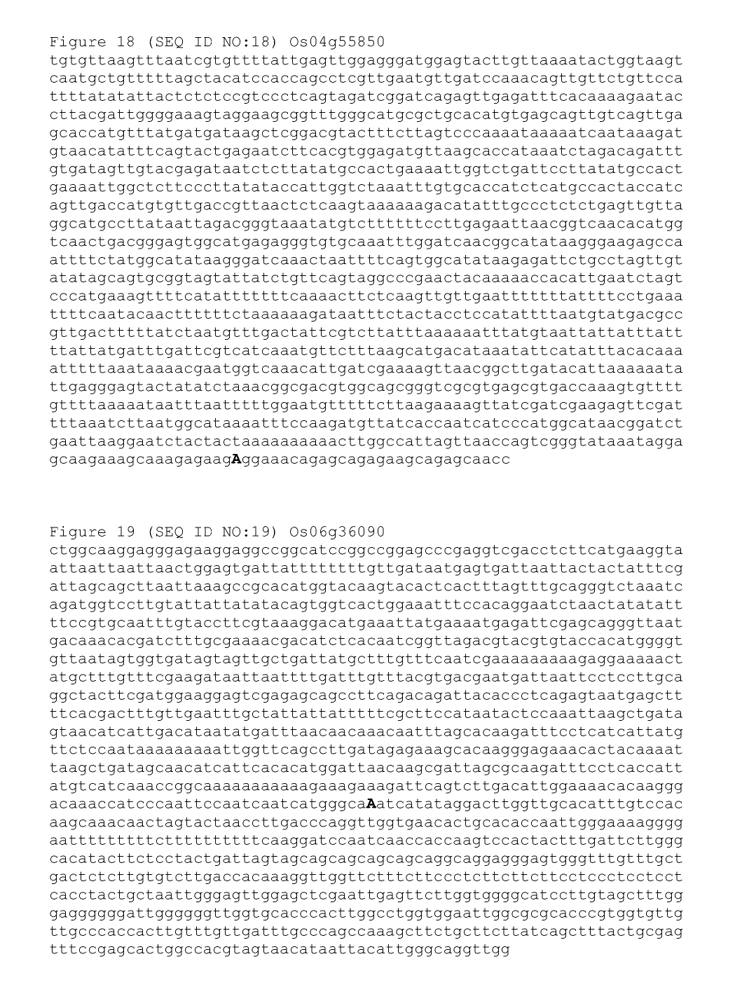

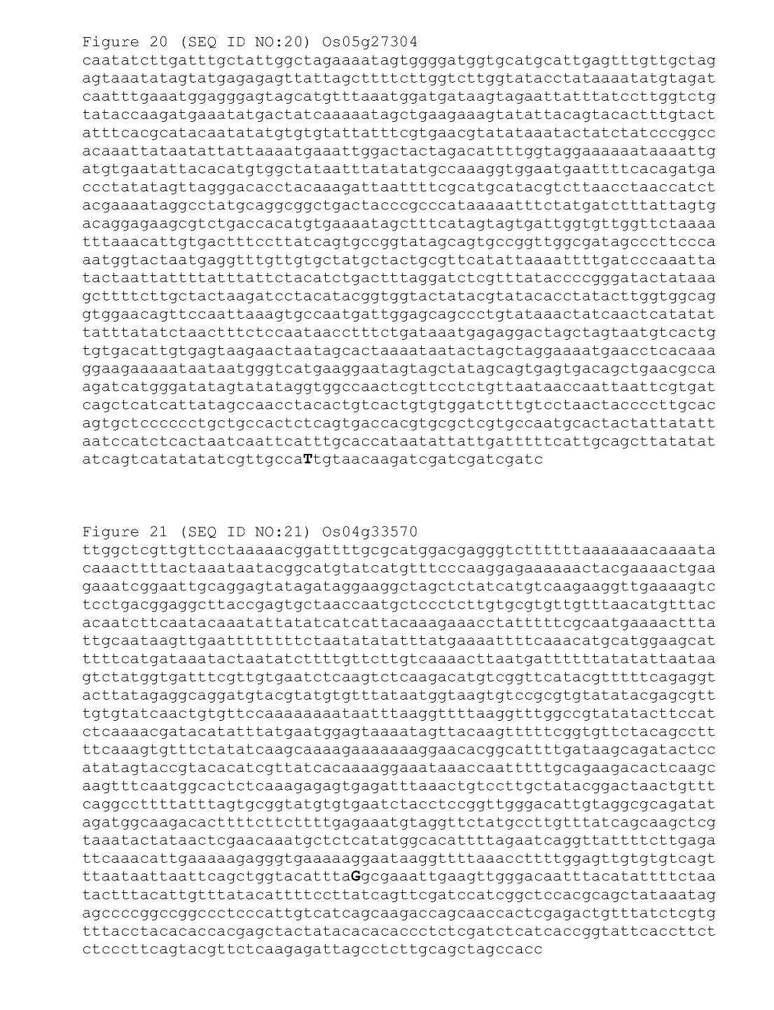

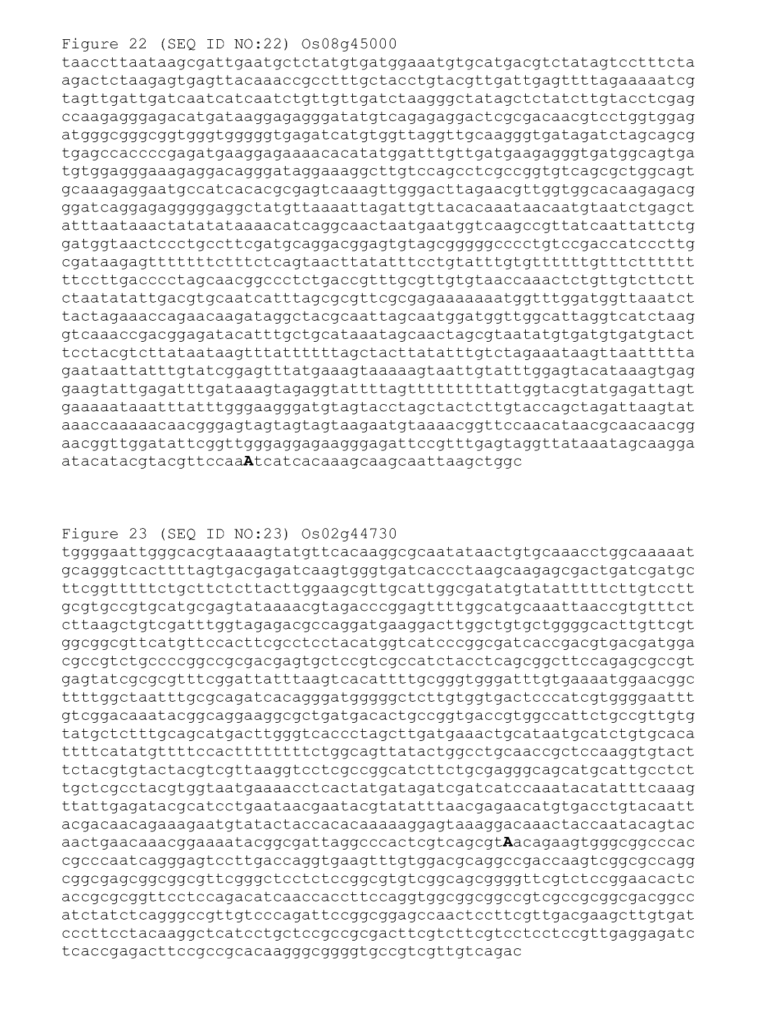

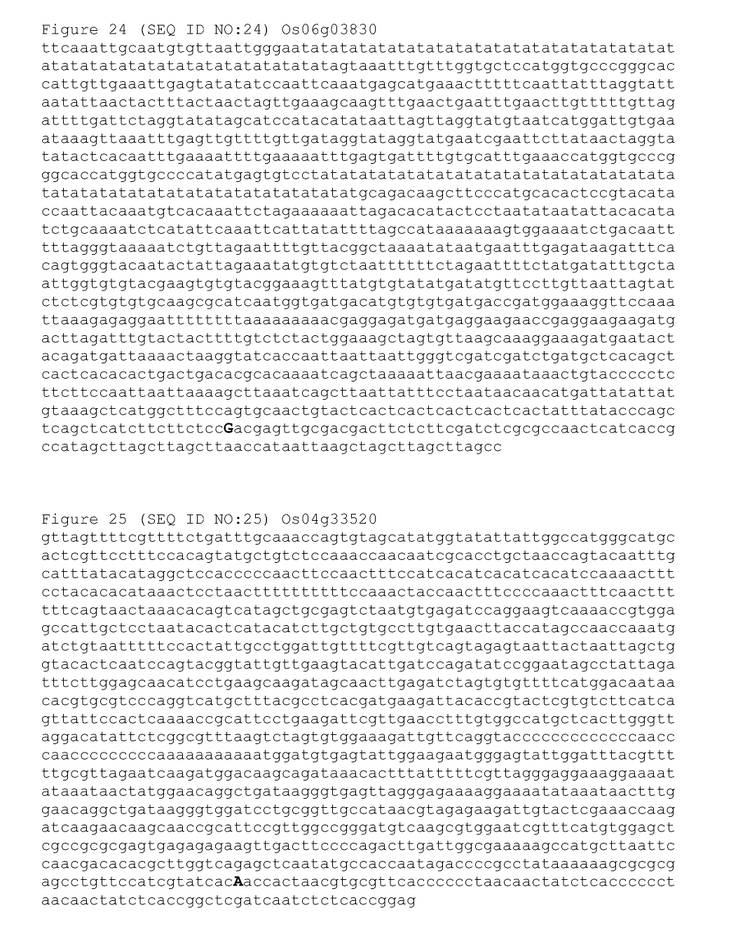

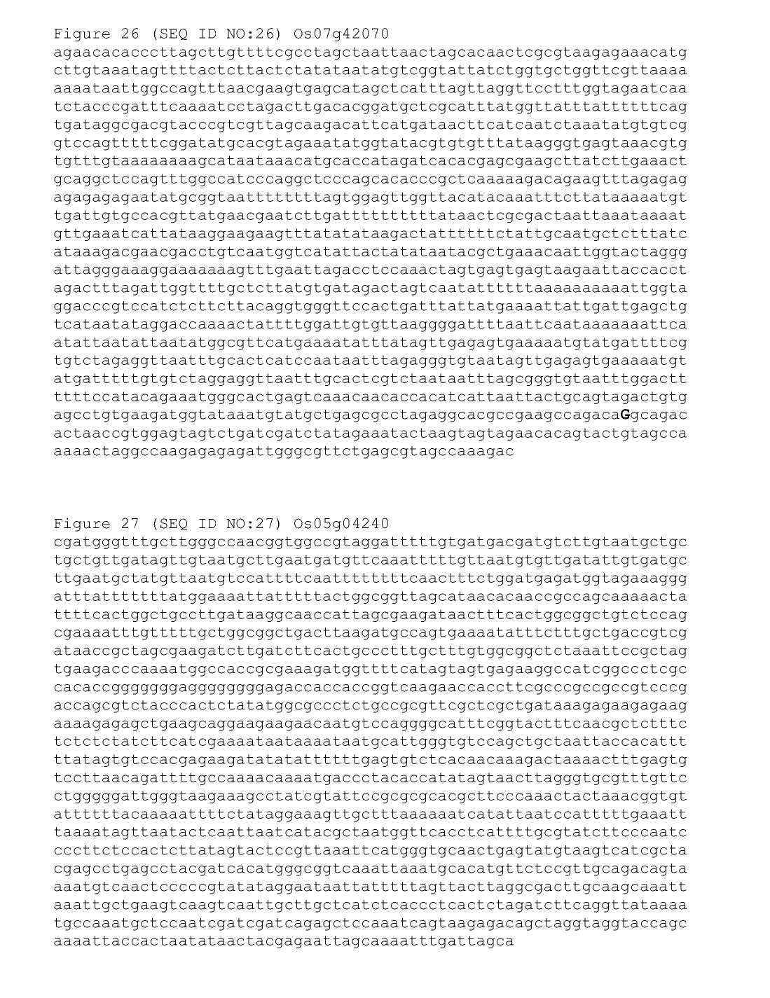

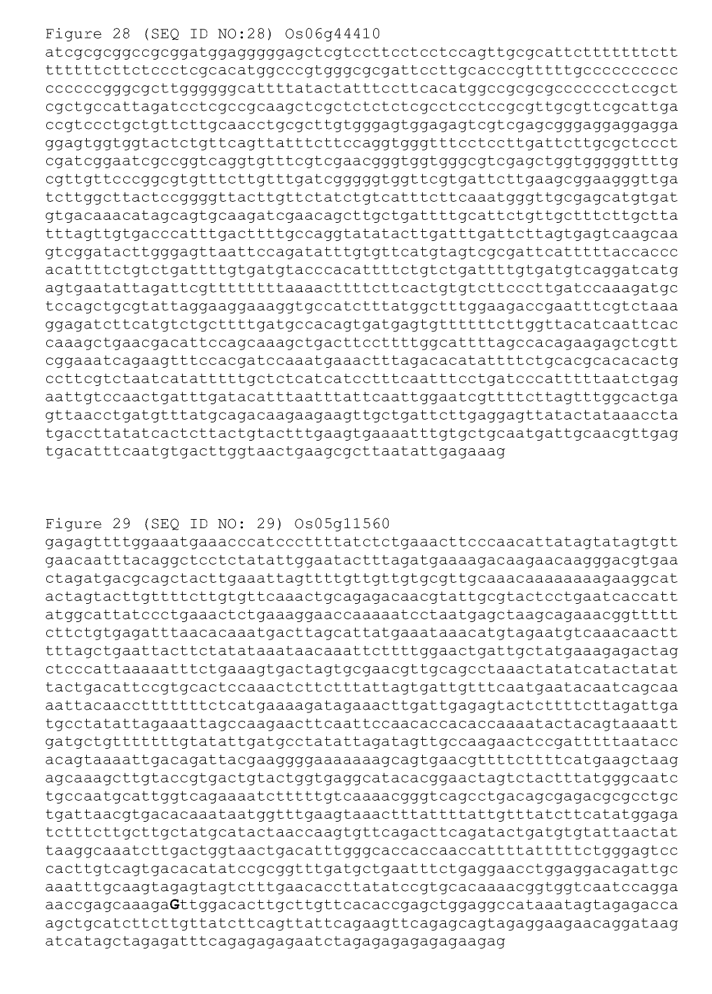

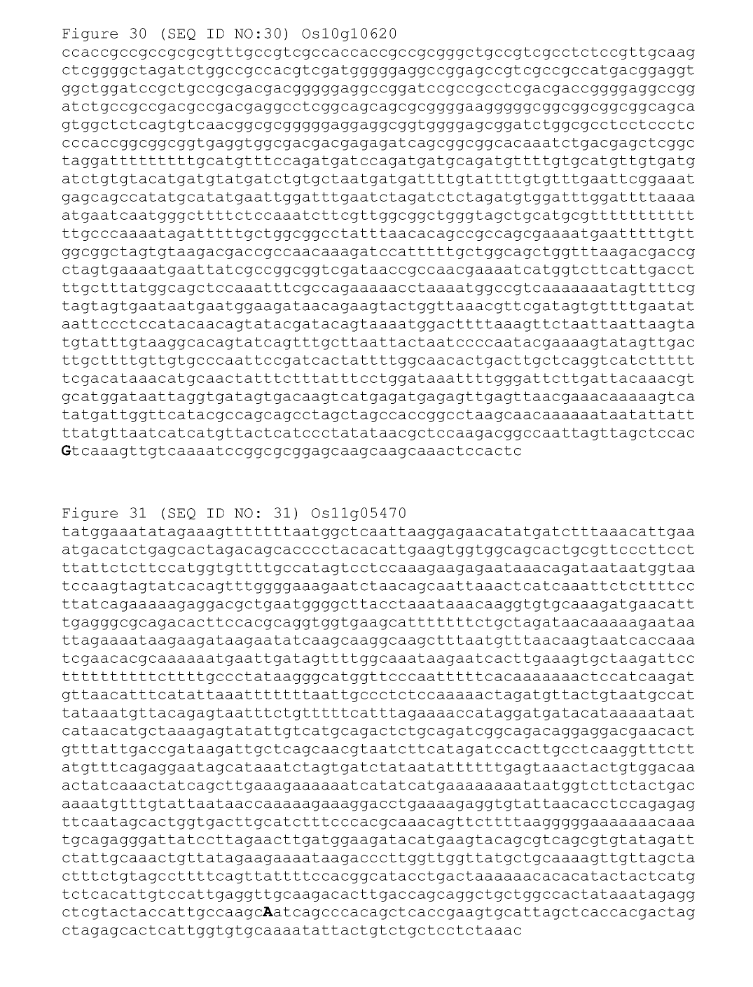

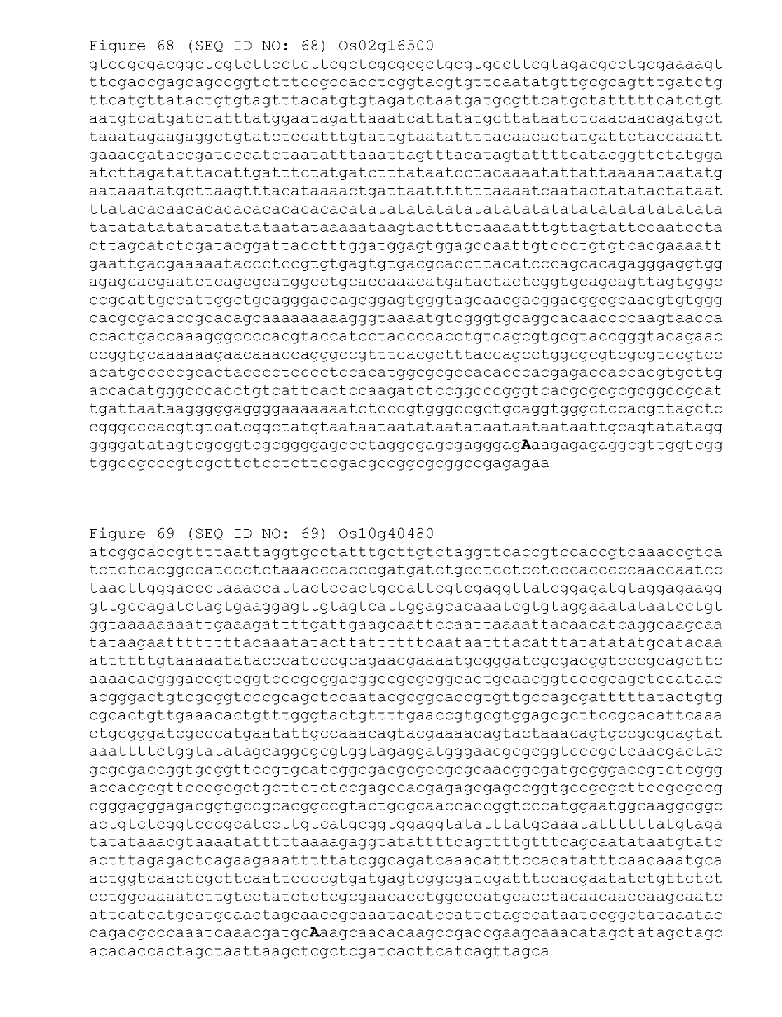

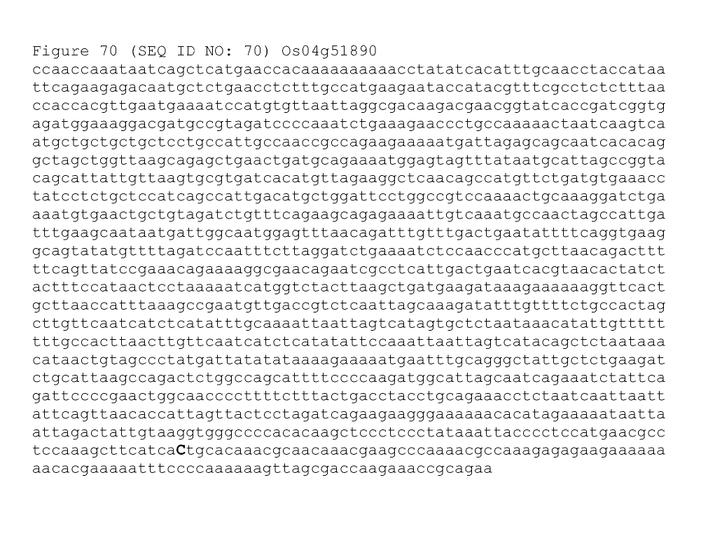

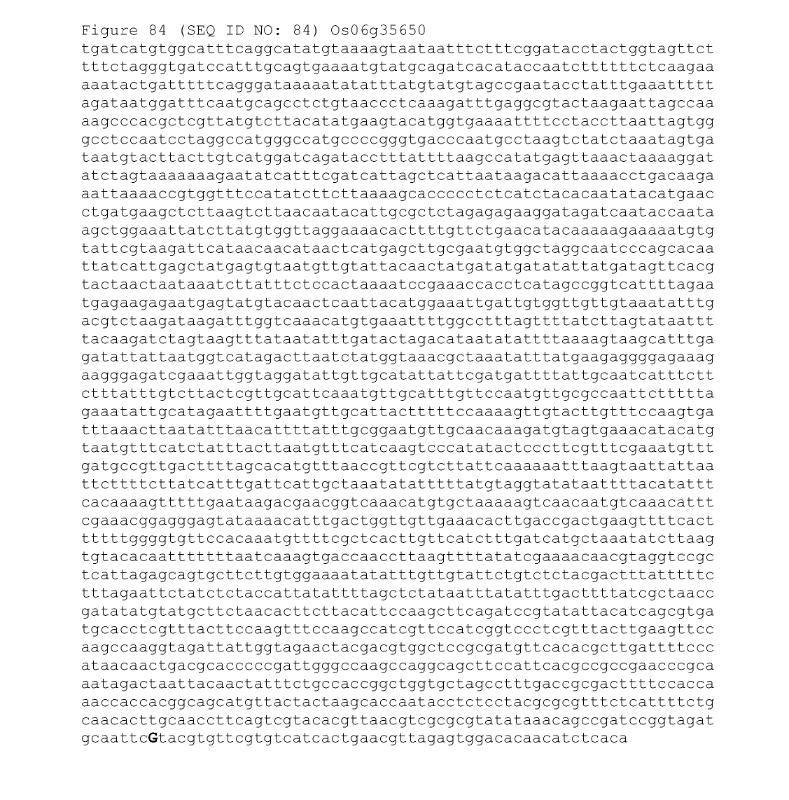

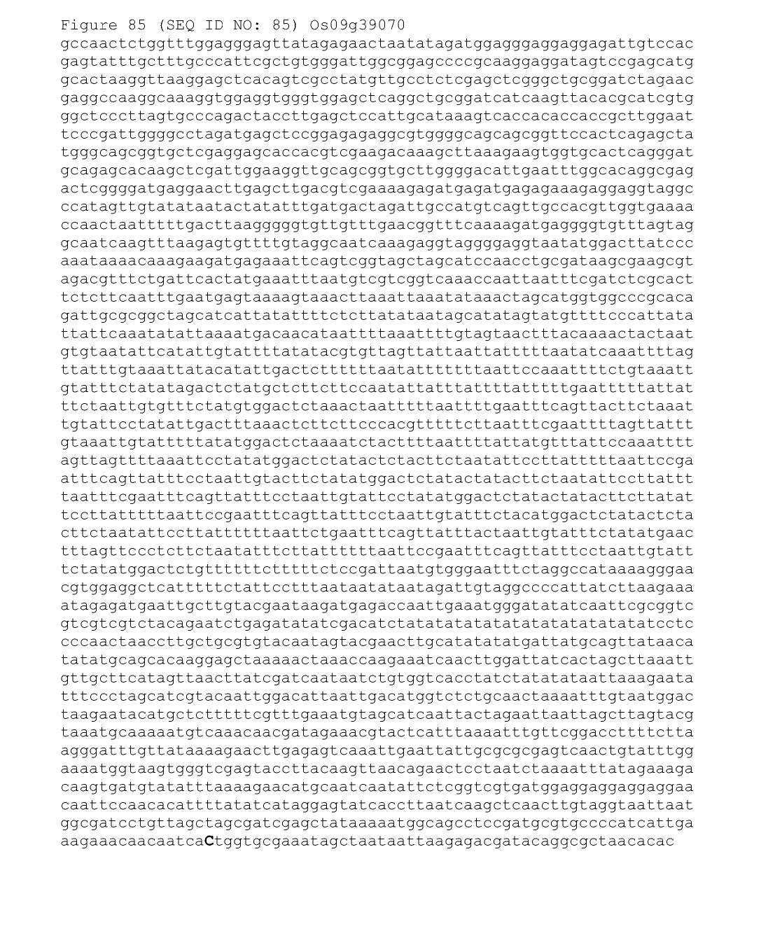

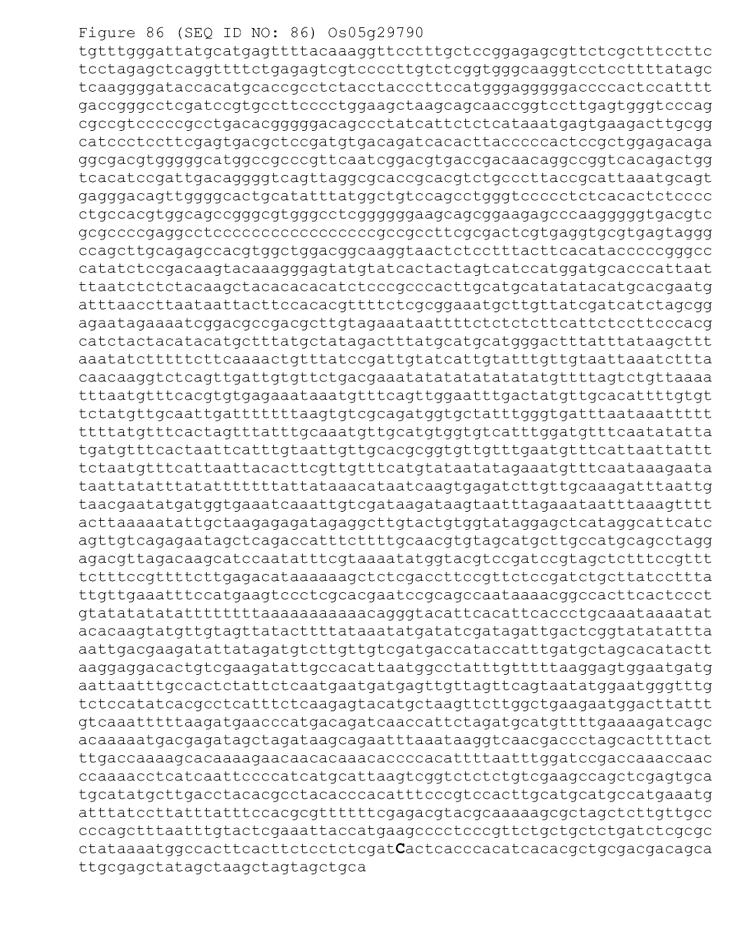

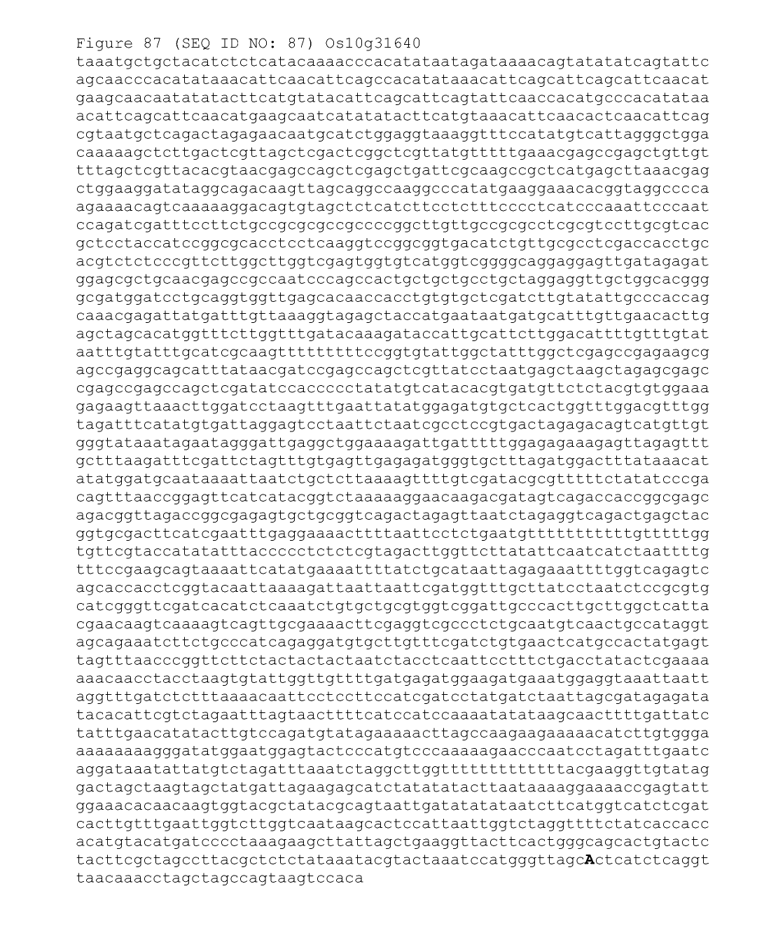

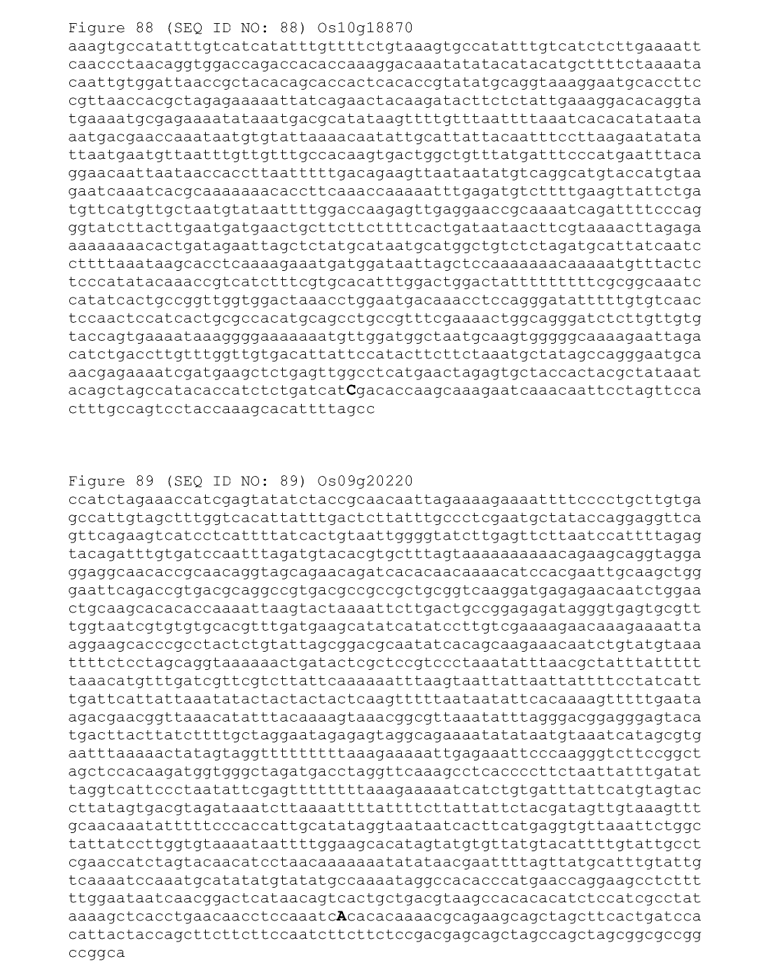

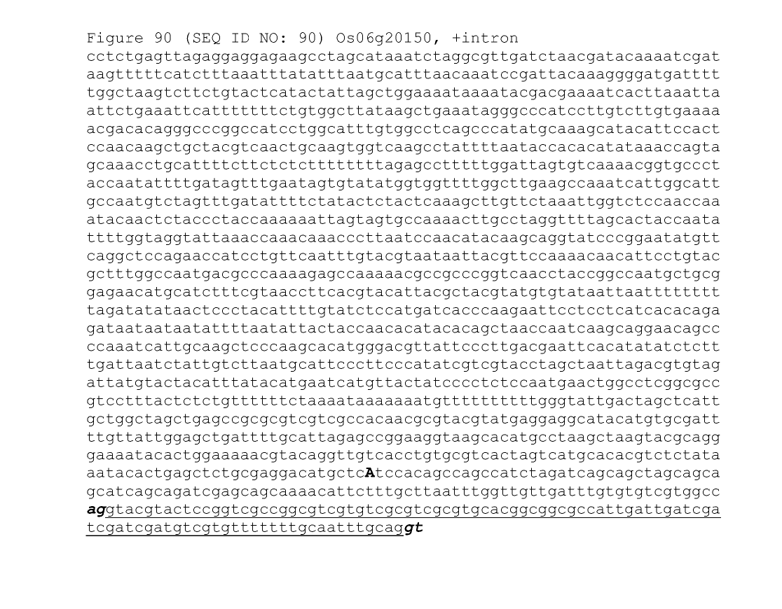

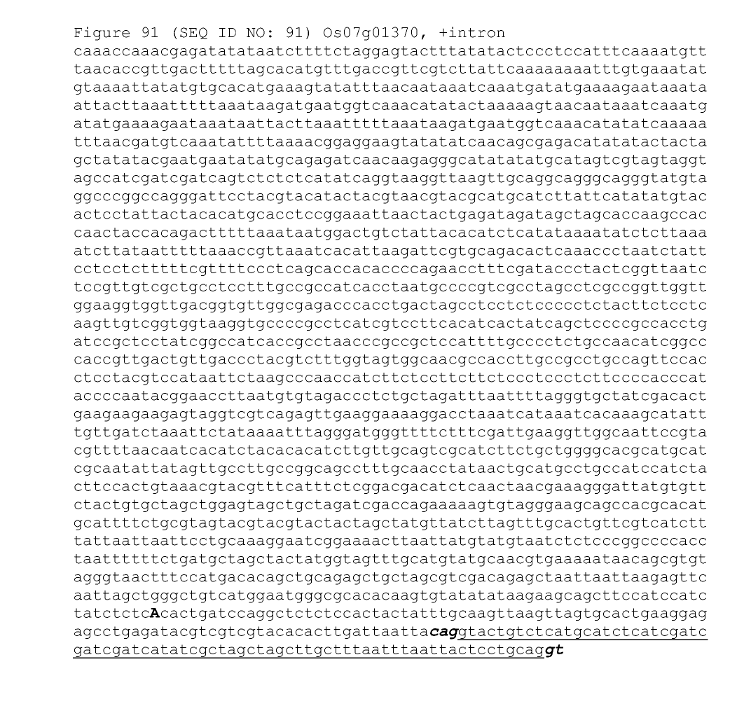

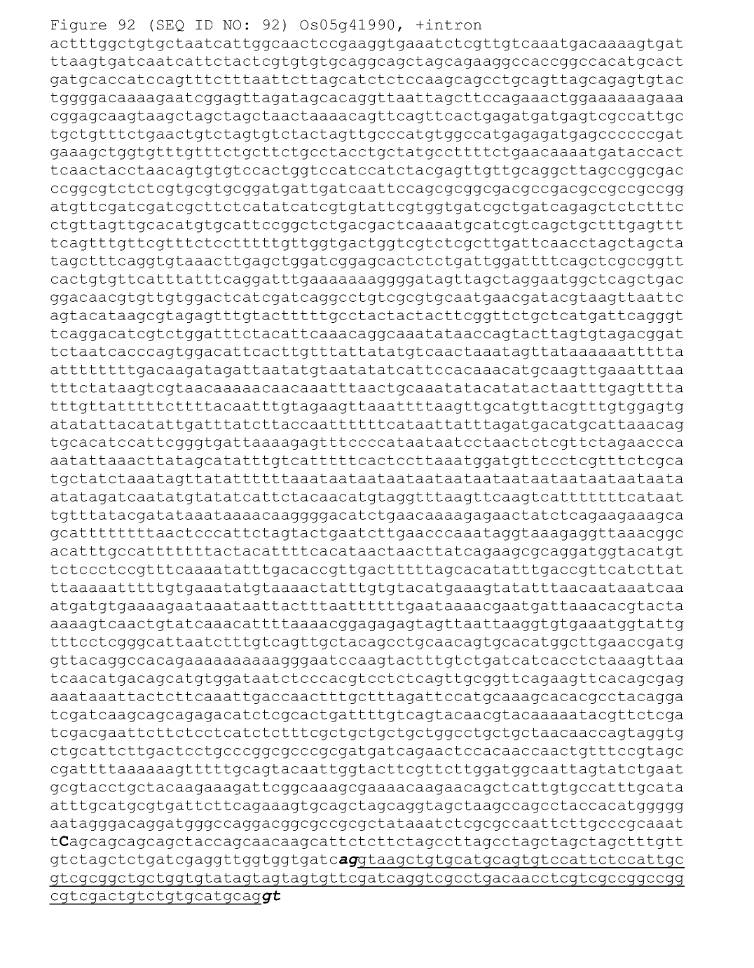

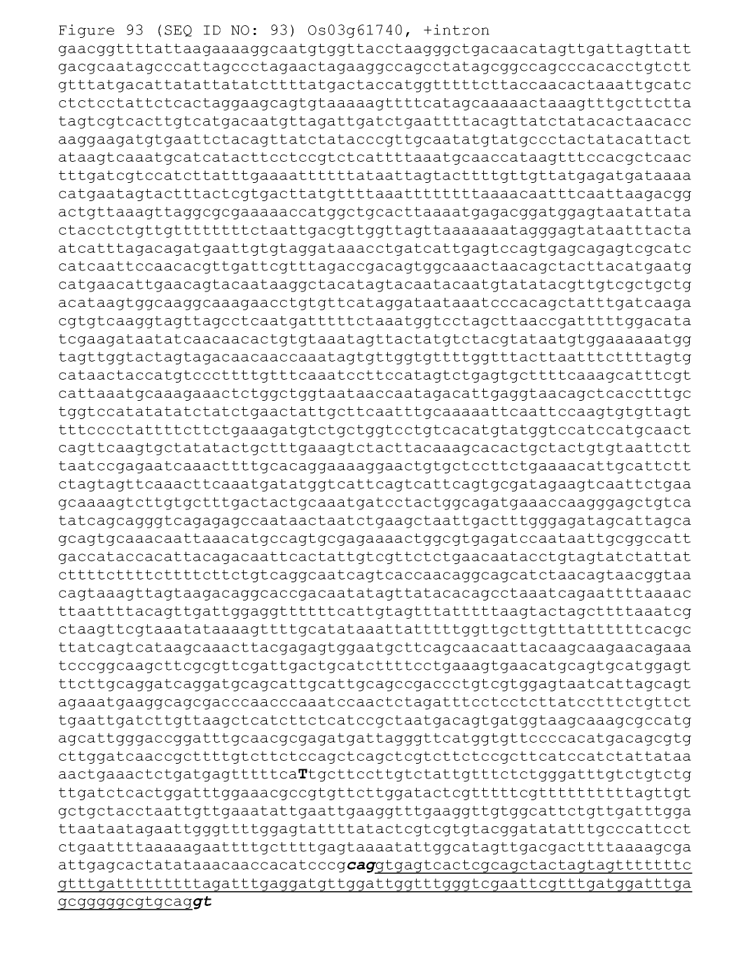

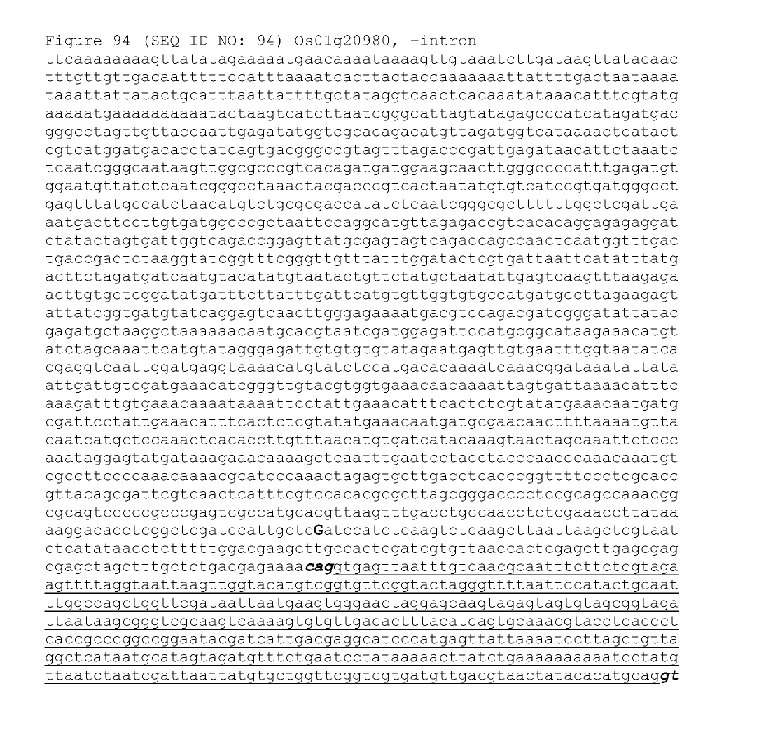

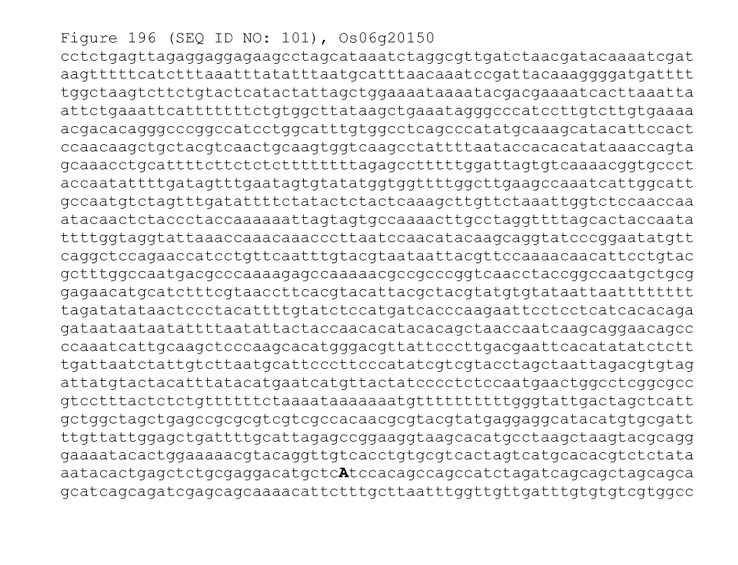

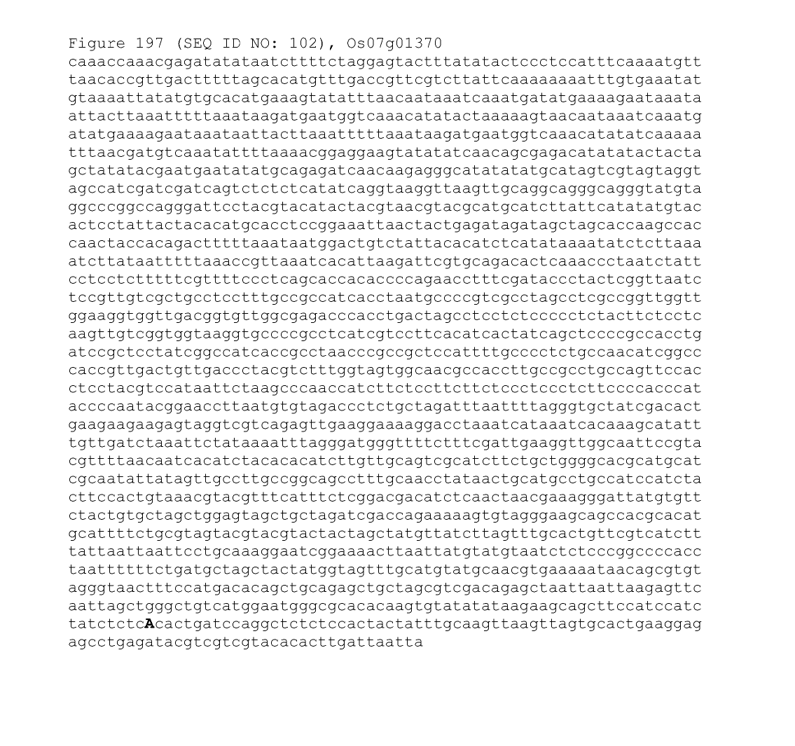

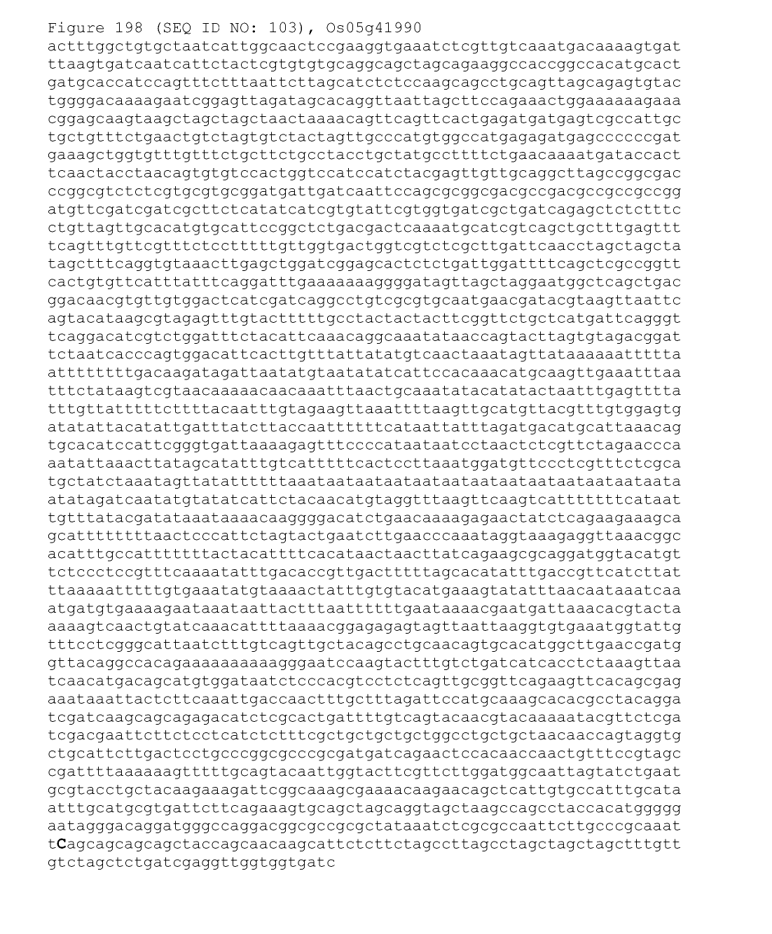

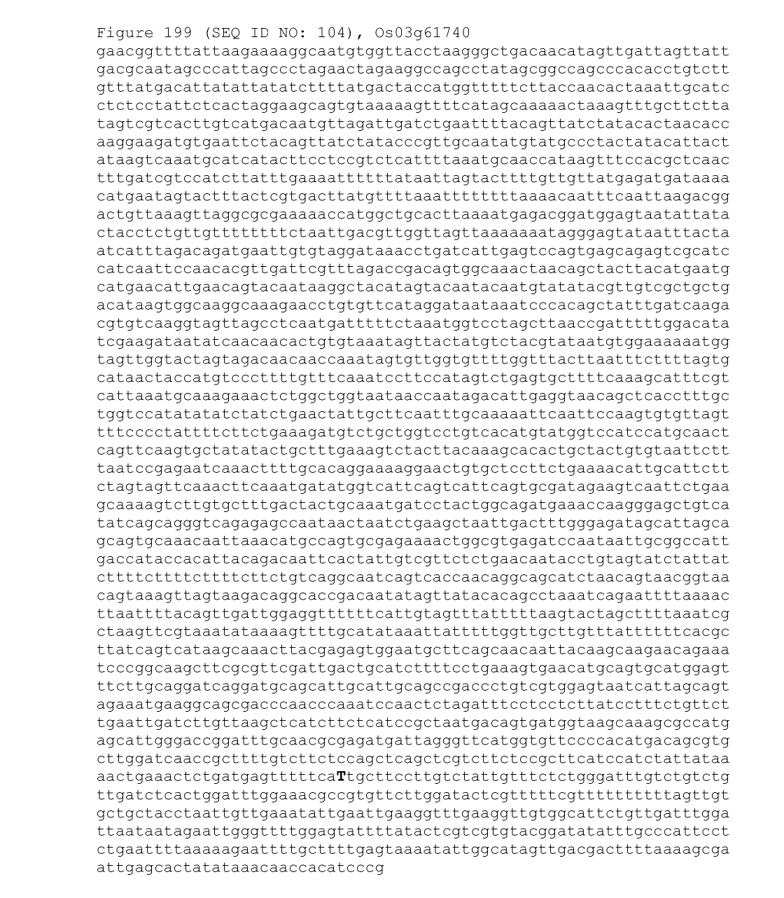

TABLE 6

| | | | | Corresponding |
|---|---|---|---|---|
| | | Corresponding | | Arabidopsis |
| Fig- | SEQ ID | Gene Acces- | Tissue-Specific | Ortholog from |
| ure | NO: | sion No. | Category | Table 1 |
| 17 | 17 | Os11g08380 | broad root (all root | AT1G77330 |
| | | | developmental zones) | |
| 18 | 18 | Os04g55850 | broad root (all root | AT4G21600 |
| | | | developmental zones) | |
| 19 | 19 | Os06g36090 | broad root (all root | AT1G15210 |
| | | | developmental zones) | |
| 20 | 20 | Os05g27304 | epidermal/vascular/ | AT3G45710 |
| | | | cortex/phloem (all root | |
| | | | developmental zones) | |
| 21 | 21 | Os04g33570 | epidermal/vascular/ | AT2G27550 |
| | | | cortex/phloem (all root | |
| | | | developmental zones) | |
| 22 | 22 | Os08g45000 | epidermal/vascular/ | AT3G54700, |
| | | | cortex/phloem (all root | AT2G38940 |
| | | | developmental zones) | |
| 23 | 23 | Os02g44730 | pericycle/endodermis/ | AT2G16970 |
| | | | cortex (all root | |
| | | | developmental zones) | |
| 24 | 24 | Os06g03830 | pericycle/endodermis/ | AT1G64590 |
| | | | cortex (all root | |
| | | | developmental zones) | |
| 25 | 25 | Os04g33520 | epidermis, including | AT1G54890 |
| | | | root hair (all root | |
| | | | developmental zones) | |
| 26 | 26 | Os07g42070 | epidermis, including | AT5G43030 |
| | | | root hair (all root | |
| | | | developmental zones) | |
| 27 | 27 | Os05g04240 | epidermis, including | AT3G62280 |
| | | | root hair (all root | |
| | | | developmental zones) | |
| 28 | 28 | Os06g44410 | meristem/root cap, | AT5G10720 |
| | | | meristematic and | |
| | | | elongation zones (root | |
| | | | meristematic and | |
| | | | elongation zones) | |
| 29 | 29 | Os05g11560 | meristem/root cap, | AT4G19030 |
| | | | meristematic and | |
| | | | elongation zones (root | |
| | | | meristematic and | |
| | | | elongation zones) | |
| 30 | 30 | Os10g10620 | meristem/root cap, | AT4G00080 |
| | | | meristematic and | |
| | | | elongation zones (root | |
| | | | meristematic and | |
| | | | elongation zones) | |
| 31 | 31 | Os11g05470 | epidermal/vascular/ | AT2G27550 |
| | | | cortex/phloem (all root | |
| | | | developmental zones) | |
| 67 | 67 | Os02g52840 | epidermal/vascular/ | AT5G63600 |
| | | | cortex/phloem (all root | |
| | | | developmental zones) | |
| 68 | 68 | Os02g16500 | epidermal/vascular/ | AT5G53250 |
| | | | cortex/phloem (all root | |
| | | | developmental zones) | |
| 69 | 69 | Os10g40480 | epidermal/vascular/ | AT4G12550 |
| | | | cortex/phloem (all root | |
| | | | developmental zones) | |
| 70 | 70 | Os04g51890 | meristem/root cap, | AT4G31320 |
| | | | meristematic and | |
| | | | elongation zones (root | |
| | | | meristematic and | |
| | | | elongation zones) | |
| 83 | 83 | Os06g35630 | broad root | AT5G44380 |
| 84 | 84 | Os06g35650 | broad root | AT5G44380 |
| 85 | 85 | Os09g39070 | broad root | |
| 86 | 86 | Os05g29790 | broad root | AT1G02810 |
| 87 | 87 | Os10g31640 | broad root | |
| 88 | 88 | Os10g18870 | broad root | |
| 89 | 89 | Os09g20220 | broad root | |
| 90 | 90 | Os06g20150 | broad root | |
| | | (+intron) | | |
| 91 | 91 | Os07g01370 | broad root | |
| | | (+intron) | | |
| 92 | 92 | Os05g41990 | broad root | |
| | | (+intron) | | |
| 93 | 93 | Os03g61740 | broad root | |
| | | (+intron) | | |
| 94 | 94 | Os01g20980 | broad root | AT5G44380 |
| | | (+intron) | | |
| 196 | 101 | Os06g20150 | broad root | |
| 197 | 102 | Os07g01370 | broad root | |
| 198 | 103 | Os05g41990 | broad root | |
| 199 | 104 | Os03g61740 | broad root | |
| 200 | 105 | Os01g20980 | broad root | AT5G44380 |

Where annotated, the nucleic acid sequences provided in FIGS. 17-31, 67-70, 83-94, and 196-200 indicate one transcription start site (Capital letter in bold), any endogenous 5′-UTR intron sequences (double underlining), any first intron added from the coding sequence (single underlining), and any added intron splice sequences (bold italics), although not all figures may be annotated. All rice genome sequence and annotation is from the Rice Genome Annotation Project (available on the worldwide web at rice.plantbiology.msu.edu/index.shtml).

### Example 5

### Endogenous Expression Analysis of Rice Genes

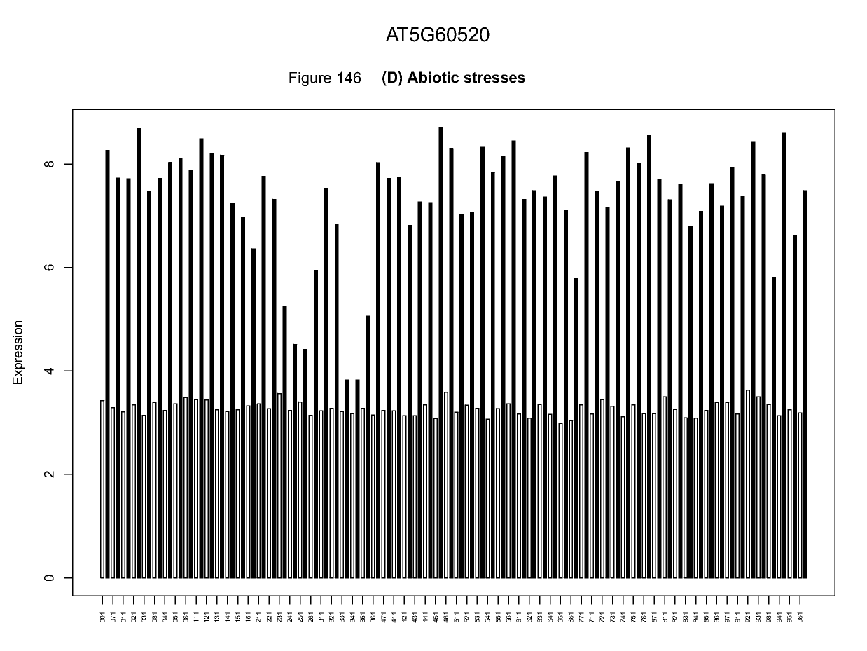

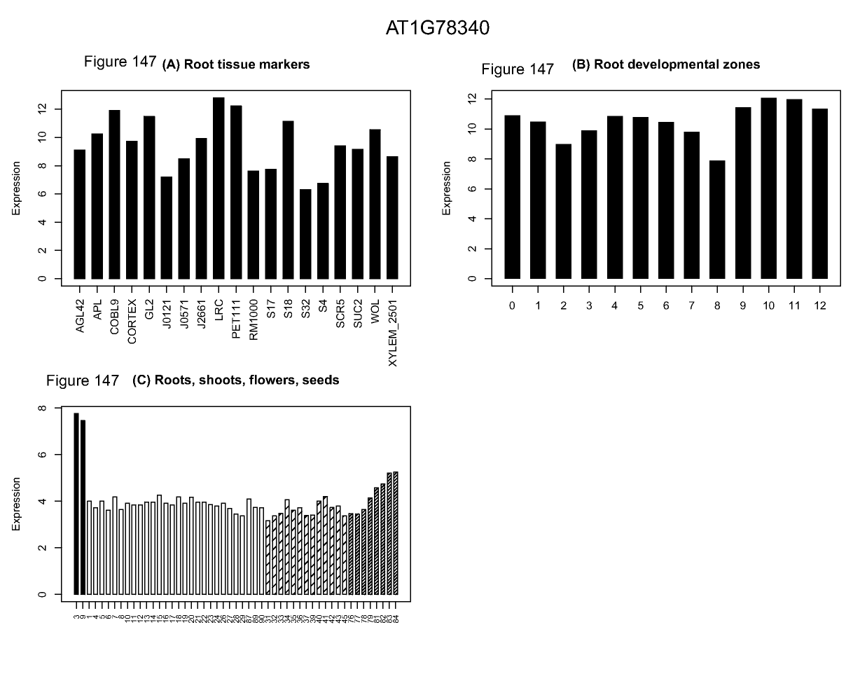

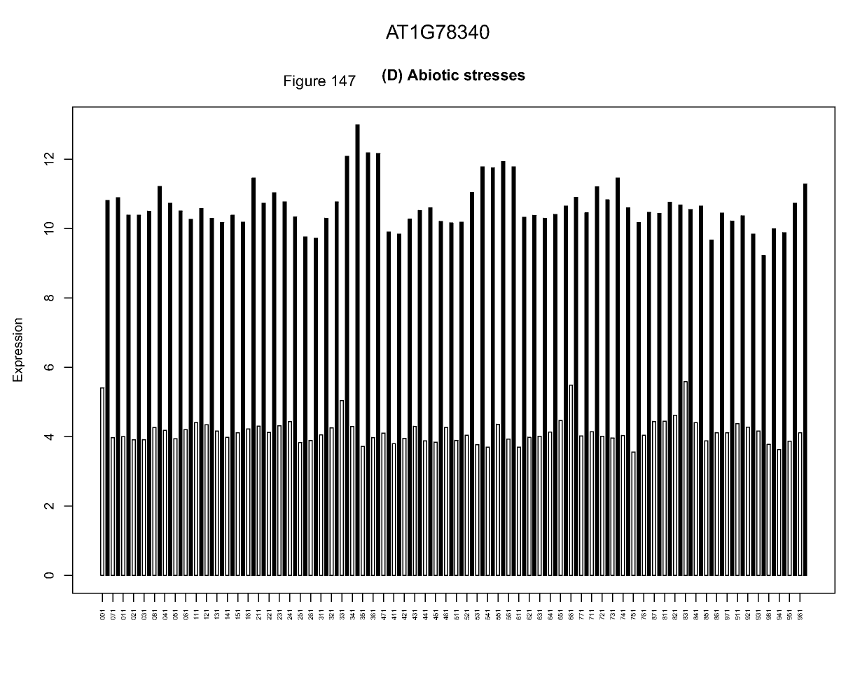

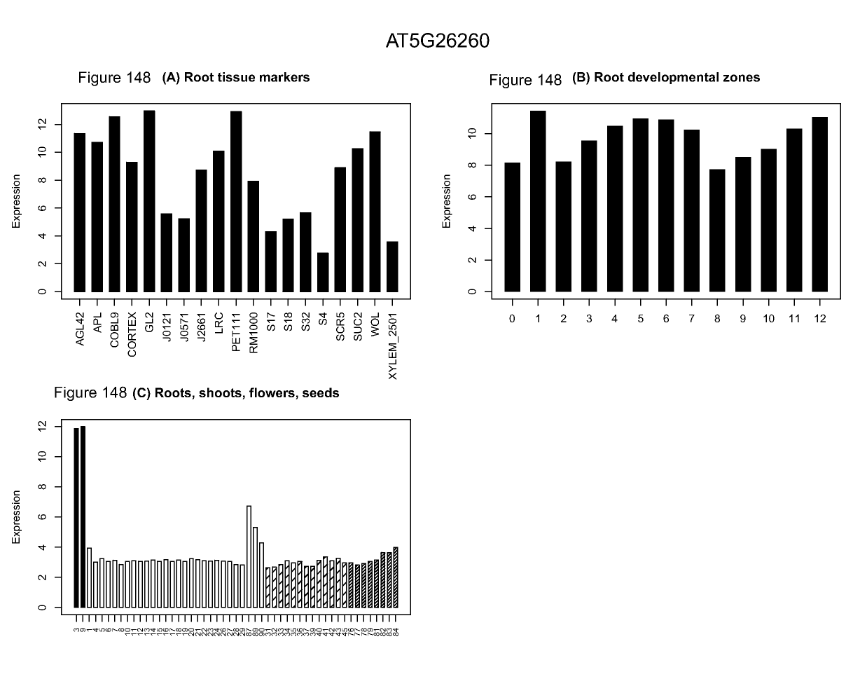

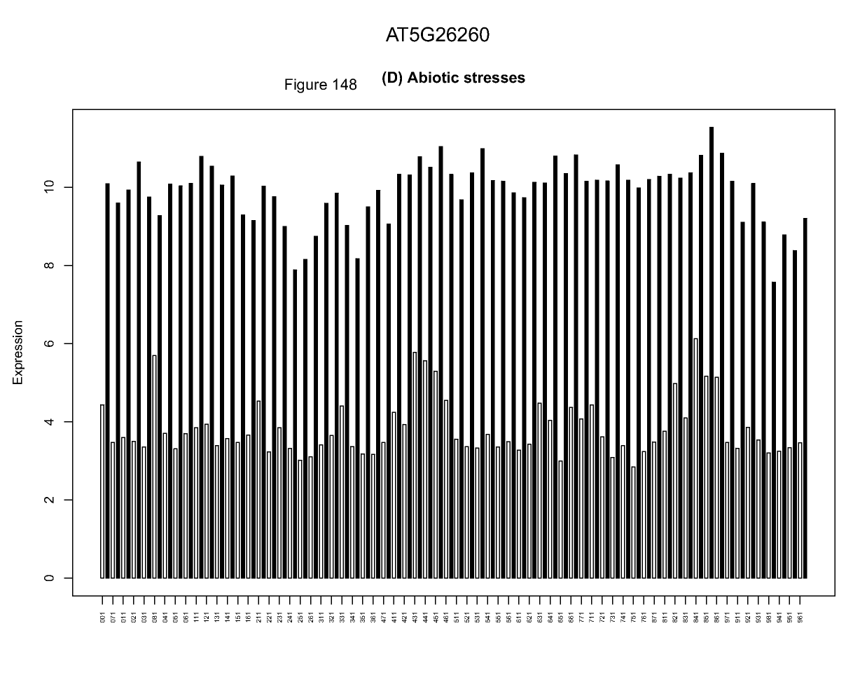

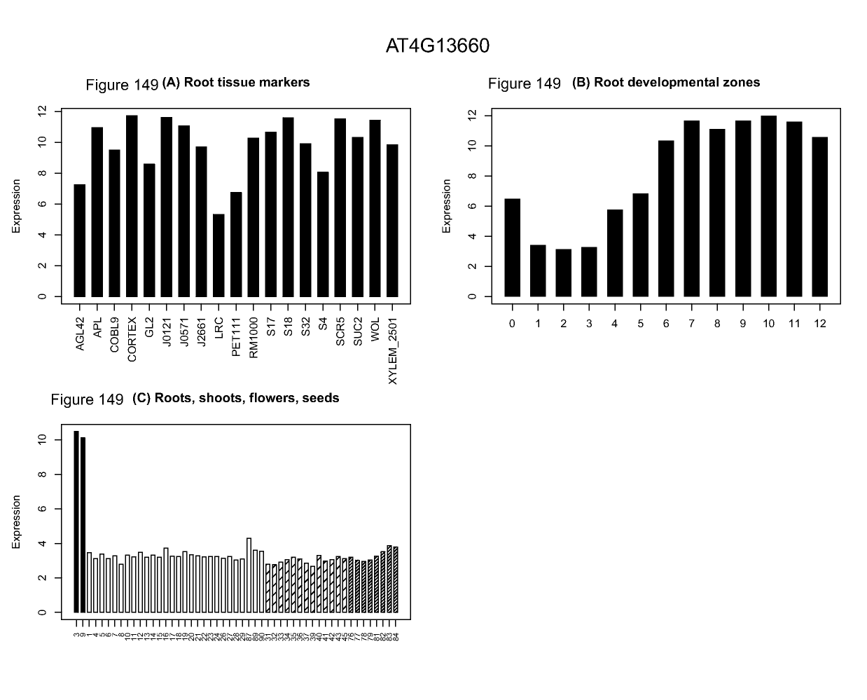

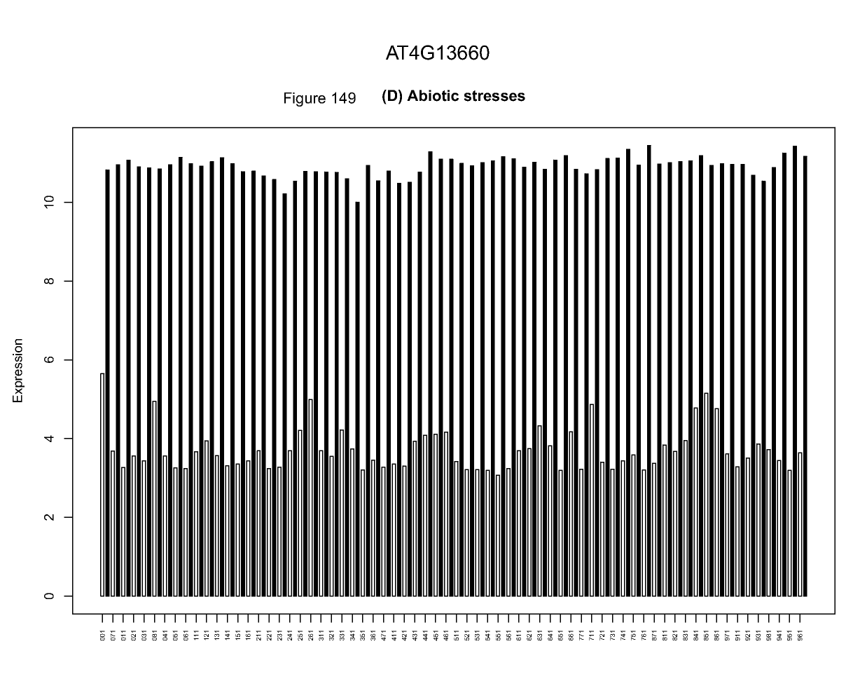

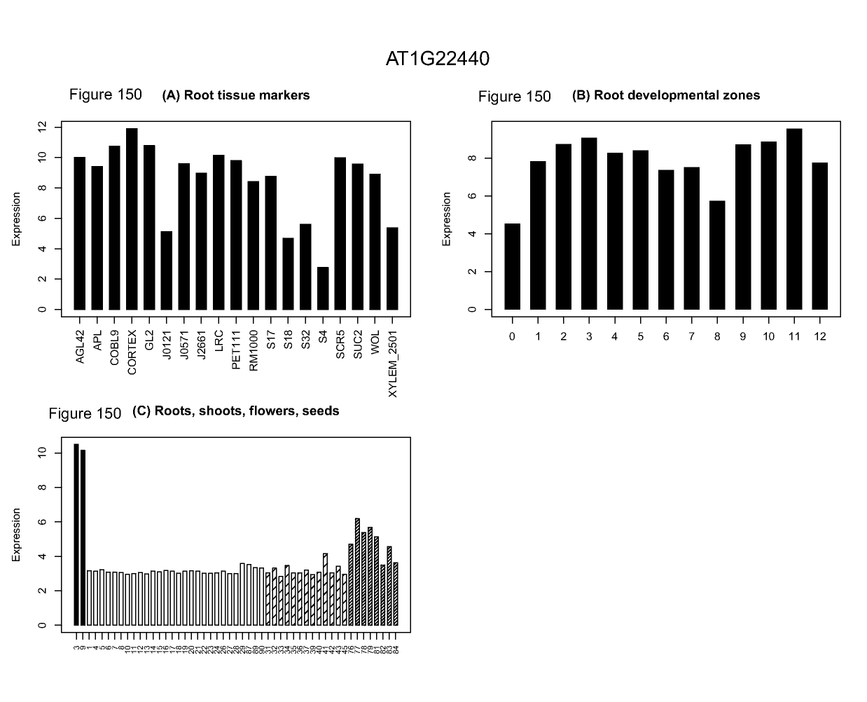

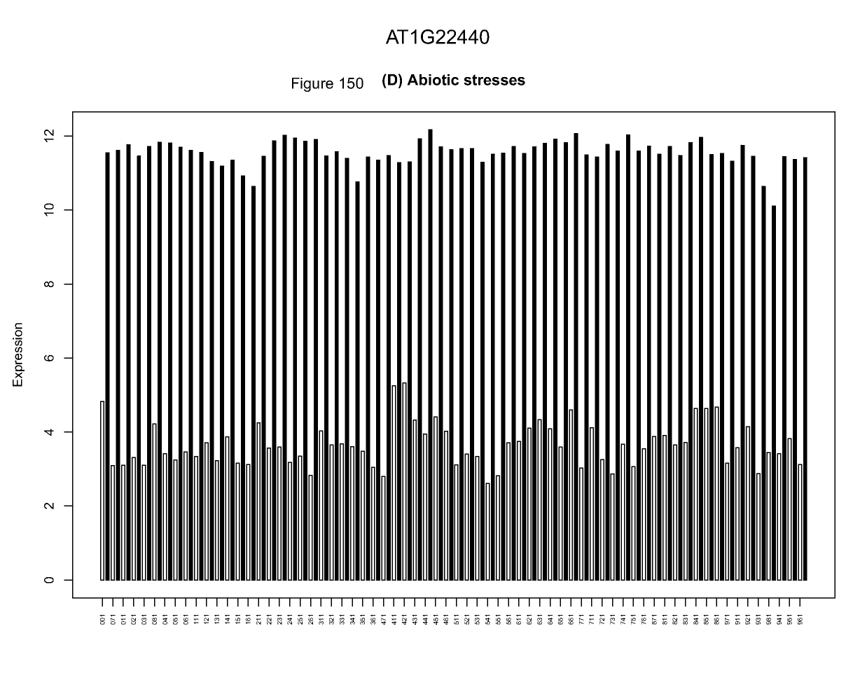

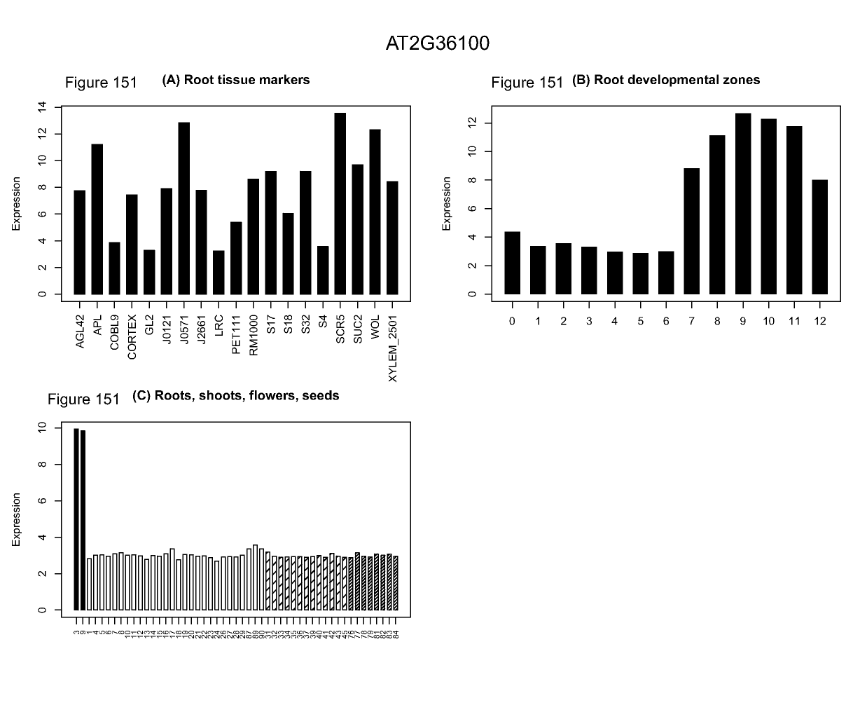

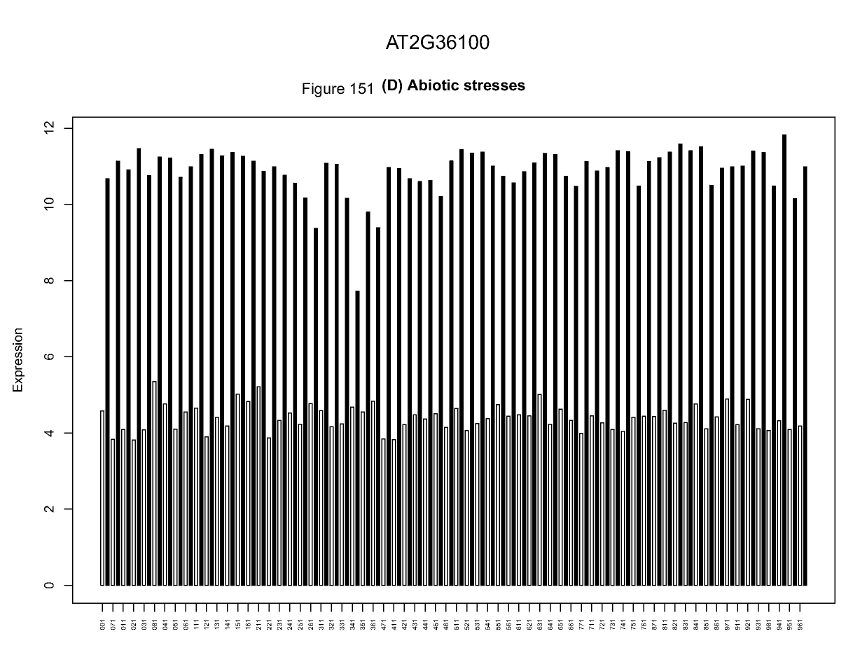

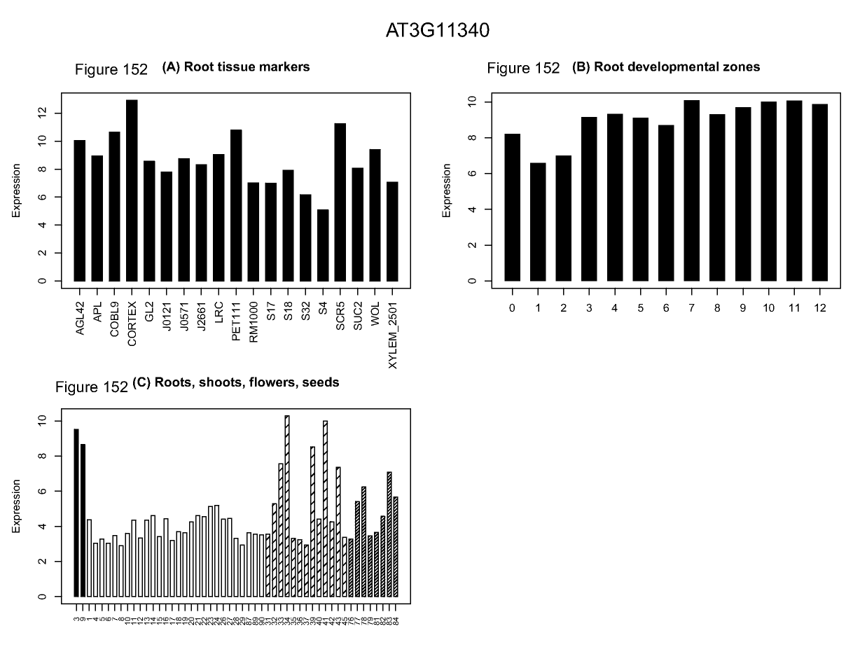

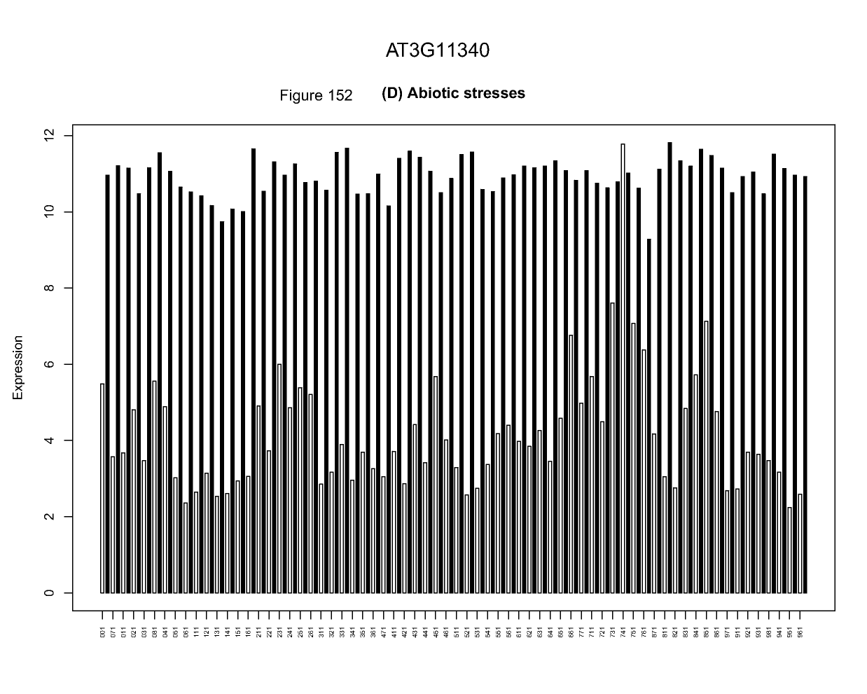

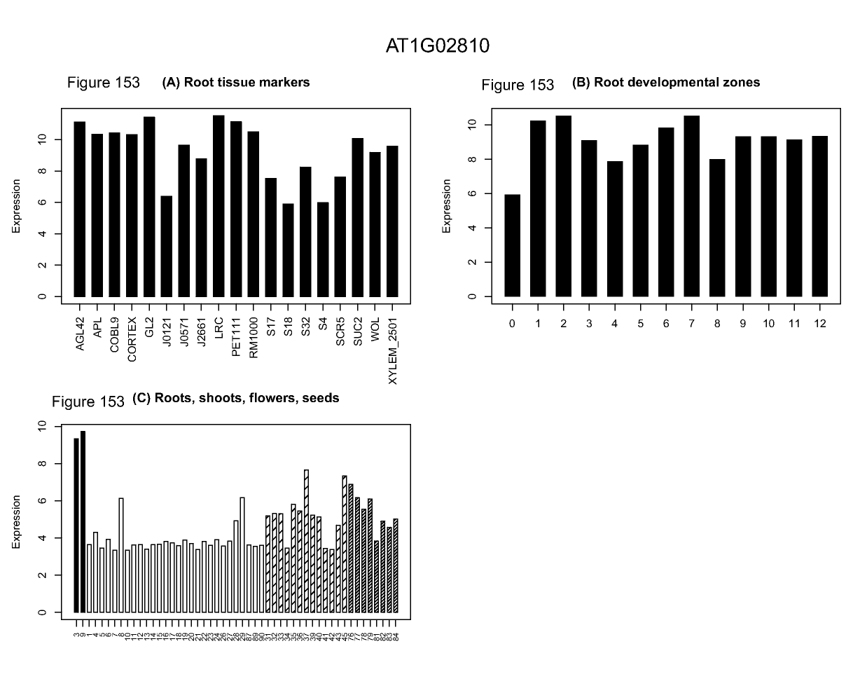

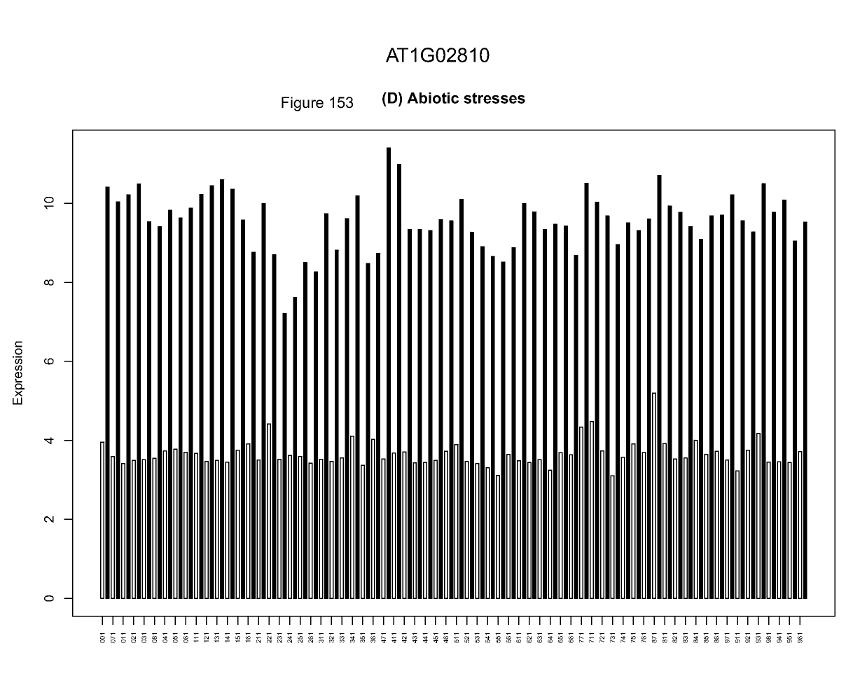

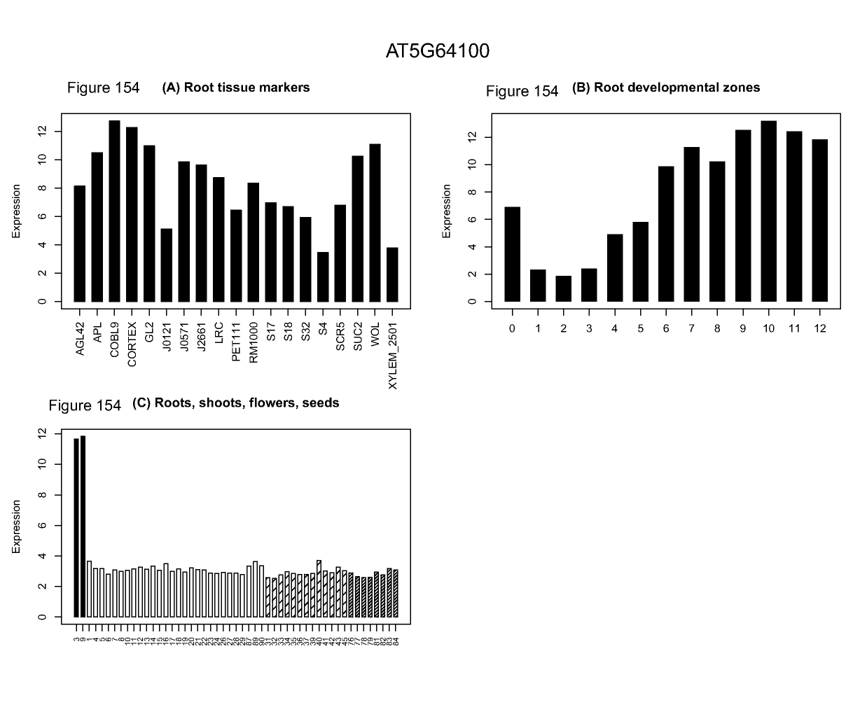

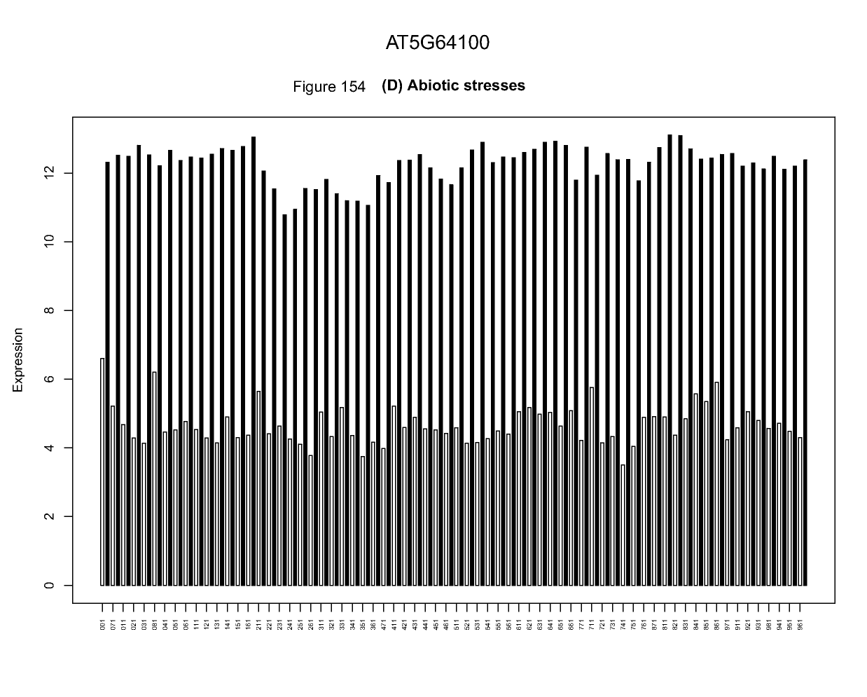

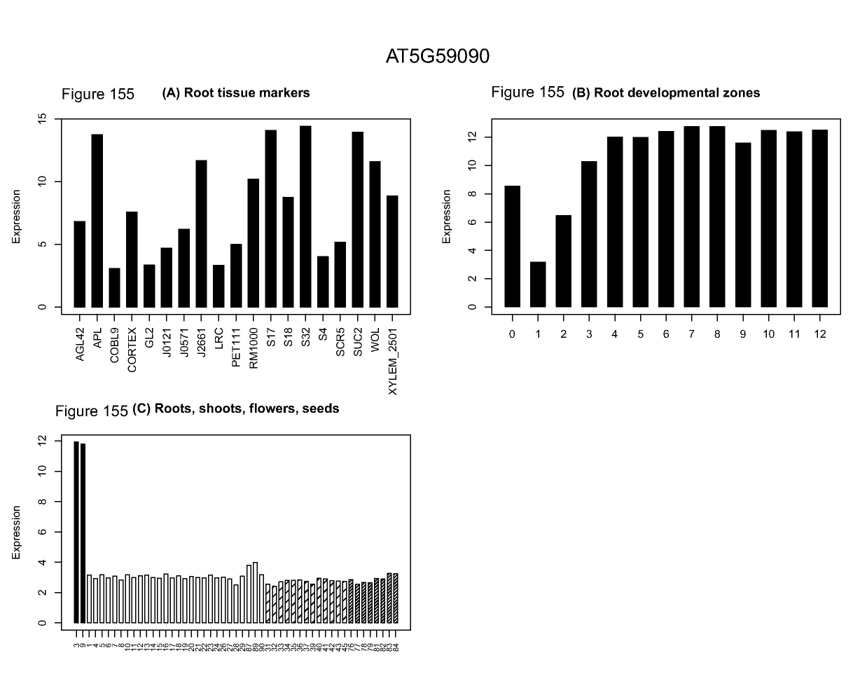

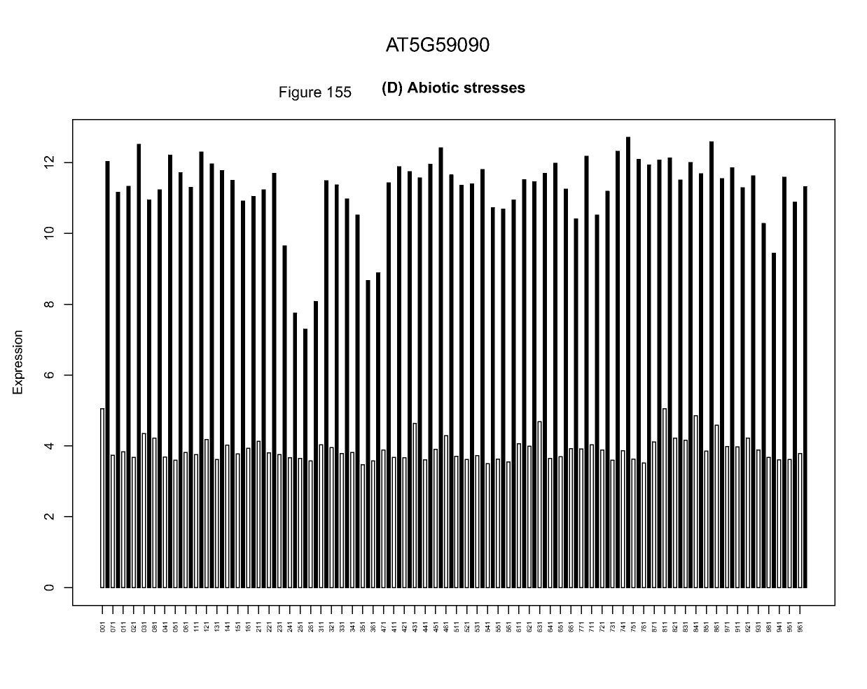

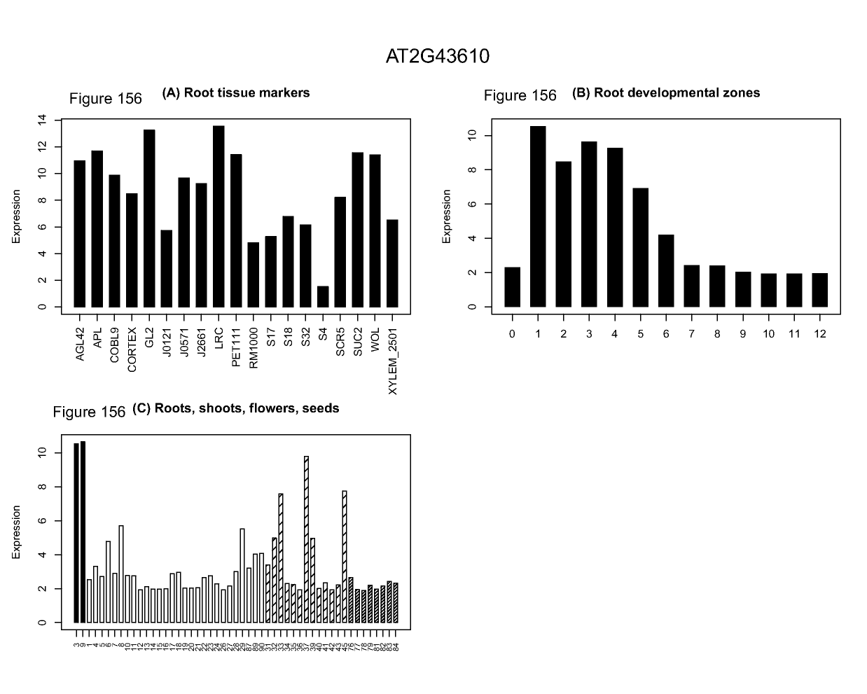

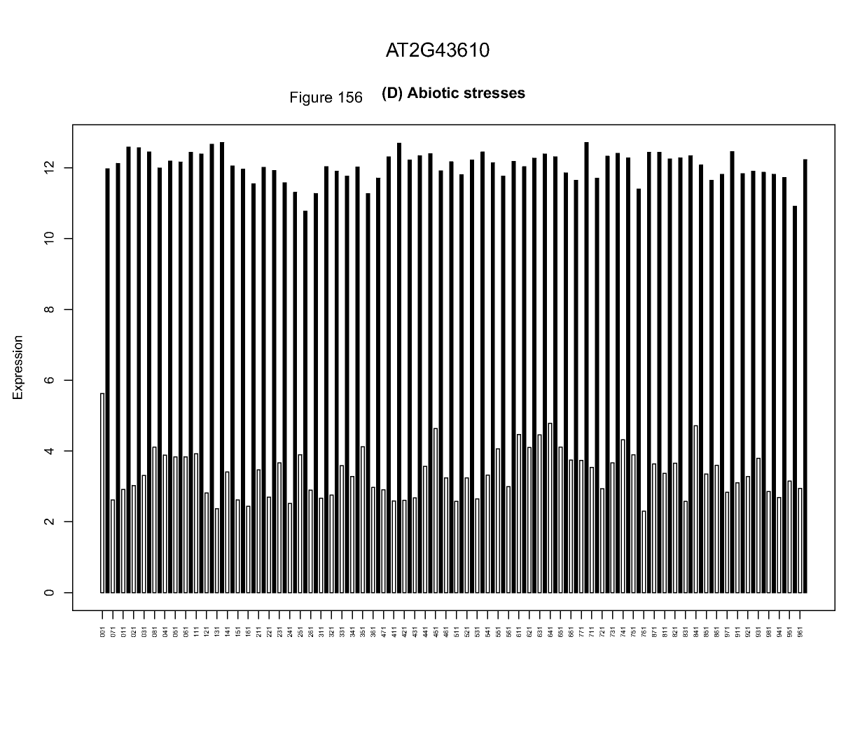

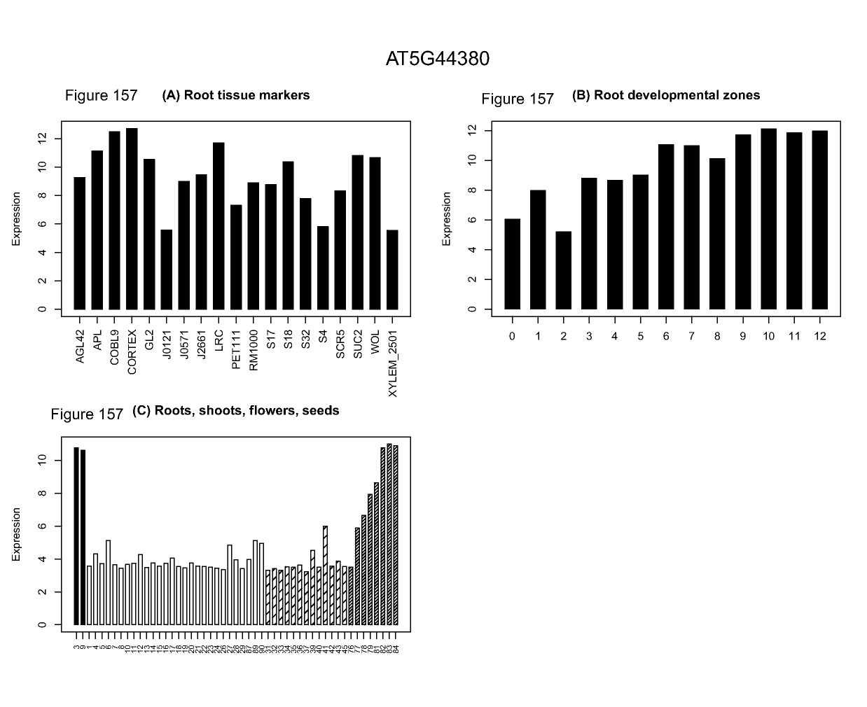

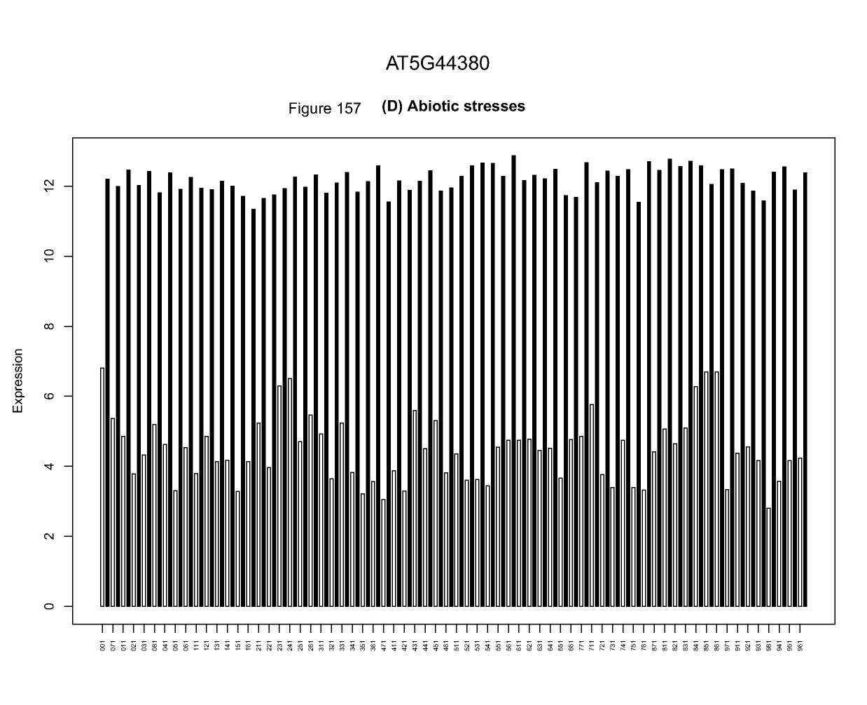

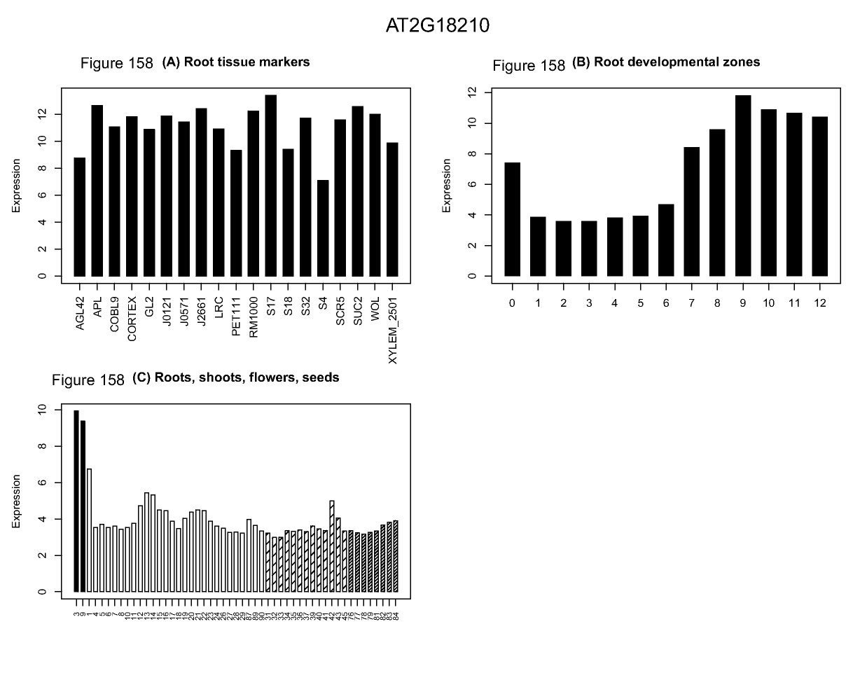

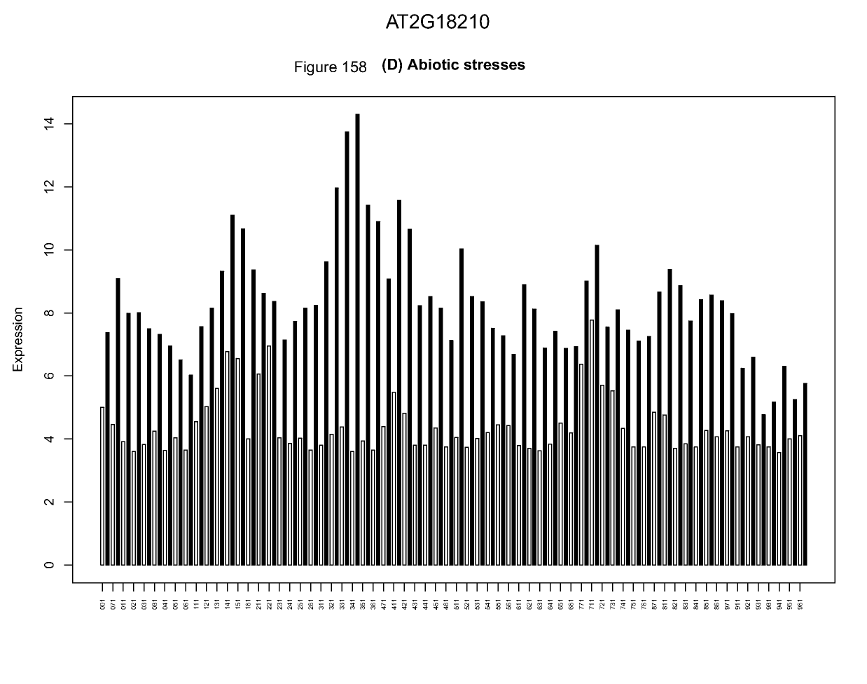

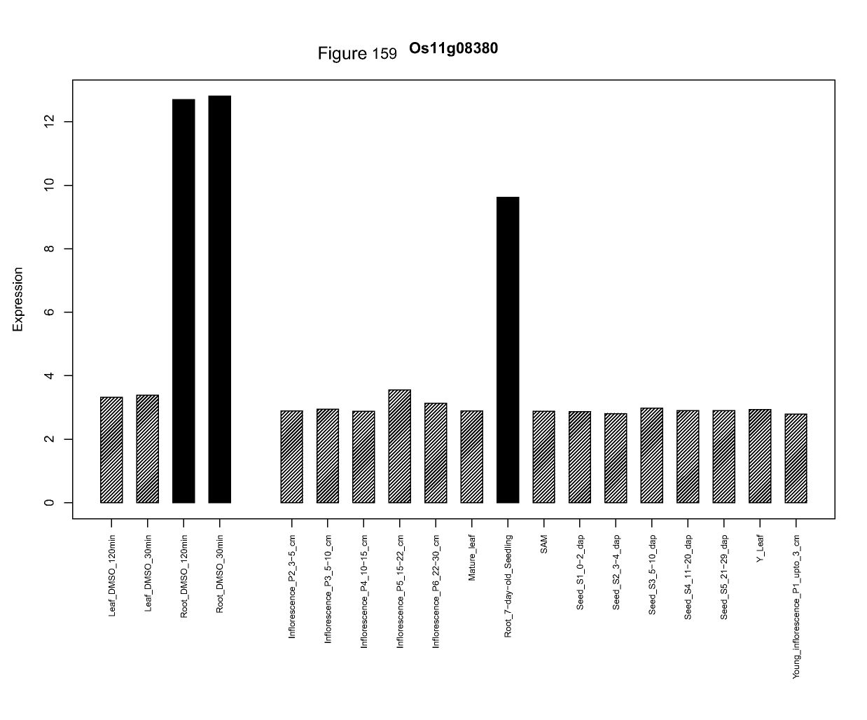

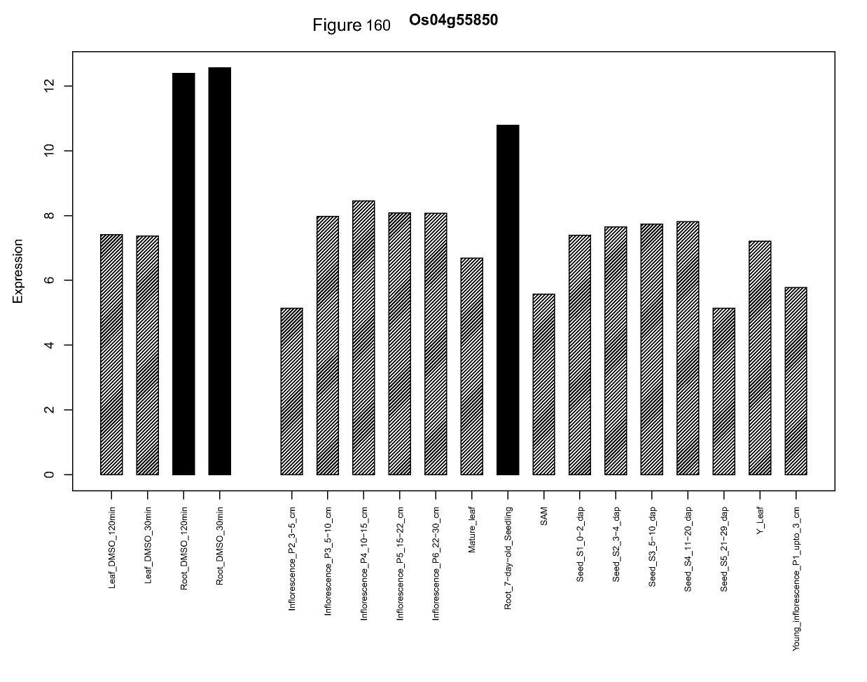

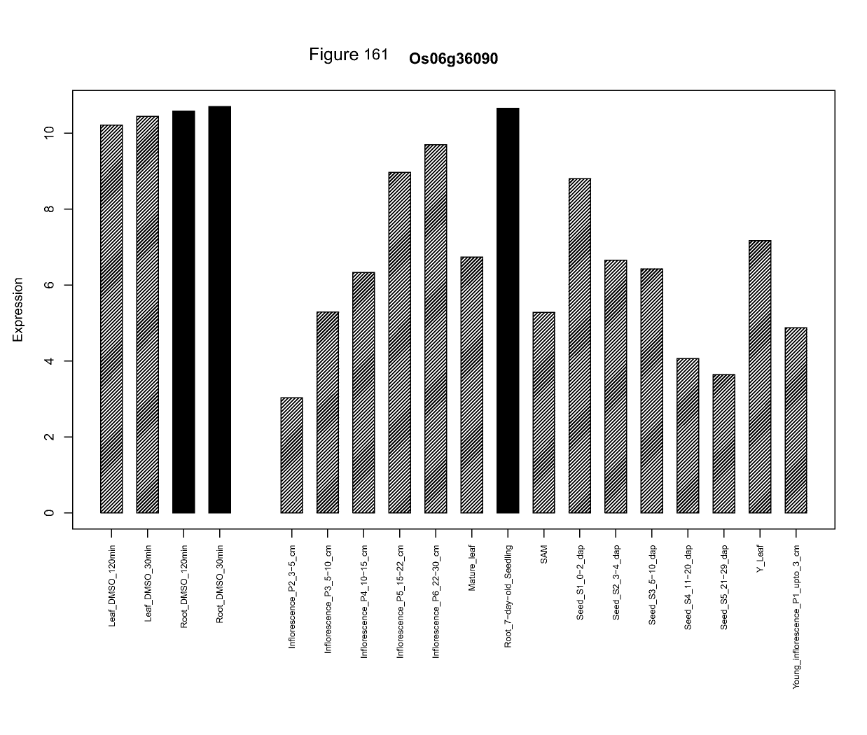

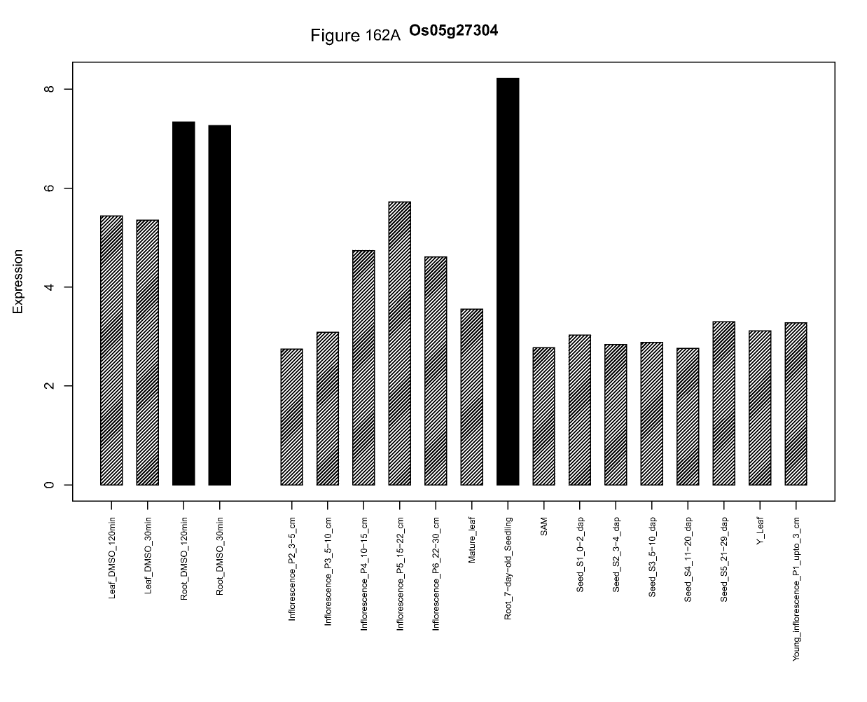

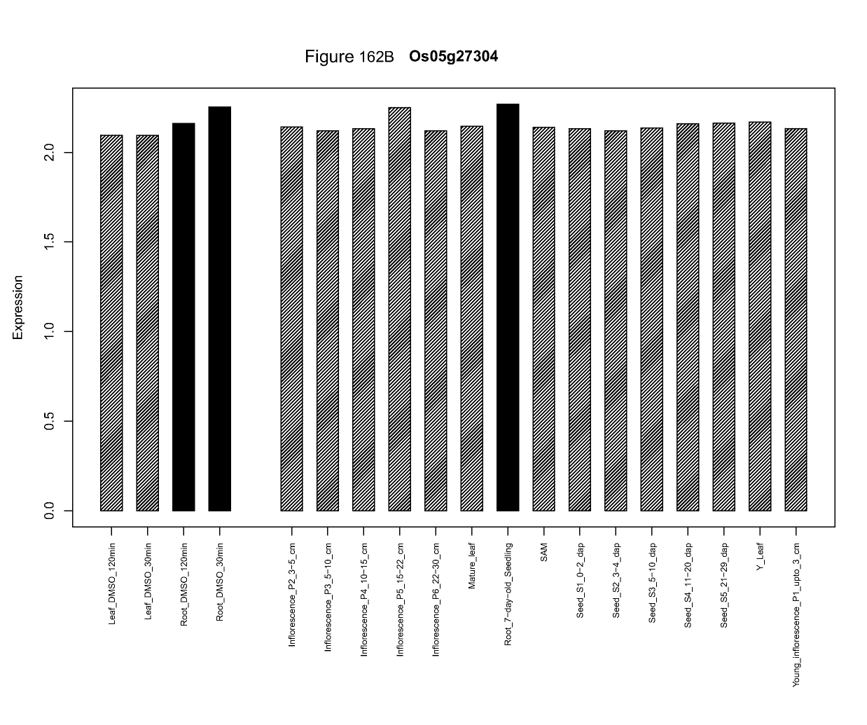

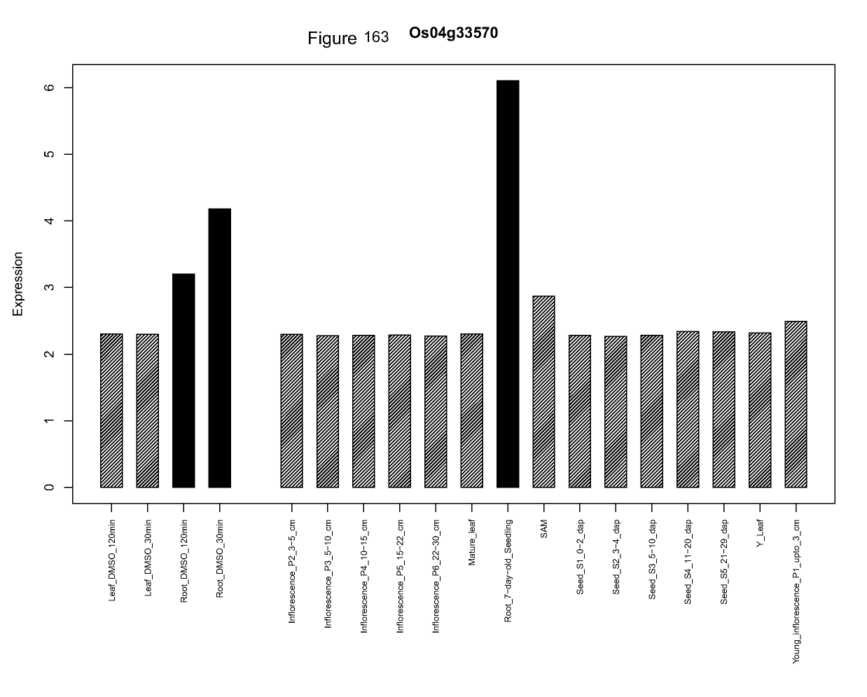

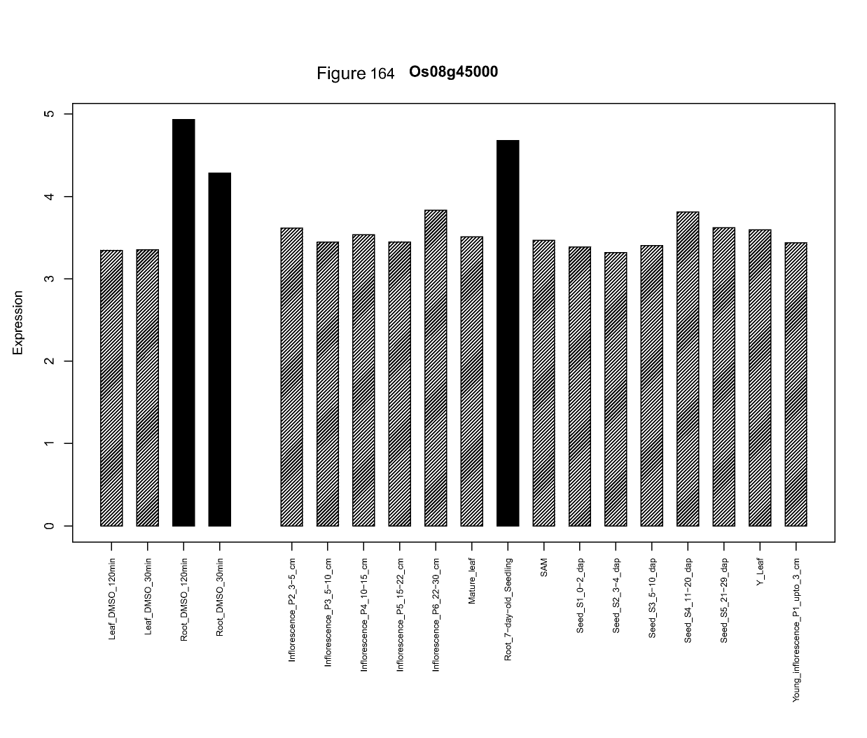

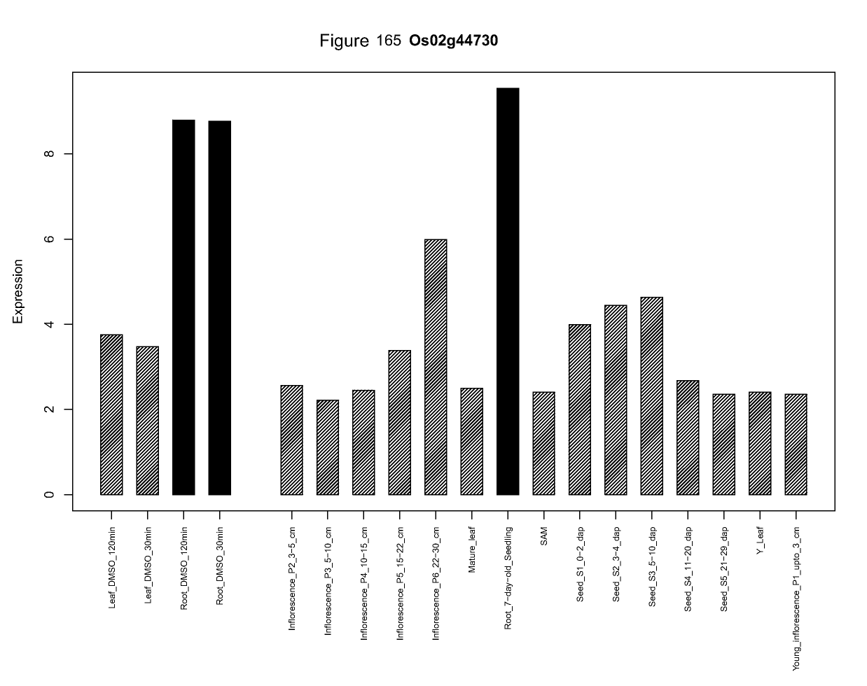

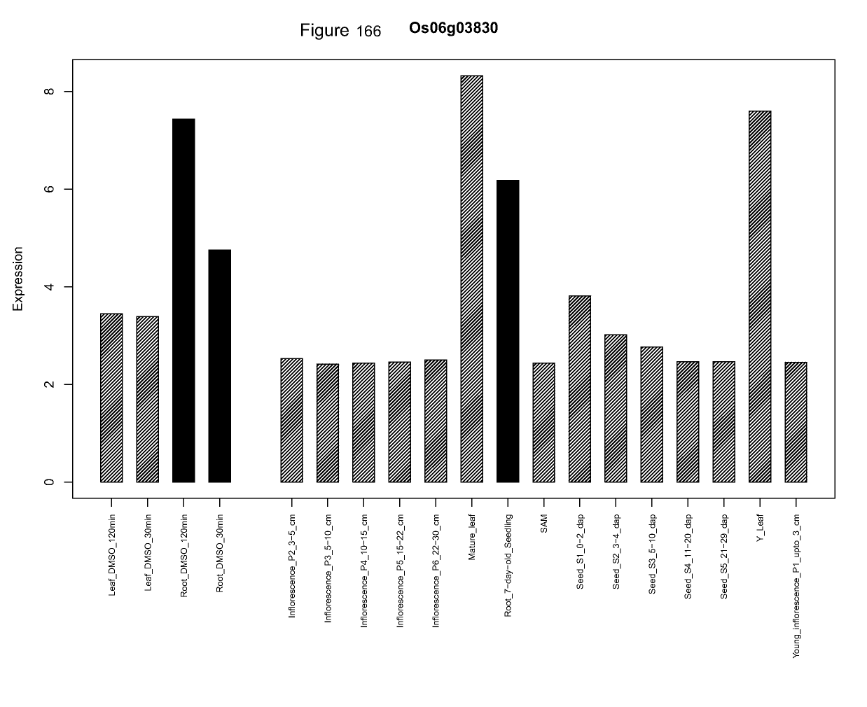

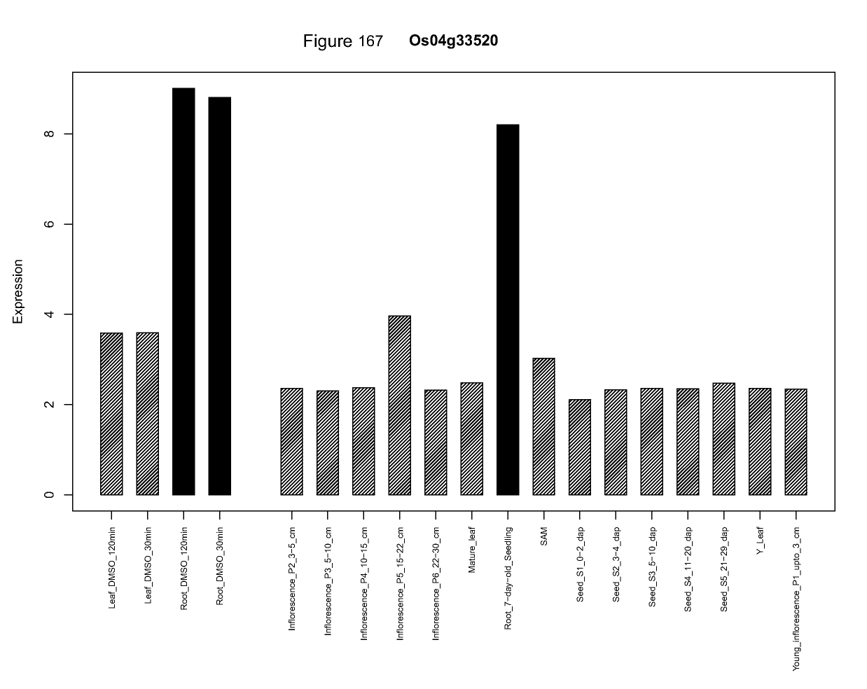

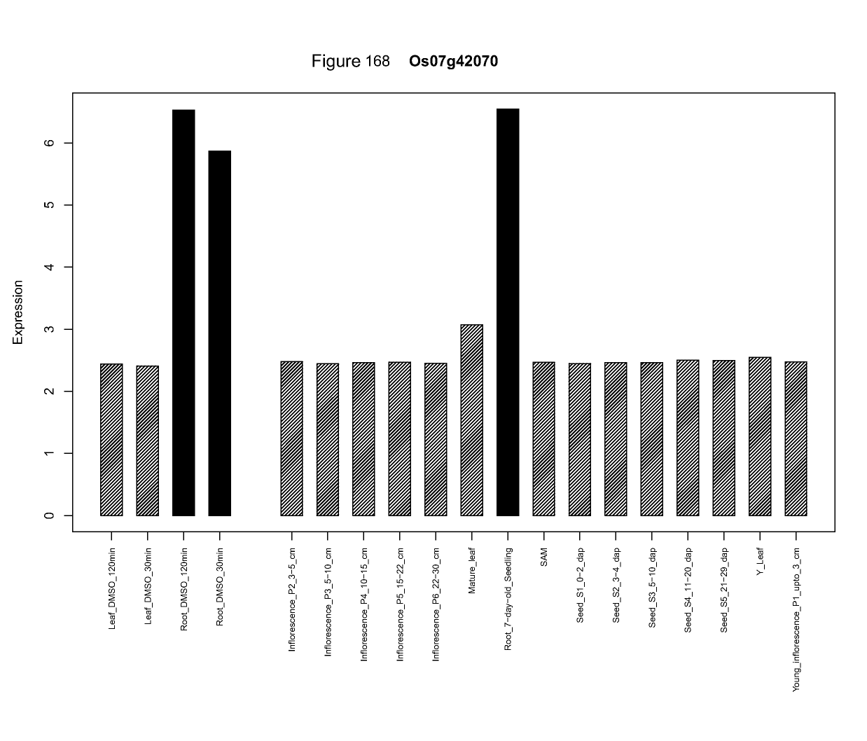

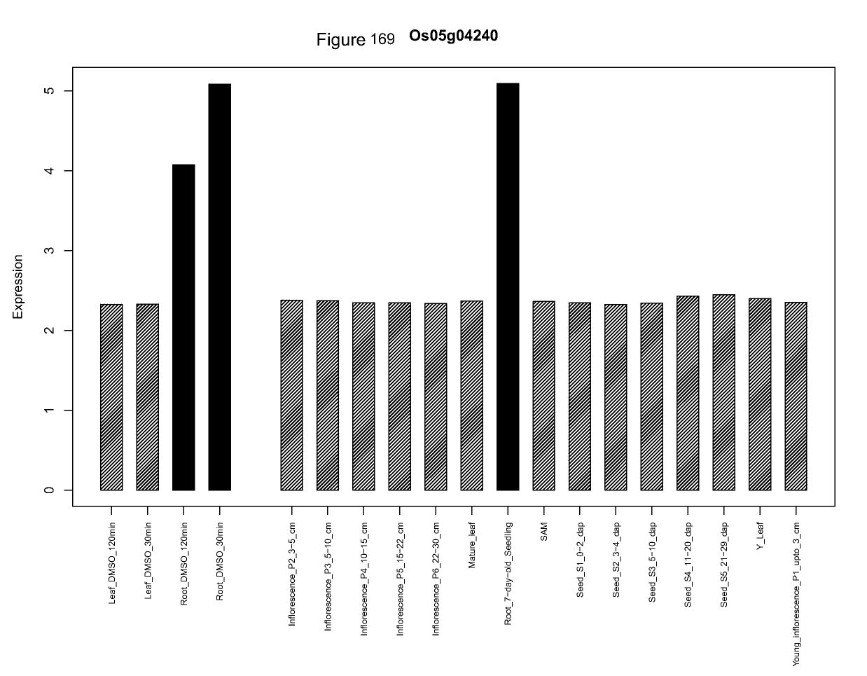

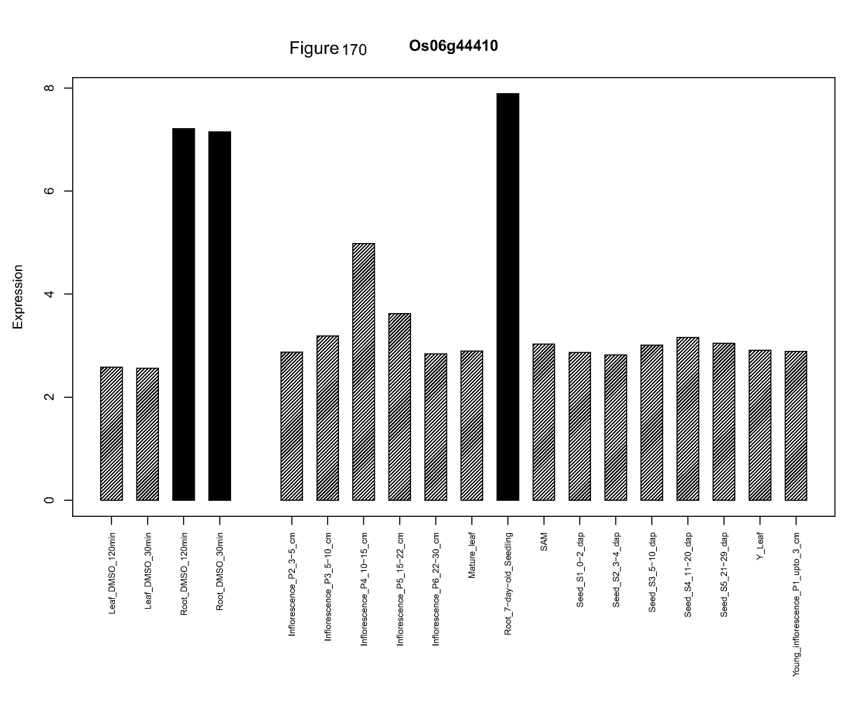

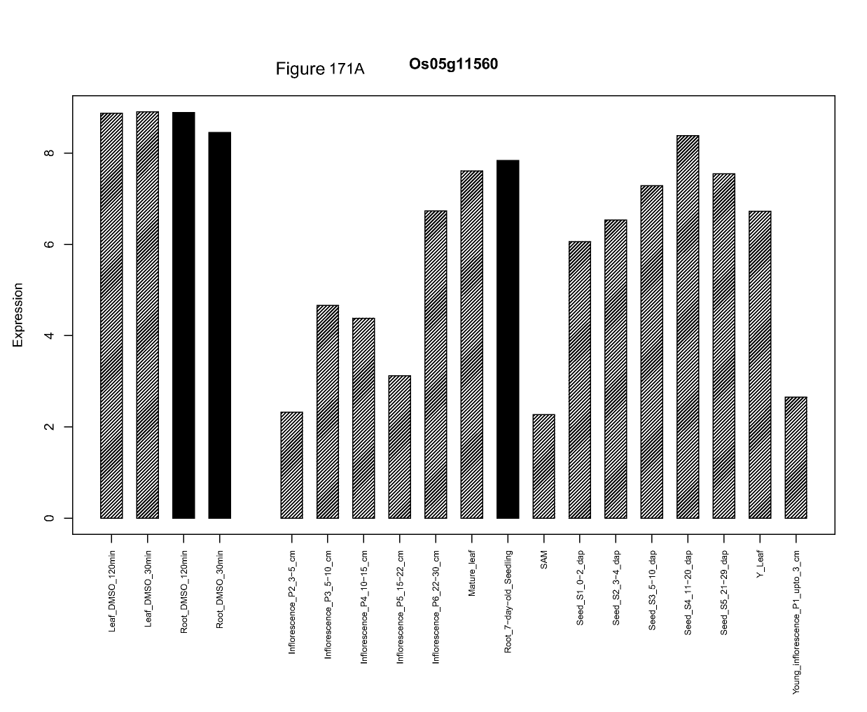

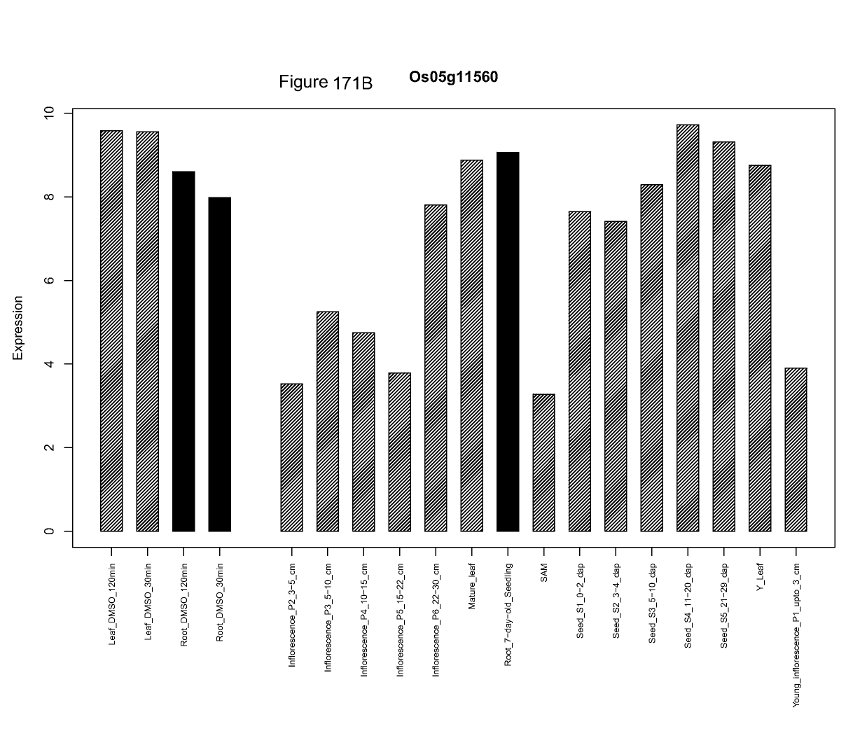

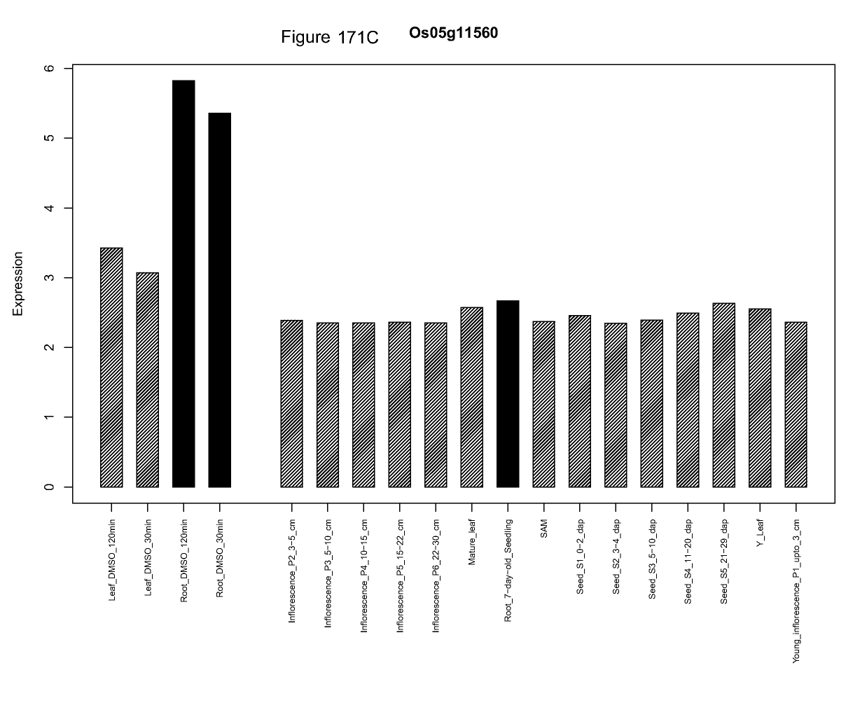

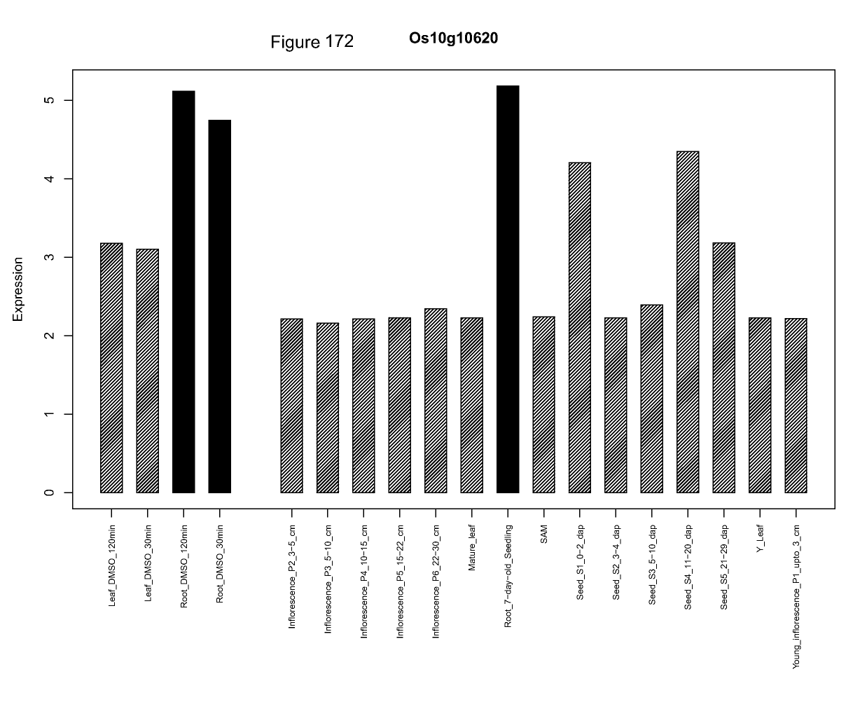

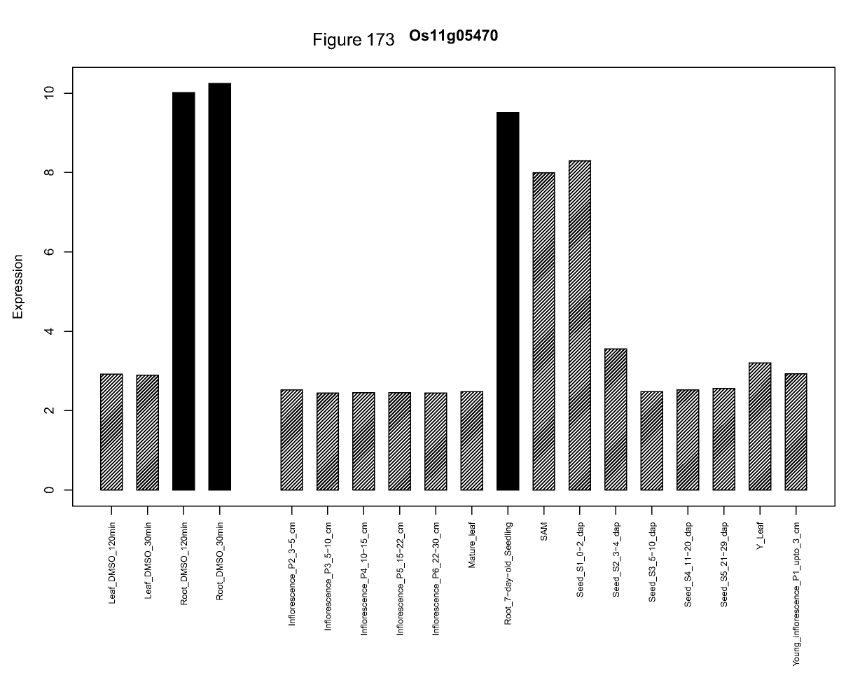

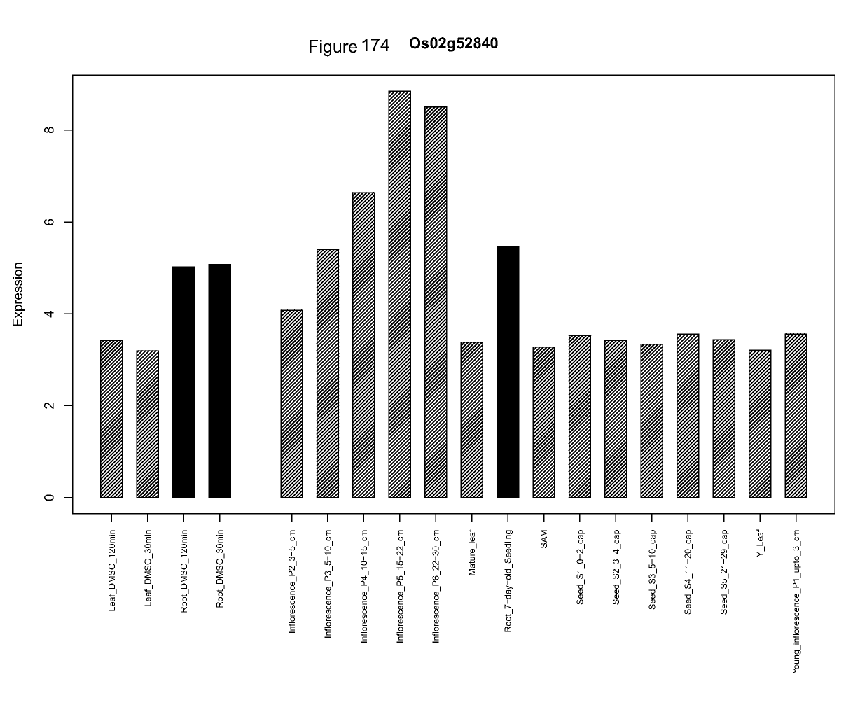

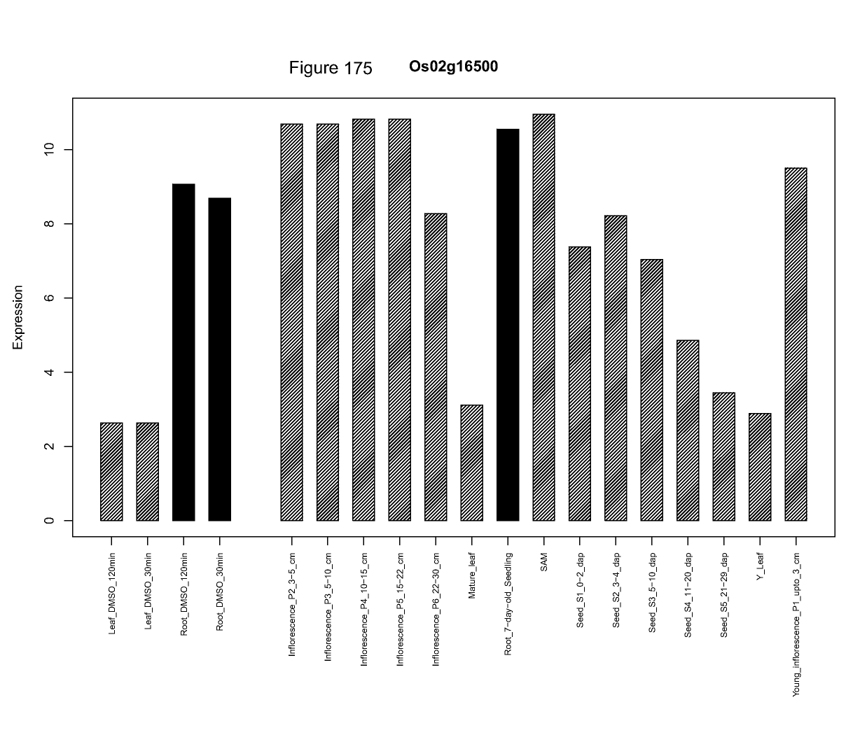

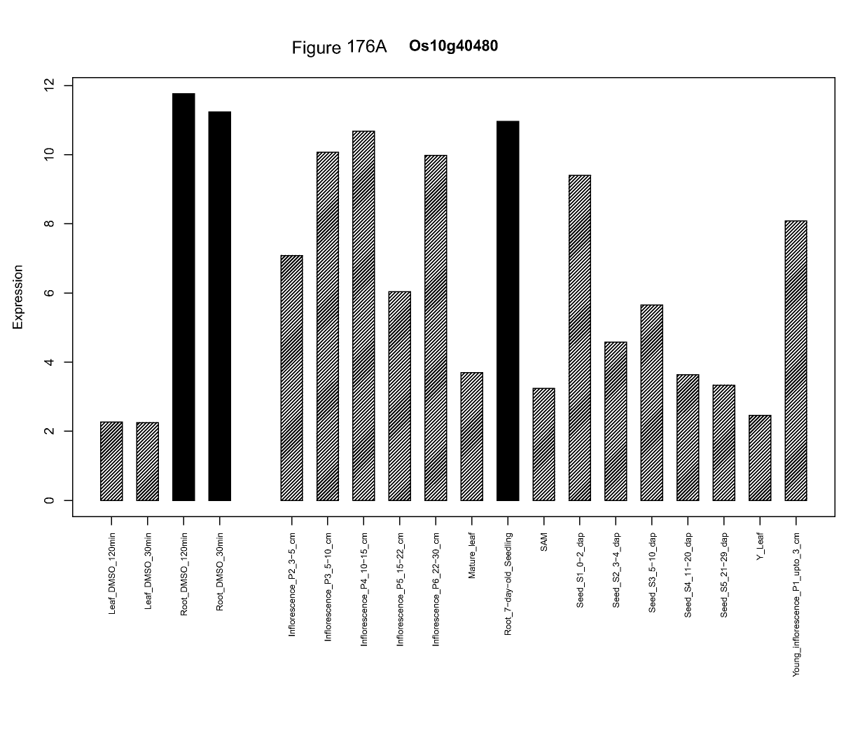

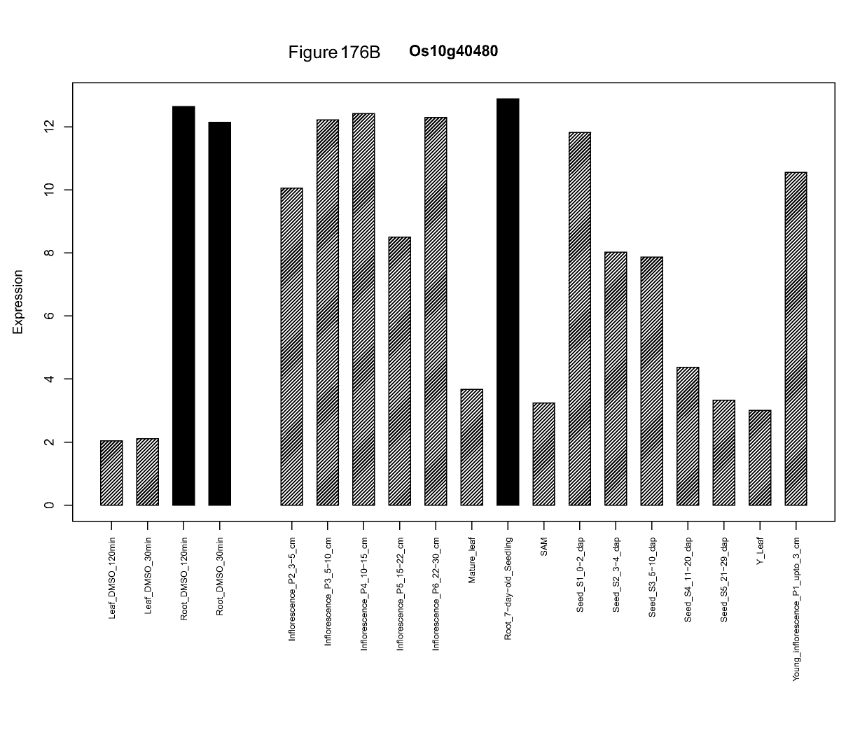

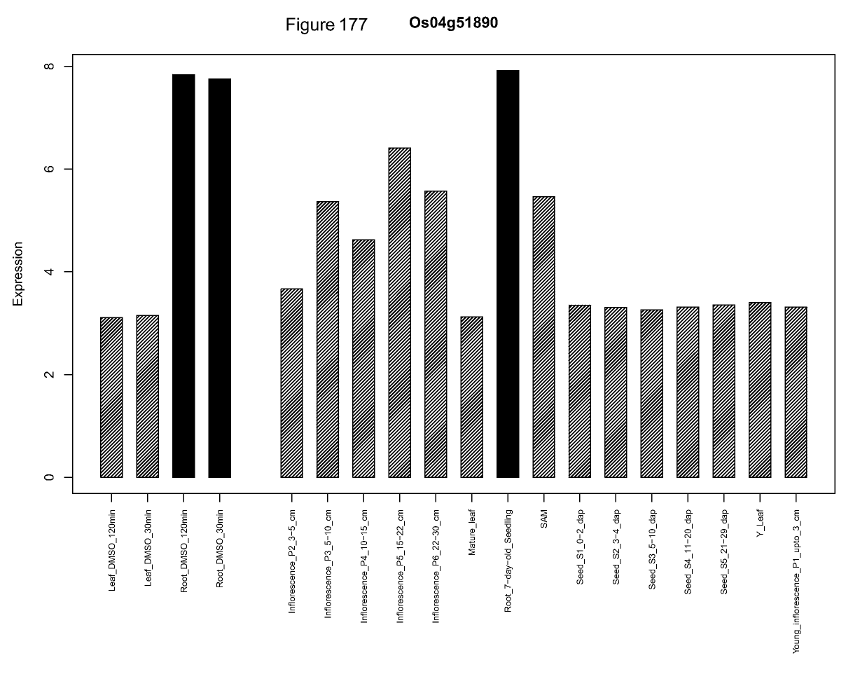

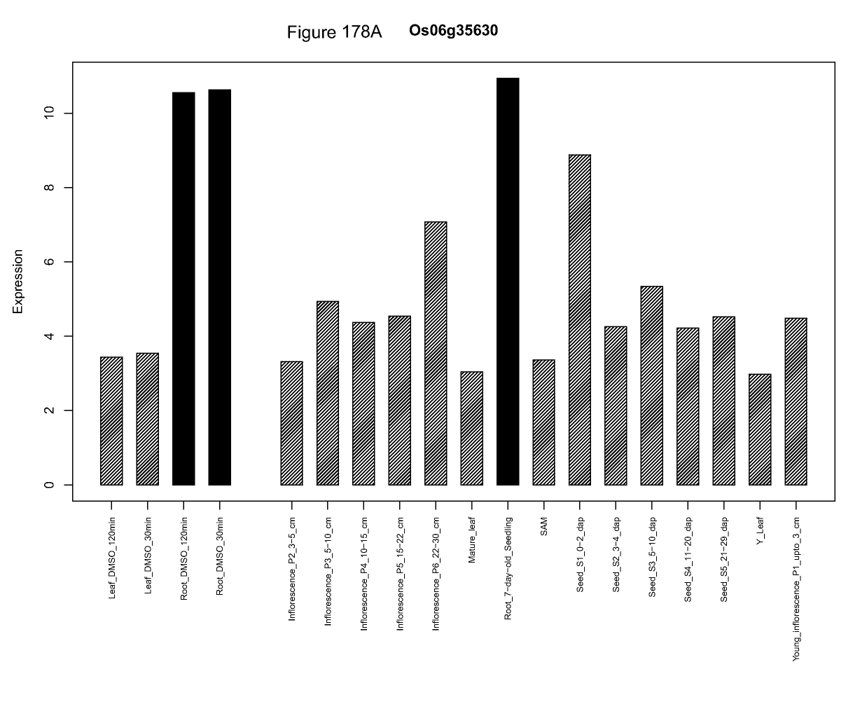

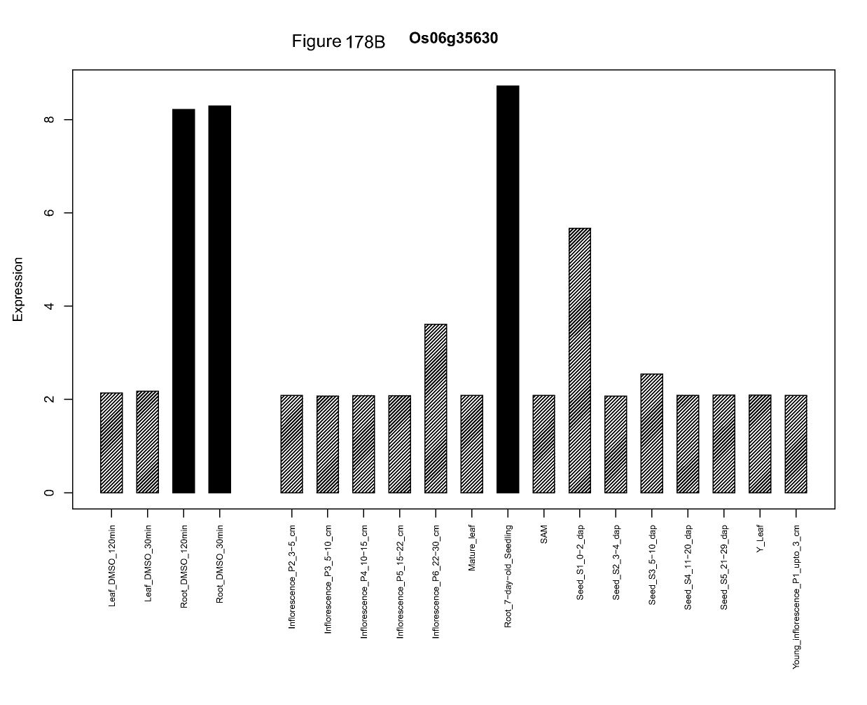

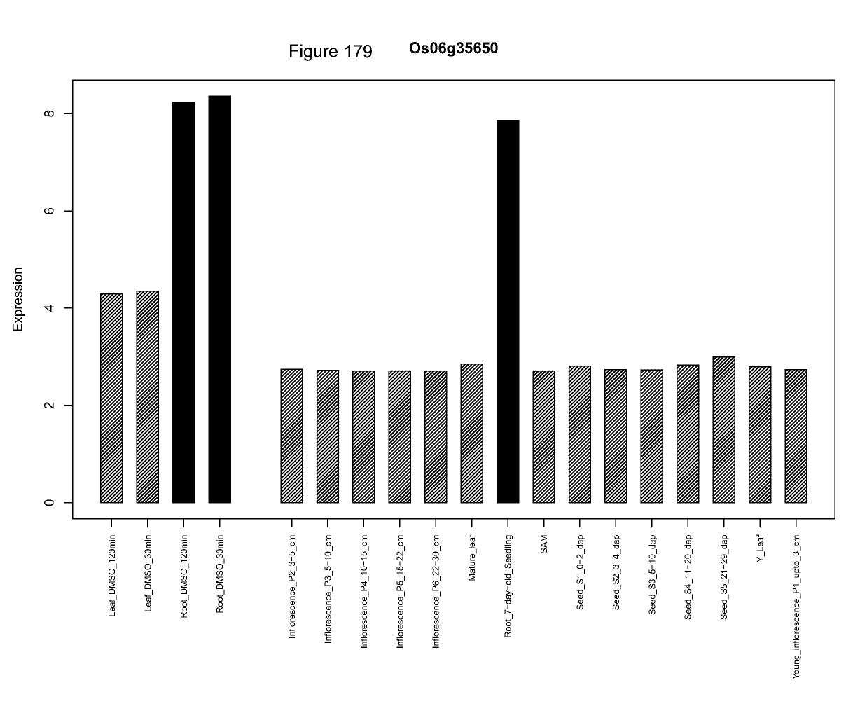

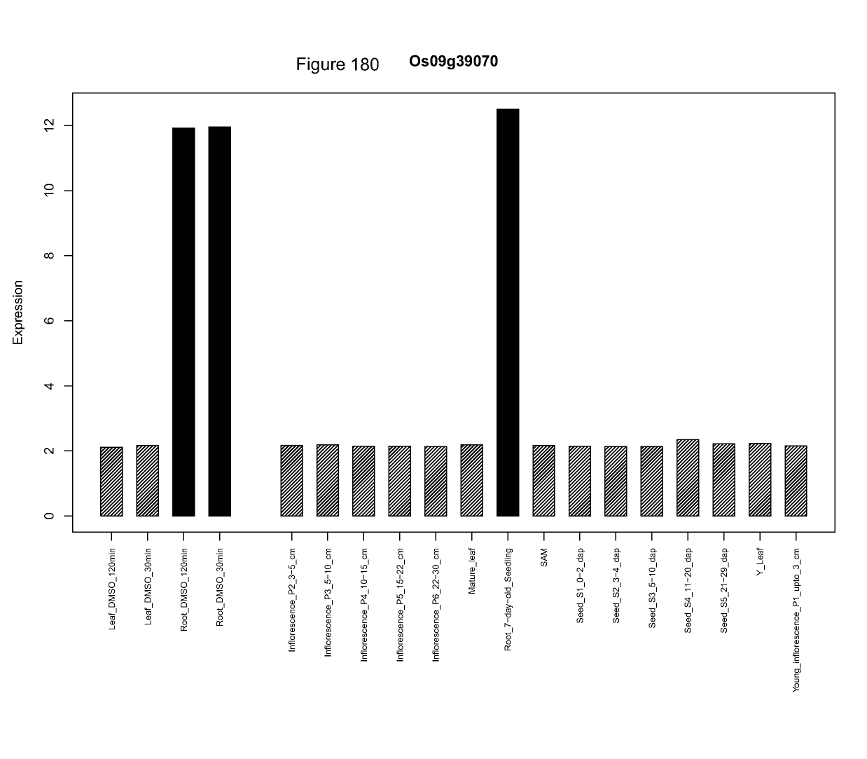

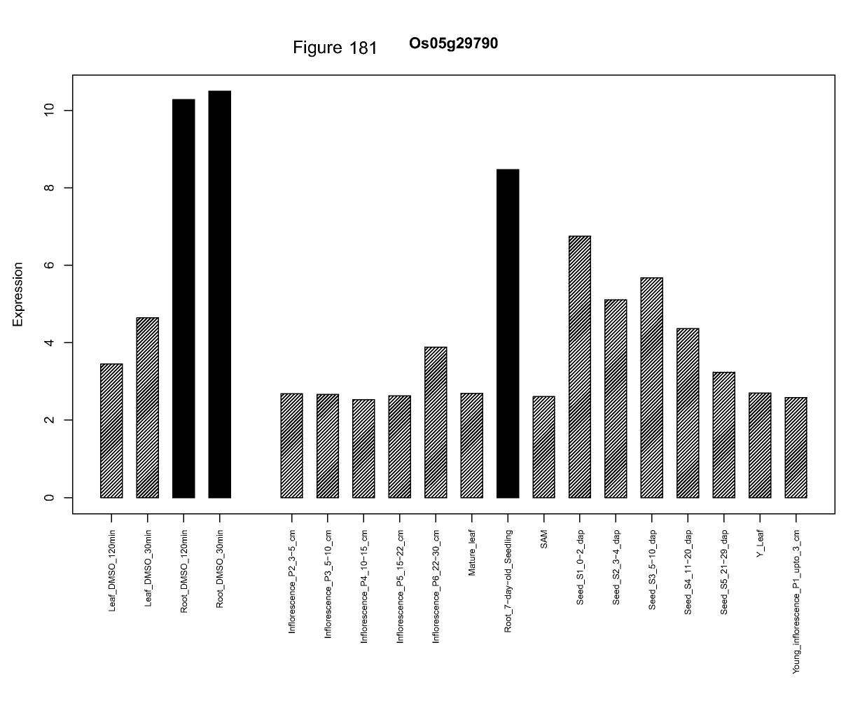

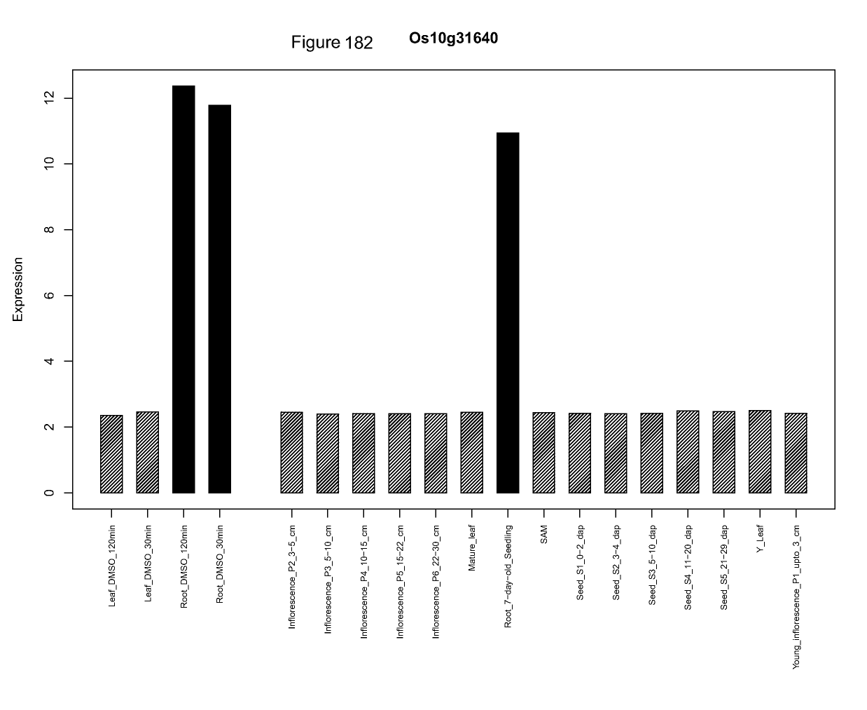

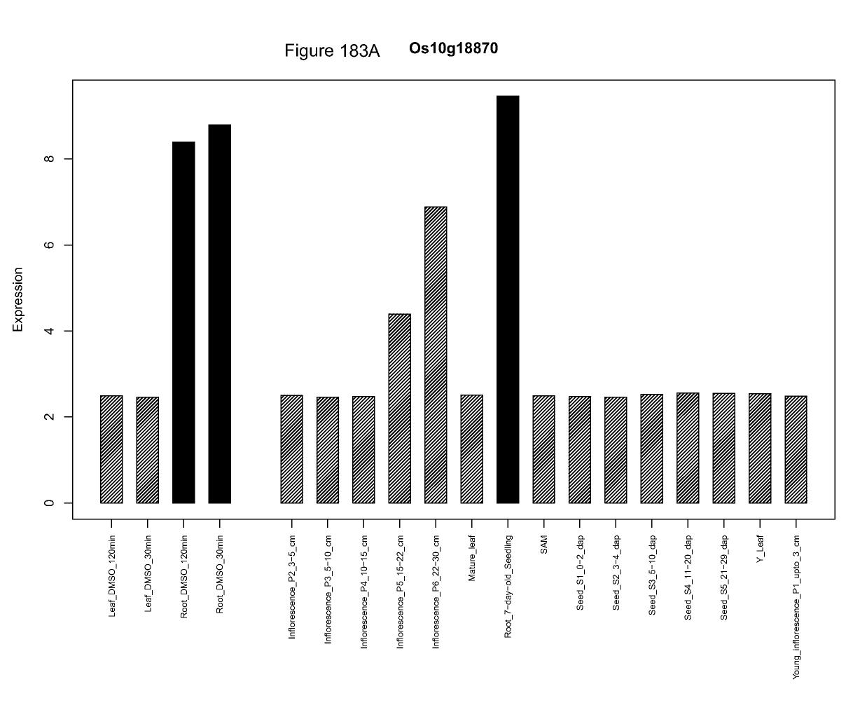

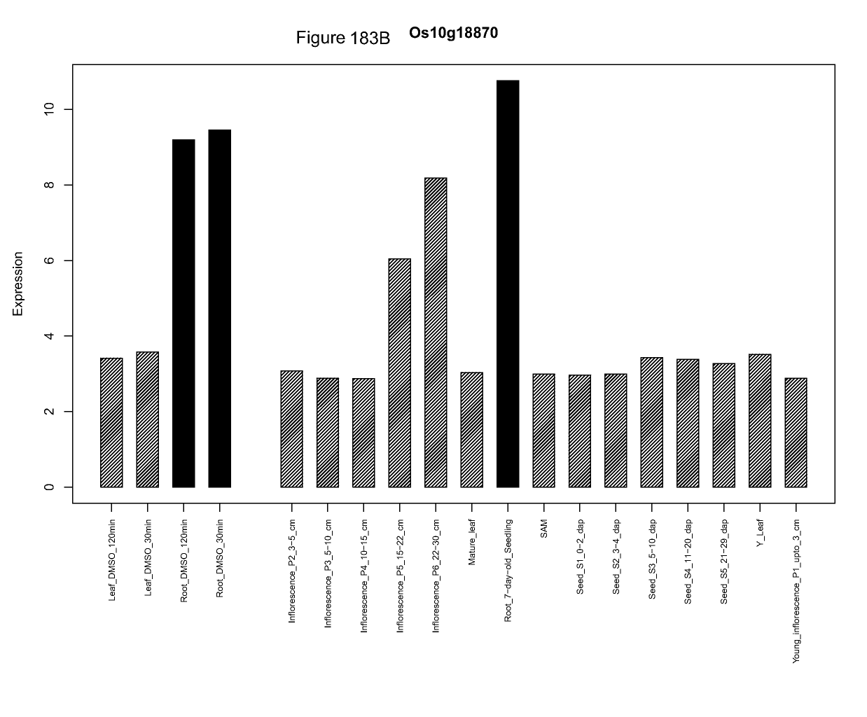

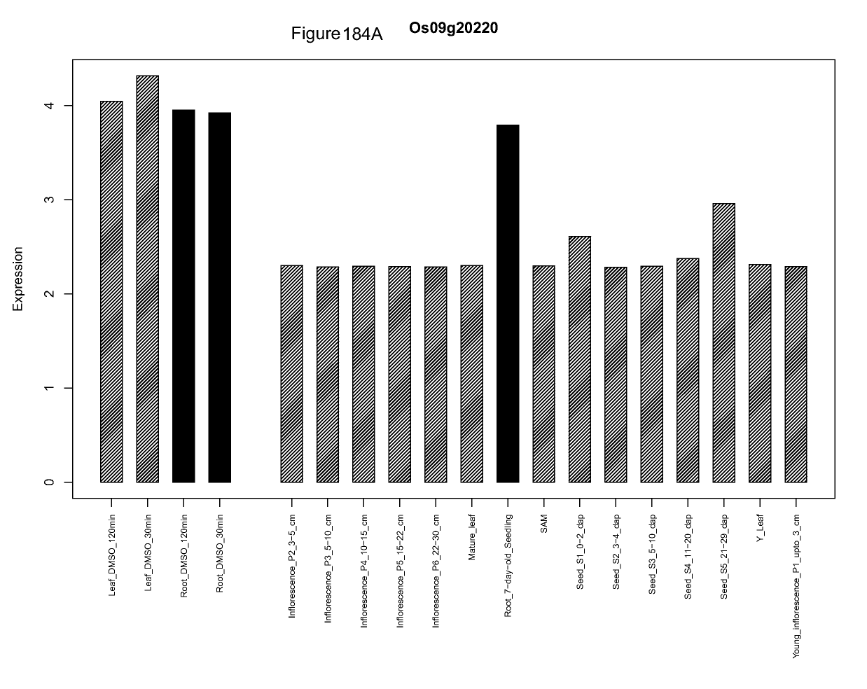

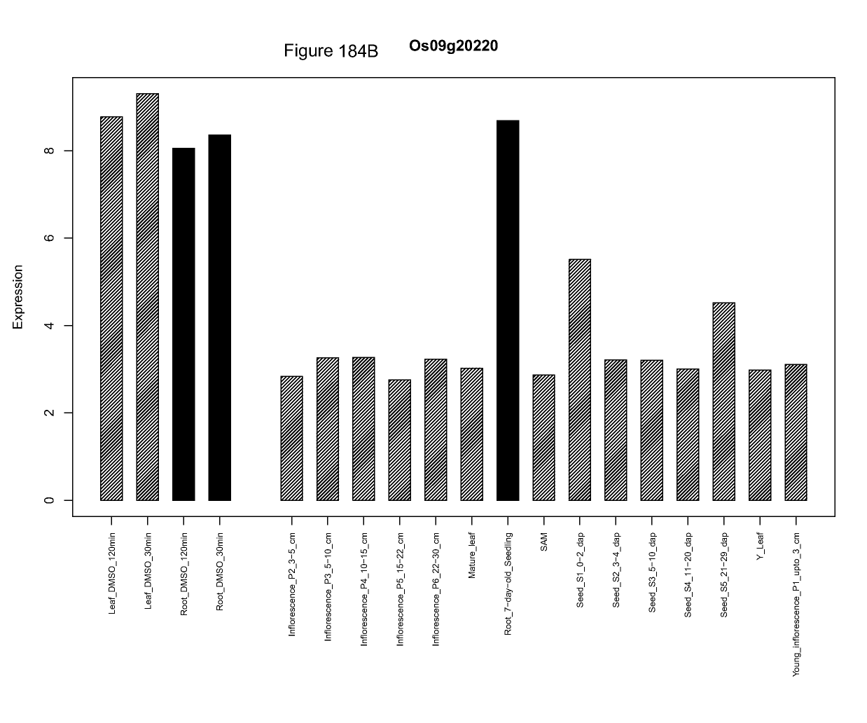

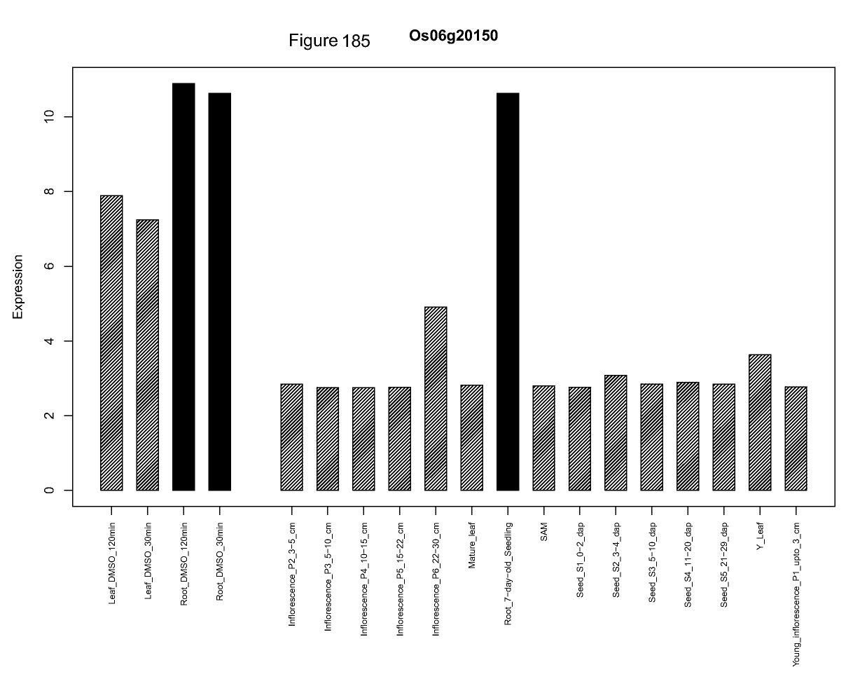

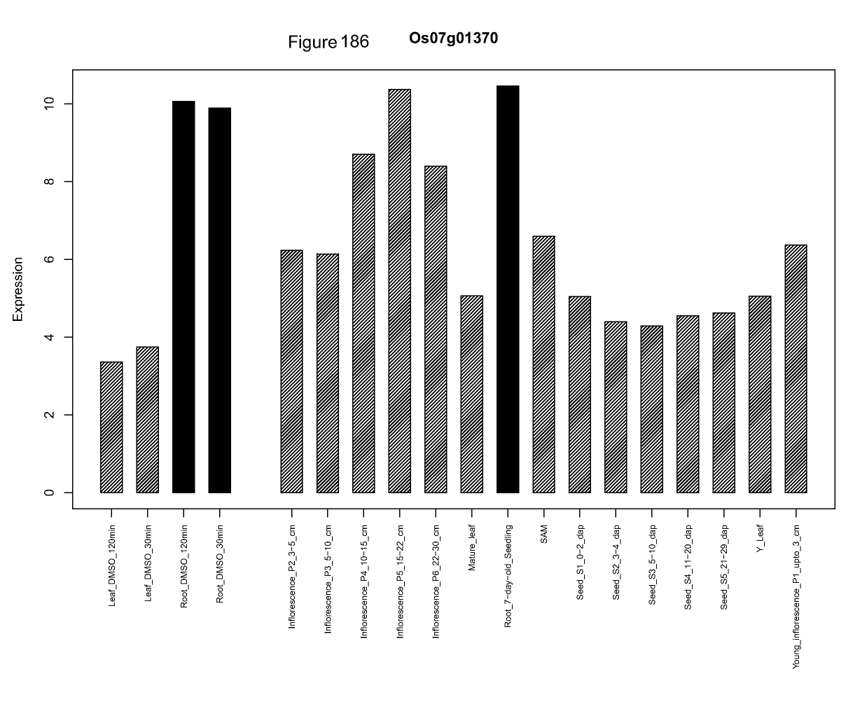

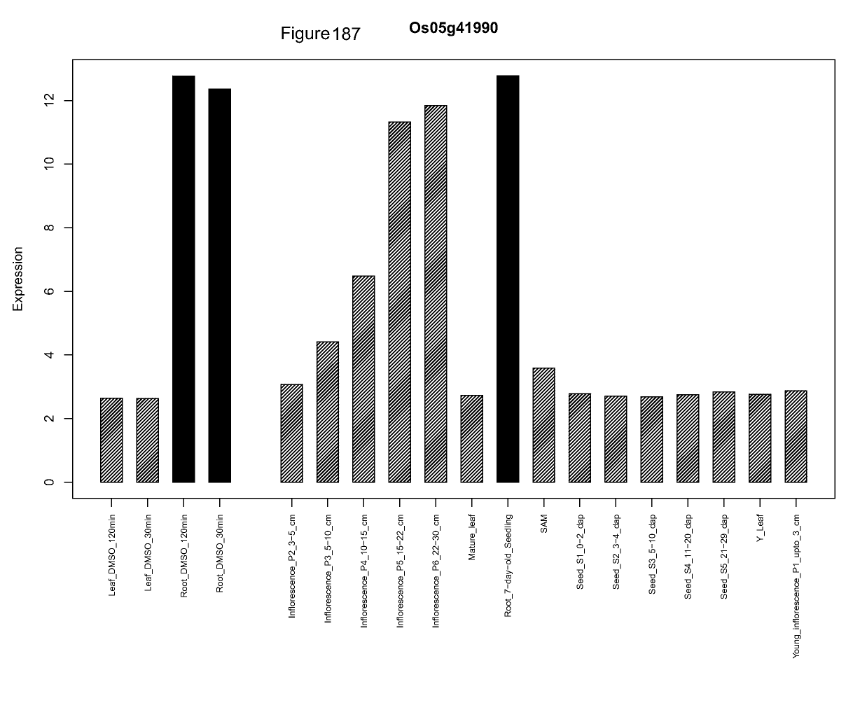

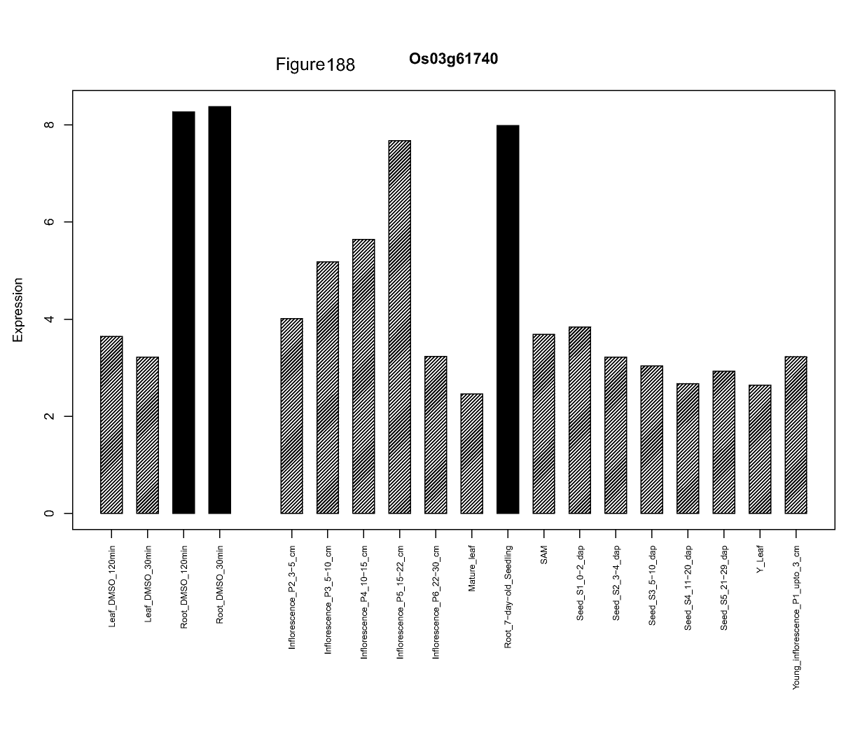

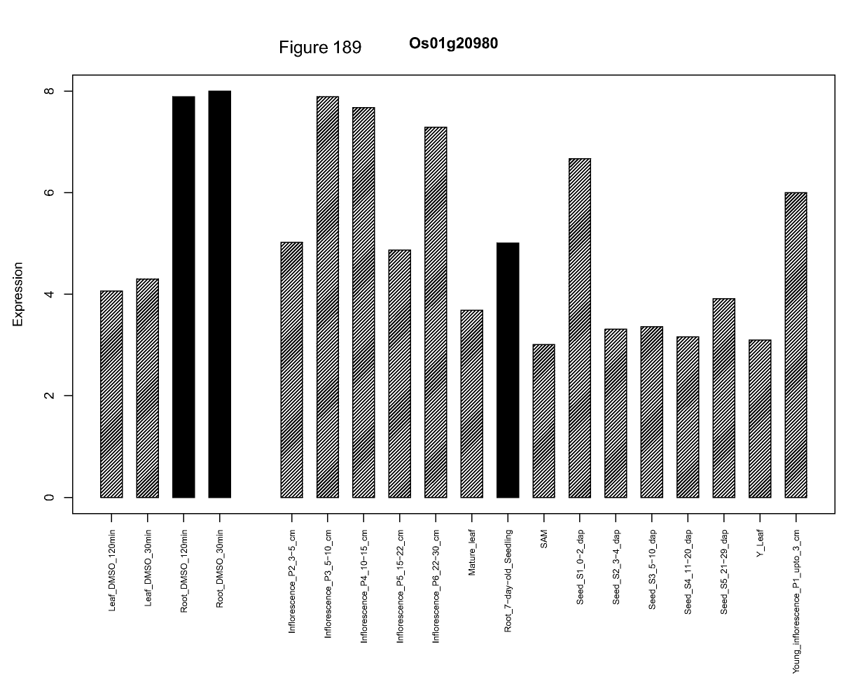

This example provides the endogenous expression data of the sequences identified in Example 4, where such data was available. The endogenous expression levels of the rice genes are provided in FIGS. 159-189. Expression data for the underlying rice genes is shown where available. Also, when more than one set of expression data was available, the further data may also be shown. All data are from Affymetrix GeneChip rice genome arrays which allow the detection of about 51,000 transcripts from Oryza sativa. Each figure provides data from two publically available datasets. The four bars on the left of each plot are derived from Hirose et al. (Plant Cell Physiol., 48: 523-539 (2007)) and show expression data from roots (black bars) and leaves (hatched bars). The roots and leaves were excised from 2-week-old seedlings dipped in distilled water containing DMSO for either 30 or 120 minutes. The bars on the right of each plot are derived from Jain et al. (Plant Physiol., 143: 1467-1483 (2007)) and show expression values in various above ground tissues (hatched bars) as well as in root tissue (black bars). Above ground tissue consisted of mature leaf, Y leaf, and different stages of influorescence (up to 0.5 mm, SAM; 0-3 cm, P1; 3-5 cm, P2; 5-10 cm, P3; 10-15 cm, P4; 15-22 cm, P5; 22-30 cm, P6) and seed (0-2 dap, 51; 3-4 dap, S2; 5-10 dap, S3; 11-20 dap, S4; 21-29 dap, S5) development, and was harvested from rice plants grown under greenhouse or field conditions. Roots were harvested from 7-d-old lightgrown seedlings grown in reverse-osmosis (RO) water.

Table 7 below shows the correspondence between the regulatory polynucleotides in Example 4 and the expression plots of FIGS. 159-189 (where data was not available and no Figure is shown, “N/A” (not applicable) is indicated).

TABLE 7

| Expression Figure | Regulatory Polynucleotide SEQ ID NOS |
|---|---|
| (Gene Accession No.) | (Corresponding Gene Accession No.) |
| 159 (Os11g08380) | 17 (Os11g08380) |
| 160 (Os04g55850) | 18 (Os04g55850) |
| 161 (Os06g36090) | 19 (Os06g36090) |
| 162A, B (Os05g27304) | 20 (Os05g27304) |
| 163 (Os04g33570) | 21 (Os04g33570) |
| 164 (Os08g45000) | 22 (Os08g45000) |
| 165 (Os02g44730) | 23 (Os02g44730) |
| 166 (Os06g03830) | 24 (Os06g03830) |
| 167 (Os04g33520) | 25 (Os04g33520) |
| 168 (Os07g42070) | 26 (Os07g42070) |
| 169 (Os05g04240) | 27 (Os05g04240) |
| 170 (Os06g44410) | 28 (Os06g44410) |
| 171A, B, C (Os05g11560) | 29 (Os05g11560) |
| 172 (Os10g10620) | 30 (Os10g10620) |
| 173 (Os11g05470) | 31 (Os11g05470) |
| 174 (Os02g52840) | 67 (Os02g52840) |
| 175 (Os02g16500) | 68 (Os02g16500) |
| 176A, B (Os10g40480) | 69 (Os10g40480) |
| 177 (Os04g51890) | 70 (Os04g51890) |
| 178A, B (Os06g35630) | 83 (Os06g35630) |
| 179 (Os06g35650) | 84 (Os06g35650) |
| 180 (Os09g39070) | 85 (Os09g39070) |
| 181 (Os05g29790) | 86 (Os05g29790) |
| 182 (Os10g31640) | 87 (Os10g31640) |
| 183A, B (Os10g18870) | 88 (Os10g18870) |
| 184A, B (Os0920220) | 89 (Os09g20220) |
| 185 (Os06g20150) | 90 (Os06g20150 + intron) |
| | 101 (Os06g20150) |
| 186 (Os07g01370) | 91 (Os07g01370 + intron) |
| | 102 (Os07g01370) |
| 187 (Os05g41990) | 92 (Os05g41990 + intron) |
| | 103 (Os05g41990) |
| 188 (Os03g61740) | 93 (Os03g61740 + intron) |
| | 104 (Os03g61740) |
| 189 (Os01g20980) | 94 (Os01g20980 + intron) |
| | 105 (Os01g20980) |

### Example 6

### Generation of Derivative Regulatory Polynucleotides

This example illustrates the utility of derivatives of the native Arabidopsis and rice ortholog regulatory polynucleotides. Derivatives of the Arabidopsis and ortholog regulatory polynucleotides are generated by introducing mutations into the nucleotide sequence of the native rice regulatory polynucleotides. A plurality of mutagenized DNA segments derived from the Arabidopsis and rice ortholog regulatory polynucleotides including derivatives with nucleotide deletions and modifications are generated and inserted into a plant transformation vector operably linked to a GUS marker gene. Each of the plant transformation vectors are prepared, for example, essentially as described in Example 3 above, except that the full length Arabidopsis or rice ortholog polynucleotide is replaced by a mutagenized derivative of the Arabidopsis or rice ortholog polynucleotide. Arabidopsis plants are transformed with each of the plant transformation vectors and analyzed for expression of the GUS marker to identify those mutagenized derivatives having regulatory activity.

### Example 7

### Identification of Regulatory Fragments

This example illustrates the utility of modified regulatory polynucleotides derived from the native Arabidopsis and rice ortholog polynucleotides. Fragments of the polynucleotides are generated by designing primers to clone fragments of the native Arabidopsis and rice regulatory polynucleotide. A plurality of cloned fragments of the polynucleotides ranging in size from 50 nucleotides up to about full length are obtained using PCR reactions with primers designed to amplify various size fragments instead of the full length polynucleotide. 3′ fragments from the 3′ end of the Arabidopsis or rice ortholog regulatory polynucleotide comprising random fragments of about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600 and 1650 nucleotides in length from various parts of the Arabidopsis or rice ortholog regulatory polynucleotides are obtained and inserted into a plant transformation vector operably linked to a GUS marker gene. Each of the plant transformation vectors is prepared essentially as described, for example, in Example 3 above, except that the full length Arabidopsis or rice polynucleotide is replaced by a fragment of the Arabidopsis or rice regulatory polynucleotide or a combination of a 3′ fragment and a random fragment. Arabidopsis plants are transformed with each of the plant transformation vectors and analyzed for expression of the GUS marker to identify those fragments having regulatory activity.

### Example 8

### Identification of Additional Orthologs

This example illustrates the identification and isolation of regulatory polynucleotides from organisms other than rice using the native Arabidopsis polynucleotide sequences and fragments to query genomic DNA from other organisms in a publicly available nucleotide data bases including GENBANK. Orthologous genes in other organisms can be identified using reciprocal best hit BLAST methods as described in Moreno-Hagelsieb and Latimer, Bioinformatics (2008) 24:319-324. Once an ortholog gene is identified, its corresponding regulatory polynucleotide sequence can be selected using methods described for Arabidopsis and rice in Examples 1 and 4. The full length polynucleotides are cloned and inserted into a plant transformation vector which is used to transform Arabidopsis plants essentially as illustrated in Example 3 above to verify regulatory activity and expression patterns.

### Example 9

### Preparation and Quantitative Root Expression Testing of Identified Regulatory Elements in Stably Transformed Arabidopsis

Candidate regulatory elements represented by SEQ ID NOS: 6-10, 1, 31-38, 40-42, 44-46, 52, 54-58, and 67 were sub-cloned into a plant transformation vector containing a right border region from Agrobacterium tumefaciens, a first transgene cassette to test the regulatory or chimeric regulatory element comprised of, a regulatory or chimeric regulatory element, operably linked to a coding sequence for Green Fluorescent Protein (GFP), operably linked to the 3′ termination region from the fiber Fb Late-2 gene from Gossypium barbadense (sea-island cotton, Genbank reference, U34401); a second transgene selection cassette used for selection of transformed plant cells that conferred resistance to the herbicide glyphosate, driven by the Arabidopsis Actin 7 promoter (Genbank accession, U27811) and a left border region from A. tumefaciens. Final constructs were transferred to Agrobacterium and transformed into Arabidopsis Columbia ecotype plants by the floral dip method (S. J. Clough, A. F. Bent, Plant J 16, 735 (December, 1998)). Transformed plants (T1 generation) were selected by resistance to glyphosate application. Sixteen glyphosate resistant T1s were selected per construct and their relative copy number was determined by qPCR. The six lowest copy T1s were selected for further analysis and allowed to set seed (T2 generation).

For a preliminary assessment of GFP expression, T2 seed from the six lines was grown in MS media in the RootArray, a device designed for confocal imaging of living plant roots under controlled conditions, and described in U.S. Patent Publication No. 2008/0141585 which is incorporated herein by reference in its entirety. After 5 days growth, the roots were stained with FM4-64 and imaged for GFP fluorescence in the meristematic zone, elongation zone and maturation zone with a Zeiss 510 confocal microscope. GFP expression was visually assessed in 3-5 seedlings per line. A construct was considered to fail expression prescreening if no GFP fluorescence was observed in any of the analyzed seedlings for each of the 6 lines per construct. No further analysis of these lines was performed. Regulatory polynucleotides contained in the lines that failed prescreening are listed in Table 8.

TABLE 8

| Gene | Promoter Sequence ID |
|---|---|
| AT3G54700 | 7 |
| AT2G38940 | 8 |
| AT2G27550 | 10 |
| AT5G26280 | 35 |
| AT3G16450 | 37 |
| AT5G48000 | 40 |
| AT4G12550 | 45 |
| AT1G74770 | 46 |
| AT2G39530 | 57 |
| Os02g52840 | 67 |

The designation of failing expression does not mean that these regulatory polynucleotides are not capable of driving expression since the prescreening procedures have low detection sensitivity. More sensitive detection methods like qRT-PCR were able to detect GFP transcripts in lines that failed to show GFP fluorescence in this presecreening procedure.

For all regulatory polynucleotides that passed prescreening, 3 to 6 of the independent T2 lines exhibited GFP fluorescence. A more thorough analysis of root GFP expression was undertaken on two representative lines that exhibited fluorescence. 50-80 T2 seed from the two representative lines was grown in MS media in the RootArray. After 5 days growth, the roots were stained with FM4-64 and imaged for GFP fluorescence in the meristematic zone, elongation zone and maturation zone with a Zeiss 510 confocal microscope. The GFP expression patterns from representative images were visually assessed and are summarized in Table 9.

TABLE 9
Expression testing of regulatory elements in stably transformed Arabidopsis

| SEQ | Corresponding | | |
|---|---|---|---|
| ID | Gene Accession No. | Tissue-Specific Category | Observed expression |
| 6 | AT1G15210 | broad root | Low expression in root cap and epidermis in |
| | | | meristematic zone; low expression in |
| | | | epidermis and cortex in elongation zone. |
| 9 | AT3G45710 | epidermal/vascular/cortex/ | Moderate constitutive expression in |
| | | phloem, all root developmental | maturation zone. |
| | | zones | |
| 1 | AT1G54890 | epidermis (including root hair), | Moderate expression in root cap and |
| | | all developmental stages | epidermis in meristematic zone; moderate |
| | | | epidermal expression in elongation zone; low |
| | | | epidermal and stele expression in maturation |
| | | | zone. |
| 31 | Os11g05470 | epidermal/vascular/cortex/ | Low expression in stele in maturation zone. |
| | | phloem, all root developmental | |
| | | zones | |
| 32 | AT3G19390 | broad root | Low expression in root cap. |
| 33 | AT4G22212 | broad root | Low expression in root cap and epidermis in |
| | | | meristematic zone; low epidermal expression |
| | | | in elongation zone; moderate stele |
| | | | expression in maturation zone. |
| 34 | AT5G23830 | broad root | Low expression in QC, root cap, and |
| | | | epidermis in meristematic zone; low |
| | | | epidermal expression in elongation zone; low |
| | | | constitutive expression in maturation zone. |
| 36 | AT1G52070 | broad root | Strong expression in the root cap; low |
| | | | constitutive expression in maturation zone. |
| 38 | AT1G66270 | broad root | Moderate expression in root cap and low |
| | | | expression in epidermis in meristematic zone; |
| | | | moderate epidermal expression in elongation |
| | | | zone; low constitutive expression in |
| | | | maturation zone. |
| 41 | AT5G53250 | epidermal/vascular/cortex/ | Low expression in the lateral root cap; low |
| | | phloem, all root developmental | constitutive expression in elongation zone; |
| | | zones | moderate constitutive expression in |
| | | | maturation zone. |
| 42 | AT5G50560 | epidermal/vascular/cortex/ | Low to moderate constitutive expression in |
| | | phloem, all root developmental | maturation zone. |
| | | zones | |
| 44 | AT4G30670 | epidermal/vascular/cortex/ | Low to moderate constitutive expression in |
| | | phloem, all root developmental | maturation zone. |
| | | zones | |
| 52 | AT3G23190 | epidermis (including root hair), | Low epidermal expression in maturation |
| | | all developmental stages | zone. |
| 54 | AT2G02680 | epidermis (including root hair), | Moderate expression in root cap; low |
| | | all developmental stages | epidermal expression in elongation zone; low |
| | | | to moderate constitutive expression in |
| | | | maturation zone. |
| 55 | AT3G46280 | epidermis (including root hair), | Strong expression in root cap; low epidermal |
| | | all developmental stages | expression in elongation zone; low to |
| | | | moderate epidermal expression in maturation |
| | | | zone. |
| 56 | AT5G17820 | epidermis (including root hair), | Moderate epidermal expression in maturation |
| | | all developmental stages | zone. |
| 58 | AT5G40510 | epidermis (including root hair), | Low expression in root cap; low epidermal |
| | | all developmental stages | expression in maturation zone. |
| 6 | AT1G15210 | broad root | Low expression in root cap and epidermis in |
| | | | meristematic zone; low expression in |
| | | | epidermis and cortex in elongation zone. |
| 9 | AT3G45710 | epidermal/vascular/cortex/ | Moderate constitutive expression in |
| | | phloem, all root developmental | maturation zone. |
| | | zones | |

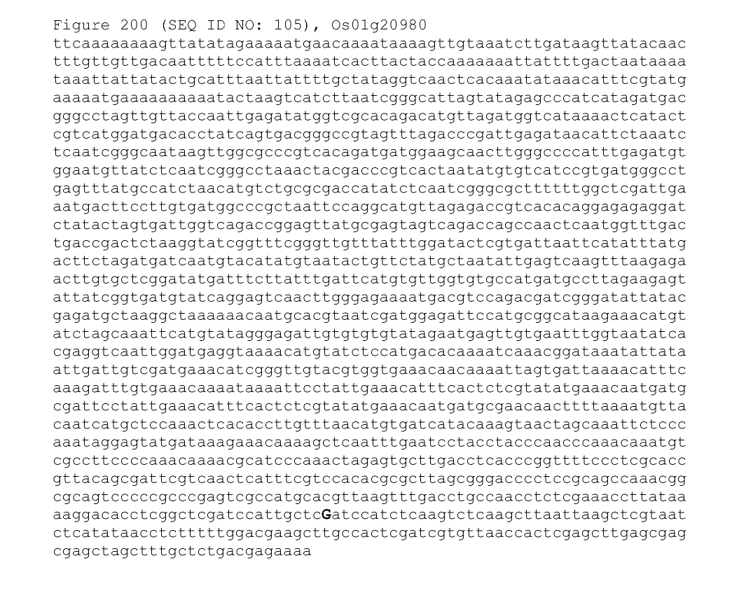

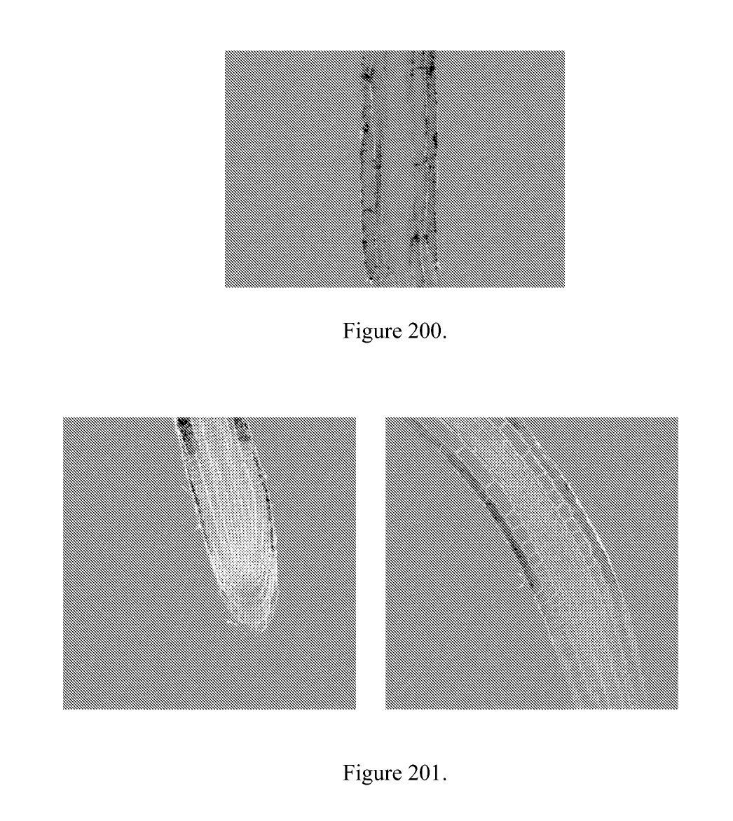

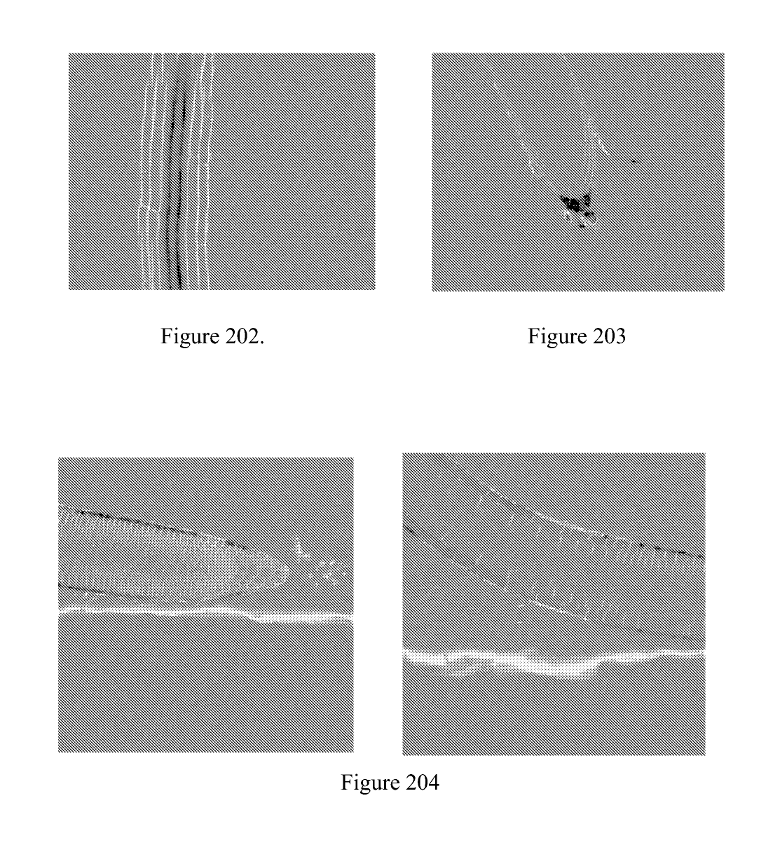

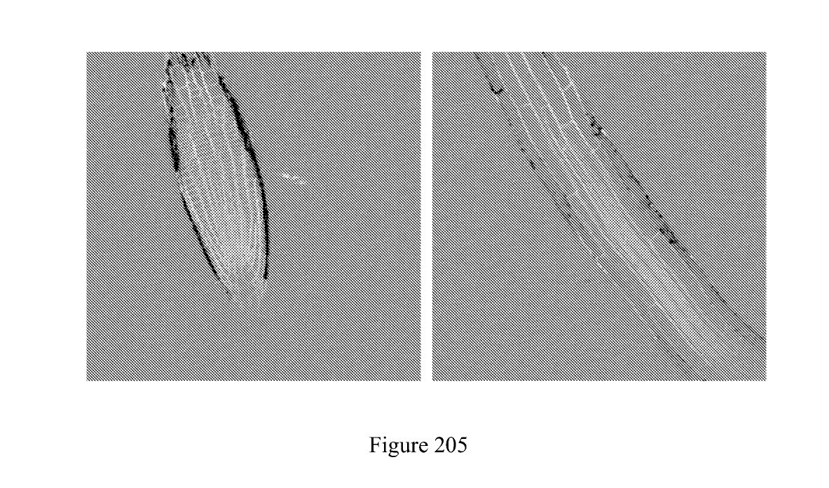

Sample images of representative individual T2 seedlings are shown in FIGS. 200-205. The images of FIGS. 200-205 show two channels, red and green, superimposed. The red channel shows cell wall staining and the green channel shows expression of GFP. The signal from the red channel was converted to white. Signal from the green channel was converted to grayscale such that the gray background outside of the root shows zero expression of GFP while any gray shade that is darker than the gray background outside of the root indicates GFP expression.

All images were taken with the same microscope settings.

While the invention has been described in detail and with reference to specific embodiments thereof, it will be apparent to one skilled in the art that various changes and modifications can be made without departing from the spirit and scope of the invention.

1. An isolated regulatory polynucleotide comprising a polynucleotide molecule selected from the group consisting of
(a) a polynucleotide molecule comprising a nucleic acid molecule having a sequence selected from the group consisting of SEQ ID NOS: 1-105 that is capable of regulating transcription of an operably linked transcribable polynucleotide molecule;
(b) a polynucleotide molecule having at least about 70% sequence identity to a sequence selected from the group consisting of SEQ ID NOS:1-105 that is capable of regulating transcription of an operably linked transcribable polynucleotide molecule; and
(c) a fragment of the polynucleotide molecule of (a) or (b) capable of regulating transcription of an operably linked transcribable polynucleotide molecule.

2. The isolated regulatory polynucleotide of claim 1, wherein the molecule is (a) a polynucleotide molecule comprising a nucleic acid molecule having the sequence selected from the group consisting of SEQ ID NOS: 1-105 that is capable of regulating transcription of an operably linked transcribable polynucleotide molecule.

3. The isolated regulatory polynucleotide of claim 1, wherein the regulatory polynucleotide is capable of regulating tissue-specific transcription.

4. The isolated regulatory polynucleotide of claim 1, wherein the regulatory polynucleotide is capable of regulating root-specific transcription.

5. (canceled)

6. The isolated regulatory polynucleotide of claim 4, wherein the regulatory polynucleotide is capable of regulating transcription in epidermal, vascular, cortex, phloem, pericycle, endodermal, meristem root cap tissues, or combinations thereof.

7-9. (canceled)

10. The isolated regulatory polynucleotide of claim 1, wherein the molecule is (b) a polynucleotide molecule having at least about 70% sequence identity to a sequence selected from the group consisting of SEQ ID NOS:1-105 that is capable of regulating transcription of an operably linked transcribable polynucleotide molecule.

11-21. (canceled)

22. The isolated regulatory polynucleotide of claim 1, wherein the polynucleotide molecule is (c) a fragment of the polynucleotide molecule of (a) or (b) capable of regulating transcription of an operably linked transcribable polynucleotide molecule.

23-28. (canceled)

29. A recombinant polynucleotide construct comprising the regulatory polynucleotide of claim 1 operably linked to a heterologous transcribable polynucleotide molecule.

30. The recombinant polynucleotide construct of claim 29, wherein the transcribable polynucleotide molecule encodes a protein of agronomic interest.

31. The recombinant polynucleotide construct of claim 29, wherein the transcribable polynucleotide molecule is operably linked to a 3′ transcription termination polynucleotide molecule.

32. A chimeric polynucleotide molecule comprising:
(a) a first polynucleotide molecule selected from the group consisting of
(i) a polynucleotide molecule comprising a nucleic acid molecule having a sequence selected from the group consisting of SEQ ID NOS: 1-105 that is capable of regulating transcription of an operably linked transcribable polynucleotide molecule;
(ii) a polynucleotide molecule having at least about 70% sequence identity to a sequence selected from the group consisting of SEQ ID NOS:1-105 that is capable of regulating transcription of an operably linked transcribable polynucleotide molecule; and
(iii) a fragment of the polynucleotide molecule of (a) or (b) capable of regulating transcription of an operably linked transcribable polynucleotide molecule, and
(b) a second polynucleotide molecule capable of regulating transcription of an operably linked polynucleotide molecule, wherein the first polynucleotide molecule is operably linked to the second polynucleotide molecule.

33. The chimeric polynucleotide of claim 32, wherein the first polynucleotide molecule comprises a core promoter molecule and the second polynucleotide molecule is selected from the group consisting of a cis-element, an enhancer element, and an intron.

34. The chimeric polynucleotide of claim 32, wherein the first polynucleotide molecule is selected from the group consisting of a cis-element, an enhancer element, and an intron and the second polynucleotide molecule comprises a core promoter molecule.

35. The chimeric polynucleotide of claim 32, wherein the first polynucleotide molecule comprises an intron.

36. The chimeric polynucleotide of claim 32, wherein the second polynucleotide molecule is heterologous to the first polynucleotide molecule.

37. The chimeric polynucleotide of claim 32, wherein the first polynucleotide molecule is (iii) a fragment of the polynucleotide molecule of (i) or (ii) capable of regulating transcription of an operably linked transcribable polynucleotide molecule and the second polynucleotide molecule is a heterologous core promoter sequence.

38. A transgenic host cell comprising the recombinant polynucleotide construct of claim 29.

39. The transgenic host cell of claim 38, wherein the host cell is a plant cell.

40. A transgenic plant stably transformed with the recombinant polynucleotide construct of claim 29.

41. The transgenic plant of claim 40, wherein the plant is selected from the group consisting of a monocotyledonous and a dicotyledonous plant.

42. The transgenic plant of claim 41, wherein the plant is a monocotyledonous plant selected from the group consisting of wheat, corn, rice, turf grass, millet, sorghum, switchgrass, miscanthus, sugarcane, and Brachypodium.

43. The transgenic plant of claim 41, wherein the plant is a dicotyledonous plant selected from the group consisting of soybean, cotton, canola, and potato.

44. Seed produced by the transgenic plant of claim 40.

45. An isolated polynucleotide molecule comprising a regulatory element derived from SEQ ID NOs: 1-105, wherein the regulatory element is capable of regulating transcription of an operably linked transcribable polynucleotide molecule.

46. The isolated polynucleotide molecule of claim 45, wherein the regulatory element is in operable linkage with a core promoter sequence.

47. (canceled)

48. The isolated polynucleotide molecule of claim 45, wherein the regulatory element is selected from the group consisting of core promoter regions, cis-elements, introns, and leader sequences.

49. The isolated polynucleotide molecule of claim 48, wherein the regulatory element is an intron capable of enhancing the transcription of the operably linked transcribable polynucleotide molecule.

50. A method of directing expression of a transcribable polynucleotide molecule in a host cell comprising:
(a) introducing the recombinant polynucleotide construct of claim 29 into a host cell to produce a transgenic host cell; and
(b) selecting a transgenic host cell exhibiting expression of the transcribable polynucleotide molecule.

51. The method of claim 50, wherein the transcribable polynucleotide molecule is selected from the group consisting of a coding sequence and a functional RNA.

52. The method of claim 50, wherein the host cell is a plant cell.

53. The method of claim 52, further comprising regenerating a plant comprising the introduced recombinant nucleic acid construct.

54. A method of directing expression of a transcribable polynucleotide molecule in a plant comprising:
(a) introducing the recombinant polynucleotide construct of claim 29 into a plant cell;
(b) regenerating a plant from the plant cell; and
(c) selecting a transgenic plant exhibiting expression of the transcribable polynucleotide molecule.

55. The method of claim 54, wherein the transcribable polynucleotide molecule is selected from the group consisting of a coding sequence and a functional RNA.
